(19) United States
(12) Patent Application Publication
(10) Pub. No.: US 2011/0237686 A1
(43) Pub. Date: Sep. 29, 2011

(54) FORMULATIONS AND METHODS OF USE

(75) Inventors: Pei-Sze Ng, Cambridge, MA (US); Jerry Zhang, Lexington, MA (US)

(73) Assignee: Cerulean Pharma Inc, Cambridge, MA (US)

(21) Appl. No.: 13/072,524

(22) Filed: Mar. 25, 2011

Related U.S. Application Data
(60) Provisional application No. 61/317,908, filed on Mar. 26, 2010.

(51) Int. Cl. A61K 47/32; A61K 47/36; A61K 47/40; A61K 47/34; A61P 35/00; B29C 35/16; B82Y 5/00

(52) U.S. Cl. 514/772.1; 514/777; 514/772.4; 264/28; 977/773; 977/906

(57) ABSTRACT

The invention relates to the liquid and lyophilized formulations of small particulates, liposomes, and micelles, and methods for making and using the formulations. In particular, at least in some embodiments, the present invention relates to the production and lyophilization of PEGylated nanoparticles, microparticles, micelles, and liposomes for use and administration in to a subject.

### RELATED APPLICATION

This application claims the benefit of U.S. Provisional Patent Application No. 61/317,908, tiled on Mar. 26, 2010, the entire contents of which are incorporated herein by reference.

### BACKGROUND

The development of new delivery vehicles that deliver certain therapeutic agents to a patient (e.g., targeted to a particular tissue or cell type or targeted to a specific diseased tissue, but not normal tissue), or that control the release of drugs in a subject has long been recognized as beneficial. For example, “small particle therapies” (e.g., microparticles, nanoparticles, liposomes, and micelles) have proven useful for such purposes, due to the small size, and their ability to evade recognition in the body allowing for targeted and controlled delivery, while remaining stable for an effective amount of time. A common disadvantage associated with small particle therapies is that after intravenous administration they are rapidly cleared from the bloodstream, which limits the duration of the therapeutic effect.

Surface modification of such small particles through the association or coating of the particles with suitable potentiating agents has been shown to modify their physicochemical characteristics and increase the stability and in vivo performance. This surface modification, for example, increases the hydrodynamic size of the particle and reduces renal clearance and/or reduces recognition and clearance or inactivation by the immune system, and results in improved pharmacokinetic and pharmacodynamic properties. One type of potentiating agent is poly(ethylene glycol) and the associated surface modification technique is referred to as “PEGylation.” PEGylation is a useful, effective strategy for reducing biospecific interactions for pharmaceuticals.

Because the shelf-life of small particle therapies is often limited, these particles may be lyophilized to a dry, stable state. Lyophilization, which is sometimes referred to as “freeze-drying,” is an attractive method to enhance the physico-chemical stability of small particles. Lyophilization has become a preferred method because it enables moisture removal at relatively low temperatures under sterile conditions. Furthermore, the technique imparts the valuable product attribute of rapid reconstitution with a suitable reconstitution reagent at the point of use.

In a typical lyophilization process, multiple vials containing a liquid drug formulation are loaded on temperature-controlled shelves within a sterile chamber and cooled to low temperatures until completely solidified. FIG. 1 illustrates a typical freezing process. A conventional lyophilization process lasts about 72 hours. Following the thermal treatment (freezing step), chamber pressure is reduced and shelf temperature is adjusted to enable removal of the frozen solvent (drying) through sublimation in a step termed “primary drying.” When sublimation is complete, the shelf temperature is raised for a “secondary drying” to remove additional unfrozen solvent still bound to the solid product. When sufficient solvent has been removed, the drying process is concluded by stoppering the vials or bottles in the chamber, generally under a sub-ambient pressure of inert gas. The final dry product is called a “lyophilized preparation” (or also a “cake”) and usually occupies roughly the same volume as Initial liquid because of its high porosity. Lyophilization produces certain types of mechanical stress that can damage small particles. For example, such stresses can alter the desired conformation of the small particles or deform the small particles. Following lyophilization and reconstitution, the size of small particles can change, or the small particles may aggregate, which reduces the effectiveness of the small particles. To combat these problems, lyoprotectants may be added to the liquid formulation to protect small particles from stress and damage during lypophilization. Conventional lyoprotectants are typically added in relatively large concentrations.

The lyophilization of small particle therapeutics is time-consuming and challenging. The lyophilization of small particles that have been subjected to a surface modification with a potentiating agent, e.g., PEGylated particles, is challenging because of the tendency of such particles to agglomerate. Particle aggregation and fusion, both during and after lyophilization, is frequently observed even in the presence of common lyoprotectants such as sugars (e.g., sucrose, trehalose, mannose). Further, more complex lyoprotectants, such as polysaccharides like dextrans, have a well-known incompatibility with PEG. It is believed that this incompatibility causes a phase separation between the dextran and the PEG, which may cause the sugar molecules to no longer act as a stabilizer resulting in aggregation of the particles. Similar stability problems occur during freeze-drying of solutions of PEGylated nanoparticles and excipients that are incompatible with PEG. Another view is that this aggregation may be the result of the crystallization of PEG polymer chains upon the surfaces of the particles upon freezing. Aggregation is particularly problematic because it can reduce effectiveness or render the small particles unsuitable for their intended use. Aggregation of PEGylated nanoparticles during lyophilization tends to increase as the concentration of the PEGylated nanoparticles increases in a liquid formulation that is to be lyophilized. It can also be difficult to resuspend lyophilized preparations of PEGylated nanoparticles to produce a solution that contains a high concentration of PEGylated nanoparticles. The ability to lyophilize liquid formulations that contain a high concentration PEGylated nanoparticles, and to resuspend the resulting lyophilized preparations to produce a solution that contains a high concentration of PEGylated nanoparticles, is desirable to allow administration of the PEGylated nanoparticles in convenient ways, for example, intravenously, e.g., as an intravenous bolus.

A need exists for improved lyophilized preparations of PEGylated nanoparticles.

### SUMMARY

Invention relates to the production, use and administration of small particulates, liposomes, and micelles for therapeutic agent delivery. In particular, at least in some embodiments, the present invention relates to the production and lyophilization of PEGylated nanoparticles, microparticles, micelles, and liposomes for use and administration into a subject.

In some aspects, the invention is a liquid (e.g., pre-lyophilization, or resuspended after lyophilization) or lyophilized formulation that comprises a particulate construct (e.g., nanoparticle or microparticle) and a lyoprotectant that comprises a cyclic oligosaccharide. The particulate construct comprises a polymer, a therapeutic agent and a potentiating agent. The therapeutic agent and the potentiating agent are associated with the polymer, and preferable are covalently bonded to the polymer, directly or through a suitable linker moiety. If desired, the particulate construct can further comprise one or more additional components, such as a stabilizing polymer, an excipient, a surfactant, and the like. If desired, the particulate construct can comprises a targeting ligand.

The polymer can be, for example, a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a polyamine acids), a poly(ethylene oxides), a poly(phosphazene), a poly etheresters, a polyester amides, a polyamide, derivatives, copolymers, blends, and other combinations thereof. In some embodiments, the polymer is a biodegradable block copolymer with a weight in the range of from about 1 kDa to about 21 kDa. For example, the biodegradable block copolymer can comprises poly-(lactide-co-glycolide).

The cyclic oligosaccharide can comprise a polysaccharide moiety selected from the group consisting of: an α-cyclodextrin, a β-cyclodextrin, a 2-hydroxypropyl-β-cyclodextrin, a β-cyclodextrin sulfobutylether, any derivative thereof, and any combination thereof. In some embodiments, the polysaccharide is incorporated within the backbone of a polymer. If desired, at least one occurrence of the cyclic oligosaccharide can be oxidized.

In preferred aspects, the formulation further comprises a wetting agent, such as a monosaccharide, a disaccharide, a surfactant, an amino acid, any derivative thereof, and any combination thereof. In some preferred embodiments, the wetting agent is a disaccharide selected from the group consisting of sucrose, trehalose, lactose and combinations thereof. If desired, the wetting agent can also include a monosaccharide, such as glucose, fructose, galactose, xylose, ribose, and combinations thereof. When the formulation contains a lyoprotectant that comprises a cyclic oligosaccharide and also contains a wetting agent, the ratio of cyclic oligosaccharide to the wetting agent (w/w) can be about 0.5:1.5 to about 1.5:0.5 and/or the ratio of cyclic oligosaccharide plus wetting agent to polymer (w/w) can be about 1:1 to about 10:1. Sucrose is a preferred wetting agent.

In some embodiments, a therapeutic agent and/or potentiating agent is bonded to the polymer through a linker. In such embodiments, the linker can be, for example, an alkanoate linker, a PEG-based linker, a linker that comprises a disulfide bond, a self-immolative linker, an amino acid, a peptide, a glutamic acid, a branched glutamic acid, a polyglutamic acid, any derivative thereof, and any combination thereof. In preferred aspects, the therapeutic agent and potentiating agent are bonded to the polymer, and the particulate construct is a dual conjugate nanoparticle or dual conjugate microparticle.

The therapeutic agent comprises a drug, such as a cancer drug, an anti-inflammatory drug, a thrombolytic drug, a cardiovascular drug, an anti-anginal drug, a diuretic drug, a antihistamine drug, a hormone-containing drug, an antibiotic drug, a pain reliever drug, an anti-diarrheal drug, an antiemetic, an anti-metabolite drug, an immunomodulator drug, a radiation drug, a chemotherapy drug, an any derivative thereof and any combination thereof. In particular embodiments, the therapeutic agent is a taxane drug. The therapeutic agent is generally present in a range of about 5% to about 20% (w/w) of the particulate construct. In some embodiments, the ratio of therapeutic agent to polymer (e.g., biodegradable block copolymer) is in the range of from about 85:15 to about 55:45 (w/w).

The potentiating agent generally comprises a hydrophilic polymer, such as a poly(alkylene glycol), any derivative thereof, or any combination thereof. Particular examples of hydrophilic polymers include polyethylene glycol, polypropylene glycol, polybutylene glycol, derivatives thereof, and combinations thereof.

In some aspects, the particulate construct further comprises a stabilizing polymer such as a poly(vinyl alcohol), a poly(vinyl pyrrolidone), a poly(vinyl acetate), a crown ether, any derivative thereof, and any combination thereof.

Liquid formulations of the invention, such as a resuspended lyophilized preparation, comprise a solvent, that is preferably physiologically acceptable, such as an aqueous solvent, an organic solvent, and any combination thereof. In some embodiments, the organic solvent comprises an organic compound selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, butyl acetate, propyl acetate, any derivative thereof, and any combination thereof. When the solvent is an aqueous-organic solvent mixture the water:organic solvent ratio can be from about 1:1 to about 1:10 (v/v). In preferred aspects, the liquid formulation comprises a polymer concentration of at least about 30 mg/mL, more preferably at least about 70 mg/mL. When the liquid formulation is a resuspended lyophilized preparation, it is preferred that particles in the liquid formulation have a Z-average diameter, poly-dispersity index, and Dv₉₀ that differ from the Z-average diameter, poly-dispersity index, and Dv₉₀ of the particles in the formulation that was lyophilized to produce said lyophilized preparation by no more than about 20%.

In more particular aspects, the invention is a liquid (e.g., pre-lyophilization, or resuspended after lyophilization) or lyophilized formulation that comprises a particulate construct (e.g., nanoparticle or microparticle), and a lyoprotectant that comprises a cyclodextrin, any derivative thereof, and any combination thereof, and a wetting agent selected from the group consisting of a monosaccharide, a disaccharide, a surfactant, an amino acid, any derivative thereof, and any combination thereof. In some embodiments, the lyoprotectant is 2-hydroxypropyl-β-cyclodextrin. In these embodiments or in alternative embodiments, the wetting agent is a disaccharide selected from the group consisting of sucrose, trehalose, lactose and combinations thereof. Preferably, the wetting agent is sucrose. The ratio of lyoprotectant to wetting agent (w/w) is preferably about 0.7:1.3 to about 1.3:0.7. The ratio of lyoprotectant plus wetting agent to polymer (w/w) is preferably about 1:1 to about 3:1.

One embodiment provides a liquid formulation comprising a lyoprotectant that comprises a cyclic oligosaccharide; and a particulate construct, the particulate construct comprising a polymer composition; a therapeutic agent; and a potentiating agent, wherein the therapeutic agent and the potentiating agent are associated with the polymer composition.

In some embodiments the particulate construct is a dual conjugate nanoparticle.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxide), a poly(phosphazene), a poly etherester, a polyester amide, a polyamide, derivatives, copolymers, blends, and other combinations thereof.

In some embodiments the particulate construct comprises a biodegradable block copolymer with a weight in the range of from about 1 kDa to about 21 kDa.

In some embodiments the therapeutic agent to biodegradable block copolymer ratio is in the range of from about 85:15 to about 55:45 by weight.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, butyl acetate, propyl acetate, any derivative thereof, and any combination thereof.

In some embodiments the liquid formulation has an organic solvent:water ratio in the range of from about 1:1 to about 1:10 by volume.

In some embodiments the liquid formulation comprises a reconstitution reagent.

One embodiment provides a lyophilized preparation that comprises a particulate construct, the particulate construct comprising a polymer composition, a therapeutic agent, and a potentiating agent; wherein the therapeutic agent and the potentiating agent are associated with the polymer composition; and lyoprotectant that comprises a cyclic oligosaccharide.

Some embodiments further comprise 0.5% water or less by weight of the lyophilized preparation by weight of the lyophilized preparation.

In some embodiments the lyophilized preparation is substantially free of aggregates.

In some embodiments the particulate construct is a dual conjugate nanoparticle.

Some embodiments further comprise a reconstitution reagent.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene), a poly etheresters, a polyester amides, a polyamide, derivatives, copolymers, blends, and other combinations thereof.

In some embodiments the particulate construct comprises a biodegradable block copolymer with a weight in the range of from about 1 kDa to about 21 kDa.

In some embodiments the therapeutic agent to biodegradable block copolymer ratio is in the range of from about 85:15 to about 55:45 by weight.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments wherein the hydrophilic polymer comprises a polymer selected from the group consisting of: a polyethylene glycol, a polyalkylene glycol, a polypropylene glycol, and polybutylene glycol.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, a multifunctional linker, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, any derivative thereof, and any combination thereof.

In some embodiments the liquid formulation has an organic solvent:water ratio in the range of from about 1:1 to about 1:10 by volume.

In some embodiments the lyophilized preparation is prepared by a lyophilization process that does not include an annealing step.

Some embodiments provide a kit comprising a lyophilized preparation and optionally one or more of a reconstitution reagent, a pharmaceutically acceptable carrier or adjuvant, a delivery device, and instructions for use.

Some embodiments provide a kit wherein the kit is used by a healthcare provider to treat a subject.

One embodiments comprises a method comprising providing a liquid formulation that comprises a lyoprotectant that comprises a cyclic oligosaccharide; and a particulate construct, the particulate construct comprising a polymer composition; a therapeutic agent; and a potentiating agent, wherein the therapeutic agent and the potentiating agent are associated with the polymer composition; and lyophilizing the liquid formulation to provide a lyophilized preparation.

Some embodiments further comprise reconstituting the lyophilized preparation by adding a reconstitution reagent.

Some embodiments further comprise administering an effective amount of the lyophilized preparation to a subject.

In some embodiments lyophilizing the liquid formulation involves a rapid cycle lyophilization.

In some embodiments lyophilizing the liquid formulation does not include an annealing step.

In some embodiments the particulate construct is a dual conjugate nanoparticle.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene), a poly etheresters, a polyester amides, a polyamide, derivatives, copolymers, blends, and other combinations thereof.

In some embodiments the particulate construct comprises a biodegradable block copolymer with a weight in the range of from about 1 kDa to about 21 kDa.

In some embodiments the therapeutic agent to biodegradable block copolymer ratio is in the range of from about 85:15 to about 55:45 by weight.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a polyalkylene glycol, a polypropylene glycol, and polybutylene glycol.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, butyl acetate, propyl acetate, any derivative thereof, and any combination thereof.

In some embodiments the liquid formulation has an organic solvent:water ratio in the range of from about 1:1 to about 1:10 by volume.

One embodiment provides a method of treating cancer comprising providing a lyophilized preparation comprising a lyoprotectant that comprises a cyclic oligosaccharide; and a particulate construct, the particulate construct comprising a polymer composition; a therapeutic agent; and a potentiating agent, wherein the therapeutic agent and the potentiating agent are associated with the polymer composition; and administering an effective amount of such preparation to a subject.

In some embodiments the administering of an effective amount of such preparation to a subject is performed by a healthcare provider.

Some embodiments further comprise allowing the particulate construct to treat a cancer cell.

In some embodiments the cancer cell comprises a cancer cell selected from the group consisting of: a colorectal cancer cell, a gastric cancer cell, a liver cancer cell, a renal cancer cell, a cystic cancer cell, a pulmonary cancer cell, a billiard tract cancer cell, a pancreatic cancer cell, a uterine cancer cell, an ovarian cancer cell, a breast cancer cell, a melanoma, any derivative thereof, and any combination thereof.

In some embodiments the lyophilized preparation is prepared by a lyophilizing process that involves a rapid cycle lyophilization.

In some embodiments the lyophilized preparation is prepared by a lyophilizing process that does not include an annealing step.

In some embodiments wherein the lyophilized preparation comprises 0.5% water or less by weight of the lyophilized preparation.

In some embodiments the lyophilized preparation is substantially free of aggregates.

In some embodiments the particulate construct is a dual conjugate nanoparticle.

In some embodiments the lyophilized preparation comprises a reconstitution reagent.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene), a poly etheresters, a polyester amides, a polyamide, derivatives, copolymers, blends, and other combinations thereof.

In some embodiments the particulate construct comprises a biodegradable block copolymer with a weight in the range of from about 1 kDa to about 21 kDa.

In some embodiments the therapeutic agent to biodegradable block copolymer ratio is in the range of from about 85:15 to about 55:45 by weight.

In some embodiments the particulate construct has a Dv₉₀, of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a polyalkylene glycol, a polypropylene glycol, and polybutylene glycol.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, a multifunctional linker, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, any derivative thereof, and any combination thereof.

In some embodiments the liquid formulation has an organic solvent:water ratio in the range of from about 1:1 to about 1:10 by volume.

Some embodiments provide a kit comprising a lyophilized preparation and optionally comprising one or more of a reconstitution reagent, a pharmaceutically acceptable carrier or adjuvant, a delivery device, and instructions for use.

In some embodiments the kit is used by a healthcare provider to treat a subject.

In some embodiments the lyophilized preparation is a component of a kit that optionally comprises one or more of a reconstitution reagent, a pharmaceutically acceptable carrier or adjuvant, a delivery device, and instructions for use.

One embodiments provides a method comprising providing a lyophilized preparation comprising a lyoprotectant that comprises a cyclic oligosaccharide; and a particulate construct, the particulate construct comprising a polymer composition; a therapeutic agent; and a potentiating agent, wherein the therapeutic agent and the potentiating agent are associated with the polymer composition; and combining the lyophilized preparation with a reconstitution reagent, to provide a reconstituted liquid formulation.

Some embodiments further comprise administering the reconstituted preparation to a subject.

In some embodiments the lyophilized preparation is prepared by a rapid cycle lyophilization process.

In some embodiments the lyophilized preparation is prepared by a lyophilization process that does not include an annealing step.

In some embodiments the reconstitution reagent comprises a liquid selected from the group consisting of: water, sterile water, a salt solution, an alcohol, any derivative thereof, and any combination thereof.

In some embodiments the lyophilized preparation comprises 0.5% water or less by weight of the lyophilized preparation by weight of the lyophilized preparation.

In some embodiments the lyophilized preparation is substantially free of aggregates.

In some embodiments the particulate construct is a dual conjugate nanoparticle.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene), a poly etheresters, a polyester amides, a polyamide, derivatives, copolymers, blends, and other combinations thereof.

In some embodiments the particulate construct comprises a biodegradable block copolymer with a weight in the range of from about 1 kDa to about 21 kDa.

In some embodiments the therapeutic agent to biodegradable block copolymer ratio is in the range of from about 85:15 to about 55:45 by weight.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a polyalkylene glycol, a polypropylene glycol, and polybutylene

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethlene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, a multifunctional linker, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, any derivative thereof, and any combination thereof.

In some embodiments the liquid formulation has an organic solvent:water ratio in the range of from about 1:1 to about 1:10 by volume.

One embodiments provides a device having disposed therein, a lyophilized preparation comprising a lyoprotectant that comprises a cyclic oligosaccharide; and a particulate construct, the particulate construct comprising a polymer composition; a therapeutic agent; and a potentiating agent, wherein the therapeutic agent and the potentiating agent are associated with the polymer composition.

In some embodiments the delivery device is a storage device, a cannula, a syringe, drip bag, an IV admixture bag, an IV infusion set, a piggy back set, or any combination thereof.

In some embodiments the delivery device is provided as part of a kit that includes a reconstitution reagent and/or operating instructions.

In some, embodiments the lyophilized preparation is reconstituted.

In some embodiments the lyophilized preparation comprises 0.5% water or less by weight of the lyophilized preparation.

Some embodiments further comprise a reconstitution reagent.

Some embodiments provide a kit comprising a device and optionally one or more of a reconstitution reagent, and instructions for use.

In some embodiments the kit is used by a healthcare provider to treat a subject.

In some embodiments the lyophilized preparation is substantially free of aggregates.

In some embodiments the particulate construct is a dual conjugate nanoparticle.

Some embodiments further comprise a reconstitution reagent.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene), a poly etheresters, a polyester amides, a polyamide, derivatives, copolymers, blends, and other combinations thereof. In some embodiments the particulate construct comprises a biodegradable block copolymer with a weight in the range of from about 1 kDa to about 21 kDa.

In some embodiments the particulate construct comprises a biodegradable block copolymer.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a polyalkylene glycol, a polypropylene glycol, and polybutylene glycol.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, a multifunctional linker, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, any derivative thereof, and any combination thereof.

In some embodiments the liquid formulation has an organic solvent:water ratio in the range of from about 1:1 to about 1:10 by volume.

In some embodiments the lyophilized preparation is prepared by a lyophilization process that does not include an annealing step.

In some embodiments the lyophilized preparation is prepared by a rapid lyophilization process.

One embodiment provides a device having disposed therein, a reconstituted lyophilized preparation comprising a lyoprotectant that comprises a cyclic oligosaccharide; and a particulate construct, the particulate construct comprising a polymer composition; a therapeutic agent; and a potentiating agent, wherein the therapeutic agent and the potentiating agent are associated with the polymer composition.

In some embodiments the delivery device is a storage device, a cannula, a syringe, drip bag, an IV admixture bag, an IV infusion set, a piggy back set, or any combination thereof.

In some embodiments the delivery device is provided as part of a kit that includes a reconstitution reagent and/or operating instructions.

In some embodiments the lyophilized preparation is reconstituted.

In some embodiments the lyophilized preparation comprises 0.5% water or less by weight of the lyophilized preparation.

Some embodiments further comprise a reconstitution reagent.

Some embodiments provide a kit comprising the device and optionally one or more of a reconstitution reagent, and instructions for use.

In some embodiments the kit is used by a healthcare provider to treat a subject.

In some embodiments the lyophilized preparation is substantially free of aggregates.

In some embodiments the particulate construct is a dual conjugate nanoparticle.

Some embodiments further comprise a reconstitution reagent.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a polyethylene oxides), a poly(phosphazene), a poly etheresters, a polyester amides, a polyamide, derivatives, copolymers, blends, and other combinations thereof. In some embodiments the particulate construct comprises a biodegradable block copolymer with a weight in the range of from about 1 kDa to about 21 kDa.

In some embodiments the particulate construct comprises a biodegradable block copolymer.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a polyalkylene glycol, a polypropylene glycol, and polybutylene glycol.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a polyvinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, a multifunctional linker, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol. 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, any derivative thereof, and any combination thereof.

In some embodiments the liquid formulation has an organic solvent:water ratio in the range of from about 1:1 to about 1:10 by volume.

In some embodiments the lyophilized preparation is prepared by a rapid lyophilization process.

One embodiment provides a lyophilized preparation comprising a lyoprotectant that comprises a cyclic oligosaccharide; and a particulate construct, the particulate construct comprising a polymer composition; a therapeutic agent; and a potentiating agent, wherein the therapeutic agent and the potentiating agent are associated with the polymer composition; and prepared by a rapid cycle lyophilization process.

Some embodiments further comprise 0.5% water or less by weight of the lyophilized preparation by weight of the lyophilized preparation.

In some embodiments the lyophilized preparation is substantially free of aggregates.

In some embodiments the particulate construct is a dual conjugate nanoparticle.

Some embodiments further comprise a reconstitution reagent.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene), a poly etheresters, a polyester amides, a polyamide, derivatives, copolymers, blends, and other combinations thereof.

In some embodiments the particulate construct comprises a biodegradable block copolymer with a weight in the range of from about 1 kDa to about 21 kDa.

In some embodiments the particulate construct comprises a biodegradable block copolymer.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a polyalkylene glycol, a polypropylene glycol, and polybutylene glycol.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, a multifunctional linker, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, any derivative thereof, and any combination thereof.

In some embodiments the liquid formulation has an organic solvent:water ratio in the range of from about 1:1 to about 1:10 by volume.

In some embodiments the lyophilized preparation is prepared by a lyophilization process that does not include an annealing step.

Some embodiments provide a kit comprising a lyophilized preparation and optionally one or more of a reconstitution reagent, a pharmaceutically acceptable carrier or adjuvant, a delivery device, and instructions for use.

In some embodiments the kit is used by a healthcare provider to treat a subject.

Some embodiments provide a kit comprising a lyophilized preparation and optionally one or more of a reconstitution reagent, a pharmaceutically acceptable carrier or adjuvant, a delivery device, and instructions for use.

In some embodiments the kit is used by a healthcare provider to treat a subject.

One embodiments provides a lyophilized preparation comprising lyoprotectant that comprises a cyclic oligosaccharide; and a particulate construct, the particulate construct comprising a polymer composition; a therapeutic agent; and a potentiating agent, wherein the therapeutic agent and the potentiating agent are associated with the polymer composition; and prepared by a lyophilization process that does not include an annealing step.

Some embodiments further comprise 0.5% water or less by weight of the lyophilized preparation by weight of the lyophilized preparation.

In some embodiments the lyophilized preparation is substantially free of aggregates.

In some embodiments the particulate construct is a dual conjugate nanoparticle.

Some embodiments further comprise a reconstitution reagent.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene), a poly etheresters, a polyester amides, a polyamide, derivatives, copolymers, blends, and other combinations thereof.

In some embodiments the particulate construct comprises a biodegradable block copolymer with a weight in the range of from about 1 kDa to about 21 kDa.

In some embodiments the therapeutic agent to biodegradable block copolymer ratio is in the range of from about 85:15 to about 55:45 by weight.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a polyalkylene glycol, a polypropylene glycol, and polybutylene glycol.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, a multifunctional linker, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, any derivative thereof, and any combination thereof.

In some embodiments the liquid formulation has an organic solvent:water ratio in the range of from about 1:1 to about 1:10 by volume.

In some embodiments the lyophilized preparation is prepared by a lyophilization process that does not include an annealing step.

Some embodiments provide a kit comprising a lyophilized preparation and optionally one or more of a reconstitution reagent, a pharmaceutically acceptable carrier or adjuvant, a delivery device, and instructions for use.

In some embodiments the kit is used by a healthcare provider to treat a subject.

Some embodiments provide a kit comprising a lyophilized preparation and optionally one or more of a reconstitution reagent, a pharmaceutically acceptable carrier or adjuvant, a delivery device, and instructions for use.

In some embodiments the kit is used by a healthcare provider to treat a subject.

One embodiment provides a lyophilizer having disposed therein, a lyophilized preparation comprising lyoprotectant that comprises a cyclic oligosaccharide; and a particulate construct, the particulate construct comprising a polymer composition; a therapeutic agent; and a potentiating agent, wherein the therapeutic agent and the potentiating agent are associated with the polymer composition.

Some embodiments further comprise a control system.

In some embodiments the lyophilizer is a bench top lyophilizer.

Some embodiments further comprise 0.5% water or less by weight of the lyophilized preparation by weight of the lyophilized preparation.

In some embodiments the lyophilized preparation is substantially free of aggregates.

In some embodiments the particulate construct is a dual conjugate nanoparticle.

Some embodiments further comprise a reconstitution reagent.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene a poly etheresters, a polyester amides, a polyamide, derivatives, copolymers, blends, and other combinations thereof.

In some embodiments the particulate construct comprises a biodegradable block copolymer with a weight in the range of from about 1 kDa to about 21 kDa.

In some embodiments the therapeutic agent to biodegradable block copolymer ratio is in the range of from about 85:15 to about 55:45 by weight.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a polyalkylene glycol, a polypropylene glycol, and polybutylene glycol.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a polyvinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, a multifunctional linker, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, any derivative thereof, and any combination thereof.

In some embodiments the liquid formulation has an organic solvent:water ratio in the range of from about 1:1 to about 1:10 by volume.

In some embodiments the lyophilizer is used in a commercial production process.

One embodiment provides a method for producing an inclusion body comprising providing a liquid formulation that comprises a particulate construct that comprises a polymer composition and a therapeutic agent, and a lyoprotectant that comprises a polysaccharide; and lyophilizing the liquid formulation to produce a lyophilized preparation that comprises an inclusion body formed between the polysaccharide of the lyoprotectant and the polymer composition of the nanoparticle.

Some embodiments further comprise reconstituting the lyophilized preparation by adding a reconstitution reagent.

Some embodiments further comprise administering an effective amount of the lyophilized preparation to a subject.

In some embodiments lyophilizing the liquid formulation involves a rapid cycle lyophilization.

In some embodiments lyophilizing the liquid formulation does not include an annealing step.

In some embodiments the particulate construct is a dual conjugate nanoparticle.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene), a poly etheresters, a polyester amides, a polyamide, derivatives, copolymers, blends, and other combinations thereof.

In some embodiments the particulate construct comprises a biodegradable block copolymer with a weight in the range of from about 1 kDa to about 21 kDa.

In some embodiments the therapeutic agent to biodegradable block copolymer ratio is in the range of from about 85:15 to about 55:45 by weight.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a polyalkylene glycol, a polypropylene glycol, and polybutylene glycol.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, butyl acetate, propyl acetate, any derivative thereof, and any combination thereof.

In some embodiments the liquid formulation has an organic solvent:water ratio in the range of from about 1:1 to about 1:10 by volume.

One method provides a method comprising providing a liquid formulation that comprises a particulate construct and a lyoprotectant that comprises a polysaccharide wherein the particulate construct comprises a therapeutic agent and a polymer composition; and lyophilizing the liquid formulation to produce a lyophilized preparation that comprises a hydrogen bond formed between the polysaccharide of the lyoprotectant and the polymer composition of the nanoparticle.

In some embodiments lyophilizing the liquid formulation involves a rapid cycle lyophilization.

Some embodiments further comprise reconstituting the lyophilized preparation by adding a reconstitution reagent.

Some embodiments further comprise administering an effective amount of the lyophilized preparation to a subject.

In some embodiments lyophilizing the liquid formulation involves a rapid cycle lyophilization.

In some embodiments lyophilizing the liquid formulation does not include an annealing step.

In some embodiments the particulate construct is a dual conjugate nanoparticle.

In some embodiments the particulate construct comprises an additional additive selected from the group consisting of: a stabilizing polymer, an excipient, a surfactant, a derivative thereof, and any combination thereof.

In some embodiments the stabilizing polymer comprises a polymer selected from the group consisting of: a poly(vinyl alcohol), a poly(vinyl pyrrolidone), a poly(vinyl acetate), a crown ether, any derivative thereof, and any combination thereof.

In some embodiments the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene), a poly etheresters, a polyester amides, a polyamide, derivatives, copolymers, blends, and other combinations thereof.

In some embodiments the particulate construct comprises a biodegradable block copolymer with a weight in the range of from about 1 kDa to about 21 kDa.

In some embodiments the therapeutic agent to biodegradable block copolymer ratio is in the range of from about 85:15 to about 55:45 by weight.

In some embodiments the particulate construct has a Dv₉₀ of less than about 200 nm.

In some embodiments the particulate construct has a weight in the range of from about 7 kDa to about 30 kDa.

In some embodiments the particulate construct is a nanoparticle or a microparticle.

In some embodiments the particulate construct comprises a targeting ligand.

In some embodiments the potentiating agent is covalently bonded to the polymer composition.

In some embodiments the potentiating agent comprises a hydrophilic polymer.

In some embodiments the hydrophilic polymer comprises a polymer selected from the group consisting of: a polyethylene glycol, a polyalkylene glycol, a polypropylene glycol, and polybutylene glycol.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a polyvinyl alcohol), a glycolipid, a polyol, a salt, a crown ether, a specific nanoparticle-aptamer bioconjugate, any derivative thereof, and any combination thereof.

In some embodiments the potentiating agent protects the particulate construct under biological conditions and/or increases the solubility of the particulate construct.

In some embodiments the cyclic oligosaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, any derivative thereof, and any combination thereof.

In some embodiments the lyoprotectant further comprises a polymer.

In some embodiments an oligosaccharide is incorporated within a backbone of the polymer.

In some embodiments at least one occurrence of the cyclic oligosaccharide is oxidized.

In some embodiments a plurality of occurrences on the cyclic oligosaccharide structure are oxidized.

In some embodiments the lyoprotectant is added in a ratio from about 0.75:1 to about 3:1 by weight of the lyoprotectant:liquid formulation.

In some embodiments the polymer composition to lyoprotectant ratio is in the range of from about 0.75:1 to about 15:1 by weight.

In some embodiments at least one therapeutic agent is covalently bonded to the polymer composition.

In some embodiments the therapeutic agent is attached to the polymer composition through a linker.

In some embodiments the linker comprises a linking compound selected from the group consisting of: an alkanoate linker, a PEG-based linker, a linker that comprises a disulfide bond, a self-immolative linker, an amino acid, a peptide, a glutamic acid, a branched glutamic acid, a polyglutamic acid, any derivative thereof, and any combination thereof.

In some embodiments the therapeutic agent comprises a taxane drug.

In some embodiments the therapeutic agent comprises a drug selected from the group consisting of: a cancer drug, an anti-inflammatory drug, a thrombolytic drug, a cardiovascular drug, an anti-anginal drug, a diuretic drug, an antihistamine drug, a hormone-containing drug, an antibiotic drug, a pain reliever drug, an anti-diarrheal drug, an antiemetic, an anti-metabolite, an immunomodulator drug, a radiation drug, a chemotherapy drug, any derivative thereof, and any combination thereof.

In some embodiments the therapeutic agent is included in a range of about 5% to about 20% by weight of the particulate construct.

In some embodiments the liquid formulation comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, butyl acetate, propyl acetate, any derivative thereof, and any combination thereof.

In some embodiments the liquid formulation has an organic solvent:water ratio in the range of from about 1:1 to about 1:10 by volume.

One embodiment provides a liquid formulation that comprises a liposome that comprises a therapeutic agent, a liposomal bilayer, and a potentiating agent, and a lyoprotectant that comprises a cyclic polysaccharide.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a polypropylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

One embodiment provides a lyophilized liposome therapeutic composition that comprises a liposome that comprises a therapeutic agent, a liposomal bilayer, and a potentiating agent, and a lyoprotectant that comprises a cyclic polysaccharide.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

Some embodiments provide a kit comprising a lyophilized liposome therapeutic composition and optionally one or more of a reconstitution reagent, a pharmaceutically acceptable carrier or adjuvant, a delivery device, and instructions for use.

Some embodiments provide a kit wherein, the kit is used by a healthcare provider to treat a subject.

In some embodiments the lyophilized liposome therapeutic composition is prepared by a rapid cycle lyophilization process.

In some embodiments the lyophilized liposome therapeutic composition is prepared by a lyophilization process that does not include an annealing step.

One embodiment provides a method comprising providing a liquid formulation that comprises a liposome that comprises a therapeutic agent, a liposomal bilayer, and a potentiating agent, and a lyoprotectant that comprises a cyclic polysaccharide; and lyophilizing the liquid formulation to provide a lyophilized liposome therapeutic composition.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a polybutylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0, 75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

Some embodiments further comprise reconstituting the lyophilized preparation by adding a reconstitution reagent to form a reconstituted preparation.

Some embodiments further comprise administering an effective dose of the reconstituted preparation to a subject.

One embodiment provides a method of treating cancer comprising providing a lyophilized liposome therapeutic composition that comprises a liposome that comprises a therapeutic agent, a liposomal bilayer, and a potentiating agent, and a lyoprotectant that comprises a cyclic polysaccharide; and administering an effective amount of such composition to a subject in need thereof.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

Some embodiments further comprise reconstituting the lyophilized preparation by adding a reconstitution reagent to form a reconstituted preparation.

Some embodiments further comprise administering an effective dose of the reconstituted preparation to a subject.

In some embodiments the lyophilized liposome therapeutic composition is prepared by a rapid cycle lyophilization process.

In some embodiments the lyophilized liposome therapeutic composition is prepared by a lyophilization process that does not include an annealing step.

Some embodiments further comprise reconstituting the lyophilized preparation by adding a reconstitution reagent, to provide a reconstituted preparation.

In some embodiments the lyophilized preparation is a component of a kit that optionally comprises one or more of a reconstitution reagent, a pharmaceutical acceptable carrier or adjuvant, a delivery device, and instructions for use.

In some embodiments the administering of an effective dose of such reconstituted preparation to a subject is performed by a healthcare provider.

Some embodiments further comprise allowing the reconstituted preparation to interact with a cancer cell.

In some embodiments the cancer cell comprises a cancer cell selected from the group consisting of: a colorectal cancer cell, a gastric cancer cell, a liver cancer cell, a renal cancer cell, a cystic cancer cell, a pulmonary cancer cell, a billiard tract cancer cell, a pancreatic cancer cell, a uterine cancer cell, an ovarian cancer cell, a breast cancer cell, a melanoma, any derivative thereof, and any combination thereof.

One embodiment provides a lyophilized liposome therapeutic composition that comprises a liposome that comprises a therapeutic agent, a liposomal bilayer, and a potentiating agent, and a lyoprotectant that comprises a cyclic polysaccharide, and prepared by a rapid cycle lyophilization process.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, phosphatidylinositol, a phosphatidylinositol phosphate, a phosphatidylinositol, a bisphosphate, a phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

Some embodiments provide a kit comprising a lyophilized liposome therapeutic composition and optionally one or more of a reconstitution reagent, a pharmaceutically acceptable carrier or adjuvant, a delivery device, and instructions for use.

In some embodiments the kit is used by a healthcare provider to treat a subject.

In some embodiments the lyophilized liposome therapeutic composition is prepared by a rapid cycle lyophilization process.

In some embodiments the lyophilized liposome therapeutic composition is prepared by a lyophilization process that does not include an annealing step.

One embodiment provides a lyophilized liposome therapeutic composition that comprises a liposome that comprises a therapeutic agent, a liposomal bilayer, and a potentiating agent, and a lyoprotectant that comprises a cyclic polysaccharide, and prepared by a lyophilization process that does not include an annealing step.

In some embodiments the lyophilized liposome therapeutic composition comprising 0.5% water or less.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

Some embodiments provide a kit comprising a lyophilized liposome therapeutic composition and optionally one or more of a reconstitution reagent, a pharmaceutically acceptable carrier or adjuvant, a delivery device, and instructions for use.

In some embodiments the kit is used by a healthcare provider to treat a subject.

In some embodiments the lyophilized liposome therapeutic composition prepared by a rapid cycle lyophilization process.

In some embodiments the lyophilized liposome therapeutic composition prepared by a lyophilization process that does not include an annealing step.

Some embodiments provide a kit comprising a lyophilized liposome therapeutic composition and optionally one or more of a reconstitution reagent, a pharmaceutically acceptable carrier or adjuvant, a delivery device, and instructions for use.

In some embodiments the kit is used by a healthcare provider to treat a subject.

In some embodiments the lyophilized liposome therapeutic composition prepared by a rapid cycle lyophilization process.

In some embodiments the lyophilized liposome therapeutic composition wherein, the lyophilized liposome therapeutic composition is in a vial.

One embodiment provides a device having disposed therein, a lyophilized liposome therapeutic composition that comprises a liposome that comprises a therapeutic agent, a liposomal bilayer, and a potentiating agent, and a lyoprotectant that comprises a cyclic polysaccharide.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a polyoxyethylene ester), a poly(vinylpyrrolidone), a polyvinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

In some embodiments the device is a storage device, a cannula, a syringe, drip bag, or any combination thereof.

In some embodiments the device is provided as part of a kit that includes a reconstitution reagent and/or operating instructions.

One embodiment provides a method of providing a liquid preparation comprising providing a lyophilized liposome therapeutic composition comprising a liposome that comprises a therapeutic agent, a liposomal bilayer, and a potentiating agent, and a lyoprotectant that comprises a polysaccharide, and combining said liquid preparation with a reconstitution reagent, to provide a reconstituted preparation.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a polyoxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrhea) drug, an antiemetic drug, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

Some embodiments further comprise reconstituting the lyophilized preparation by adding a reconstitution reagent to form a reconstituted preparation.

Some embodiments further comprise administering an effective dose of the reconstituted preparation to a subject.

In some embodiments the reconstitution reagent comprises a liquid selected from the group consisting of: water, sterile water, a salt solution, an alcohol, any derivative thereof, and any combination thereof.

One embodiment provides a method of treating cancer comprising providing a lyophilized liposome therapeutic composition that comprises a liposome that comprises a therapeutic agent, a liposomal bilayer, and a potentiating agent, and a lyoprotectant that comprises a cyclic polysaccharide; and administering an effective amount of such composition to a subject.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

Some embodiments further comprise reconstituting the lyophilized preparation by adding a reconstitution reagent to form a reconstituted preparation.

Some embodiments further comprise administering an effective dose of the reconstituted preparation to a subject.

In some embodiments the lyophilized liposome therapeutic composition is prepared by a rapid cycle lyophilization process.

In some embodiments the lyophilized liposome therapeutic composition is prepared by a lyophilization process that does not include an annealing step.

Some embodiments further comprise reconstituting the lyophilized preparation by adding a reconstitution reagent to form a reconstituted preparation.

Some embodiments further comprise administering an effective dose of the reconstituted preparation to a subject.

Some embodiments further comprise allowing the reconstituted preparation to interact with a cancer cell.

In some embodiments the cancer cell comprises a cancer cell selected from the group consisting of: a colorectal cancer cell, a gastric cancer cell, a liver cancer cell, a renal cancer cell, a cystic cancer cell, a pulmonary cancer cell, a billiard tract cancer cell, a pancreatic cancer cell, a uterine cancer cell, an ovarian cancer cell, a breast cancer cell, a melanoma, any derivative thereof, and any combination thereof.

One embodiments provides a lyophilizer having disposed therein, a lyophilized preparation of a lyophilized liposome therapeutic composition that comprises a liposome that comprises a therapeutic agent, a liposomal bilayer, and a potentiating agent, and a lyoprotectant that comprises a cyclic polysaccharide.

In some embodiments the lyophilizer comprises a control system.

In some embodiments the lyophilizer is a bench top lyophilizer.

In some embodiments the lyophilized preparation comprises 0.5% water or less by weight of the lyophilized preparation.

In some embodiments the lyophilized liposome therapeutic composition comprises a targeting ligand.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

In some embodiments the lyophilizer is capable of a rapid cycle lyophilization.

One embodiment provides a method for producing an inclusion body comprising providing a liquid formulation that comprises a lyophilized liposome composition comprising a liposome that comprises a therapeutic agent, a liposomal bilayer, and a potentiating agent, and a lyoprotectant that comprises a cyclic polysaccharide, and lyophilizing the liquid formulation to produce a lyophilized preparation that comprises an inclusion body formed between the cyclic polysaccharide of the lyoprotectant and the liposome.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a polyoxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

One embodiments provides a method comprising providing a liquid formulation that comprises a lyophilized liposome composition comprising a liposome that comprises a therapeutic agent, a liposomal bilayer, and a potentiating agent, and a lyoprotectant that comprises a cyclic polysaccharide, and lyophilizing the liquid formulation to produce a lyophilized preparation that comprises a hydrogen bond formed between the cyclic polysaccharide of the lyoprotectant and the liposome.

In some embodiments the liposomal bilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative thereof, and any combination thereof.

In some embodiments the liposome comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the liposome and/or increases the solubility of the liposome.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the liposome further comprises a targeting ligand.

In some embodiments at least one occurrence of the cyclic polysaccharide is oxidized.

In some embodiments a plurality of occurrences on the cyclic polysaccharide are oxidized.

In some embodiments the liposome to lyoprotectant ratio is a ratio in the range of from about 0.75:1 to about 3:1 by weight of the lyoprotectant:liquid formulation.

In some embodiments the liposome comprises a tangled network of hydrophobic tails and a hydrophilic head region.

In some embodiments the therapeutic agent comprises a drug.

In some embodiments at least a portion of the drug is located within a tangled network of hydrophobic tails in the liposome.

In some embodiments the drug comprises a taxane

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, any derivative thereof, and any combination thereof.

In some embodiments the therapeutic agent is included in a range of about 5% to about 20% by weight of the liposome.

In some embodiments the liposome comprises an excipient.

In some embodiments the liposome has a Dv₉₀ of less than about 200 nm.

In some embodiments the lyoprotectant forms at least one hydrogen bond with the liposome.

In some embodiments the lyoprotectant forms at least one inclusion body with the liposome.

One embodiment provides a liquid formulation that comprises a micelle that comprises a therapeutic agent, a micellular unilayer, and a potentiating agent, and a lyoprotectant that comprises a cyclic polysaccharide.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle

In some embodiments the lyoprotectant forms at least one inclusion body with the micelle.

One embodiment provides a lyophilized micelle therapeutic composition that comprises a micelle that comprises a therapeutic agent, a micellular unilayer, and a potentiating agent, and a lyoprotectant that comprises a cyclic polysaccharide.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant forms at least one inclusion body with the micelle.

Some embodiments provide a kit comprising a lyophilized micelle therapeutic composition and optionally one or more of a reconstitution reagent, a pharmaceutically acceptable carrier or adjuvant, a delivery device, and instructions for use.

In some embodiments the kit is used by a healthcare provider to treat a subject.

In some embodiments the lyophilized micelle therapeutic composition prepared by a rapid cycle lyophilization process.

In some embodiments the lyophilized micelle therapeutic composition is prepared by a lyophilization process that does not include an annealing step.

One embodiment provides a method comprising providing a liquid formulation that comprises a micelle that comprises a therapeutic agent, a micellular unilayer, and a potentiating agent, and a lyoprotectant that comprises a cyclic polysaccharide; and lyophilizing the liquid formulation to provide a lyophilized micelle therapeutic composition.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a polyoxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments the least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant forms at least one inclusion body with the micelle.

Some embodiments further comprise reconstituting the lyophilized preparation by adding a reconstitution reagent to form a reconstituted preparation.

Some embodiments further comprise administering an effective dose of the reconstituted preparation to a subject.

One embodiments provides a method of treating cancer comprising providing a lyophilized micelle therapeutic composition that comprises a micelle that comprises a therapeutic agent, a micellular unilayer, and a potentiating agent, and a lyoprotectant that comprises a cyclic polysaccharide; and administering an effective amount of such composition to a subject in need thereof.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant forms at least one inclusion body with the micelle.

Some embodiments further comprise reconstituting the lyophilized preparation by adding a reconstitution reagent to form a reconstituted preparation.

Some embodiments further comprise administering an effective dose of the reconstituted preparation to a subject.

In some embodiments the lyophilized micelle therapeutic composition is prepared by a rapid cycle lyophilization process.

In some embodiments the lyophilized micelle therapeutic composition is prepared by a lyophilization process that does not include an annealing step.

Some embodiments further comprise reconstituting the lyophilized preparation by adding a reconstitution reagent.

In some embodiments the lyophilized preparation is a component of a kit that optionally comprises one or more of a reconstitution reagent, a pharmaceutical acceptable carrier or adjuvant, a delivery device, and instructions for use.

In some embodiments the administering of an effective dose of such preparation to a subject is performed by a healthcare provider.

Some embodiments further comprise allowing the micelle to interact with a cancer cell.

In some embodiments the cancer cell comprises a cancer cell selected from the group consisting of: a colorectal cancer cell, a gastric cancer cell, a liver cancer cell, a renal cancer cell, a cystic cancer cell, a pulmonary cancer cell, a billiard tract cancer cell, a pancreatic cancer cell, a uterine cancer cell, an ovarian cancer cell, a breast cancer cell, a melanoma, any derivative thereof, and any combination thereof.

One embodiment provides a lyophilized micelle therapeutic composition that comprises a micelle that comprises a therapeutic agent, a micellular unilayer, and a potentiating agent, and a lyoprotectant that comprises a cyclic polysaccharide, and prepared by a rapid cycle lyophilization process.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant forms at least one inclusion body with the micelle.

Some embodiments provide a kit comprising a lyophilized micelle therapeutic and optionally one or more of a reconstitution reagent, a pharmaceutically acceptable carrier or adjuvant, a delivery device, and instructions for use.

Some embodiments provide a kit wherein, the kit is used by a healthcare provider to treat a subject.

In some embodiments the lyophilized micelle therapeutic composition is prepared by a rapid cycle lyophilization process.

In some embodiments the lyophilized micelle therapeutic composition is prepared by a lyophilization process that does not include an annealing step.

A lyophilized micelle therapeutic composition that comprises a micelle that comprises a therapeutic agent, a micellular unilayer, and a potentiating agent, and a lyoprotectant that comprises a cyclic polysaccharide, and prepared by a lyophilization process that does not include an annealing step.

In some embodiments the lyophilized micelle therapeutic composition comprises 0.5% water or less.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant forms at least one inclusion body with the micelle.

Some embodiments provide a kit comprising a lyophilized micelle therapeutic composition and optionally one or more of a reconstitution reagent, a pharmaceutically acceptable carrier or adjuvant, a delivery device, and instructions for use.

Some embodiments provide a kit wherein, the kit is used by a healthcare provider to treat a subject.

In some embodiments the lyophilized micelle therapeutic composition is prepared by a rapid cycle lyophilization process.

In some embodiments the lyophilized micelle therapeutic composition is prepared by a lyophilization process that does not include an annealing step.

Some embodiments provide a kit comprising a lyophilized micelle therapeutic composition and optionally one or more of a reconstitution reagent, a pharmaceutically acceptable carrier or adjuvant, a delivery device, and instructions for use.

Some embodiments provide a kit wherein, the kit is used by a healthcare provider to treat a subject.

In some embodiments the lyophilized micelle therapeutic composition is prepared by a rapid cycle lyophilization process.

In some embodiments the lyophilized micelle therapeutic composition is in a vial.

One embodiments provides a device having disposed therein, a lyophilized micelle therapeutic composition that comprises a micelle that comprises a therapeutic agent, a micellular unilayer, and a potentiating agent, and a lyoprotectant that comprises a cyclic polysaccharide.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2, 5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a polybutylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a polyvinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant forms at least one inclusion body with the micelle.

In some embodiments the device is a storage device, a cannula, a syringe, drip bag, or any combination thereof.

In some embodiments the device is provided as part of a kit that includes a reconstitution reagent and/or operating instructions.

One embodiment provides a method of providing a liquid preparation comprising providing a lyophilized micelle therapeutic composition comprising a micelle that comprises a therapeutic agent, a micellular unilayer, and a potentiating agent, and a lyoprotectant that comprises a polysaccharide, and combining said liquid preparation with a reconstitution reagent, to provide a reconstituted preparation.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant forms at least one inclusion body with the micelle.

Some embodiments further comprise reconstituting the lyophilized preparation by adding a reconstitution reagent to form a reconstituted preparation.

Some embodiments further comprise administering an effective dose of the reconstituted preparation to a subject.

In some embodiments the reconstitution reagent comprises a liquid selected from the group consisting of: water, sterile water, a salt solution, an alcohol, and any derivative thereof, and any combination thereof.

One embodiment comprises a method of treating cancer comprising providing a lyophilized micelle therapeutic composition that comprises a micelle that comprises a therapeutic agent, a micellular unilayer, and a potentiating agent, and a lyoprotectant that comprises a cyclic polysaccharide; and administering an effective amount of such composition to a subject.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant forms at least one inclusion body with the micelle.

Some embodiments further comprise reconstituting the lyophilized preparation by adding a reconstitution reagent to form a reconstituted preparation.

Some embodiments further comprise administering an effective dose of the reconstituted preparation to a subject.

Some embodiments further comprise a lyophilized micelle therapeutic composition prepared by a rapid cycle lyophilization process.

Some embodiments further comprise a lyophilized micelle therapeutic composition prepared by a lyophilization process that does not include an annealing step.

Some embodiments further comprise reconstituting the lyophilized preparation by adding a reconstitution reagent to form a reconstituted preparation.

Some embodiments further comprise administering an effective dose of the reconstituted preparation to a subject.

Some embodiments further comprise allowing the reconstituted preparation to interact with a cancer cell.

In some embodiments the cancer cell comprises a cancer cell selected from the group consisting of: a colorectal cancer cell, a gastric cancer cell, a liver cancer cell, a renal cancer cell, a cystic cancer cell, a pulmonary cancer cell, a billiard tract cancer cell, a pancreatic cancer cell, a uterine cancer cell, an ovarian cancer cell, a breast cancer cell, a melanoma, any derivative thereof, and any combination thereof.

One embodiment provides a lyophilizer having disposed therein, a lyophilized preparation of a lyophilized micelle therapeutic composition that comprises a micelle that comprises a therapeutic agent, a micellular unilayer, and a potentiating agent, and a lyoprotectant that comprises a cyclic polysaccharide.

In some embodiments the lyophilizer comprises a control system.

In some embodiments the lyophilizer is a bench top lyophilizer.

In some embodiments the lyophilized preparation comprises 0.5% water or less by weight of the lyophilized preparation.

In some embodiments the lyophilized micelle therapeutic composition comprises a targeting ligand.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a polyoxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant ms at least one inclusion body with the micelle.

In some embodiments the lyophilizer is capable of a rapid cycle lyophilization.

One embodiment provides a method for producing an inclusion body comprising providing a liquid formulation that comprises a lyophilized micelle composition comprising a micelle that comprises a therapeutic agent, a micellular unilayer, and a potentiating agent, and a lyoprotectant that comprises a cyclic polysaccharide, and lyophilizing the liquid formulation to produce a lyophilized preparation that comprises an inclusion body formed between the cyclic polysaccharide of the lyoprotectant and the micelle.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile. 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a polypropylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments the plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant forms at least one inclusion body with the micelle.

One embodiment provides a method comprising providing a liquid formulation that comprises a lyophilized micelle composition comprising a micelle that comprises a therapeutic agent, a micellular unilayer, and a potentiating agent, and a lyoprotectant that comprises a cyclic polysaccharide, and lyophilizing the liquid formulation to produce a lyophilized preparation that comprises a hydrogen bond formed between the cyclic polysaccharide of the lyoprotectant and the micelle.

In some embodiments the micellular unilayer comprises a lipid selected from the group consisting of: a phospholipid, a glycerophospholipid, a phosphatidic acid, a phosphatidylethanolamine, a phosphatidylcholine, a phosphatidylserine, a phosphoinositide, a phosphatidylinositol, a phosphatidylinositol phosphate, a phosphatidylinositol, a bisphosphate, a phosphatidyinositol triphosphate, a sphingomyelin, any derivative, and any combination thereof.

In some embodiments the micelle comprises a physiologically acceptable liquid selected from the group consisting of: an aqueous liquid, an organic liquid, and any combination thereof.

In some embodiments the organic liquid comprises an organic solvent selected from the group consisting of: acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsulfoxide, hexane, benzene, methyl ether, carbon tetrachloride, methylene chloride, butyl acetate, propyl acetate, any derivative thereof, and any combination thereof.

In some embodiments the ratio of the potentiating agent to the lipid ratio is greater than or equal to 2.5:1.

In some embodiments the potentiating agent comprises a hydrophilic polymer.

In some embodiments the hydrophilic polymer comprises a polymer selected from the group consisting of: a polyethylene glycol, a poly(alkylene glycol), a poly(propylene glycol), a poly(butylene glycol), any derivative thereof, and any combination thereof.

In some embodiments the potentiating agent comprises a compound selected from the group consisting of: a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof.

In some embodiments the potentiating agent stabilizes the micelle and/or increases the solubility of the micelle.

In some embodiments the cyclic polysaccharide further comprises a linear, branched, or grafted polysaccharide.

In some embodiments the cyclic polysaccharide comprises a cyclodextrin moiety selected from the group consisting of: an α-cyclodextrin, a β-cyclodextrin, a 2-hydroxypropyl-β-cyclodextrin, a β-cyclodextrin sulfobutylethers sodium, and any derivative or combination thereof.

In some embodiments the lyoprotectant further comprises a polymer.

In some embodiments an oligosaccharide is incorporated within a backbone of the polymer.

In some embodiments the micelle further comprises a targeting ligand.

In some embodiments at least one occurrence of the cyclic polysaccharide is oxidized.

In some embodiments a plurality of occurrences on the cyclic polysaccharide are oxidized.

In some embodiments the micelle to lyoprotectant ratio is a ratio in the range of from about 0.75:1 to about 3:1 by weight of the lyoprotectant:liquid formulation.

In some embodiments the micelle comprises a tangled network of hydrophobic tails and a hydrophilic head region.

In some embodiments the therapeutic agent comprises a drug.

In some embodiments at least a portion of the drug is located within a tangled network of hydrophobic tails in the micelle.

In some embodiments the drug comprises a taxane.

In some embodiments the drug comprises a compound selected from the group consisting of: a cancer drug, an anti-inflammatory drug, a thrombolytic drug, a cardiovascular drug, an anti-anginal drug, a diuretic drug, an anti-histamine drug, a hormone-containing drug, an antibiotic drug, a pain reliever drug, an anti-diarrheal drug, an antiemetic drug, an anti-metabolite drug, an immunomodulator drug, a radiation drug, a chemotherapy drug, and any derivative thereof, and any combination thereof.

In some embodiments the therapeutic agent is included in a range of about 5% to about 20% by weight of the micelle.

In some embodiments the micelle comprises an excipient.

In some embodiments the micelle has a Dv₉₀ of less than about 200 nm.

In some embodiments the lyoprotectant forms at least one hydrogen bond with the micelle.

In some embodiments the lyoprotectant forms at east one inclusion body with the micelle.

The features and advantages of the present invention will be readily apparent to those skilled in the art upon a reading of the description of the embodiments that follows.

The features and advantages of the present invention will be readily apparent to those skilled in the art upon a reading of the description of the embodiments that follows.

### BRIEF DESCRIPTION OF THE DRAWINGS

These drawings illustrate certain aspects of some of the embodiments of the present invention, and should not be used to limit or define the invention.

FIG. 1 illustrates the steps of lyophilization reaction.

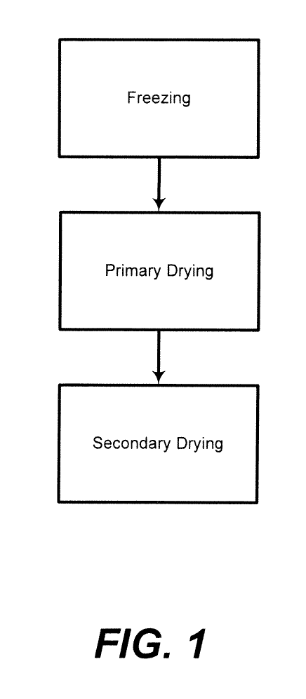

FIG. 2 illustrates a hydrogen bond between a lyoprotectant that comprises a cyclic oligosaccharide and a potentiating agent.

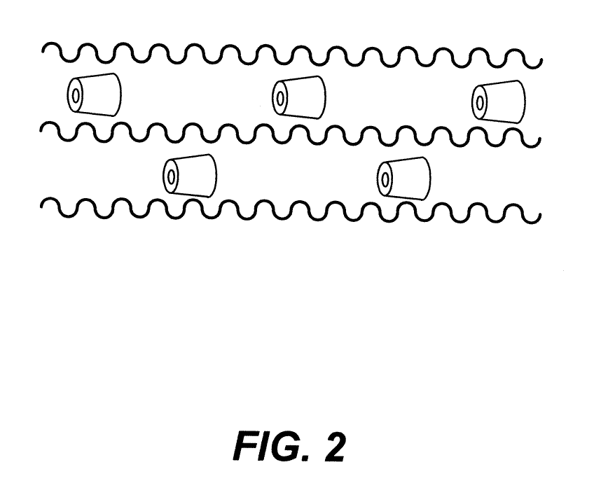

FIG. 3 illustrates an inclusion body between a lyoprotectant that comprises a cyclic oligosaccharide and a potentiating agent.

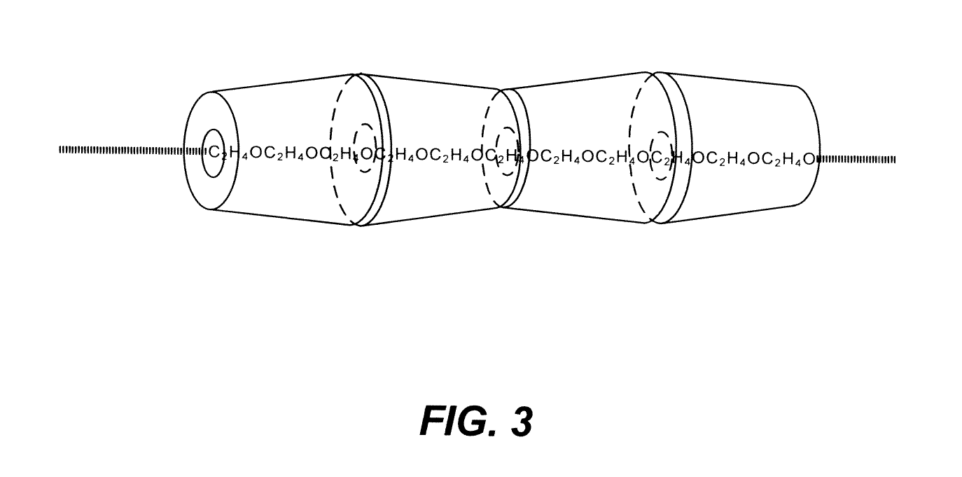

While the present invention is susceptible to various modifications and alternative forms, specific embodiments thereof have been shown by way of example in the figures and are herein described in detail. It should be understood, however, that the description herein of specific embodiments is not intended to limit Invention to the particular forms disclosed, but on the contrary, intention is to cover all modifications, equivalents, and alternatives falling within the spirit and scope of invention as defined by the appended claims.

### DETAILED DESCRIPTION

The present invention relates to the production, use and administration of nanoparticles, liposomes, and micelles for therapeutic agent delivery. In particular, at least in some embodiments, the present invention relates to the production and lyophilization of PEGylated nanoparticles, micelles, and liposomes for use and administration in a subject. Among the many potential advantages of the present invention, the methods and compositions of the present invention may, among other things, provide lyophilized preparations comprising potentiated therapeutic particles (e.g., microparticles and/or nanoparticles), liposomes, and micelles wherein there is minimal aggregation of the particles (e.g., microparticles and/or nanoparticles), liposomes, and micelles. Therefore, the compositions of the present invention are thought to be more stable than certain other formulations, and preferably can be easily reconstituted at high concentration (e.g., at least 10 mg polymer/mL, at least 20 mg polymer/mL, at least 30 mg polymer/mL, at least 40 mg polymer/mL, at least 50 mg polymer/mL, at least 60 mg polymer/mL, at least 70 mg polymer/mL, at least 80 mg polymer/mL, at least 90 mg polymer/mL or at least 100 mg polymer/mL) in a short period of time (e.g., less than about 1 hour, about 30 minutes, about 20 minutes, about 15 minutes, about 10 minutes, or about 5 minutes). Thus, the invention provides therapeutics that contain particles e.g., microparticles and/or nanoparticles), liposomes, and micelles in stable lyophilized form that can be reconstituted quickly and easily prior to use to provide a reconstituted formulation that can be administered to a patient in need thereof in a volume that is appropriate and convenient for the intended mode of administration, e.g., as a bolus injection.

In addition, the present invention provides the use of oligosaccharides, such as cyclic oligosaccharides, as lyoprotectants during lyophilization allowing the therapeutic particles (e.g., microparticles and/or nanoparticles), liposomes, and micelles to maintain their particle size during the lyophilization reaction. In some embodiments, this can be achieved using a minimal lyoprotectant/polymer weight ratio of as low as about 0.75:1 lyoprotectant:polymer. Moreover, another advantage of the present invention includes the use of a novel lyophilization method that utilizes single-step slow ramping rather than multi-step ramping and shortens the lyophilization time to one third of most conventional lyophilization methods.

### DEFINITIONS

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain to the polymer backbone. In certain embodiments, one or the other or both general types of biodegradation may occur during use of a polymer.

The term “biodegradable polymer composition” as used herein refers to an assembly of one or more biodegradable polymers, and can optionally contain a stabilizing polymer, and an excipient. The biodegradable polymer composition is held together by attractive physical forces, which may include a chemical or physical bond in some instances.

The term “conjugate,” as used herein refers to a chemical compound that has been formed by the joining of two or more compounds through a covalent or non-covalent bond.

The term “copolymer,” as used herein refers to polymer consisting of two or more different monomers. The term “copolymer” as used herein is not limited to the combination of two polymers, but includes any combination of polymers, e.g., terpolymers, block copolymers, and the like.

The term “cyclodextrin moiety,” as used herein refers to cyclodextrin (e.g., an α, β, or γ cyclodextrin) radical that is incorporated into, or a part of a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

The term “delivery device,” as used herein refers to a vehicle with which a therapeutic agent is administered. Examples include, but are not limited to, a storage device, a cannula, a syringe, or a drip bag.

The term “derivative,” as used herein refers to any compound that is made from one of the identified compounds, for example, by replacing one atom in the listed compound with another atom or group of atoms, rearranging two or more atoms in the listed compound, or ionizing the listed compounds. Derivatives may be obtained through chemical functionalization or derivatization. A derivative may also refer to compound derived from another compound while maintaining its structural features.

The term “derivatized carbohydrate,” refers to an entity which differs from the subject non-derivatized carbohydrate by at least one atom. For example, instead of the —OH present on a non-derivatized carbohydrate the derivatized carbohydrate can have —OX, wherein X is other than H. Derivatives may be obtained through chemical functionalization and/or substitution or through de novo synthesis the term “derivative” implies no process-based limitation.

The term “drug,” as used herein refers to a therapeutic agent that is pharmacologically active.

The term “dual conjugate nanoparticle” or “dual conjugate microparticle.” as used herein refers to a nanoparticle or microparticle comprising a polymer composition to which a potentiating agent is covalently or non-covalently conjugated, and a therapeutic agent is covalently or non-covalently conjugated. The potentiating agent and the therapeutic agent can be conjugated on a single polymer in the biodegradable polymer composition, on separate polymers in the biodegradable polymer composition, or combinations thereof, as desired. In some embodiments, these conjugates may be in the form of non-covalent molecular associations, while in others these conjugates may be in the form of a covalent bond.

The term “Dv₅₀,” as used herein refers to the analytical measurement of particle volume below which 50% of the particles exist.

The term “Dv₉₀,” as used herein refers to the analytical measurement of particle volume below which 90% of the particles exist.

The term “effective amount” or “an amount effective,” as used herein refers to an amount of the liquid formulations and lyophilized preparations, compound or composition which is effective, upon single or multiple dose administrations to a subject, in treating a cell, or curing, alleviating, relieving or improving a symptom of a disorder. An effective amount of the composition may vary according to factors such as the disease state, age, sex, and weight of Individual, and the ability of the compound to elicit a desired response in Individual. An effective amount is also one in which any toxic or detrimental effects of the composition is outweighed by the therapeutically beneficial effects.

The term “excipient,” as used herein refers to a relatively inert ingredient included in a therapeutic agent preparation, e.g., for the purpose of improving its physical qualities or vehicle.

The term “fluidic liposomal core,” as used herein refers to the fluidic core of a liposome that is entrapped by one or more bilayers composed of natural or synthetic lipids.

The term “healthcare provider,” as used herein refers to individuals or organizations that provide healthcare services to a person, community, etc. Examples include doctors, hospitals, continuing care retirement communities, skilled nursing facilities, subacute care facilities, clinics, multi-specialty clinics, free-standing ambulatory centers, home health agencies, and HMOs.

The term “hydrogen bond,” as used herein refers to an attractive interaction between a hydrogen atom and a electronegative atom.

The term “hydrophilic,” as used herein, refers to a moiety that has a solubility in aqueous solution of at least about 0.05 mg/mL or greater (e.g., at least about 1.0 mg/mL or greater).

The term “hydrophobic,” as used herein, refers to a moiety that can be dissolved in an aqueous solution at physiological ionic strength only to the extent of about 0.05 mg/mL or less (preferably about 0.001 mg/mL or less).

The term “inclusion body,” as used herein refers to a complex in which one component (the host) forms a cavity containing spaces in the shape of a tunnel or channel in which molecular entities of a second chemical species (the guest) are location. There is no covalent bonding between guest and host, for example, the interaction being due to Van der Waals forces.

The term “kit,” as used herein, refers to a collection of at least two components comprising the kit. Taken together, the components constitute a functional unit for a given purpose. Individual member components may be physically packaged together or separately. For example, a kit comprising an instruction for using the kit may or may not physically include Instruction with other individual member components. Instead, Instruction can be supplied as a separate member component, either in a paper form or an electronic form, which may be supplied on a computer readable memory device, or downloaded from an internet website, or as a recorded presentation.

The term “linker,” as used herein, is a moiety having at least two functional groups. One functional group is capable of reacting with a functional group on a polymer described herein, and a second functional group is capable of reacting with a functional group on agent described herein. In some embodiments the linker has just two functional groups. A linker may have more than two functional groups (e.g., 3, 4, 5, 6, 7, 8, 9, 10 or more functional groups), which may be used, e.g., to link multiple agents to a polymer. Depending on the context, linker can refer to a linker moiety before attachment to either of a first or second moiety (e.g., agent or polymer), after attachment to one moiety but before attachment to a second moiety, or the residue of the linker present after attachment to both the first and second moiety.

The term “liquid formulation,” as used herein refers to a formulations in aqueous, organic and aqueous organic solvents. Preferably, the liquid formulation is physiologically acceptable.

As used herein, the term “lipid bilayer” refers to a lipid layer of a liposome composed of a natural and/or a synthetic lipid.

As used herein, the term “liposome” refers to a microscopic vesicle composed of a phospholipid bilayer that is capable of encapsulating a therapeutic agent. A fluidic liposomal core is entrapped by one or more lipid bilayers composed of natural or synthetic lipids. Liposomes composed of natural phospholipids are biologically inert and weakly immunogenic, and they possess low intrinsic toxicity. Further, drugs with different lipophilicities can be encapsulated into liposomes: strongly lipophilic drugs are entrapped almost completely in the lipid bilayer, strongly hydrophilic drugs are located exclusively in the aqueous compartment, and drugs with intermediate logP easily partition between the lipid and aqueous phases, both in the bilayer and in the aqueous core.

The term “lyophilization,” as used herein refers to a process for the creation of a stable preparation of a substance by rapid freezing and dehydration of the frozen substance under high vacuum.

The term “lyophilizer,” as used herein refers to a device or a piece of equipment that is capable of performing a lyophilization process.

The term “lyoprotectant,” as used herein refers to a substance present in a lyophilized preparation. Typically it is present prior to the lyophilization process and persists in the resulting lyophilized preparation. It can be used to protect nanoparticles, liposomes, and/or micelles during lyophilization, for example to reduce or prevent aggregation, particle collapse and/or other types of damage. Typically a lyoprotectant is added after the formation of the particles. If a concentration step is present, e.g., between formation of the particles and lyophilization, a lyoprotectant can be added before or after the concentration step. In an embodiment the lyoprotectant is a cryoprotectant.

In an embodiment the lyoprotectant is a carbohydrate. The term “carbohydrate,” as used herein refers to and encompasses monosaccharides, disaccharides, oligosaccharides and polysaccharides.

In an embodiment, the lyoprotectant is a monosaccharide. The term “monosaccharide,” as used herein refers to a single carbohydrate unit (e.g., a simple sugar) that can not be hydrolyzed to simpler carbohydrate units. Exemplary monosaccharide lyoprotectants include glucose, fructose, galactose, xylose, ribose and the like.

In an embodiment, the lyoprotectant is a disaccharide. The term “disaccharide,” as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide lyoprotectants include sucrose, trehalose, lactose, maltose and the like.

In an embodiment, the lyoprotectant is an oligosaccharide. The term “oligosaccharide,” as used herein refers to a compound or a chemical moiety formed by 3 to about 15, preferably 3 to about 10 monosaccharide units that are bonded together through glycosidic linkages, for example through 1-4 linkages or 1-6 linkages, to form a linear, branched or cyclic structure. Exemplary oligosaccharide lyoprotectants include cyclodextrins, raffinose, melezitose, maltotriose, stachyose acarbose, and the like. An oligosaccharide can be oxidized or reduced.

In an embodiment, the lyoprotectant is a cyclic oligosaccharide. The term “cyclic oligosaccharide,” as used herein refers to a compound or a chemical moiety formed by 3 to about 15, preferably 6, 7, 8, 9, or 10 monosaccharide units that are bonded together through glycosidic linkages, for example through 1-4 linkages or 1-6 linkages, to form a cyclic structure. Exemplary cyclic oligosaccharide lyoprotectants include cyclic oligosaccharides that are discrete compounds, such as α cyclodextrin, β cyclodextrin, or γ cyclodextrin.

Other exemplary cyclic oligosaccharide lyoprotectants include compounds which include a cyclodextrin moiety in a larger molecular structure, such as a polymer that contains a cyclic cyclodextrin moiety. A cyclic oligosaccharide can be oxidized or reduced, for example, oxidized to dicarbonyl forms. The term “cyclodextrin moiety,” as used herein refers to cyclodextrin (e.g., an α, β, or γ cyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms,

Carbohydrate lyoprotectants, e.g., cyclic oligosaccharide lyoprotectants, can be derivatized carbohydrates. For example, in an embodiment, the lyoprotectant is a derivatized cyclic oligosaccharide, e.g., a derivatized cyclodextrin, e.g., 2 hydroxy propyl-β cyclodextrin, e.g., partially etherified cyclodextrins (e.g., partially etherified βcyclodextrins) disclosed in U.S. Pat. No. 6,407,079, the contents of which are incorporated herein by this reference.

An exemplary lyoprotectant is a polysaccharide. The term “polysaccharide,” as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide lyoprotectants include glycogen, amylase, cellulose, dextran, maltodextrin and the like.

The term “micelle,” as used herein refers to a lipid sphere that contains no aqueous material.

The term “microparticle” as used herein refers to a material structure whose size in any dimension (e.g., x, y, and z Cartesian dimensions) is about 1 micrometer (micron) or more, but less than 1000 micrometers (microns). A microparticle can have a variety of geometrical shapes, e.g., spherical, ellipsoidal, etc. The term “microparticles” is used as the plural of the term “microparticle.”

The term “nanoparticle” is used herein to refer to a material structure whose size in any dimension (e.g., x, y, and z Cartesian dimensions) is about 1 micrometer or less, e.g., less than about 500 nm or less than about 200 nm or less than about 100 nm, and greater than about 5 nm. A nanoparticle can have a variety of geometrical shapes, e.g., spherical, ellipsoidal, etc. The term “nanoparticles” is used as the plural of the term “nanoparticle.”

The term “nanosome,” as used herein refers to a liposome designed on the nanoscale.

The term “pharmaceutically acceptable carrier or adjuvant,” as used herein, refers to a carrier or adjuvant that may be administered to a patient, together with a polymer-agent conjugate, particle or composition described herein, and which does not destroy the pharmacological activity thereof and is nontoxic when administered in doses sufficient to deliver a therapeutic amount of the particle. Some examples of materials which can serve as pharmaceutically acceptable carriers include: sugars, such as lactose, glucose, mannitol and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical compositions.

The term “polymer,” as used herein, is given its ordinary meaning as used in the art, i.e., a molecular structure featuring one or more repeat units (monomers), connected by covalent bonds. The repeat units may all be identical, or in some cases, there may be more than one type of repeat unit present within the polymer. In some cases, the polymer is biologically derived, i.e., a biopolymer. Non-limiting examples of biopolymers include peptides or proteins (i.e., polymers of various amino acids), or nucleic acids such as DNA or RNA. The terms “polymer” or “polymers” as used herein do not imply any particular degree of polymerization; for instance, oligomers and copolymers are encompassed within this definition.

The term “potentiating agent,” as used herein refers to a chemical moiety that when incorporated into conjugated nanoparticles of the present invention, as defined herein, that serves to shield the nanoparticle from inactivating processes in the body. In some instances, the potentiating agent may increase the hydrodynamic radius of the nanoparticle and shield its surfaces towards the periphery. In some instances, these may increase the half-life of the nanoparticle inside a subject's body, enabling less frequent dosing and more efficient therapies. Thus, the stability of the nanoparticles that contain a potentiating agent is increased, for example, proteases resistance is increased, immunogenicity is reduced, and their renal excretion is decelerated. Examples include, but are not limited to, enzymes, peptides, polysaccharides, phospholipid analogs, polyphosphazenes, polyethylene glycol, hydrophilic polymers, glycolipids, and specific nanoparticle-aptamer bioconjugates. In some embodiments, the potentiating agent forms a conjugate with a drug and/or a polymer in the polymer composition in the nanoparticles of the present invention. Preferably, a potentiating agent suitable for use in the nanoparticles of the present invention has: monodispersity or at least a dispersity index close to 1 to assure a reproducible high quality; availability of one single terminal reactive group for the coupling reaction to avoid a crosslinking between the therapeutic agent molecules; non-toxicity and non-immunogenic characteristics; optionally branching for optimal surface protection, and options for site-specific reactions, if desired.

The term “protect,” as used herein with respect to the potentiating agent refers to the process wherein the potentiating agent prolongs the lifespan of circulation, alters therapeutic agent elimination pathway(s), and/or changes the biodistribution profile of a therapeutic agent.

The term “rapid cycle lyophilization process,” as used herein, refers to a lyophilization process that is completed within twenty-five hours or less that involves the steps of freezing, primary drying, and secondary drying.

The term “stabilizing polymer” as used herein refers to a polymer that is added to the polymer composition of the dual conjugated nanoparticles of the present invention to provide stability to the nanoparticle structure.

The term “stealth liposome” as used herein refers to a PEG-coated liposome. In some embodiments, the PEG may be physically adsorbed to the polymer on the surface of the vesicles, incorporated within a PEG-lipid conjugate during liposome preparation, or covalently attached to reactive groups on the surface of preformed liposomes. In some embodiments, the PEG may be grafted onto the surface of the liposomes. In some embodiments, the PEG may be detachable from the liposome. The ability of the hydrophilic shell of PEG to avoid aggregation between liposomal particles and to decrease the extent of particle-protein interaction in biological fluids is due not only to the molecular mass of the bound polymer and its uniformity (“molecular cloud”) but also to its considerable conformational flexibility.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

The term “substantia free.” as used herein means measurably free, but not necessarily completely free.

The term “targeting ligand,” as used herein refers to any material or substance that may promote targeting of receptors, cells, and/or tissues in vivo or in vitro with the compositions of the present invention.

The term “taxane drug,” as used herein refers to a drug with antimitotic activity and refers to a class of agents having a mechanism of microtubular action and having a structure that includes a taxane ring. One of the most prominent representatives of this group Paclitaxel. The term should be understood to refer not only to the commonly available Paclitaxel, but also, analogs such as Docetaxel or other taxane derivatives and Paclitaxel conjugates, such as Paclitaxel PEG, Paclitaxel Dextrin, or Paclitaxel xylose or other derivatives that were derivatized from Paclitaxel itself. Suitable examples include, but are not limited to, Paclitaxel, Docetaxel, Rapamycin, Doxorubicin, Daunorubicin, Idarubicin. Epirubicin, Capecitabine, Mitomycin C, Amsacrine, Busulfan, Tretinoin, Etoposide, Chlorambucil, Chlormethine, Melphalan, Gemcitabine, 5-fluorouracil, (5FU), Benzylphenylurea (BPU) compounds, Curcumin, Curcuminoids, Cyclopamine, Aciclovir, Indinavir, Lamivudine, Stavudine, Nevirapine, Ritonavir, Ganciclovir, Saquinavir, Lopinavir, Nelfinavir, Itraconazole, Ketoconazole, Miconazole, Oxiconazole, Sertaconazole, Amphotericin B, Griseofulvin Ciprofloxacin, Moxifloxacin, Ofloxacin, Methoxyfloxacin, Pefloxacin, Norfloxacin, Sparfloxacin, Temafloxacin, Levofloxacin, Lomefloxacin, Cinoxacin. Cloxacillin, Benzylpenicillin, Phenylmethoxypenicillin, Erythromycin, Rifampicin, Rifapentin, Ibuprofen, Indomethacin, Ketoprofen, Naproxen, Oxaprozin, Piroxicam, and Sulinda.

The term “therapeutic agent.” as used herein refers to a species that reduces the extent of a pathology of a disease, such as cancer. Such a compound may, for example, reduce tumor growth and preferably the metastatic potential of cancer. Alternatively, such a compound may reduce tumor vascularity by reducing microvessel size or number or by decreasing the blood vessel density ratio.

As used herein, the term “treat” or “treating” a subject having a disorder refers to subjecting the subject to a regimen, e.g., the administration of a polymer-agent conjugate, particle or composition, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “wetting agent,” as used herein refers to a compound that aids resuspension of a lyophilized preparation. In general, including a wetting agent in a liquid formulation that is to be lyophilized can promote uptake of water by the resulting lyophilized preparation, and promote disintegration of the lyophilized preparation. A variety of suitable wetting agents are known in the art, and include, for example, carbohydrates, such as glucose, sucrose, trehalose, and modified starches (e.g., hydroxyethyl starch and the like); polyols, such as mannitol, sorbitol, polyethylene glycols and derivatives, and the like; amino acids, such as alanine, arginine, threonine, glycine, cysteine, serine, histidine, lysine, valine, asparagine, glutamine, proline, and the like; and surfactants, such as, sorbitan esters, polysorbates, PVP. Preferred wetting agents include disaccharides, such as sucrose, trehalose, lactose, maltose, and derivatives thereof; monosaccharides, such as glucose; polyols, such as mannitol; surfactants such as PVP and polysorbates; and amino acids, such as glycine. Particularly preferred wetting agents are also lyoprotectants. For example, carbohydrates, including disaccharides such as sucrose, trehalose, lactose, maltose are preferred wetting agents.

### Formulations—Liquid and Lyophilized

In one aspect, the present invention provides liquid formulations that comprises a lyoprotectant that comprises an oligosaccharide and a particulate construct. The particulate construct comprises a polymer composition, a therapeutic agent, and a potentiating agent. The therapeutic agent and the potentiating agent may be associated with the polymer composition through covalent or non-covalent interactions. In some embodiments, the liquid formulation comprises a reconstitution reagent, and it a reconstituted preparation.

In one aspect, the present invention provides a lyophilized preparation that comprises a lyoprotectant that comprises an oligosaccharide and a particulate construct. The particulate construct comprises a polymer composition, a therapeutic agent, and a potentiating agent. The therapeutic agent and the potentiating agent may be associated with the polymer composition through covalent or non-covalent interactions.

In another aspect, the present invention provides liquid formulations and lyophilized preparations that comprise a lyoprotectant that comprises an oligosaccharide, a liposome comprising a therapeutic agent, and a potentiating agent. The therapeutic agent and the potentiating agent may be associated with the liposome through covalent or non-covalent interactions, for example, the therapeutic agent can be encapsulated within the lumen of the liposome.

In yet another aspect, the present invention provides liquid formulations and lyophilized preparations that comprise a lyoprotectant that comprises an oligosaccharide, a micelle comprising a therapeutic agent, and a potentiating agent. The therapeutic agent and the potentiating agent may be associated with the liposome through covalent or non-covalent interactions, for example, the therapeutic agent can be encapsulated within the core of the micelle.

The liquid formulations of the present invention generally include a liquid solvent, continuous phase, or dispersion medium. Suitable liquids include any liquid solution compatible with the components of the particulate construct (e.g., nanoparticles, microparticles), liposomes and micelles, and preferably also suitable to be used in pharmaceutical compositions, such as a physiologically acceptable liquid. Suitable physiologically acceptable liquids include, but are not limited to, an aqueous liquid, an organic liquid and mixtures thereof (e.g., alcohol/water mixtures). Examples of suitable aqueous liquids include, but are not limited to, water, sucrose solutions, dextrose solutions, saline solutions, buffered aqueous solutions any derivative thereof, and any combination thereof. Examples of suitable organic liquids include, but are not limited to, acetone, methanol, ethanol, tert-butylmethyl ether, heptane, dichloromethane, dimethylformamide, acetylnitrile, 1-propanol, 1-butanol, formic acid, acetic acid, formamide, tetrahydrofuran, methyl ethyl ketone, ethyl acetate, acetonitrile, dimethylsufoxide, hexane, benzene, methyl ether, carbon tetrachloride, methylene chloride, butyl acetate, propyl acetate, any derivative thereof, and any combination thereof. In certain embodiments, the liquid formulation has an organic solvent:water ratio in the range of from about 1:1 to about 1:10 by volume.

Once lyophilized, the lyophilized preparation of the present invention may comprise about 0.5% physiologically acceptable liquid or less by weight of the lyophilized preparation.

In certain embodiments, the lyophilized preparations may be reconstituted with a reconstitution reagent. In some embodiments, a suitable reconstitution reagent may be any physiologically acceptable liquid. Suitable reconstitution reagents include, but are not limited to, water. 5% Dextrose Injection, Lactated Ringer's and Dextrose Injection, or a mixture of equal parts by volume of Dehydrated Alcohol, USP and a nonionic surfactant, such as a polyoxyethylated castor oil surfactant available from GAF Corporation, Mount Olive. N.J., under the trademark, Cremophor EL. To minimize the amount of surfactant in the reconstituted solution, only a sufficient amount of the vehicle may be provided to form a solution of the lyophilized preparation. Once dissolution of the lyophilized preparation is achieved, the resulting solution may be further diluted prior to injection with a suitable parenteral diluent. Such diluents are well known to those of ordinary skill in the art. These diluents are generally available in clinical facilities. Examples of typical diluents include, but are not limited to, Lactated Ringer's Injection, 5% Dextrose Injection, Sterile Water for Injection, and the like. However, because of its narrow pH range, pH 6.0 to 7.5, Lactated Ringer's Injection is most typical. Per 100 mL, Lactated Ringer's Injection contains Sodium Chloride USP 0.6 g, Sodium Lactate 0.31 g, Potassium chloride USP 0.03 g and Calcium Chloride₂H₂O USP 0.02 g. The osmolarity is 275 m Osmol/L, which is very close to isotonicity. One of ordinary skill in the art with the benefit of this disclosure will recognize the appropriate amount and type of reconstitution reagent and diluent that should be used for a given application.

Accordingly, a liquid formulation can be a resuspended or rehydrated lyophilized preparation in a suitable reconstitution reagent. Suitable reconstitution reagents include physiologically acceptable carriers, e.g., a physiologically acceptable liquids, as described herein. Preferably, resuspension or rehydration of the lyophilized preparations forms a solution or suspension of nanoparticles, micelles, or liposomes which have substantially the same properties (e.g., average particle diameter (Zave), size distribution (Dv₉₀, Dv₅₀), polydispersity, drug concentration) and morphology of the original nanoparticles, micelles, or liposomes in the liquid formulation of the present invention before lyophilization, and further maintains the therapeutic agent to polymer ratio and/or the therapeutic agent to lipid ratio of the original liquid formulation before lyophilization. In certain embodiments, about 50% to about 100%, preferably about 80% to about 100%, of the nanoparticles, micelles, and/or liposomes in the resuspended or rehydrated lyophilized preparation maintain the size distribution and/or drug to lipid ratio of the nanoparticles, micelles, and/or liposomes in the original liquid formulation. Preferably, the Zave, Dv₉₀, and polydispersity of the nanoparticles, micelles, and/or liposomes in the formulation produced by resuspending a lyophilized preparation do not differ from the Zave, Dv₉₀, and polydispersity of the nanoparticles, micelles, and/or liposomes in the original solution or suspension prior to lyophilization by more than about 5%, more than about 10%, more than about 15%, more than about 20%, more than about 15%, more than about 30%, more than about 35%, more than about 40%, more than about 45%, or more than about 50%.

In one aspect, the present invention provides liquid formulations and lyophilized preparations that comprise a lyoprotectant that comprises an oligosaccharide and further comprises a wetting agent. In some embodiments, the liquid formulations also comprise a reconstitution reagent.

The lyoprotectant and the wetting agent can be present in any suitable relative amounts, and preferably, each is independently present in an amount from about 0.5 to 1.5 by weight relative to the particulate construct (e.g., polymer weight), liposome or micelle present in the formulation. Preferably, the ratio of lyoprotectant to wetting agent (w/w) is from about 0.5:1.5 to about 1.5:0.5, and more preferably from 0.7:1.3 to 1.3:0.7.

For clarity, when determining ratios of components of the liquid formulations and lyophilized preparations described herein, the weight of the particulate construct (e.g., polymer weight), liposome or micelle, is set at 1, and the weight of additional components are expressed in proportion to the weight of the particle. Thus, for example, when the ratio of lyoprotectant to wetting agent is 0.5:1.5 (w/w), the formulation contains one-half as much lyoprotectant relative to particulate construct (polymer) by weight, and 50% more wetting agent relative to particulate construct (polymer) by weight.

In particular examples, the ratio of lyoprotectant to wetting agent (w/w) is 0.7:1.3. 1:0.7, 1:1, 1.3:1 or 1.3:0.7. When the liquid or lyophilized formulation comprises a particulate construct (e.g., microparticle and/or nanoparticle) the ratio of lyoprotectant plus wetting agent to polymer (w/w) is from about 1:1 to about 10:1, and preferably, from about 1:1 to about 3:1.

Preferably liquid formulations of this aspect contain a particulate construct, and are characterized by a higher polymer concentration (the concentration of polymer(s) that form the particulate construct) than can be lyophilized and resuspended using either a lyoprotectant that comprises an oligosaccharide or a wetting agent alone. For example, the polymer concentration can be at least about 20 mg/mL, at least about 25 mg/mL, at least about 30 mg/mL, at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 45 mg/mL, at least about 50 mg/mL, at least about 55 mg/mL, at least about 60 mg/mL, at least about 65 mg/mL, at least about 70 mg/mL, at least about 75 mg/mL, at least about 80 mg/mL, at least about 85 mg/mL, at least about 90 mg/mL, at least about 95 mg/mL, are at least about 100 mg/mL. For example, the liquid formulation can be a reconstituted lyophilized preparation.

In preferred embodiments of this aspect, the lyoprotectant comprises a cyclic oligosaccharide, such as an α-cyclodextrin. β-cyclodextrin, γ-cyclodextrin, any derivative thereof, and any combination thereof. In such embodiments, the wetting agent can be any suitable wetting agent as described herein. In more preferred embodiments, the wetting agent is a disaccharide, such as sucrose or trehalose. In particularly preferred embodiments, the lyoprotectant comprises a β-cyclodextrin or derivative thereof, such as 2-hydroxypropyl-β-cyclodextrin, or β-cyclodextrin sulfobutylether; and the wetting agent is a disaccharide, such as sucrose. The β-cyclodextrin or derivative thereof and the wetting agent can be present in any suitable relative amounts. Preferably, the ratio of β-cyclodextrin or derivative thereof to wetting agent (w/w) is from about 0.5:1.5 to about 1.5:0.5, and more preferably from 0.7:1.3 to 1.3:0.7. In particular examples, the ratio of β-cyclodextrin or derivative thereof to wetting agent (w/w) is 0.7:1.3, 1:0.7, 1:1, 1.3:1 or 1.3:0.7. When the liquid or lyophilized formulation comprises a particulate construct (e.g., microparticle and/or nanoparticle) the ratio of β-cyclodextrin or derivative thereof plus wetting agent to polymer (w/w) is from about 1:1 to about 10:1, and preferably, from about 1.1 to about 3:1. Liquid formulations of these preferred embodiments preferably contain a particulate construct, and are characterized by a higher polymer concentration (the concentration of polymer(s) that form the particulate construct) than can be lyophilized and resuspended using either a lyoprotectant that comprises an oligosaccharide or a wetting agent alone. For example, the polymer concentration can be at least about 20 mg/mL, at least about 25 mg/mL, at least about 30 mg/mL, at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 45 mg/mL, at least about 50 mg/mL, at least about 55 mg/mL, at least about 60 mg/mL, at least about 65 mg/mL, at least about 70 mg/mL, at least about 75 mg/mL, at least about 80 mg/mL, at least about 85 mg/mL, at least about 90 mg/mL, at least about 95 mg/mL, are at least about 100 mg/mL. For example, the liquid formulation can be a reconstituted lyophilized preparation.

In some embodiments, the liquid formulation or lyophilized preparation comprises a non-particle component, e.g., a carbohydrate component, or a stabilizer or lyoprotectant, e.g., a carbohydrate component, stabilizer or lyoprotectant described herein. While not wishing to be bound be theory, the carbohydrate component may act as a stabilizer or lyoprotectant. In some embodiments, the carbohydrate component, stabilizer or lyoprotectant, comprises one or more carbohydrates (e.g., one or more carbohydrates described herein, such as, e.g., sucrose, cyclodextrin or a derivative of cyclodextrin (e.g. 2-hydroxypropyl-β-cyclodextrin, sometimes referred to herein as HP-β-CD)), salt, PEG, PVP or crown ether. In some embodiments, the carbohydrate component, stabilizer or lyoprotectant comprises two or more carbohydrates, e.g., two or more carbohydrates described herein. In one embodiment, the carbohydrate component, stabilizer or lyoprotectant includes a cyclic carbohydrate (e.g., cyclodextrin or a derivative of cyclodextrin, e.g., an α-, β-, or γ-, cyclodextrin (e.g. 2-hydroxypropyl-β-cyclodextrin)) and a non-cyclic carbohydrate. Exemplary non-cyclic carbohydrates include non-cyclic oligosaccharides including those of less than 10, 8, 6 or 4 monosaccharide subunits (e.g., a monosaccharide or a disaccharide (e.g., sucrose, trehalose, lactose, maltose) or combinations thereof).

In an embodiment, the carbohydrate component, stabilizer or lyoprotectant comprises a first and a second component, for example, a cyclic carbohydrate and a non-cyclic carbohydrate, e.g., a mono-, di, or tetra saccharide.

In one embodiment, the weight ratio of cyclic carbohydrate to non-cyclic carbohydrate in the composition is a weight ratio described herein, e.g., 0.5:1.5 to 1.5:0.5.

In an embodiment, the carbohydrate component, stabilizer or lyoprotectant comprises a first and a second component (designated here as A and B) as follows:
(A) comprises a cyclic carbohydrate and (B) comprises a disaccharide;
(A) comprises more than one cyclic carbohydrate, e.g., a β-cyclodextrin (sometimes referred to herein as β-CD) or a β-CD derivative, e.g., HP-β-CD, and (B) comprises a disaccharide;
(A) comprises a cyclic carbohydrate, e.g., a β-CD or a β-CD derivative, HP-β-CD, and (B) comprises more than one disaccharide:
(A) comprises more than one cyclic carbohydrate, and (B) comprises more than one disaccharide;
(A) comprises a cyclodextrin, e.g., a β-CD or a β-CD derivative, e.g., HP-β-CD, and (B) comprises a disaccharide;
(A) comprises a β-cyclodextrin, e.g., a β-CD derivative, e.g., HP-β-CD, and (B) comprises a disaccharide;
(A) comprises a β-cyclodextrin. e.g., a β-CD derivative, e.g., HP-β-CD, and (B) comprises sucrose;
(A) comprises a β-CD derivative, e.g., HP-β-CD, and (B) comprises sucrose;
(A) comprises a β-cyclodextrin, e.g., a β-CD derivative, e.g., HP-β-CD, and (B) comprises trehalose;
(A) comprises a β-cyclodextrin, e.g., a β-CD derivative, e.g., HP-β-CD, and (B) comprises sucrose and trehalose.
(A) comprises HP-β-CD, and (B) comprises sucrose and trehalose.

In an embodiment, components A and B are present in the following ratio: 0.5:1.5 to 1.5:0.5. In an embodiment, components A and B are present in the following ratio: 3-1:0.4-2; 3-1:0.4-2.5; 3-1:0.4-2; 3-1:0.5-1.5; 3-1:0.5-1; 3-1:1; 3-1:0.6-0.9; and 3:1:0.7. In an embodiment, components A and B are present in the following ratio: 2-1:0.4-2; 3-1:0.4-2.5; 2-1:0.4-2; 2-1:0.5-1.5; 2-1:0.5-1; 2-1:1; 2-1:0.6-0.9; and 2-1:0.7. In an embodiment, components A and B are present in the following ratio: 2-1.5:0.4-2; 2-1.5:0.4-2.5; 2-1.5:0.4-2; 2-1.5:0.5-1.5; 2-1.5:0.5-1; 2-1.5:1; 2-1.5:0.6-0.9; 2:1.5; 0.7. In an embodiment, components A and B are present in the following ratio; 2.5-1.5:0.5-1.5; 2.2-1.6:0.7-1.3; 2.0-1.7:0.8-1.2; 1.8:1; 1.85:1 and 1.9:1.

In an embodiment, component A comprises a cyclodextin, e.g., a β-cyclodextrin. e.g., a β-CD derivative, e.g., HP-β-CD, and (B) comprises sucrose, and they are present in the following ratio: 2.5-1.5:0.5-1.5; 2.2-1.6:0.7-1.3; 2.0-1.7:0.8-1.2; 1.8:1; 1.85:1 and 1.9:1.

To facilitate a clear and concise description of the formulations, additional details of the components and amounts of components that can be present in a formulation are provided herein. It is intended and will be appreciated by those skilled in the art that the formulations can contain any of the components, such as lyoprotectants, wetting agents, polymers, therapeutic agents, potentiating agents, excipients, diluents and the like, that are disclosed herein, and any combination of the components.

### Lyoprotectants and Wetting Agents

As described and exemplified herein, aggregation of PEGylated small structures (e.g., particulate constructs, liposomes, and micelles) during lyophilization may be reduced or minimized by the use of lyoprotectants comprising an oligosaccharide. Using suitable lyoprotectants provides lyophilized preparations that have extended shelf-lives.

In one aspect, the present invention provides liquid formulations and lyophilized preparations that comprise a lyoprotectant that comprises an oligosaccharide. In some embodiments, the liquid formulations also comprise a reconstitution reagent.

Suitable oligosaccharides include, but are not limited to, linear oligosaccharides and cyclic oligosaccharides. Examples, include, but are not limited to cyclodextrins, raffinose, melezitose, maltotriose, stachyose acarbose, any derivatives thereof, and any combination thereof. Suitable cyclic oligosaccharides for use in the present invention include, but are not limited to, α-cyclodextrins, β-cyclodextrins, such as 2-hydroxypropyl-β-cyclodextrins, β-cyclodextrin sulfobutylethers sodiums, γ-cyclodextrins, any derivative thereof, and any combination thereof.

In certain embodiments, the lyoprotectant may further comprise a polymer. Suitable polymers are disclosed herein with respect to the polymer composition of the particulate construct. In such embodiments, the cyclic oligosaccharide may be incorporated within a backbone of the polymer. See, e.g., U.S. Pat. No. 7,270,808 and U.S. Pat. No. 7,091,192, which disclose exemplary polymers that contain cyclodextrin moieties in the polymer backbone that can be used an lyoprotectants in accordance with the invention. The entire teachings of U.S. Pat. No. 7,270,808 and U.S. Pat. No. 7,091,192 are incorporated herein by reference. In some embodiments, the cyclic oligosaccharide may contain at least one oxidized occurrence.

The lyoprotectant comprising an oligosaccharide, such as a cyclic oligosaccharide, may inhibit the rate of intermolecular aggregation of particulate constructs, liposomes, and micelles that are associated with a potentiating agent (e.g., through a covalent or noncovalent bond) during their lyophilization and/or storage, and therefore, provide for extended shelf-life. Without wishing to be limited by theory, the mechanism for the cyclic oligosaccharide to prevent particle aggregation is likely due to the lyoprotectant reducing or preventing the crystallization of the potentiating agent (e.g. PEG) during lyophilization. This may occur through the formation of an inclusion complex between a cyclic oligosaccharide and the potentiating agent (e.g., PEG). Such a complex may be formed between a cyclodextrin and, for example, the chain of polyethylene glycol. The inside cavity of cyclodextrin is lipophilic, while the outside of the cyclodextrin is hydrophilic. These properties may allow for the formation of inclusion complexes with other components of the formulations in the present invention. For the purpose of stabilizing the formulations during lyophilization, the poly(ethyleneglycol) chain may fit into the cavity of the cyclodextrins. An additional mechanism that may allow the lyoprotectant to reduced or minimized or prevent particle degradation relates to the formation of hydrogen bonds between the cyclic oligosaccharide and the potentiating agent (PEG) during lyophilization. For example, hydrogen bonding between cyclodextrin and poly(ethyleneglycol) chains may prevent ordered polyethylene glycol structures such as crystals.

The lyoprotectant of the present invention may be present in varying amounts in the formulations described herein. In certain embodiments, the lyoprotectant to liquid formulation ratio is in the range of from about 0.75:1 to about 3:1 by weight. In preferred embodiments, the lyoprotectant to total polymer ratio is in the range of from about 0.75:1 to about 3:1 by weight. One of ordinary skill in the art, with the benefit of this disclosure, will recognize the appropriate lyoprotectant and amount of lyoprotectant to use for a chosen application.

In preferred aspects, the formulation contains a lyoprotectant and a wetting agent. As described herein, including a wetting agent in a liquid formulation that is to be lyophilized can promote uptake of water by the resulting lyophilized preparation, and promote disintegration of the lyophilized preparation.

In preferred aspects, the lyoprotectant comprises a cyclic oligosaccharide, such as an α-cyclodextrin. β-cyclodextrin, γ-cyclodextrin, any derivative thereof, and any combination thereof, and the wetting agent can be any suitable wetting agent as described herein. In more preferred embodiments, the lyoprotectant comprises a cyclic oligosaccharide, such as an α-cyclodextrin. β-cyclodextrin, γ-cyclodextrin, any derivative thereof, and any combination thereof, and the wetting agent is a disaccharide, such as sucrose or trehalose. In particularly preferred embodiments, the lyoprotectant comprises a β-cyclodextrin or derivative thereof, such as 2-hydroxypropyl-β-cyclodextrin or β-cyclodextrin sulfobutylether and the wetting agent is a disaccharide, such as sucrose. The β-cyclodextrin or derivative thereof and the wetting agent can be present in any suitable relative amounts. Preferably, the ratio of lyoprotectant to wetting agent (w/w) is from about 0.5:1.5 to about 1.5:0.5, and more preferably from 0.7:1.3 to 1.3:0.7. In particular examples, the ratio of lyoprotectant to wetting agent (w/w) is 0.7:1.3, 1:0.7, 1:1, 1.3:1 or 1.3:0.7. When the liquid or lyophilized formulation comprises a particulate construct (e.g., microparticle and/or nanoparticle) the ratio of lyoprotectant plus wetting agent to polymer (w/w) is from about 1:1 to about 10:1, and preferably, from about 1.1 to about 3:1.

### Particulate Constructs

The particulate constructs of the present invention may be nanoparticles or microparticles that comprise a polymer composition, a therapeutic agent, and a potentiating agent. In some embodiments, the particulate construct may be a dual conjugate nanoparticle or a dual conjugate microparticle, wherein the therapeutic agent is covalently or non-covalently conjugated to the polymer composition, for example, conjugated through a covalent bond or non-covalently associated, and the polymer composition is covalently or non-covalently conjugated to the potentiating agent, for example, conjugated through a covalent bond or non-covalently associated. In certain embodiments, the particulate construct conjugate may comprise a plurality of conjugations to any number of therapeutic agents. The therapeutic agent(s) attached to the particulate construct may be the same or different. The therapeutic agent can be conjugated on a single polymer in the polymer composition or to separate polymers in the polymer composition, or combinations thereof, as desired.

Particulate constructs suitable for use in the present invention comprise a polymer composition, a therapeutic agent, and a potentiating agent, wherein the therapeutic agent and the potentiating agent are associated with the polymer composition. The particulate construct can be of any desired size, but preferably is a nanoparticle or a microparticle. The particulate constructs may have any desired shape or configuration. For example, the particulate construct can be a nanoparticle or microparticle that is substantially spherical, ellipsoidal, or non-spherical in configuration. For example, the nanoparticles and microparticles may adopt a non-spherical configuration upon swelling or shrinkage.

In some embodiments, the particulate construct may be a nanoparticle that has a characteristic dimension of one micrometer or less. The disclosed particulate construct may be a microparticle that has a characteristic dimension of one micrometer or more. The characteristic dimension may be determined by measuring the diameter of a perfect sphere having the same volume as a nanoparticle and/or microparticle.

In certain embodiments, the particulate construct may have a characteristic dimension in the range of about 1 nm to about 200 μm. When the particulate construct is a nanoparticle, the particulate construct may have a characteristic dimension in the range of about 1 nm to about 999 nm, preferable about 50 nm to about 300 nm, more preferably about 50 nm to about 200 nm. In such embodiments, the particulate construct of the present invention may have a Dv₉₀ of less than about 300 nm or less than about 200 nm. One of ordinary skill in the art with the benefit of this disclosure will recognize the appropriate size for a particulate construct for a chosen application.

### Polymer Composition

The liquid formulations and lyophilized preparations of the present invention may comprise a particulate construct comprising a polymer composition. The polymer composition may comprise a polymer composition, a hydrophilic polymer, or any combination thereof. In some embodiments, the polymer composition may comprise a copolymer that comprises both hydrophobic and hydrophilic blocks. Preferably, the polymer composition comprises a biodegradable polymer. In certain embodiments, the particulate constructs and polymer compositions biodegrade within a period that is acceptable in the desired application. For example, when the particulate constructs are intended for in vivo therapy, such degradation preferably occurs in less than about five years, less than about one year, less than about six months, less than about three months, less than about one month, less than about fifteen days, less than about five days, less than about three days, or even less than about one day upon exposure to In vivo environment. If desired, stability and/or degradation can be evaluated by exposing a particulate construct to a physiological solution with a pH between 4 and 8 having a temperature of between 25° C. and 37° C. In other embodiments, the polymer degrades in a period in the range of about one hour and several weeks, depending on the desired application.

A wide variety of suitable polymer compositions and suitable methods for forming therapeutic particulate constructs are known in the art. The polymer compositions used in the particulate constructs may be homopolymers or copolymers containing two or more monomers, including block copolymers, stereoblock copolymers, alternating copolymers, periodic copolymers, dendritic copolymers, and grafted polymers. Suitable polymer compositions may be linear or branched, and include dendrimers, dendronized polymers, and brush polymers. Examples of suitable polymer compositions, include, but are not limited to, polyesters, polysaccharides, polyamides, polyethers, polycarbonates, acrylates, polylactic acids, polyglycolic acids, polydioxanones, poly-(lactide-co-glycolides), polyethylenimines, copolymers of methoxy-poly(ethylene glycol)-block-poly(lactide-co-glycolides), chitins, chitosans, poly(glycolides), poly(ε-caprolactones), poly(hydroxy ester ethers), poly(hydroxybutyrates), poly(anhydrides), poly(orthoesters), poly(amino acids), poly(ethylene oxides), poly(phosphazenes), polyetheresters, polyester amides, vinyls, styrenes, any derivatives, any copolymers, any blends, and any combinations thereof. Examples of polymer compositions may comprise an acrylate group, and may include, but are not limited to, polymers that include methyl acrylate, ethyl acrylate, propyl acrylate, n-butyl acrylate (BA), isobutyl acrylate, 2-ethyl acrylate, and t-butyl acrylate, methacrylates, ethyl methacrylate, n-butyl methacrylate, and isobutyl methacrylate, acrylonitriles, methacrylonitrile. Examples of polymer compositions comprising vinyls, include, but are not limited to, polymers that include vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides. In certain embodiments, the polymer composition may be a polyester synthesized from monomers selected from the group consisting of D,L-lactide, D-lactide. L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone. δ-hydroxy valeric acid, hydroxybutyric acids, malic acid and any combination thereof. A copolymer may also be used in the particulate construct described herein.

In some embodiments, the polymer composition may be poly(lactic-co-glycolic acid) (PLGA), which is a biodegradable random copolymer of lactic acid and glycolic acid. A PLGA polymer may have varying ratios of lactic acid:glycolic acid, e.g., ranging from about 0.1:99.9 to about 99.9:0.1 (e.g., from about 75:25 to about 25:75, from about 60:40 to 40:60, or about 55:45 to 45:55). In some embodiments, e.g., in PLGA, the ratio of lactic acid monomers to glycolic acid monomers may be in a range from about 50:50 to about 75:25. If desired, the ratio of lactic acid to glycolic acid monomers in the PLGA polymer of the particulate construct may be optimized to form particles with desired characteristics. Particle characteristics that may be effected by this optimization, include, but are not limited to, water uptake, agent release (e.g., “controlled release”) and polymer degradation kinetics. Furthermore, tuning the ratio will also affect the hydrophobicity of the copolymer, which may in turn affect drug loading.

Since the polymer compositions may be used for delivery of therapeutic agents in vivo, it is important that the polymers themselves be nontoxic under the conditions of use so as to provide a therapeutic window, and that they degrade into non-toxic degradation products as the polymer is eroded by the body fluids. Unfortunately, many synthetic biodegradable polymers, however, yield oligomers and monomers upon erosion in vivo that adversely interact with the surrounding tissue. To minimize the toxicity of an intact polymer carrier and its degradation products, polymers have been designed based on naturally occurring metabolites. Exemplary biodegradable polymers include polyesters derived from lactic and/or glycolic acid and polyamides derived from amino acids.

A number of biodegradable polymers are known and used for controlled release of therapeutic agents. Such polymer compositions are described in, for example, U.S. Pat. Nos. 4,291,013; 4,347,234; 4,525,495; 4,570,629; 4,572,832; 4,587,268; 4,638,045; 4,675,381; 4,745,160; and 5,219,980; and PCT publication WO2006/014626, each of which is hereby incorporated by reference in its entirety. In some embodiments, disclosed polymers release therapeutic agents when placed in a liquid. The rate of release for the therapeutic agent depends on the type of biodegradable polymer used, the type of therapeutic agent used, and the type and temperature of the liquid. In certain embodiments, at least about 75% of the therapeutic agent may be released within 24 hours of contact with a liquid. One of ordinary skill in the art with the benefit of this disclosure will recognize the type of biodegradable polymer to use to optimize the control release of the therapeutic agents for a chosen application.

The polymer composition described herein may have a variety of end groups. In some embodiments, the end group of the polymer is not further modified, e.g., when the end group is a carboxylic acid, a hydroxy group or an amino group. In some embodiments, the end group may be further modified. For example, a polymer with a hydroxyl end group may be derivatized with an acyl group to yield an acyl-capped polymer (e.g., an acetyl-capped polymer or a benzoyl capped polymer), an alkyl group to yield an alkoxy-capped polymer (e.g., a methoxy-capped polymer), or a benzyl group to yield a benzyl-capped polymer.

In certain embodiments, the polymer composition may comprise hydrophilic polymers. Examples of suitable hydrophilic polymers include, but are not limited to, carboxylic acids including acrylic acid, methacrylic acid, itaconic acid, maleic acids, polyoxyethylenes, polyethylene oxides, polyacrylamides, dimethylaminoethylmethacrylates, diallyldimethylammonium chlorides, vinylbenzylthrimethylammonium chlorides, acrylic acids, methacrylic acids, 2-acrylamido-2-methylpropane sulfonic acids, styrene sulfonates, poly(vinylpyrrolidones), starches, dextrans, polypeptides, polylysines, polyarginines, polyglutamic acids, polyhyaluronic acids, alginic acids, polylactides, polyethyleneimines, polyionenes, polyacrylic acids, polyiminocarboxylates, gelatins, unsaturated ethylenic mono or dicarboxylic acids, any copolymers, any derivatives, and any combinations thereof. A listing of suitable hydrophilic polymers may also be found in Handbook of Water-Soluble Gums and Resins, R. Davidson, McGraw-Hill (1980).

In certain embodiments, the polymer compositions described herein may include a polymer containing a hydrophilic portion and a hydrophobic portion. A polymer containing a hydrophilic portion and a hydrophobic portion may be a copolymer of a hydrophilic block coupled with a hydrophobic block. These copolymers may have a molecular weight in the range of about 5 kDa and about 30 kDa. A polymer composition containing a hydrophilic portion and a hydrophobic portion may be a block copolymer, e.g., a diblock or triblock copolymer. In some embodiments, the polymer may be a diblock copolymer containing a hydrophilic block and a hydrophobic block. In some embodiments, the polymer may be a triblock copolymer containing a hydrophobic block, a hydrophilic block and another hydrophobic block. The two hydrophobic blocks may be the same polymer composition or different polymer compositions. The block copolymers used herein may have varying ratios of the hydrophilic portion to the hydrophobic portion, e.g., ranging from about 1:1 to about 1:40 by weight (e.g., about 1:1 to about 1:10 by weight, about 1:1 to about 1:2 by weight, or about 1:3 to about 1:6 by weight).

A polymer composition containing a hydrophilic portion and a hydrophobic portion, and may have a variety of end groups, provided that the polymer as a whole is hydrophobic. In some embodiments, the end group may be a hydroxy group or an alkoxy group. In some embodiments, the end group of the polymer is not further modified. In some embodiments, the end group may be further modified. For example, the end group may be capped with an alkyl group, to yield an alkoxy-capped polymer (e.g., a methoxy-capped polymer), or may be derivatized with a targeting agent (e.g., folate) or a dye (e.g., rhodamine). A polymer containing a hydrophilic portion and a hydrophobic portion may include a linker between the hydrophilic portion and the hydrophobic portion (e.g., between blocks of the copolymer). Any suitable linker can be used, such as an amide, ester, ether, amino, carbamate or carbonate linkage, for example.

In certain embodiments, the polymer composition may have a molecular weight in the range from about 1 kDa to about 21 kDa. The molecular weight of the polymer composition in the particulate construct can be substantially homogenous or can vary. One of ordinary skill in the art with the benefit of this disclosure will recognize the appropriate size or size range for a given application.

The polymer composition described herein may have a polymer polydispersity index (PDI) of less than or equal to about 2.5. In some embodiments, this PDI may vary. One of ordinary skill in the art with the benefit of this disclosure will recognize the appropriate PDI for a given polymer. The polymer compositions of the present invention may be further purified prior to conjugation of other components onto the polymer or incorporation into a particulate composition (e.g., nanoparticle) described herein. In some embodiments, purification may reduce the polydispersity of the polymer sample. A polymer may be purified by precipitation from solution, or precipitation onto a solid such as Celite. A polymer may also be further purified by size exclusion chromatography (SEC).

In certain embodiments, the polymer composition may be present in amount in the range of about 40% to about 90% by weight of the nanoparticle or microparticle. A person of ordinary skill in the art, with the benefit of this disclosure, will recognize the necessary amount of polymer composition to include in a particular application of the present invention depending on, among other factors, the other components of the nanoparticles and/or microparticles, the desired properties of the nanoparticle and/or microparticle in the liquid formulation or lyophilized preparation, and the like.

The polymer composition may be commercially available, or may be synthesized. Methods of synthesizing polymers are known in the art. Such methods include, but are not limited to, polycondensation, radical polymerization, ionic polymerization (e.g., cationic or anionic polymerization), or ring-opening metathesis polymerization. A block copolymer may be prepared by synthesizing the two polymer units separately and then conjugating the two portions using established methods. For example, the blocks may be linked using a coupling agent such as EDC (1-ethyl-3-(3-dimethylaminopropyl)carbodiimide hydrochloride). Following conjugation, the two blocks may be linked via an amide, ester, ether, amino, carbamate or carbonate linkage.

### Therapeutic Agents

The therapeutic agent in the particulate construct of the present invention may comprise small molecules, organometallic compounds, nucleic acids, proteins, peptides, metals, isotopically labeled chemical compounds, drugs, vaccines, immunological agents, and the like. Preferably, the therapeutic agent is a compound with pharmacologic activity, such as a drug or active pharmaceutical ingredient. In certain embodiments, the therapeutic agent may be a taxane drug. In some embodiments, the therapeutic agent may be approved by the U.S. Food and Drug Administration for use in humans or other animals.

In some embodiments, the therapeutic agent may be an anti-cancer agent, an anti-inflammatory agent, a thrombolytic agent, a cardiovascular agent, an anti-anginal agent, a diuretic agent, an antihistamine agent, a hormone-containing agent, an antibiotic agent, a pain reliever agent, an anti-diarrheal agent, an antiemetic, an anti-metabolite agent, an immunomodulator agent, a radiation agent, a chemotherapy agent, any derivative thereof, and any combination thereof.

In embodiments where the therapeutic agent is an anti-cancer drug, suitable classes of chemotheraupeutic drugs for use with the present invention may include the following therapeutic agents: alkylating agents (including, without limitation, nitrogen mustards, ethylenimine derivatives, alkyl sulfonates, nitrosoureas, and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

Other suitable drugs for use in the present invention include, but are not limited to, anti-EGFR antibodies (e.g., cetuximab (Erbitux®), panitumumab (Vectibix®), and gefitinib (Iressa®)), anti-Her-2 antibodies (e.g., trastuzumab (Herceptin®) and other antibodies from Genentech), antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®), vinca alkaloids: vinblastine (Velban®, Velsar®), vincristine (Vincasar®, Oncovin®), vindesine (Eldisine®), vinorelbine (Navelbine®), platinum-based agents: carboplatin (Paraplat®, Paraplatin®), cisplatin (Platinol®), oxaliplatin (Eloxatin®), anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®), topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101), taxanes: paclitaxel (Taxol®), docetaxel (Taxotere®), larotaxel, cabazitaxel, antibiotics: actinomycin (Cosmegen®), bleomycin (Blenoxane®), hydroxyurea (Droxia®, Hydrea®), mitomycin (Mitozytrex®, Mutamycin®), immunomodulators: lenalidomide (Revlimid®), thalidomide (Thalomid®), immune cell antibodies: alemtuzamab (Campath®), gemtuzumab (Myelotarg®), rituximab (Rituxan®), tositumomab (Bexxar®), proteosome inhibitors: bortezomib (Velcade®), interferons (e.g., IFN-alpha (Alferon®, Roferon-A®, Intron®-A) or IFN-gamma (Actimmune®)), interleukins: IL-1, IL-2 (Proleukin®), IL-24, IL-6 (Sigosix®), IL-12, HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”), anti-androgens which include, without limitation nilutamide (Nilandron®) and bicalutamide (Caxodex®), antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride, anti-hypercalcaemia agents which include without limitation gallium (III) nitrate hydrate (Ganite®) and pamidronate disodium (Aredia®), apoptosis inducers which include without limitation ethanol, 2-[[3-(2,3-dichlorophenoxy)propyl]amino]-(9Cl), gambogic acid, embelin and arsenic trioxide (Trisenox®), Aurora kinase inhibitors which include without limitation binucleine 2, Bruton's tyrosine kinase inhibitors which include without limitation terreic acid, calcineurin inhibitors which include without limitation cypermethrin, deltamethrin, fenvalerate and tyrphostin 8, CaM kinase II inhibitors which include without limitation 5-Isoquinolinesulfonic acid, 4-[(2S)-2-[(5-isoquinolinylsulfonyl)methylamino]-3-oxo-3-{(4-phenyl-1-piperazinyl)propyl]phenyl ester and benzenesulfonamide, CD45 tyrosine phosphatase inhibitors which include without limitation phosphonic acid, CDC25 phosphatase inhibitors which include without limitation 1,4-naphthalene dione, 2,3-bis[(2-hydroxyethyl)thio]-(9Cl), CHK kinase inhibitors which include without limitation debromohymenialdisine, cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid), cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl), cyclin dependent kinase inhibitors which include without limitation olomoucine and its derivatives, purvalanol B, roascovitine (Seliciclib®), indirubin, kenpaullone, purvalanol A and indirubin-3′-monooxime, cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl), DNA intercalators which include without limitation plicamycin (Mithracin®) and daptomycin (Cubicin®). DNA strand breakers which include without limitation bleomycin (Blenoxane®), E3 ligase inhibitors which include without limitation N-((3,3,3-trifluoro-2-trifluoromethyl)propionyl)sulfanilamide, EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980, farnesyltransferase inhibitors, which include without limitation, A-hydroxyfarnesylphosphonic acid, butanoic acid, 2-[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-1-(2S)-(9Cl), and manumycin A, Elk-1 kinase inhibitors, which include without limitation. 2-propenamide, 2-cyano-3-[4-hydroxy-3,5-bis(1-methylethyl)phenyl]-N-(3-phenylpropyl)-(2E)-(9Cl), glycogen synthase kinase-3 (GSK3) inhibitors which include without limitation indirubin-3′-monooxime, histone deacetylase (HDAC) inhibitors, which include without limitation, suberoylanilide hydroxamic acid (SAHA), [4-(2-amino-phenylcarbamoyl)-benzyl]-carbamic acid pyridine-3-ylmethylester, butyric acid, pyroxamide, trichostatin A, oxamflatin, apicidin, depsipeptide, depudecin, trapoxin and compounds disclosed in WO 02/22577, I-kappa B-alpha kinase inhibitors (IKK), which include without limitation, 2-propenenitrile, 3-[(4-methylphenyl)sulfonyl]-(2E)-(9Cl), imidazotetrazinones, which include without limitation, temozolomide (Methazolastone®, Temodar® and its derivatives (e.g., as disclosed generically and specifically in U.S. Pat. No. 5,260,291) and Mitozolomide, insulin tyrosine kinase inhibitors, which include without limitation, hydroxyl-2-naphthalenylmethylphosphonic acid, c-Jun-N-terminal kinase (JNK) inhibitors, which include without limitation, pyrazoleanthrone and epigallocatechin gallate, mitogen-activated protein kinase (MAP) inhibitors, which include without limitation, benzenesulfonamide, N-[2-[[[3-(4-chlorophenyl)-2-propenyl]methyl]amino]methyl]phenyl]-N-(2-hydroxyethyl)-4-methoxy-(9Cl), MDM2 inhibitors, which include without limitation, trans-4-iodo, 4′-boranyl-chalcone, MEK inhibitors, which include without limitation, butanedinitrile, bis[amino[2-aminophenyl)thio]methylene]-(9Cl), MMP inhibitors, which include without limitation, Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996, mTor inhibitors, which include without limitation, rapamycin (Rapamune®), and analogs and derivatives thereof, AP23573 (also known as ridaforolimus, deforolimus, or MK-8669), CCI-779 (also known as temsirolimus) (Torisel®) and SDZ-RAD, NGFR tyrosine kinase inhibitors, which include without limitation, tyrphostin AG 879, p38 MAP kinase inhibitors, which include without limitation, Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl), p56 tyrosine kinase inhibitors, which include without limitation, damnacanthal and tyrphostin 46. PDGF pathway inhibitors, which include without limitation, tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854, phosphatidylinositol 3-kinase inhibitors, which include without limitation, wortmannin, and quercetin dehydrate, phosphatase inhibitors, which include without limitation, cantharidic acid, cantharidin, and L-leucinamide, protein phosphatase inhibitors, which include without limitation, cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid, PKC inhibitors, which include without limitation, 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin, PKC delta kinase inhibitors, which include without limitation, rottlerin, polyamine synthesis inhibitors which include without limitation DMFO, proteasome inhibitors which include, without limitation aclacinomycin A, gliotoxin and bortezomib (Velcade®), PTP1B inhibitors, which include without limitation, L-leucinamide, protein tyrosine kinase inhibitors, which include, without limitation, tyrphostin Ag 216, tyrphostin Ag 1288, tyrphostin Ag 1295, geldanamycin, genistein, 7H-pyrollo[2,3-d]pyrimidine derivatives, SRC family tyrosine kinase inhibitors, which include without limitation, PP1 and PP2, Syk tyrosine kinase inhibitors, which include without limitation, piceatannol, Janus (JAK-2 and/or JAK-3) tyrosine kinase inhibitors, which include without limitation, tyrphostin AG 490 and 2-naphthyl vinyl ketone Retinoids, which include without limitation, isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®), RNA polymerase II elongation inhibitors, which include without limitation, 5,6-dichloro-1-beta-D-ribofuranosylbenzimidazole, serine/threonine kinase inhibitors, which include without limitation, 2-aminopurine, sterol biosynthesis inhibitors, which include without limitation, squalene epoxidase and CYP2D6. VEGF pathway inhibitors, which include without limitation, anti-VEGF antibodies, e.g., bevacizumab, and small molecules, e.g. sunitinib (Sutent®), soratinib (Nexavar®), ZD6474 (also known as vandetanib) (Zactima™), SU6668, CP-547632 and AZD2171 (also known as cediranib) (Recentin™), and any derivatives, and any combination thereof.

In some embodiments, the therapeutic agent may be an anti-inflammatory/autoimmune agent including, but not limited to, steroids, nonsteroidal anti-inflammatory drugs (NSAIDs), PDE4 inhibitors, antihistamines, COX-2 inhibitors, and any derivatives, and any combination thereof. Exemplary anti-inflammatory/autoimmune agents include [alpha]-bisabolol, 1-naphthyl salicylate, 2-amino-4-picoline. 3-amino-4-hydroxybutyric acid, 5-bromosalicylic acid acetate, 5′-nitro-2′-propoxyacetanilide, 6[alpha]-methylprednisone, aceclofenac, acemetacin, acetaminophen, acetaminosalol, acetanilide, acetylsalicylic acid, alclofenac, alclometasone, alfentanil, algestone, allylprodine, alminoprofen, aloxiprin, alphaprodine, aluminum bis(acetylsalicylate), amcinonide, amfenac, aminochlorthenoxazin, aminopropylon, aminopyrine, amixetrine, ammonium salicylate, ampiroxicam, amtolmetin guacil, anileridine, antipyrine, antrafenine, apazone, artemether, artemisinin, artsunate, aspirin, atovaquone, beclomethasone, bendazac, benorylate, benoxaprofen, benzpiperylon, benzydamine, benzylmorphine, bermoprofen, betamethasone, betamethasone-17-valerate, bezitramide, bromfenac, bromosaligenin, bucetin, bucloxic acid, bucolome, budesonide, bufexamac, bumadizon, buprenorphine, butacetin, butibufen, butorphanol, caiprofen, carbamazepine, carbiphene, carsalam, celecoxib, chlorobutanol, chloroprednisone, chloroquine phosphate, chlorthenoxazin, choline salicylate, cinchophen, cinmetacin, ciramadol, clidanac, clobetasol, clocortolone, clometacin, clonitazene, clonixin, clopirac, cloprednol, clove, codeine, codeine methyl bromide, codeine phosphate, codeine sulfate, cortisol, cortisone, cortivazol, cropropamide, crotethamide, cyclazocine, cyclizine, deflazacort, dehydrotestosterone, deoxycorticosterone, deracoxib, desomorphine, desonide, desoximetasone, dexamethasone, dexamethasone-21-isonicotinate, dexoxadrol, dextromoramide, dextropropoxyphene, dezocine, diamorphone, diampromide, diclofenac, difenamizole, difenpiramide, diflorasone, diflucortolone, diflunisal, difluprednate, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, diphenhydramine, dipipanone, diprocetyl, dipyrone, ditazol, doxycycline hyclate, drotrecogin alfa, droxicam, e-acetamidocaproic acid, emorfazone, enfenamic acid, enoxolone, epirizole, eptazocine, etersalate, ethenzamide, ethoheptazine, ethoxazene, ethylmethylthiambutene, ethylmorphine, etodolac, etofenamate, etonitazene, etoricoxib, eugenol, felbinac, fenbufen, fenclozic acid, fendosal, fenoprofen, fentanyl, fentiazac, fepradinol, feprazone, floctafenine, fluazacort, flucloronide, fludrocortisone, flufenamic acid, flumethasone, flunisolide, flunixin, flunoxaprofen, fluocinolone acetonide, fluocinonide, fluocoitolone, fluocortin butyl, fluoresone, fluorometholone, fluperolone, flupirtine, fluprednidene, fluprednisolone, fluproquazone, flurandrenolide, flurbiprofen, fluticasone, formocortal, fosfosal, gentisic acid, glafenine, glucametacin, glycol salicylate, guaiazulene, halcinonide, halobetasol, halofantrine, halometasone, haloprednone, heroin, hydro cortamate, hydrocodone, hydrocortisone, hydrocortisone 21-lysinate, hydrocortisone acetate, hydrocortisone cypionate, hydrocortisone hemisuccinate, hydrocortisone succinate, hydromorphone, hydroxypethidine, hydroxyzine, ibufenac, ibuprofen, ibuproxam, imidazole salicylate, indomethacin, indoprofen, isofezolac, isoflupredone, isoflupredone acetate, isoladol, isomethadone, isonixin, isoxepac, isoxicam, ketobemidone, ketoprofen, ketorolac, lefetamine, levallorphan, levophenacyl-morphan, levorphanol, lofentanil, lonazolac, lornoxicam, loxoprofen, lumiracoxib, lysine acetylsalicylate, mazipredone, meclofenamic acid, medrysone, mefenamic acid, mefloquine hydrochloride, meloxicam, meperidine, meprednisone, meptazinol, mesalamine, metazocine, methadone, methotrimeprazine, methylprednisolone, methylprednisolone acetate, methylprednisolone sodium succinate, methylprednisolone suleptnate, metiazinic acid, metofoline, metopon, mofebutazone, mofezolac, mometasone, morazone, morphine, morphine hydrochloride, morphine sulfate, morpholine salicylate, myrophine, nabumetone, nalbuphine, nalorphine, naproxen, narceine, nefopam, nicomorphine, nifenazone, niflumic acid, nimesulide, norlevorphanol, normethadone, normorphine, norpipanone, olsalazine, opium, oxaceprol, oxametacine, oxaprozin, oxycodone, oxymorphone and oxyphenbutazone, p-lactophenetide, papaveretum, paramethasone, paranyline, parecoxib, parsalmide, p-bromoacetanilide, pentazocine, perisoxal, phenacetin, phenadoxone, phenazocine, phenazopyridine hydrochloride, phenocoll, phenomorphan, phenoperidine, phenopyrazone, phenyl acetylsalicylate, phenyl salicylate, phenylbutazone, phenyramidol, piketoprofen, piminodine, pipebuzone, piperylone, pirazolac, piritramide, piroxicam, pirprofen, pranoprofen, prednicarbate, prednisolone, prednisone, prednival, prednylidene, proglumetacin, proguanil hydrochloride, proheptazine, promedol, promethazine, propacetamol, properidine, propiram, propoxyphene, propyphenazone, proquazone, protizinic acid, proxazole, ramifenazone, remifentanil, rimazolium metilsulfate, rofecoxib, roflumilast, rolipram, S-adenosylmethionine, salacetamide, salicin, salicylamide, salicylamide O-acetic acid, salicylic acid, salicylsulfuric acid, salsalate, salverine, simetride, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tixocortol, tolfenamic acid, tolmetin, tramadol, triamcinolone, triamcinolone acetonide, tropesin, valdecoxib, viminol, xenbucin, ximoprofen, zaltoprofen, and zomepirac.

In some embodiments, the therapeutic agent is an agent for the treatment of cardiovascular disease. Suitable therapeutic agents for the treatment of cardiovascular disease may include, but are not limited to, α-receptor blocking drugs, β-adrenaline receptor blocking drugs, AMPA antagonists, angiotensin converting enzyme inhibitors, angiotensin II antagonists, animal salivary gland plasminogen activators, anti-anginal agents, anti-arrhythmic agents, anti-hyperlipidemic drugs, anti-hypertensive agents, anti-platelet drugs, calcium antagonists, calcium channel blocking agents, cardioglycosides, cardioplegic solutions, cardiotonic agents, catecholamine formulations, cerebral protecting drugs, cyclooxygenase inhibitors, digitalis formulations, diuretics (e.g., a K+ sparing diuretic, loop diuretic, nonthiazide diuretic, osmotic diuretic, or thiazide diuretic), endothelin receptor blocking drugs, fibrinogen antagonists, fibrinolytic agents, GABA agonists, glutamate antagonists, growth factors, heparins, K+ channel opening drugs, kainate antagonists, naturiuretic agents, nitrate drugs, nitric oxide donors, NMDA antagonists, nonsteroidal anti-inflammatory drugs, opioid antagonists, PDE III inhibitors, phosphatidylcholine precursors, phosphodiesterase inhibitors, platelet aggregation inhibitors, potassium channel blocking agents, prostacyclin derivatives, sclerosing solutions, sedatives, serotonin agonists, sodium channel blocking agents, statin, sympathetic nerve inhibitors, thrombolytic agents, thromboxane receptor antagonists, tissue-type plasminogen activators, vasoconstrictor agents, vasodilator agent, xanthine formulation, acebutolol, adenosine, alacepril, alprenolol, alteplase, amantadine, amiloride, amiodarone, amlodipine, amosulalol, anisoylated plasminogen streptokinase activator complex, aranidipine, argatroban, arotinolol, artilide, aspirin, atenolol, azimilide, bamidipine, batroxobin, befunolol, benazepril, bencyclane, bendrofluazide, bendroflumethiazide, benidipine, benzthiazide, bepridil, beraprost sodium, betaxolol, bevantolol, bisoprolol, bopindolol, bosentan, bretylium, bucumolol, buferalol, bumetanide, bunitrolol, buprandolol, butofilolol, butylidine, candesartan, captopril, carazolol, carteolol, carvedilol, celiprolol, ceronapril, cetamolol, chlorothiazide, chlorthalidone, cilazapril, cilnidipine, cilostazol, cinnarizine, citicoline, clentiazem, clofilium, clopidogrel, cloranolol, cyclandelate, cyclonicate, dalteparin calcium, dalteparin sodium, danaparoid sodium, delapril, diazepam, digitalis, digitoxin, digoxin, dilazep hydrochloride, dilevalol, diltiazem, dipyridamole, disopyramide, dofetilide, dronedarone, ebumamonine, edaravone, efonidipine, elgodipine, Eminase, enalapril, encainide, enoxaparin, eprosartan, ersentilide, esmolol, etafenone, ethacrynic acid, ethyl icosapentate, felodipine, flunarizine, flecainide, flumethiazide, flunarizine, flurazepam, fosinopril, furosemide, galopamil, gamma-aminobutyric acid, glyceryl trinitrate, heparin calcium, heparin potassium, heparin sodium, hydralazine, hydrochlorothiazide, hydroflumethiazide, ibudilast, ibutilide, ifenprodil, ifetroban, iloprost, imidapril, indenolol, indobufene, indomethacin, irbesartan, isobutilide, isosorbide nitrate, isradipine, labetalol, lacidipine, lercanidipine, lidocaine, lidoflazine, lignocaine, lisinopril, lomerizine, losartan, magnesium ions, manidipine, methylchlorthiazide, metoprolol, mexiletine, mibefradil, mobertpril, monteplase, moricizine, musolimine, nadolol, naphlole, nasaruplase, nateplase, nicardipine, nickel chloride, nicorandil, nifedipine, nikamate, nilvadipine, nimodipine, nipradilol, nisoldipine, nitrazepam, nitrendipine, nitroglycerin, nofedoline and nosergoline, pamiteplase, papaverine, parnaparin sodium, penbutolol, pentaerythritol tetranitrate, pentifylline, pentopril, pentoxifylline, perhexiline, perindopril, phendilin, phenoxezyl, phenyloin, pindolol, polythiazide, prenylamine, procainaltide, procainamide, propafenone, propranolol, prostaglandin 12, prostaglandin E1, prourokinase, quinapril, quinidine, ramipril, randolapril, rateplase, recombinant tPA, reviparin sodium, sarpogrelate hydrochloride, semotiadil, sodium citrate, sotalol, spirapril, spironolactone, streptokinase, tedisamil, temocapril, terodiline, tiapride, ticlopidene, ticrynafen, tilisolol, timolol, tisokinase, tissue plasminogen activator (tPA), tocainide, trandolapril, trapidil, trecetilide, triamterene, trichloromethiazide, urokinase, valsartan, verapamil, vichizyl, vincamin, vinpocetine, vitamin C, vitamin E, warfarin, zofenopril, any derivatives thereof, and any combinations thereof.

In some embodiments, the therapeutic agent is a derivative of a compound with pharmaceutical activity, such as an acetylated derivative or a pharmaceutically acceptable salt. In some embodiments, the agent is a pro-drug such as a hexanoate conjugate. A therapeutic agent may also include a combination of agents that have been combined and attached to a polymer and/or loaded into the particle. Any combination of therapeutic agents may be used. For example, therapeutic agents may be combined with diagnostic agents, therapeutic agents may be combined with prophylactic agents, therapeutic agents may be combined with one or more other therapeutic agents, diagnostic agents may be combined with prophylactic agents, diagnostic agents may be combined with other diagnostic agents, and prophylactic agents may be combined with other prophylactic agents. In certain embodiments for treating cancer, at least two traditional chemotherapeutic agents are attached to a polymer and/or loaded into the nanoparticle and/or microparticle.

In some embodiments, the therapeutic agent may be doxorubicin and may be covalently attached to the first polymer through an amide bond.

In some embodiments, the therapeutic agent may be conjugated to the particulate construct, e.g., the nanoparticle as shown in Structure 1 below:

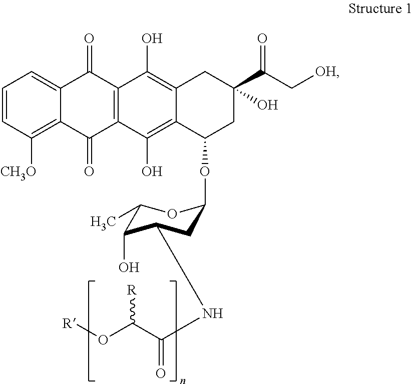

wherein about 30% to about 70%, 35% to about 65%. 40% to about 60%, 45% to about 55% of R substituents are hydrogen (e.g. about 50%) and about 30% to about 70%, 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the therapeutic agent may be paclitaxel and may be covalently attached to the particulate construct through an ester bond. In some embodiments, the agent may be paclitaxel and may be attached to the particulate construct via the hydroxyl group at the 2′ position.

In some embodiments, the therapeutic agent may be conjugated to the particulate construct to form Structure 2 below:

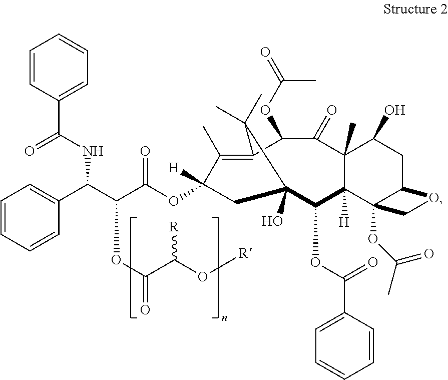

wherein about 30% to about 70%, about 35% to about 65%, about 40% to about 60%, about 45% to about 55% of R substituents are hydrogen (e.g., about 50%) and about 30% to about 70%, about 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent may be paclitaxel and may be attached to the polymer via the hydroxyl group at the 7 position.

In some embodiments, the therapeutic agent may be conjugated to the particulate construct to form Structure 3 below:

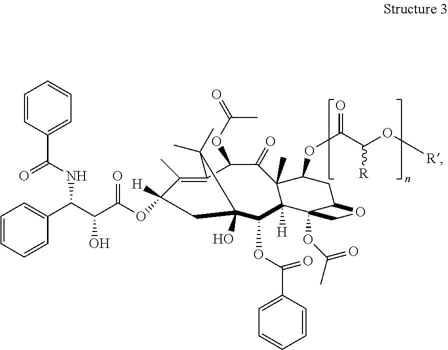

wherein about 30% to about 70%, about 35% to about 65%, about 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the particulate construct includes a combination of polymer composition-paclitaxel conjugates described herein, e.g., the polymer composition-paclitaxel conjugates illustrated above.

In some embodiments, the therapeutic agent may be conjugated to the particulate construct to form the nanoparticle as shown in Formula I below:

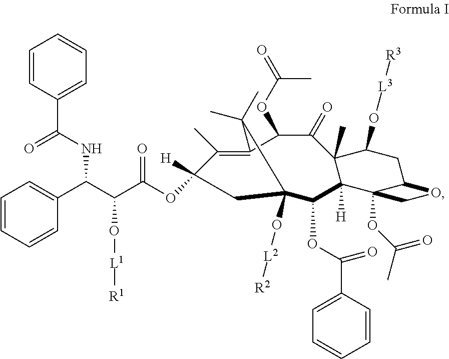

wherein L¹, L² and L³ are each independently a bond or a linker, e.g., a linker described herein; wherein R¹, R² and R³ are each independently hydrogen, C₁-C₆ alkyl, acyl, or a polymer composition of Formula II

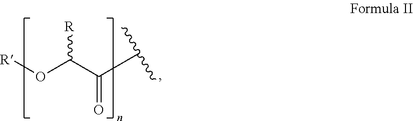

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and wherein at least one of R¹, R² and R³ is a polymer of formula (II). In some embodiments, L² is a bond and R² is hydrogen.

In some embodiments, the therapeutic agent may be paclitaxel and may be covalently attached to the polymer via a carbonate bond.

In some embodiments, the therapeutic agent may be docetaxel and may be covalently attached to the polymer through an ester bond. In some embodiments, the therapeutic agent may be docetaxel and may be attached to the polymer via the hydroxyl group at the 2′ position.

In some embodiments, the therapeutic agent may be conjugated to the particulate construct to form the structure as shown in Structure 4 below:

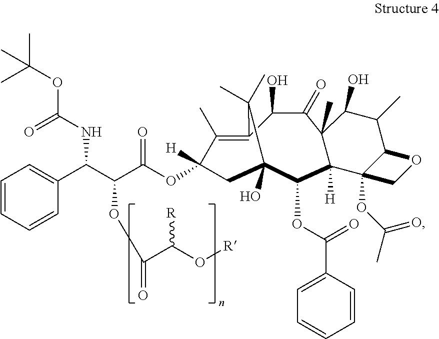

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g. n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the therapeutic agent may be docetaxel and may be attached to the polymer via the hydroxyl group at the 7 position.

In some embodiments, the therapeutic agent may be conjugated to the particulate construct to form the nanoparticle as shown in Structure 6 below:

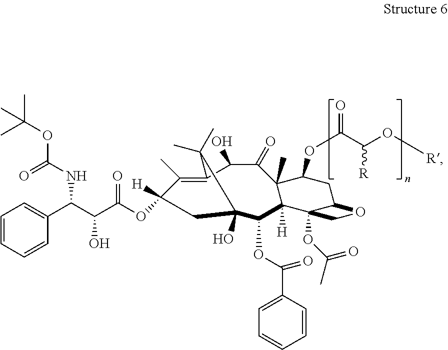

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 10 position.

In some embodiments, the therapeutic agent may be conjugated to the particulate construct to form Structure 7 below:

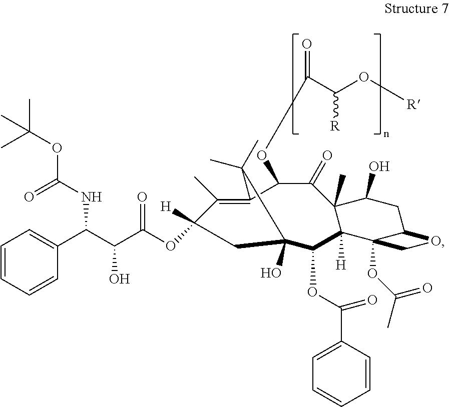

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the therapeutic agent may be docetaxel and may be covalently attached to the polymer through a carbonate bond.

In some embodiments, the particulate construct includes a combination of polymer composition-docetaxel conjugates described herein, e.g., polymer composition-docetaxel conjugates illustrated above.

In some embodiments, the therapeutic agent may be attached to the particulate construct through a linker. In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent.)

In some embodiments, the therapeutic agent may be conjugated to the particulate construct to form Structure 8 below:

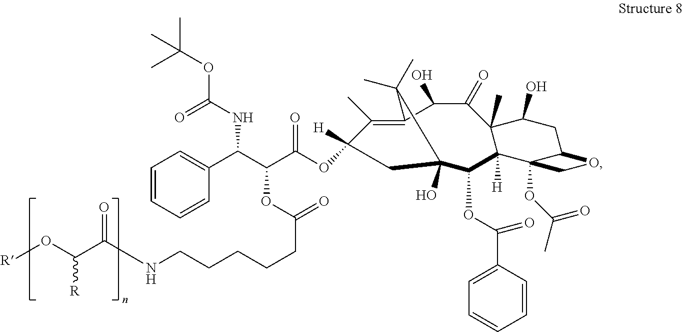

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308. e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the therapeutic agent may be conjugated to the particulate construct to form Structure 9 below:

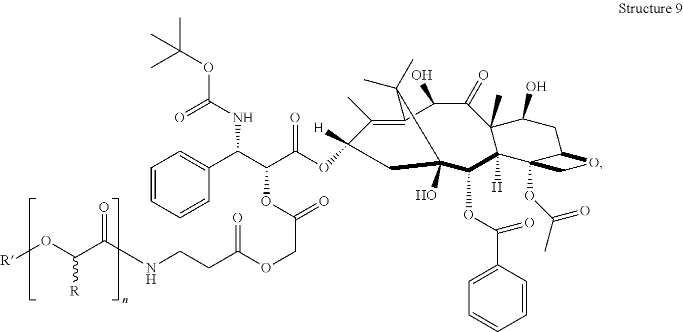

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the therapeutic agent may be conjugated to the particulate construct to form the nanoparticle as shown in Formula III below:

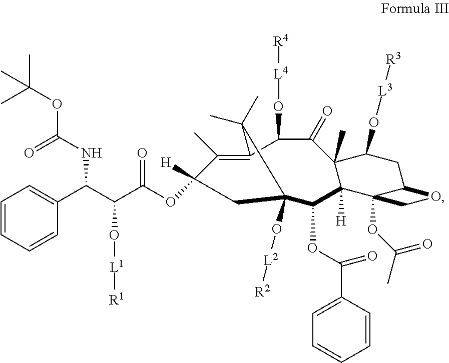

wherein L¹, L², L³ and L⁴ are each independently a bond or a linker, e.g., a linker described herein; R¹, R², R³ and R⁴ are each independently hydrogen, C₁-C₆ alkyl, acyl, a hydroxy protecting group, or a polymer of Formula IV:

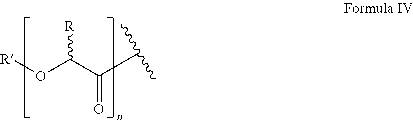

wherein about 30% to about 70%. e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g. about 50%); R′ is selected from hydrogen and acyl (e.g., acetyl); and wherein n is an integer from about 15 to about 308, e.g., about 77 to about 232, e.g. about 105 to about 170 (e.g. n is an integer such that the weight average molecular weight of the polymer is from about 1 kDa to about 20 kDa (e.g., from about 5 to about 15 kDa, from about 6 to about 13 kDa, or from about 7 to about 11 kDa)); and wherein at least one of R¹, R², R³ and R⁴ is a polymer of formula (IV).

In some embodiments, L² is a bond and R² is hydrogen.

In some embodiments, two therapeutic agents may be attached to a polymer composition via a multifunctional linker. In some embodiments, the two therapeutic agents may be the same therapeutic agent. In some embodiments, the two therapeutic agents may be different therapeutic agents. In some embodiments, the therapeutic agent may be docetaxel and is covalently attached to the polymer via a glutamate linker.

In some embodiments, the therapeutic agent may be conjugated to the particulate construct to form Structure 10 below:

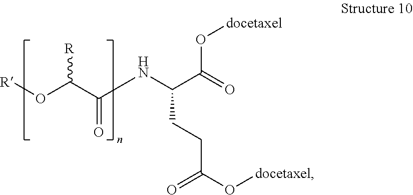

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g. acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel may be attached to the polymer composition via the hydroxyl group at the 2′ position. In some embodiments, at least one docetaxel may be attached to the polymer composition via the hydroxyl group at the 7 position. In some embodiments, at least one docetaxel may be attached to the polymer via the hydroxyl group at the 10 position. In some embodiments, at least one docetaxel may be attached to the polymer via the hydroxyl group at the 1 position. In some embodiments, each docetaxel may be attached via the same hydroxyl group, e.g., the hydroxyl group at the 2′ position, the hydroxyl group at the 7 position or the hydroxyl group at the 10 position. In some embodiments, each docetaxel may be attached via the 2′ hydroxyl group at the position. In some embodiments, each docetaxel may be attached via the hydroxyl group at the 7 position. In some embodiments, each docetaxel may be attached via the hydroxyl group at the 10 position. In some embodiments, each docetaxel may be attached via a different hydroxyl group, e.g., one docetaxel is attached via the hydroxyl group at the 2′ position and the other is attached via the hydroxyl group at the 7 position.

In some embodiments, four therapeutic agents may be attached to a particulate construct via a multifunctional linker. In some embodiments, the four therapeutic agents may be the same therapeutic agent. In some embodiments, the four therapeutic agents may be different therapeutic agents. In some embodiments, the therapeutic agent may be docetaxel, and may be covalently attached to the polymer via a tri(glutamate) linker.

In some embodiments, the therapeutic agent may be conjugated to the particulate construct to form the nanoparticle as shown in Structure 11 below:

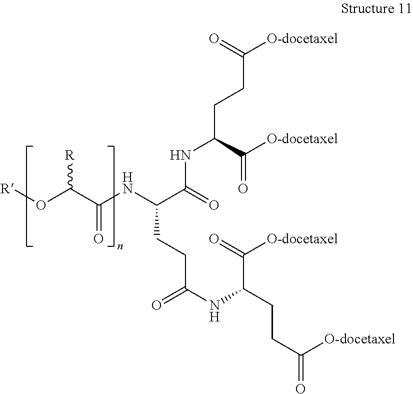

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel is attached to the polymer composition via the hydroxyl group at the 2′ position. In some embodiments, at least one docetaxel is attached to the polymer composition via the hydroxyl group at the 7 position. In some embodiments, at least one docetaxel is attached to the polymer composition via the hydroxyl group at the 10 position. In some embodiments, at least one docetaxel may be attached to the polymer via the hydroxyl group at the 1 position. In some embodiments, each docetaxel may be attached via the same hydroxyl group, e.g., the hydroxyl group at the 2′ position, the hydroxyl group at the 7 position or the hydroxyl group at the 10 position. In some embodiments, each docetaxel may be attached via the hydroxyl group at the 2′ position. In some embodiments, each docetaxel may be attached via the hydroxyl group at the 7 position. In some embodiments, each docetaxel may be attached via the hydroxyl group at the 10 position. In some embodiments, docetaxel molecules may be attached via different hydroxyl groups, e.g., three docetaxel molecules may be attached via the hydroxyl group at the 2′ position and the other may be attached via the hydroxyl group at the 7 position.

The therapeutic agent may be present in the particulate constructs described herein in any desired amount. The therapeutic agent may be present in an amount, e.g., from about 2% to about 50%, about 5% to about 30%, about 10% to about 30% by weight of the particulate construct (e.g., from about 3% to about 13% by weight). In some embodiments, the therapeutic agent to polymer composition ratio is in the range of from about 85:15 to about 55:45 by weight. In some embodiments, the polymer composition comprises a biodegradable block copolymer, and the therapeutic agent to polymer composition ratio is in the range of from about 85:15 to about 55:45 by weight.

### Potentiating Agents

In certain embodiments, the particulate construct comprises a potentiating agent that is covalently or non-covalently conjugated, for example, through a covalent bond or non-covalently association. In certain embodiments, the particulate construct may comprise a plurality of conjugations to a plurality of potentiating agents. The potentiating agent(s) attached to the particulate construct may be the same or different. The potentiating agent may be conjugated on a single polymer in the polymer composition, or to separate polymers in the polymer composition, or combinations thereof, as desired.

The potentiating agent may be any compound capable of modifying the polymer in the polymer composition of the particulate construct, and improving its pharmacokinetic and/or pharmacodynamic properties. In certain embodiments, the particulate construct of the present invention may further include a potentiating agent for optimizing the properties of the particulate construct, inter alia, to obtain a desired clearance time for the desired operation

The potentiating agent may comprise a hydrophilic polymer that increases solubility and/or stabilizes the particulate construct, particularly under biological conditions. A preferred type of hydrophilic potentiating agents may be polyalkylene glycols, such as polyethylene glycol (PEG), polypropylene glycols, polybutylene glycols, any derivative thereof, and any combination thereof. Particularly preferred is mPEG, PEG, a PEG-containing copolymer, or a PEG-containing compound (e.g., a polysorbate). Preferred polyethylene glycols have the formula CH₃—(CH₂CH₂O)zH, where z is from about 2 to about 500, preferably about 10 to about 300. PEG 600, PEG 2000, PEG 3400, and PEG 5000 are representative of the polyethylene glycols which may be used in the present invention. Preferred PEG-containing compounds for use in the present invention include, but are not limited to, polysorbates such as Tween 20, Tween 60, and Tween 80. In general, the higher the molecular weight of the PEG in the potentiating agent the greater the stabilization of the particulate construct. Higher molecular weight PEGs are generally preferred.

The invention relates to method for preparing and lyophilized formulations of particulate constructs, liposomes and micelles that contain PEG-containing compounds as potentiating agents. Such as Tween 20, Tween 60 and Tween 80 that can be covalently or noncovalently associated with the particulate constructs, liposomes and micelles.

Other suitable potentiating agents include any potentiating agent that does not adversely react with the other components of the particulate construct. In certain embodiments, the potentiating agent may be a compound including, but not limited to, a polysorbate, an enzyme, a peptide, a lecithin, a polysaccharide, a phospholipid analog, a poly(phosphazene), a poloxamer, a poly(oxyethylene ester), a poly(vinylpyrrolidone), a poly(vinyl alcohol), a glycolipid, a polyol, a salt, a crown ether, a specific nanoparticle-aptamer bioconjugate, any derivative thereof, and any combination thereof. The appropriate potentiating agent and amount thereof may depend upon the particulate construct characteristics, treatment conditions, and other factors known to individuals skilled in the art with the benefit of this disclosure. As can be seen from this discussion, the potentiating agent may be used to introduce a desired property into the composition. In some embodiments, the potentiating agent may be present in an amount, e.g., from about 2% to about 50%, about 5% to about 30%, about 10% to about 30% by weight of the particulate construct (e.g., from about 3% to about 13% by weight). The potentiating agent may be prepared using standard techniques. Employing mixtures of different potentiating agents may allow for greater variation and specificity in achieving desired composition properties.

### Additional Components of the Particulate Construct

Optionally, additional components may be included in the particulate construct of the present invention as desired for a particular application, including, but not limited to, stabilizing polymers, excipients, surfactants, targeting ligands, any derivatives thereof, and any combinations thereof. For example, stabilizing polymers may be included in the particulate construct and may improve stability of the nanoparticle and/or microparticle during a particular application. When additional compounds are included in the particulate construct, the compounds may be selected to provide advantages for an intended use, as deemed appropriate by one skilled in the art, with the benefit of this disclosure. For example, a targeting ligand can be included when delivery of the particulate construct to a particular tissue or cell is desired.

In some embodiments, the particulate constructs of the present invention may have increased stability when in the presence of a stabilizing polymer. Examples of suitable stabilizing polymers, include, but are not limited to, poly(vinyl alcohols), poly(vinyl pyrrolidones), poly(vinyl acetates), crown ethers, any derivatives thereof, and any combinations thereof. These stabilizing polymers may be reacted with a polymer in the polymer composition. The stabilizing polymer may be present in a particulate constructs of the present invention in an amount sufficient to maintain the stability of the nanoparticle and/or microparticle at a desired level. One of ordinary skill in the art, with the benefit of this disclosure, will recognize the appropriate stabilizing polymer and amount of stabilizing polymer to use for a chosen application.

In certain embodiments, the particulate construct may further comprise a targeting ligand. The targeting ligand may be introduced upon (e.g., as a component of the polymer composition) or after formation of the particulate construct, for example, via ligand modification of the polymer composition of the particulate construct. The targeting ligand may be any ligand that allows for targeting and/or binding to a desired cell. As would be understood by one of skill in the art, targeting and binding to a cell may include cell receptor attachment resulting in receptor mediated endocytosis. If two or more targeting ligands are attached, the ligands may be the same or different. Examples of suitable ligands include, but are not limited to, vitamins (e.g. folic acid), proteins (e.g. transferrin, and antibodies and antigen-binding fragments of antibodies such as Fab, Fab′ F(ab′)2, Fv, scFv, dAb), monosaccharides (e.g. galactose), peptides, and polysaccharides. The choice of a particulate targeting ligand, as one of ordinary skill appreciates, may vary depending upon the type of delivery desired. As another example, the ligand may be a membrane permeabilizing or membrane permeable agent such as the TAT protein from HIV-1. The TAT protein is a viral transcriptional activation that is actively imported into the cell nucleus.

In some embodiments, the particulate construct may optionally be bound to a targeting ligand having a molecular weight of less than about 1000 g/mol, for example, a low-molecular weight targeting ligand e.g., a PSMA targeting ligand. Such low-molecular weight PSMA targeting ligand may be selected from the group consisting of compounds I, II, III and IV:

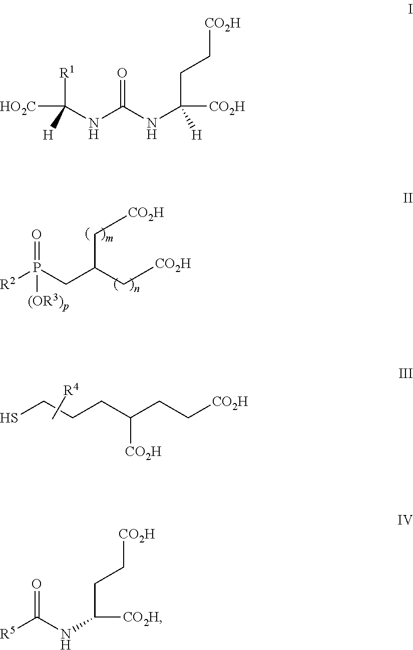

and enantiomers, stereoisomers, rotamers, tautomers, diastereomers, or racemates thereof; wherein m and n are each, independently, 0, 1, 2 or 3; p is 0 or 1; R¹, R², R⁴ and R⁵ are each, independently, selected from the group consisting of substituted or unsubstituted alkyl, substituted or unsubstituted aryl, and any combination thereof; and R³ is H or CH₃: wherein R¹, R², R⁴ or R⁵ comprise a point of covalent attachment to the nanoparticle. For example, R¹, R², R⁴ and R⁵ may be each, independently, C₁₋₆-alkyl or phenyl, or any combination of C₁₋₆-alkyl or phenyl, which are independently substituted one or more times with OH, SH, NHz, or CO₂H, and wherein the alkyl group may be interrupted by N(H), S or O.

In some embodiments, for example, R¹, R², R⁴ and R⁵ are each, independently, CH₂-Ph, (CH₂)₂—SH, CH₂—SH, (CH₂)₂C(H)(NH₂)CO₂H, CH₂C(H)(NH₂)CO₂H, CH(NH₂)CH₂CO₂H, (CH₂)₂C(H)(SH)CO₂H, CH₂—N(H)-Ph, O—CH₂-Ph, or O—(CH₂)₂-Ph, wherein each Ph may be independently substituted one or more times with OH, NH₂, CO₂H or SH. Exemplary low-molecular weight PSMA targeting ligand may be selected from the group consisting of:

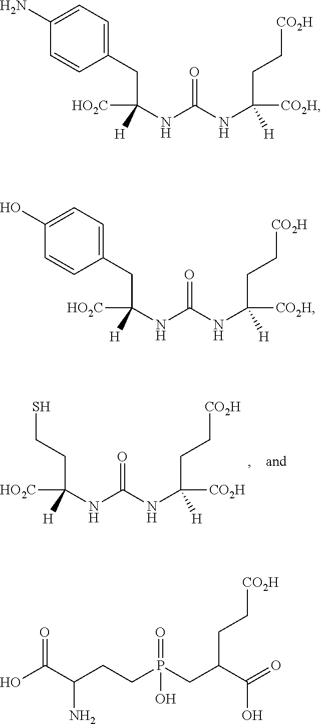

and enantiomers, stereoisomers, rotamers, tautomers, diastereomers, or racemates thereof; and wherein the NH₂, OH or SH groups serve as the point of covalent attachment to the first particle, or may be selected from the group consisting of:

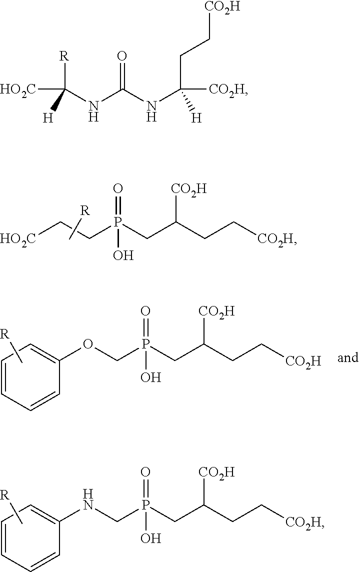

and enantiomers, stereoisomers, rotamers, tautomers, diastereomers, or racemates thereof; wherein R is independently selected from the group consisting of NH₂, SH, OH, CO₂H, C₁₋₆-alkyl that is substituted with NH₂, SH, OH or CO₂H, and phenyl that is substituted with NH₂ SH, OH or CO₂H, and wherein R serves as the point of covalent attachment to the first polymer.

Exemplary targeting ligands include, but are not limited to:

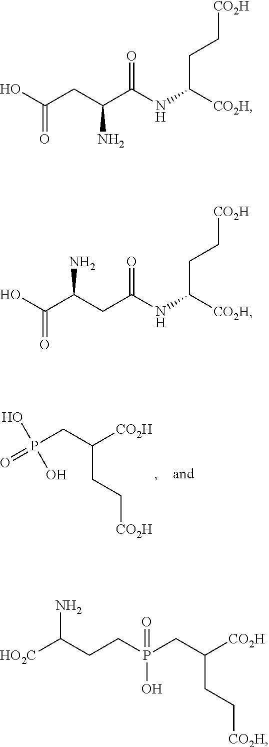

and enantiomers, stereoisomers, rotamers, tautomers, diastereomers, or racemates thereof; any of which may be further substituted with NH₂, SH, OH, CO₂H, C₁₋₆-alkyl that is substituted with NH₂, SH, OH or CO₂H, or phenyl that is substituted with NH₂, SH, OH or CO₂H, wherein these functional groups serve as the point of covalent attachment to the first polymer, for example, a low-molecular weight PSMA targeting ligand may be:

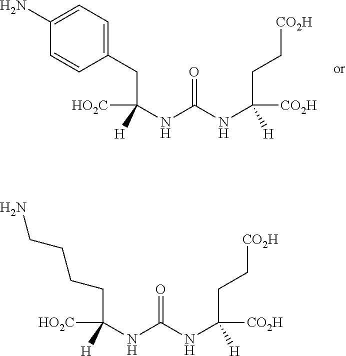

and enantiomers, stereoisomers, rotamers, tautomers, diastereomers, or racemates thereof; wherein the NH₂ groups serve as the point of covalent attachment to the first polymer.

The skilled addressees attention is directed to WO 2010/005723 which discloses suitable target moieties for prostate and breast cancer in ¶[0014] on page 4 and is incorporated herein by reference as suitable targeting moieties for the particulate construct, liposomes, and micelles of the current application.

Optionally, the particulate construct of the present invention may comprise one or more excipients. Suitable excipients include, but are not limited to sugars, surfactants, stabilizers, pH modifiers, reducing agents, antioxidants, animal and vegetable fats, oils, waxes, paraffins, starch, tragacanth, cellulose derivatives, silicones, bentonites, silicic acid, talc, zinc oxide, and any combinations thereof. One of ordinary skill in the art, with the benefit of this disclosure, will recognize the appropriate excipient and amount of excipient to use for a chosen application.

In one aspect of the present invention, both the therapeutic agent and the potentiating agent may be attached to the particulate construct, e.g., covalently bonded directly or indirectly to the hydrophobic polymer. Several modes of attaching the therapeutic agent and/or the potentiating agent to the particular construct are known in the art and may be used in the present invention. In certain embodiments, the therapeutic agent and/or potentiating agent may be directly attached to the particulate construct via a covalent bond, optionally through a linker. In other embodiments, the therapeutic agent and/or potentiating agent may be attached to the particulate construct via non-covalent associations. For example, the particulate construct can contain biotin moieties (e.g., be biotinylated) and the potentiating agent can contain avidin or streptavidin, and the potentiating agent is bound to the particulate construct through the noncovalent binding of the avidin or streptavidin to the biotin moieties.

In certain embodiments, a therapeutic agent and/or potentiating agent may be attached to the particulate construct via non-covalent associations, such as an association described herein. In certain embodiments, a plurality of non-covalent associations may attach a polymer of the polymer composition to a plurality of therapeutic agents and/or potentiating agents. The non-covalent associations may be disassociated under biological conditions. Suitable non-covalent associations include, but are not limited to, Van der Waals forces, electrostatic interactions, hydrophobic interactions, and any combination thereof.

In an embodiment, both the therapeutic agent and the potentiating agent may be attached to the particulate construct via the same mode.

In other embodiments, the therapeutic agent and the potentiating agent may be attached to the particulate construct by different modes. The therapeutic agent and/or potentiating agent may be attached to the polymer in the polymer composition of the particulate construct. In certain embodiments, the therapeutic agent and/or potentiating agent may be attached to either end of a polymer in the polymer composition. In other embodiments, the therapeutic agent and/or potentiating agent may be attached to a polymer backbone of a polymer in the polymer composition.

In certain embodiments, the therapeutic agent and the potentiating agent described herein may be directly attached to the particulate construct of the present invention via a covalent bond. A reactive functional group of the therapeutic agent and/or the potentiating agent may be directly attached to a functional group on a polymer composition. The therapeutic agent and/or the potentiating agent may be attached to the polymer composition of the particulate construct with a variety of linkages including, but not limited to, an amide linkage, an ester linkage, a succinimide linkage, a carbonate linkage, a carbamate linkage, and any combination thereof. For example, in an embodiment, the hydroxy group of a therapeutic agent and/or a potentiating agent may be reacted with a carboxylic acid group of the hydrophilic polymer, forming a direct ester linkage between the therapeutic agent and/or potentiating agent and the particulate construct. In an embodiment, an amino group of a therapeutic agent and/or potentiating agent may be linked to a carboxylic acid group of the polymer composition, forming an amide bond between the therapeutic agent and/or potentiating agent and the particulate construct. In another embodiment, a therapeutic agent and/or a potentiating agent may be directly attached to a terminal end of the polymer composition. For example, the polymer composition having a carboxylic acid moiety at its terminus may be covalently linked to a hydroxy or amino moiety of the therapeutic agent and/or potentiating agent, forming an ester or amide bond between the therapeutic agent and/or potentiating agent and the particulate construct.

In certain embodiments, to covalently bond the therapeutic agent and/or potentiating agent to a polymer composition, the polymer composition or the therapeutic agent and/or potentiating agent may be chemically activated using any technique known in the art. The activated component may be mixed with the other, under suitable conditions to allow a covalent bond to form between the polymer composition and the therapeutic agent and/or potentiating agent. In some embodiments, more than one component may be activated. In some embodiments, a nucleophile, such as a thiol, hydroxyl group, or amino group, on the therapeutic agent and/or potentiating agent attacks an electrophile (e.g., activated carbonyl group) to create a covalent bond.

In certain embodiments, a therapeutic agent and/or potentiating agent may be attached to the particulate construct via a linker, such as a linker described herein. In certain embodiments, a plurality of the linker moieties may be attached to a polymer in the polymer composition, allowing attachment of a plurality of therapeutic agents and/or potentiating agents to the linkers. The agent may be released from the linker under biological conditions. In other embodiments, a single linker may be attached to a polymer composition. Suitable linkers may include an alkylenyl (divalent alkyl) group. In certain embodiments, one or more carbon atoms of the alkylenyl linker may be replaced with one or more heteroatoms, such as oxygen, nitrogen or sulfur. In certain embodiments, one or more carbon atoms may be substituted with a substituent (e.g., alkyl, amino, or oxo substituents). In some embodiments, the linker, prior to attachment to the therapeutic agent and/or potentiating agent and the particulate construct, may comprise one or more of the following functional groups: amines, amides, hydroxyls, carboxylic acids, esters, halogens, thiols, carbonates, carbamates, and any combination thereof.

In some embodiments, the linker may comprise an amino acid linker or a peptide linker. Frequently, in such embodiments, the peptide linker is cleavable by hydrolysis, under reducing conditions, or by a specific enzyme. Such cleavage of the linker may be either within the linker itself, or it may be at one of the bonds that couples the linker to the remainder of the conjugate.

In some embodiments, a linker may be selected from one of the following exemplary linker schemes wherein, m is 1-10, n is 1-10, p is 1-10, and R is an amino acid side chain:

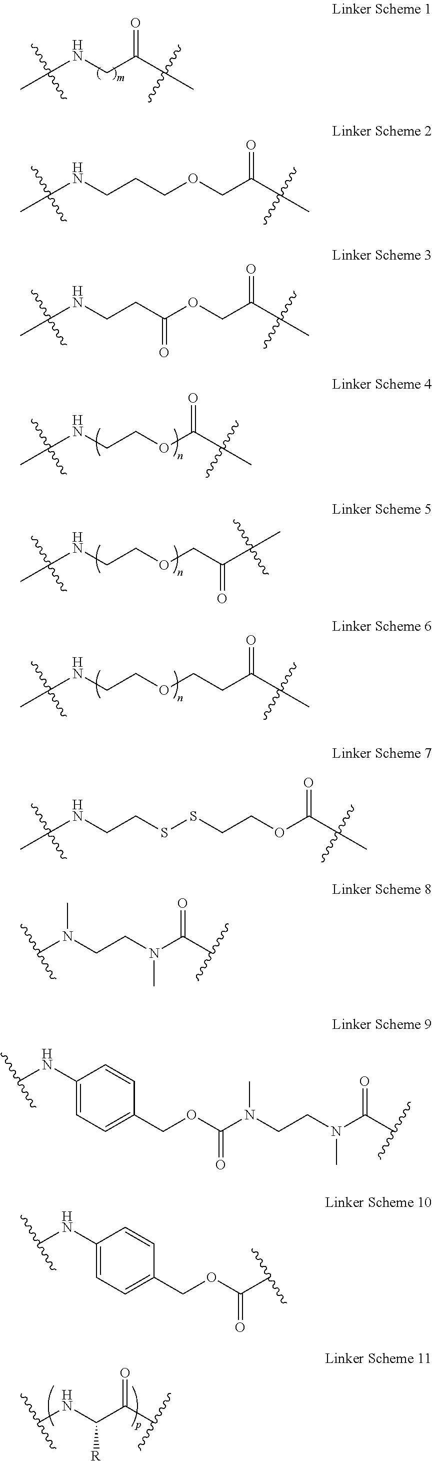

A linker may be cleaved by many reactions including, but not limited to, hydrolysis, reduction reactions, oxidative reactions, pH shifts, photolysis, any combinations thereof, or by an enzyme reaction. The linker may also comprise a bond that is cleavable under oxidative or reducing conditions, or may be sensitive to acids.

In certain embodiments, a particulate construct conjugate may comprise a plurality of conjugations to any number of therapeutic agents and/or potentiating agents. The therapeutic agent(s) attached to the particulate construct may be the same or different. The potentiating agent(s) attached to the particulate construct may be the same or different. In some embodiments, the particulate construct may be conjugated to a single therapeutic agent and a single potentiating agent to form a dual conjugate. One of ordinary skill in the art, with the benefit of this disclosure, will recognize the appropriate type and number of conjugates to use for a chosen application.

### Methods of Making Particulate Constructs

In certain embodiments, the present invention is directed to methods of making the disclosed particulate constructs exemplified by nanoparticles and/or microparticles. The polymer compositions and lyoprotectant disclosed above may be used to form the particulate constructs of the present invention using any suitable method. Exemplary methods include spray drying, emulsion (e.g., emulsion-solvent evaporation or double emulsion), precipitation (e.g., nanoprecipitation) and phase inversion.

In one embodiment, the particulate construct described herein can be prepared by precipitation (e.g., nanoprecipitation). This method involves dissolving the components of the particle (i.e., one or more polymers, an optional additional component or components, and an agent), individually or combined, in one or more solvents to form one or more solutions. For example, a first solution containing one or more of the components may be poured into a second solution containing one or more of the components (at a suitable rate or speed). The solutions may be combined, for example, using a syringe pump, a MicroMixer, or any device that allows for vigorous, controlled mixing. In some cases, nanoparticles can be formed as the first solution contacts the second solution, e.g., precipitation of the polymer upon contact causes the polymer to form nanoparticles. The control of such particle formation can be readily optimized.

In some embodiments, the particulate construct may be formed by providing one or more solutions containing one or more polymers and additional components, and contacting the solutions with certain solvents to produce the particle. In a non-limiting example, a polymer composition (e.g., PLGA), is conjugated to an agent to form a conjugate. This polymer-agent conjugate, a polymer containing a hydrophilic portion and a hydrophobic portion (e.g., PEG-PLGA), and optionally a third polymer (e.g., a biodegradable polymer, e.g., PLGA) are dissolved in a partially water miscible organic solvent (e.g., acetone). This solution is added to an aqueous solution containing a surfactant, forming the desired particles. These two solutions may be individually sterile filtered prior to mixing/precipitation.

The formed nanoparticles and/or microparticles can be exposed to further processing techniques to remove the solvents or purify the nanoparticles (e.g., dialysis). For purposes of the aforementioned process, water miscible solvents include acetone, ethanol, methanol, and isopropyl alcohol; and partially water miscible organic solvents include acetonitrile, tetrahydrofuran, ethyl acetate, isopropyl alcohol, isopropyl acetate or dimethylformamide.

Another method that can be used to generate the particulate construct described herein is a process termed “flash nanoprecipitation” as described by Johnson, B. K., et al, AlChE Journal (2003) 49:2264-2282 and U.S. 2004/0091546, each of which is incorporated herein by reference in its entirety. This process is capable of producing controlled size, polymer-stabilized and protected nanoparticles of hydrophobic organics at high loadings and yields. The flash nanoprecipitation technique is based on amphiphilic diblock copolymer arrested nucleation and growth of hydrophobic organics. Amphiphilic diblock copolymers dissolved in a suitable solvent can form micelles when the solvent quality for one block is decreased. In order to achieve such a solvent quality change, a tangential flow mixing cell (vortex mixer) is used. The vortex mixer consists of a confined volume chamber where one jet stream containing the diblock copolymer and active agent dissolved in a water-miscible solvent is mixed at high velocity with another jet stream containing water, an anti-solvent for the active agent and the hydrophobic block of the copolymer. The fast mixing and high energy dissipation involved in this process may provide timescales that are shorter than the timescale for nucleation and growth of particles, which leads to the formation of nanoparticles with active agent loading contents and size distributions not provided by other technologies. When forming the nanoparticles via flash nanoprecipitation, mixing occurs fast enough to allow high supersaturation levels of all components to be reached prior to the onset of aggregation. Therefore, the active agent(s) and polymers precipitate simultaneously, and overcome the limitations of low active agent incorporations and aggregation found with the widely used techniques based on slow solvent exchange (e.g., dialysis). The flash nanoprecipitation process is insensitive to the chemical specificity of the components, making it a universal nanoparticle formation technique.

A nanoparticle and/or microparticle described herein may also be prepared using a mixer technology, such as a static mixer or a micro-mixer (e.g., a split-recombine micro-mixer, a slit-interdigital micro-mixer, a star laminator interdigital micro-mixer, a superfocus interdigital micro-mixer, a liquid-liquid micro-mixer, or an impinging jet micro-mixer).

A split-recombine micromixer uses a mixing principle involving dividing the streams, folding/guiding over each other and recombining them per each mixing step, consisting of 8 to 12 such steps. Mixing finally occurs via diffusion within milliseconds, exclusive of residence time for the multi-step flow passage. Additionally, at higher-flow rates, turbulences add to this mixing effect, improving the total mixing quality further.

A slit interdigital micromixer combines the regular flow pattern created by multi-lamination with geometric focusing, which speeds up liquid mixing. Due to this double-step mixing, a slit mixer is amenable to a wide variety of processes.

Microfluidics Reaction Technology (MRT) may also be used to prepare the particulate construct of the present invention. At the core of MRT is a continuous, impinging jet microreactor scalable to at least 50 lit/min. In the reactor, high-velocity liquid reactants are forced to interact inside a microliter scale volume. The reactants mix at the nanometer level as they are exposed to high shear stresses and turbulence. MRT provides precise control of the feed rate and the mixing location of the reactants. This ensures control of the nucleation and growth processes, resulting in uniform crystal growth and stabilization rates.

The particulate construct described herein may also be prepared by emulsion. An exemplary emulsification method is disclosed in U.S. Pat. No. 5,407,609, which is incorporated herein by reference. This method involves dissolving or otherwise dispersing agents, liquids or solids, in a solvent containing dissolved wall-forming materials, dispersing the agent/polymer-solvent mixture into a processing medium to form an emulsion and transferring all of the emulsion immediately to a large volume of processing medium or other suitable extraction medium, to immediately extract the solvent from the microdroplets in the emulsion to form a microencapsulated product, such as microcapsules or microspheres. The most common method used for preparing polymer delivery vehicle formulations is the solvent emulsification-evaporation method. This method involves dissolving the polymer and drug in an organic solvent that is completely immiscible with water (for example, dichloromethane). The organic mixture is added to water containing a stabilizer, most often poly(vinyl alcohol) (PVA) and then typically sonicated.

Another suitable method of making the particulate construct includes providing a liquid formulation comprising the polymer composition and the lyoprotectant, and contacting such liquid formulation with a polymer nonsolvent to produce a particulate construct. The solution may be miscible or immiscible with the polymer nonsolvent. For example, a water-miscible liquid such as acetonitrile may contain the particulate construct and the lyoprotectant. The particulate constructs may be formed as the acetonitrile is contacted with a polymer nonsolvent, such as water, at a controlled rate causing the polymeric components of the particulate construct and lyoprotectant to precipitate and form particles.

Typically, an organic solvent (e.g., dichloromethane, acetonitrile, chloroform, tetrahydrofuran, acetone, formamide, dimethylformamide, pyridines, dioxane, dimethysulfoxide, etc.) and an aqueous liquid (e.g., water, or water containing dissolved salts or other species, cell or biological media, ethanol, etc.) are immiscible with respect to each other, meaning that the solutions are not soluble in the other to a level of at least 10% by weight at ambient temperature and pressure.

In some embodiments, particulate constructs may be formed as the first solution contacts the immiscible second liquid, e.g., precipitation of the polymer upon contact causes the polymer to form nanoparticles while the first solution poured into the second liquid, and in some cases, for example, when the rate of introduction is carefully controlled and kept at a relatively slow rate, nanoparticles may form. The control of such particulate construct formation can be readily optimized by one of ordinary skill in the art using only routine experimentation. Properties such as surface functionality, surface charge, size, zeta (ζ) potential, hydrophobicity, ability to control immunogenicity, and the like, may be highly controlled using a disclosed process to yield nanoparticles and/or microparticles suitable for a desired use.

In some embodiments, already-formed nanoparticles or microparticles may be functionalized with a targeting ligand. For example, a first copolymer (PLGA-PEG, poly(lactide-co-glycolide) and poly(ethylene glycol)) is mixed with a therapeutic agent to form particulate constructs. The particulate constructs may then associate with a low-molecular weight targeting ligand to form particulate constructs that can be used for the treatment of a specific tissue, such as tumor cells. Controlling parameters such as molecular weight of the particulate constructs, the molecular weight of the potentiating agent, and the particulate construct surface charge may lead to very precisely controlled particulate constructs for a particular use.

In some embodiments, a nanoemulsion process may also be used to make the particulate constructs disclosed herein. For example, a therapeutic agent, a potentiating agent, and a polymer may be mixed with an organic solution to form an organic phase. Such organic phase may include about 5 to about 50% weight solids. The organic phase may be combined with an aqueous solution to form a second phase. The organic solution can include, for example, toluene, methyl ethyl ketone, acetonitrile, tetrahydrofuran, ethyl acetate, isopropyl alcohol, isopropyl acetate, dimethylformamide, methylene chloride, dichloromethane, chloroform, acetone, benzyl alcohol, Tween 80, Span 80, or the like, and combinations thereof. In an embodiment, the organic phase may include benzyl alcohol, ethyl acetate, and combinations thereof. The second phase can be between about 1 and 50 weight % solids. The aqueous solution can be water, optionally in combination with one or more of sodium cholate, ethyl acetate, polyvinyl acetate and benzyl alcohol.

For example, the organic phase may use solvent that is only partially miscible with the nonsolvent (aqueous phase). Therefore, when mixed at a low enough ratio and/or when using water pre-saturated with the organic solvents, the organic phase remains liquid. The organic phase may be emulsified into an aqueous solution and, as liquid droplets, sheared into particulate constructs using, for example, high energy dispersion systems, such as homogenizers or sonicators. The aqueous portion of the emulsion, otherwise known as the “water phase,” may be surfactant solution consisting of sodium cholate and pre-saturated with ethyl acetate and benzyl alcohol. Emulsifying the second phase to form an emulsion phase may be performed in one or two emulsification steps. For example, a primary emulsion may be prepared, and then emulsified to form a fine emulsion. The primary emulsion can be formed, for example, using simple mixing, a high pressure homogenizer, probe sonicator, stir bar, or a rotor stator homogenizer. The primary emulsion may be formed into a fine emulsion through the use of e.g. probe sonicator or a high pressure homogenizer. For example, when a high pressure homogenizer is used, the pressure used may be about 1000 to about 8000 psi, about 2000 to about 4000 psi to about 8000 psi, or preferably about 4000 to about 5000 psi.

Either solvent evaporation or dilution may be needed to complete the extraction of the solvent and solidify the particulate construct. For better control over the kinetics of extraction and a more scalable process, a solvent dilution via aqueous quench may be used. For example, the emulsion can be diluted into cold water to a concentration sufficient to dissolve all of the organic solvent to form a quenched phase. Quenching may be performed at least partially at a temperature of about 5° C. or less. For example, water used in the quenching may be at a temperature that is less than room temperature. In some embodiments, not all of the therapeutic agent may be encapsulated in the particles at this stage, and a drug solubilizer is added to the quenched phase to form a solubilized phase.

The drug solubilizer may comprise a surfactant. Suitable drug solubilizers include, but are not limited to, Tween 80, Tween 20, polyvinyl pyrrolidone, cyclodextran, sodium dodecyl sulfate, or sodium cholate. For example, Tween 80 may added to the quenched nanoparticle suspension to solubilize the free drug and prevent the formation of drug crystals. In some embodiments, a ratio of drug solubilizer to therapeutic agent is about 100:1 to about 10:1.

The solubilized phase may be filtered to recover the particulate constructs. For example, ultrafiltration membranes may be used to concentrate the suspension and substantially eliminate organic solvent, free drug, and other processing aids (surfactants). Exemplary filtration may be performed using a tangential flow filtration system. For example, by using a membrane with a pore size suitable to retain particulate constructs while allowing solutes, micelles, and organic solvent to pass, particulate constructs can be selectively separated. Exemplary membranes with molecular weight cut-offs of about 300-500 kDa (5-25 nm) may be used. Diafiltration may be performed using a constant volume approach, meaning the diafiltrate (cold deionized water) may be added to the feed suspension at the same rate as the filtrate is removed from the suspension. After purifying and concentrating the suspension, the particulate constructs may be passed through one, two or more sterilizing and/or depth filters.

After the nanoparticles and/or microparticles are prepared by any of the methods described above, they may be fractionated by filtering, sieving, extrusion, or ultracentrifugation to recover particles within a specific size range. One sizing method involves extruding an aqueous suspension of the particles through a series of polycarbonate membranes having a selected uniform pore size; the pore size of the membrane will correspond roughly with the largest size of particles produced by extrusion through that membrane. See, e.g., U.S. Pat. No. 4,737,323, incorporated herein by reference. Another method is serial ultracentrifugation at defined speeds (e.g., 8,000, 10,000, 12,000, 15,000, 20,000, 22,000, and 25,000 rpm) to isolate fractions of defined sizes. Another method is tangential flow filtration, wherein a solution containing the particles is pumped tangentially along the surface of a membrane. An applied pressure serves to force a portion of the fluid through the membrane to the filtrate side. Particles that are too large to pass through the membrane pores are retained on the upstream side. The retained components do not build up at the surface of the membrane as in normal flow filtration, but instead are swept along by the tangential flow. Tangential flow filtration may thus be used to remove excess surfactant present in the aqueous solution or to concentrate the solution via diafiltration.

In certain embodiments, the particulate construct may be prepared to be substantially homogeneous in size within a selected size range. The amounts of polymer in the particulate construct, therapeutic agent or potentiating agent that are used in the preparation of the liquid formulation or lyophilized preparations of the present invention may differ from a final formulation. For example, some active agent may not become completely incorporated in a nanoparticle and/or microparticle and such free therapeutic agent may be e.g. filtered away. One of ordinary skill in the art, with the benefit of this disclosure, will know the appropriate amounts of each component to add to the starting compositions in order to obtain nanoparticles and/or microparticles with the appropriate amounts of each component for a desired application.

### Liposomes and Micelles

The present invention provides liquid formulations and lyophilized preparations that comprise a cryprotectant that comprises an oligosaccharide and a liposome and/or micelle that comprises a therapeutic agent and a potentiating agent. The therapeutic agent and the potentiating agent may be associated with the liposome or micelle through covalent or non-covalent interactions, for example, the therapeutic agent can be encapsulated within the lumen of the liposome or the core of the micelle.

Any liposome and/or micelle capable of carrying the therapeutic agents disclosed herein may be used in the present invention. Various methods may also be used to encapsulate therapeutic agents and excipients in liposomes or micelles. Examples of encapsulating techniques include, but are not limited to, conventional passive and active entrapment methods. Passive methods of encapsulating therapeutic agents in liposomes involve encapsulating the therapeutic agent during the preparation of the liposomes. This includes a passive entrapment method described by Bangham, et al., (J. Mol. Biol. (1965) 12:238). Additional methods of passive encapsulation include an ether injection technique described by Deamer and Bangham (Biochim. Biophys. Acta (1976) 443:629) and the Reverse Phase Evaporation technique as described by Szoka and Paphadjopoulos (P.N.A.S. (1978) 75:4194). Drug entrapment is another technique that may be used and relies on the formation of a drug-metal complex to drive uptake of a drug into the liposome. One of ordinary skill in the art, with the benefit of this disclosure, will recognize the appropriate method to use for incorporating a therapeutic agent into a liposome for a chosen application.

Suitable therapeutic agents and potentiating agents for use in the liposome and micelle aspects of the present invention are the same as those for the particulate construct aspects described above. As described herein for particulate constructs, Incorporation of a potentiating agent in such liposomal and/or micellar structures lead to improved phamacokinetic and pharmacodynamic properties. For example, such liposomes can extravasate from the systemec circulation selectively into specific tissues (ex. tumor), for example through the tumor neovasculature which is “leaky” in comparison to other vasculature. Therefore, such liposomes and micelles can be used for targeted drug delivery. The liposomes and/or micelles used contain a potentiating agent and, in general, can be prepared by coupling a potentiating agent to the surface molecules of traditional liposomes and/or micelles.

In some embodiments, the liposomes or micelles have a potentiating agent to lipid ratio in a range from about 1:1 to about 5:1, preferably about 2:1 to about 3:1, and more preferable about 2.5:1. The liposomes and/or micelles of the present invention may have a characteristic dimension in the range of about 25 nm to about 999 nm, preferably about 50 nm to about 300 nm, more preferably about 100 nm to about 200 nm. One of ordinary skill in the art with the benefit of this disclosure will recognize the appropriate size for a liposome for a chosen application.

The liposomes and/or micelles of the present invention may comprise any suitable lipid. Suitable lipids for use in the liposome compositions described herein include synthetic, semi-synthetic and naturally occurring phospholipids. Examples of such lipids include, but are not limited to, phospholipids, glycerophospholipids, phasphatidic acids, phosphatidylethanolamines, phosphatidylcholines, phosphatidylserines, phosphoinositides, phosphatidylinositols, phosphatidylinositol phosphates, bisphosphates, phosphatidyinositol triphosphates, sphingomyelins, any derivative thereof, and any combination thereof.

In certain embodiments, the additional liposomes and/or micelles may include at least one phospholipid and, optionally, an additional lipid such as cholesterol or a cholesterol derivative. In certain embodiments, the liposomes and/or micelles may include one or more of phospholipids. In particular embodiments, the liposomes and/or micelles may include two neutral lipids. In certain embodiments, the liposomes of the present invention are PEGylated liposomes, which are sometimes referred to as stealth liposomes. Examples of commercially-available liposomes suitable for use in the present invention may include, but are not limited to, liposomes composed of N-(carbonyl-methoxypolyethylene glycol 2000)-1,2-distearoyl-sn-glycero-3 phosphoethanolamine sodium salt (MPEG-DSPE), fully hydrogenated soy phosphatidylcholine (HSPC), and cholesterol, the liposome delivery agent in the product Doxil™ (Docorubicin HCl liposome injection; Alza Corporation). One of ordinary skill in the art with the benefit of this disclosure will recognize the appropriate type of liposome and/or micelle for a given application.

Optionally, the liquid formulation and/or lyophilized preparation comprising liposomes and/or micelles may further comprise one or more excipients. Suitable excipients include, but are not limited to, surfactants, stabilizers, pH modifiers, reducing agents, antioxidants, animal and vegetable fats, oils, waxes, paraffins, starch, tragacanth, cellulose derivatives, silicones, bentonites, silicic acid, talc, zinc oxide, and any combinations thereof. One of ordinary skill in the art, with the benefit of this disclosure, will recognize the appropriate excipient and amount of excipient to use for a chosen application.

The particulate constructs, micelles or liposomes disclosed in any of the following documents can be used in the present invention, and a stable lyophilized preparation that contains the particulate constructs, micelles or liposomes disclosed in any of the following documents can be prepared in accordance with this invention and the teachings herein: WO 2010/005721, WO 2010/005723, WO 2010/005725, WO 2010/005726, WO 2010/005740, U.S. Pat. No. 5,877,419, US Patent Application 2008/0081074. US Patent Application 2006/0216231, U.S. Pat. No. 7,550,441, US Patent Application 2004/0247680, WO 2008/019142, WO 2008/019142, WO 2007/150030, WO2007/133807, WO 2007/137117, WO 2005/046572, WO 2007/070682, WO 2006/105367, WO 2005/046572, WO 2004/026120, WO 2004/0261120, U.S. Pat. No. 7,427,605, US Patent Application 2009/0169638, US Patent Application 2009/0123428, US Patent Application 2007/0148255, U.S. Pat. No. 7,108,863, U.S. Pat. No. 7,276,248, U.S. Pat. No. 7,592,307, U.S. Pat. No. 6,120,798, U.S. Pat. No. 7,112,337, U.S. Pat. No. 6,465,008, US Patent Application 2008/0193509, US Patent Application 2008/0038316, US Patent Application 2003/0215492, US Patent Application 2005/0238706, US Patent Application 2005/0277611, US Patent Application 2008/0193510, U.S. Pat. No. 7,632,814, US Patent Application 2007/0286897, U.S. Pat. No. 7,238,367, US Patent Application 2009/0258071, U.S. Pat. No. 7,070,796, U.S. Pat. No. 6,599,519, U.S. Pat. No. 7,153,520, U.S. Pat. No. 6,916,788, U.S. Pat. No. 7,311,901, US Patent Application 2008/0188638, US Patent Application 2006/0089410, US Patent Application 2006/0057219, US Patent Application 2009/0191152, U.S. Pat. No. 6,060,518, U.S. Pat. No. 6,733,755, and U.S. Pat. No. 7,112,654.

In particular, the inventin provides a method for preparing lyophilized preparations and lyophilized preparations that contain any of the particles disclosed in WO 2010/005726, WO 2010/005725, WO 2010/005723, WO 2010/005721, or WO 2010/005740, which contain a potentiating PEG moiety, a therapeutic moiety and an optional targeting moiety.

### Methods of Lyophilizing

In certain embodiments, the liquid formulations of the present invention can be stably stored for relevant periods of time. Lyophilization extracts water from a solution to form a granular solid or powder. The process is carried out by freezing a liquid formulation and subsequently extracting any water or moisture by sublimation under vacuum. Lyophilizing the liquid formulations of the present invention may have many advantages including increased storage time and stability by maintaining substance quality and minimizing degradation. Lyophilization may be particularly useful for developing pharmaceutical drug products that are reconstituted and administered to a patient by injection, for example parenteral drug products. Alternatively, lyophilization is useful for developing oral drug products, especially fast melts or flash dissolve formulations.

The lyophilized preparations described herein may be stably stored for a period of time ranging from about 1 hour to about 3 years or more. Preferably, the lyophilized preparations may be stably storable for at least about 1 to about 24 months.

The lyophilized preparations may be stored under a variety of conditions, including ambient conditions (e.g., at room temperature, ambient humidity, and atmospheric pressure). The lyophilized preparations may also be stored at low temperature, e.g., at a temperature less than or equal to about 5° C. (e.g., less than or equal to about 4° C. or less than or equal to about 0° C.). A polymer-agent conjugate, particle or composition may also be frozen and stored at a temperature of less than about 0° C. (e.g., between −80° C. and −20° C.). The lyophilized preparations may also be stored under an inert atmosphere, e.g., an atmosphere containing an inert gas such as nitrogen or argon. Such an atmosphere may be substantially free of atmospheric oxygen and/or other reactive gases, and/or substantially free of moisture.

The lyophilized preparations may be stored in a variety of containers, including a light-blocking container such as an amber vial. A container may be a vial, e.g., a sealed vial having a rubber or silicone enclosure (e.g., an enclosure made of polybutadiene or polyisoprene). A container may be substantially free of atmospheric oxygen and/or other reactive gases, and/or substantially free of moisture.

The lyophilization of the liquid formulations may be performed by any appropriate method. A suitable technique may be a simple freeze drying technique where a liquid formulation is frozen, for example with liquid nitrogen, followed by drying under vacuum overnight at room temperature. During this simple lyophilization technique a Labconco® (available from Labocona Corp. of Kansas City, Mo.) freeze dryer, or any other suitable equipment may be used. A second suitable technique may involve a rapid cycle lyophilization method. Another suitable technique may involve a conventional cycle lyophilization method. Both the rapid cycle and conventional cycle lyophilization reactions may be performed using a VirTis® (available from SP Industries Inc. of Warminster, Pa.) advantage freeze dryer or any suitable freeze dryer.

In other embodiments, the present invention provides a rapid cycle lyophilization method where the multi-step ramping of temperatures is replaced with a slow ramping step. In such embodiments, the length of the lyophilization cycle is shortened to less than about 25 hours.

After freezing, the liquid formulations may be dried by any suitable method. In one embodiment, the liquid formulation may be dried in an available freeze dryer as noted above under a vacuum for an appropriate time. Exemplary conditions for the lyophilization methods are given in the Examples below. One of ordinary skill in the art, with the benefit of this disclosure, will know what conditions and equipment are suitable for lyophilizing a chosen composition.

The lyophilized preparations may then be resuspended in a reconstitution reagent for use. Suitable reconstitution reagents include a physiologically acceptable liquid as described above. Preferably, rehydration of the lyophilized preparations forms a suspension of nanoparticles, micelles, or liposomes which maintain the size distribution and morphology of the original nanoparticle, micellular, or liposomal suspension in the liquid formulation of the present invention before lyophilization, and further maintains the therapeutic agent to polymer ratio and/or the therapeutic agent to lipid ratio of the original liquid formulation before lyophilization. In certain embodiments, about 50 to about 100%, preferably about 80% to about 100%, of the nanoparticles, micelles, and/or liposomes maintain the size distribution and/or drug to lipid ratio of the original liquid formulation.

### Kits

The liquid formulations and/or lyophilized preparations described herein may be provided in a kit.

The kit may include the liquid formulation or lyophilized preparation and, optionally, a reconstitution reagent, a pharmaceutically acceptable carrier or adjuvant, a delivery device, and instructions for use.

Instructions for use may include informational material that can be descriptive, instructional, marketing or other material that relates to the methods described herein and/or the use of the particles for the methods described herein. Informational material of the kits is not limited in its form. In one embodiment, Informational material can include information about production of the liquid formulations and/or lyophilized preparations, physical properties of the components included in the liquid formulation and/or lyophilized preparation, concentration, date of expiration, batch or production site information, and so forth. In one embodiment. Informational material relates to methods for reconstituting and/or administering the liquid formulations and/or lyophilized preparations.

In one embodiment, Informational material can include instructions to administer the liquid formulation and/or lyophilized preparation described herein in a suitable manner to perform the methods described herein, e.g., in a suitable dose, dosage form, or mode of administration (e.g., a dose, dosage form, or mode of administration described herein). In another embodiment. Informational material can include instructions to administer a polymer-agent conjugate, particle or composition described herein to a suitable subject, e.g., a human, e.g., a human having or at risk for a disorder described herein. In another embodiment, Informational material can include instructions to reconstitute the lyophilized preparation described herein into a liquid pharmaceutical composition.

In one embodiment, the kit may include instructions to use the pharmaceutical composition for treatment of a subject. Instructions may include methods for diluting the pharmaceutical composition for use with a particular subject or in combination with a particular chemotherapeutic agent. Instructions may also include methods for reconstituting and/or diluting the pharmaceutical composition for use with a particular means of administration, such as by intravenous infusion.

In another embodiment, the kit includes instructions for treating a subject with a particular indication, such as a particular cancer, or a cancer at a particular stage. For example, Instructions can be for a cancer or cancer at stage described herein. Instructions may also address first line treatment of a subject who has a particular cancer, or cancer at a stage described herein. Instructions can also address treatment of a subject who has been non-responsive to a first line therapy or has become sensitive (e.g., has one or more unacceptable side effect) to a first line therapy, such as a taxane, an anthracycline, an alkylating agent, a platinum based agent, a vinca alkaloid.

In certain embodiments, Informational material of the kits may not be limited in its form. In many cases, Informational material. e.g., instructions, is provided in printed matter, e.g., a printed text, drawing, and/or photograph, e.g., a label or printed sheet. However, Informational material can also be provided in other formats, such as Braille, computer readable material, video recording, or audio recording. In another embodiment, Informational material of the kit may be contact information, e.g. a physical address, email address, website, or telephone number, where a user of the kit can obtain substantive information about a particle described herein and/or its use in the methods described herein. Informational material can also be provided in any combination of formats.

In addition to liquid formulation and/or lyophilized preparation described herein, the composition of the kit can include other ingredients, such as a surfactant, a stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a particle described herein. In such embodiments, the kit can include instructions for admixing a liquid formulation and/or lyophilized preparation described herein and the other ingredients, or for using a liquid formulation and/or lyophilized preparation described herein together with the other ingredients.

In another embodiment, the kit includes a second therapeutic agent, such as a second chemotherapeutic agent, e.g., a chemotherapeutic agent or combination of chemotherapeutic agents described herein. In one embodiment, the second agent is in lyophilized or in liquid form. In one embodiment, the liquid formulation and/or lyophilized preparation and the second therapeutic agent are in separate containers, and in another embodiment, the liquid formulation and/or lyophilized preparation and the second therapeutic agent are packaged in the same container.

In some embodiments, a component of the kit is stored in a sealed vial, e.g., with a rubber or silicone enclosure (e.g., a polybutadiene or polyisoprene enclosure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

In certain embodiments, the liquid formulation and/or lyophilized preparation described herein may be substantially pure and/or sterile. In embodiments comprising the liquid formulation, the liquid solution preferably is an aqueous solution, with a sterile aqueous solution being preferred. In embodiment comprising the lyophilized preparation, optionally, a reconstitution reagent is provided for reconstituting the lyophilized agent. The reconstitution reagent can include for example, a salt or saline solution, e.g., a sodium chloride solution having a pH between 6 and 9, lactated Ringer's injection solution. D5W, or PLASMA-LYTE A Injection pH 7.4® (Baxter, Deerfield, Ill.).

The kit may include one or more containers for containing the liquid formulation and/or lyophilized preparation described herein. In some embodiments, the kit contains separate containers, dividers or compartments for each element and Informational material. Suitable containers include, but are not limited to, bottles, vials, IV admixture bags, IV infusion sets, piggyback sets, syringes, and any combination thereof. Informational material may be contained in a plastic sleeve or packet. In other embodiments, the separate elements of the kit are contained within a single, undivided container. For example, the composition is contained in a bottle, vial or syringe that has attached thereto Informational material in the form of a label. In some embodiments, the kit includes a plurality (e.g., a pack) of individual containers, each containing one or more unit dosage forms (e.g., a dosage form described herein) of a polymer-agent conjugate, particle or composition described herein. For example, the kit includes a plurality of syringes, ampules, foil packets, or blister packs, each containing a single unit dose of a particle described herein. The containers of the kits may be air tight, waterproof (e.g., impermeable to changes in moisture or evaporation), and/or light-tight.

In some embodiments, the kit optionally includes a device suitable for administration of the pharmaceutical composition, e.g., a syringe, inhalant, pipette, forceps, measured spoon, dropper (e.g. eye dropper), swab (e.g., a cotton swab or wooden swab), or any such delivery device. In one embodiment, the device is a medical implant device, e.g., packaged for surgical insertion.

### Methods of Treatment

The liquid formulations and lyophilized preparations suitable for parenteral administration may be reconstituted using a reconstitution reagent with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of Intended recipient or suspending or thickening agents to form pharmaceutical compositions comprising the nanoparticles, micelles, or liposomes that comprise a therapeutic agent.

Examples of suitable aqueous and nonaqueous carriers which may be employed in the reconstitution reagent may include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

The pharmaceutical compositions described herein may be administered to a subject by any means known in the art including, but not limited to, orally, parenterally (e.g., via intravenous, subcutaneous, intracutaneous, intramuscular, intraarticular, intraarterial, intrasynovial, intrasternal, intrathecal, intralesional or intracranial injection), topically, mucosally (e.g., rectally or vaginally), nasally, buccally, ophthalmically, via inhalation spray (e.g., delivered via nebulization, propellant or a dry powder device) or via an implanted reservoir, and any combination thereof. In certain embodiments parenteral routes may be preferred since they avoid contact with digestive enzymes found in the alimentary canal. In certain embodiments, the subject may be a human or a non-human including, but not limited to, mammals, birds, reptiles, amphibians, fish, and any combination thereof.

These pharmaceutical compositions may also contain adjuvants such as preservatives, wetting agents, emulsifying agents and dispersing agents. Prevention of the action of microorganisms may be ensured by Inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of Injectable pharmaceutical form may be brought about by Inclusion of agents which delay absorption such as aluminum monostearate and gelatin.

In some embodiments, in order to prolong the effect of a drug, it is desirable to slow the absorption of the agent from subcutaneous or intramuscular injection. This may be accomplished by the use of a liquid suspension of crystalline or amorphous material having poor water solubility. The rate of absorption of the nanoparticles, liposomes and/or micelles comprising the therapeutic agents of the present invention may depend upon the rate of dissolution which, in turn, may depend upon crystal size and crystalline form. Alternatively, delayed absorption of a parenterally administered drug form is accomplished by dissolving or suspending the compositions of the present invention in an oil vehicle.

In certain embodiments, the pharmaceutical compositions of the present invention may be administered as solid dosage forms. Suitable solid dosage forms for administration include, but are not limited to, capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A compound may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile for the compositions of the present invention. Prior to administration, the compositions may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient may also be in micro-encapsulated form, if appropriate, with one or more of the above-described excipients.

Liquid dosage forms for oral administration may include pharmaceutically acceptable liquids, emulsions, microemulsions, solutions, suspensions, syrups and elixirs. In addition to the nanoparticles, micelles, or liposomes comprising the therapeutic agent, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the nanoparticles, micelles, or liposomes comprising the therapeutic agent, may contain suspending agents. Suitable suspending agents include, but are not limited to, ethoxylated isostearyl alcohols, polyoxyethylene sorbitol and sorbitan esters, microcrystalline cellulose, aluminum metahydroxide, bentonite, agar-agar and tragacanth, and any combinations or mixtures thereof.

In certain embodiments the pharmaceutical compositions of the present invention may be administered topically. The compositions may be suitable for topical administration when the desired treatment involves areas or organs readily accessible by topical application. For application topically to the skin, the pharmaceutical composition may be formulated with a suitable ointment containing the active components suspended or dissolved in a carrier. Suitable carriers for topical administration of the particle described herein include, but are not limited to, mineral oil, liquid petroleum, white petroleum, propylene glycol, polyoxyethylene polyoxypropylene compound, emulsifying wax and water. Alternatively, the composition may be formulated with a suitable lotion or cream containing the active particle suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions described herein may also be topically applied to the lower intestinal tract by rectal suppository formulation or in a suitable enema formulation. Topically-transdermal patches may also be suitable for use in the present with the methods of the present invention.

In certain embodiments, the compositions described herein may be administered by nasal aerosol or inhalation. Such compositions are prepared according to techniques well-known in the art and may be prepared as solutions in saline, employing benzyl alcohol or other suitable preservatives, absorption promoters to enhance bioavailability, fluorocarbons, and/or other solubilizing or dispersing agents known in the art.

In some embodiments, the pharmaceutical compositions described herein may also be administered in the form of suppositories for rectal or vaginal administration. Suppositories may be prepared by mixing one or more polymer-agent conjugate, particle or composition described herein with one or more suitable non-irritating excipients which is solid at room temperature, but liquid at body temperature. The pharmaceutical composition may therefore melt in the rectum or vaginal cavity and release the polymer-agent conjugate, particle or composition. Such materials includes, but is not limited to, cocoa butter, polyethylene glycol, a suppository wax or a salicylate. Pharmaceutical compositions of the present invention which are suitable for vaginal administration may also include pessaries, tampons, creams, gels, pastes, foams or spray formulations containing such carriers as are known in the art to be appropriate.

Ophthalmic formulations, eye ointments, powders, solutions and the like, may also be suitable for use with the methods of the present invention.

In certain embodiments, the dosage of the pharmaceutical compositions of the present invention may be chosen by the healthcare provider in order to administer an effective amount of the therapeutic agent to a subject in need thereof. As will be appreciated by those of ordinary skill in the art, the effective amount may vary depending on factors such as the desired biological endpoint, the type of therapeutic agent being delivered, the target tissue, the route of administration, etc. For example, the effective amount of an anti-cancer therapeutic agent may be the amount that results in a reduction in tumor size by a desired amount over a desired period of time. Additional factors that may be taken into consideration include, but are not limited to, the severity of the condition being treated, age of the subject, weight of the subject, gender of the subject, timing and frequency of administration, other treatments, tolerance by the subject, and response to treatment.

The nanoparticles, micelles, or liposomes comprising a therapeutic agent of the present invention, may be formulated into pharmaceutically acceptable dosage forms by conventional methods known to those of skill in the art. In certain embodiments the pharmaceutical compositions of the present invention may be formulated in dosage unit form for ease of administration and uniformity of dosage. Actual dosage levels of the pharmaceutic compositions of this invention may be varied so as to obtain an amount of the active ingredient which is effective to achieve the desired therapeutic response for a particular subject, composition, and mode of administration, without being toxic to the subject. Therapeutic efficacy and toxicity of the pharmaceutical compositions of the present invention may be determined by standard procedures in cell cultures or experimental animals. The data obtained from such experimentation may be used to formulate a range of doses for use in human subjects.

In one embodiment, the pharmaceutical composition is administered to a subject at a dosage of e.g., about 0.1 to 300 mg/m², about 5 to 275 mg/m², about 10 to 250 mg/m², e.g., about 15, 20, 25, 30, 35, 40, 45, 50, 55, 60, 65, 70, 80, 90, 100, 110, 120, 130, 140, 150, 160, 170, 180, 190, 200, 210, 220, 230, 240, 250, 260, 270, 280, 290 mg/m². Administration can be at regular intervals, such as every 1, 2, 3, 4, or 5 days, or weekly, or every 2, 3, 4, 5, 6, or 7 or 8 weeks. The administration can be over a period of from about 10 minutes to about 6 hours, e.g., from about 30 minutes to about 2 hours, from about 45 minutes to 90 minutes, e.g., about 30 minutes, 45 minutes, 1 hour, 2 hours, 3 hours, 4 hours. 5 hours or more. In one embodiment, the polymer-agent conjugate, particle or composition is administered as a bolus infusion or intravenous push, e.g., over a period of 15 minutes, 10 minutes, 5 minutes or less. In one embodiment, the polymer-agent conjugate, particle or composition is administered in an amount such the desired dose of the agent is administered. Preferably the dose of the polymer-agent conjugate, particle or composition is a dose described herein.

In some embodiments, the subject receives 1, 2, 3, up to 10 treatments, or more, or until the disorder or a symptom of the disorder is cured, healed, alleviated, relieved, altered, remedied, ameliorated, palliated, improved or affected. For example, the subject receives an infusion once every 1, 2, 3 or 4 weeks until the disorder or a symptom of the disorder are cured, healed, alleviated, relieved, altered, remedied, ameliorated, palliated, improved or affected. Preferably, the dosing schedule is a dosing schedule described herein.

In certain embodiments, the pharmaceutical composition can be administered as a first line therapy, e.g., alone or in combination with an additional agent or agents. In other embodiments, a pharmaceutical composition may be administered after a subject has developed resistance to, has tiled to respond to or has relapsed after a first line therapy. In some embodiments, the pharmaceutical composition is administered in combination with a second therapeutic agent.

In certain embodiments, the pharmaceutical composition of the present invention may be used to treat, alleviate, ameliorate, relieve, delay onset, inhibit progression, reduce severity of reduce incidence of one or more symptoms or features of a disease, disorder, and/or condition. In some embodiments, the liquid formulations and/or lyophilized preparations comprising the nanoparticles, liposomes or micelles of the present invention may be used to treat solid tumors. In certain embodiments, the liquid formulations and/or lyophilized preparations of the present invention may be used to treat any cancer cell including, but not limited to, colorectal cancer cells, gastric cancer cells, liver cancer cells, renal cancer cells, cystic cancer cells, pulmonary cancer cells, billiard tract cancer cells, pancreatic cancer cells, uterine cancer cells, ovarian cancer cells, breast cancer cells, a melanoma, and any combination thereof.

In some embodiments, the liquid formulations and/or lyophilized preparations of the present invention may be used to inhibit growth of the cancer cells. Suitable methods for inhibiting growth of the cancer cell include, but are not limited to, slowing the rate of the cancer cell proliferation and/or migration, arresting the cancer cell, or killing the cancer cell, such that the rate of the cancer cell growth is reduced in comparison with an untreated control cancer cell. Preferably, such an inhibition at a cellular level may reduce the size, deter the growth, reduce the aggressiveness, or prevent metastasis of cancer in a subject. One of ordinary skill in the art, with the benefit of this disclosure, can determine whether cancer cell growth is inhibited.

In some embodiments, inhibition of cancer cell growth may be evidenced by arrest of the cancer cell in a particular phase of the cell cycle, e.g., arrest at the G2/M phase of the cell cycle. Inhibition of cancer cell growth can also be evidenced by direct or indirect measurement of cancer cell or tumor size. In human subjects, such measurements may be made using well known imaging techniques such as magnetic resonance imaging, computerized axial tomography and X-rays. Inhibition of cancer growth may also be generally correlated to prolonged survival and/or increased health and well-being in a subject.

In some embodiments of the present invention, a liquid formulation comprises a lyoprotectant that comprises a cyclic oligosaccharide, and a particulate construct, the particulate construct comprising: a polymer composition; a therapeutic agent; and a potentiating agent, wherein the therapeutic agent and the potentiating agent are associated with the polymer composition.

In some embodiments of the present invention, a lyophilized preparation comprises: a particulate construct, the particulate construct comprising: a polymer composition; a therapeutic agent; and a potentiating agent, wherein the therapeutic agent and the potentiating agent are associated with the polymer composition; and lyoprotectant that comprises a cyclic oligosaccharide.

In some embodiments of the present invention, a method comprises providing a liquid formulation that comprises a lyoprotectant that comprises a cyclic oligosaccharide; a particulate construct, the particulate construct comprising a polymer composition; a therapeutic agent; and a potentiating agent, wherein the therapeutic agent and the potentiating agent are associated with the polymer composition; and lyophilizing the liquid formulation to provide a lyophilized preparation.

In some embodiments of the present invention, a method of treating cancer comprises: providing a lyophilized preparation comprising: a lyoprotectant that comprises a cyclic oligosaccharide; and a particulate construct, the particulate construct comprising: a polymer composition; a therapeutic agent; a potentiating agent, wherein the therapeutic agent and the potentiating agent are associated with the polymer composition; administering an effective amount of such preparation to a subject.

In some embodiments of the present invention, a method providing a lyophilized preparation comprising a lyoprotectant that comprises a cyclic oligosaccharide; and a particulate construct, the particulate construct comprising: a polymer composition; a therapeutic agent; and a potentiating agent, wherein the therapeutic agent and the potentiating agent are associated with the polymer composition; and combining the lyophilized preparation with a reconstitution reagent, to provide a reconstituted liquid formulation.

In some embodiments of the present invention, a device has disposed therein, a lyophilized preparation comprising a: lyoprotectant that comprises a cyclic oligosaccharide; and a particulate construct, the particulate construct comprising: a polymer composition; a therapeutic agent; and a potentiating agent, wherein the therapeutic agent and the potentiating agent are associated with the polymer composition.

In some embodiments of the present invention, a device has disposed therein, a reconstituted lyophilized preparation comprising: a lyoprotectant that comprises a cyclic oligosaccharide; and a particulate construct, the particulate construct comprising: a polymer composition; a therapeutic agent; and a potentiating agent, wherein the therapeutic agent and the potentiating agent are associated with the polymer composition.

In some embodiments of the present invention, a lyophilized preparation comprises a lyoprotectant that comprises a cyclic oligosaccharide; and a particulate construct, the particulate construct comprising: a polymer composition; a therapeutic agent; and a potentiating agent, wherein the therapeutic agent and the potentiating agent are associated with the polymer composition; and prepared by a rapid cycle lyophilization process.

In some embodiments of the present invention, a lyophilized preparation comprises: lyoprotectant that comprises a cyclic oligosaccharide; and a particulate construct, the particulate construct comprising: a polymer composition; a therapeutic agent; and a potentiating agent, wherein the therapeutic agent and the potentiating agent are associated with the polymer composition; and prepared by a lyophilization process that does not include an annealing step.

In some embodiments of the present invention, a lyophilizer has disposed therein, a lyophilized preparation comprising: lyoprotectant that comprises a cyclic oligosaccharide; and a particulate construct, the particulate construct comprising: a polymer composition; a therapeutic agent; and a potentiating agent, wherein the therapeutic agent and the potentiating agent are associated with the polymer composition.

In some embodiments of the present invention, a method for producing an inclusion body comprises: providing a liquid formulation that comprises a particulate construct that comprises a polymer composition and a therapeutic agent, and a lyoprotectant that comprises a cyclic polysaccharide; and lyophilizing the liquid formulation to produce a lyophilized preparation that comprises an inclusion body formed between the polysaccharide of the lyoprotectant and the polymer composition of the nanoparticle.

In some embodiments of the present invention, a method comprises: providing a liquid formulation that comprises a particulate construct and a lyoprotectant that comprises a polysaccharide wherein, the particulate construct comprises a therapeutic agent and a polymer composition; and lyophilizing the liquid formulation to produce a lyophilized preparation that comprises a hydrogen bond formed between the polysaccharide of the lyoprotectant and the polymer composition of the nanoparticle

In some embodiments of the present invention, a liquid formulation comprises a micelle that comprises a therapeutic agent, a micellular unilayer, and a potentiating agent, and a lyoprotectant that comprises a cyclic polysaccharide.

In some embodiments of the present invention, a lyophilized micelle therapeutic composition comprises: a micelle that comprises a therapeutic agent, a micellular unilayer, and a potentiating agent, and a lyoprotectant that comprises a cyclic polysaccharide.

In some embodiments of the present invention, a method comprises providing a liquid formulation that comprises a micelle that comprises a therapeutic agent, a micellular unilayer, and a potentiating agent, and a lyoprotectant that comprises a cyclic polysaccharide; and lyophilizing the liquid formulation to provide a lyophilized micelle therapeutic composition.

In some embodiments of the present invention, a method of treating cancer comprises providing a lyophilized micelle therapeutic composition that comprises a micelle that comprises a therapeutic agent, a micellular unilayer, and a potentiating agent, and a lyoprotectant that comprises a cyclic polysaccharide; and administering an effective amount of such composition to a subject in need thereof.

In some embodiments of the present invention, a lyophilized micelle therapeutic composition comprises a micelle that comprises a therapeutic agent, a micellular unilayer, and a potentiating agent, and a lyoprotectant that comprises a cyclic polysaccharide, and prepared by a rapid cycle lyophilization process.

In some embodiments of the present invention, a lyophilized micelle therapeutic composition comprises a micelle that comprises a therapeutic agent, a micellular unilayer, and a potentiating agent, and a lyoprotectant that comprises a cyclic polysaccharide, and prepared by a lyophilization process that does not include an annealing step.

In some embodiments of the present invention, a device has disposed therein, a lyophilized micelle therapeutic composition that comprises a micelle that comprises a therapeutic agent, a micellular unilayer, and a potentiating agent, and a lyoprotectant that comprises a cyclic polysaccharide.

In some embodiments of the present invention, a method of providing a liquid preparation comprises providing a lyophilized micelle therapeutic composition comprising a micelle that comprises a therapeutic agent, a micellular unilayer, and a potentiating agent, and a lyoprotectant that comprises a polysaccharide, and combining said liquid preparation with a reconstitution reagent, to provide a reconstituted preparation.

In some embodiments of the present invention, a method of treating cancer comprises providing a lyophilized micelle therapeutic composition that comprises a micelle that comprises a therapeutic agent, a micellular unilayer, and a potentiating agent, and a lyoprotectant that comprises a cyclic polysaccharide; and administering an effective amount of such composition to a subject.

In some embodiments of the present invention, a lyophilizer has disposed therein, a lyophilized preparation of a lyophilized micelle therapeutic composition that comprises a micelle that comprises a therapeutic agent, a micellular unilayer, and a potentiating agent, and a lyoprotectant that comprises a cyclic polysaccharide.

In some embodiments of the present invention, a method for producing an inclusion body comprises providing a liquid formulation that comprises a lyophilized micelle composition comprising a micelle that comprises a therapeutic agent, a micellular unilayer, and a potentiating agent, and a lyoprotectant that comprises a cyclic polysaccharide, and lyophilizing the liquid formulation to produce a lyophilized preparation that comprises an inclusion body formed between the cyclic polysaccharide of the lyoprotectant and the micelle.

In some embodiments of the present invention, a method comprises providing a liquid formulation that comprises a lyophilized micelle composition comprising a micelle that comprises a therapeutic agent, a micellular unilayer, and a potentiating agent, and a lyoprotectant that comprises a cyclic polysaccharide, and lyophilizing the liquid formulation to produce a lyophilized preparation that comprises a hydrogen bond formed between the cyclic polysaccharide of the lyoprotectant and the micelle.

In some embodiments of the present invention, a liquid formulation comprises a liposome that comprises a therapeutic agent, a liposomal bilayer, and a potentiating agent, and a lyoprotectant that comprises a cyclic polysaccharide.

In some embodiments of the present invention, a lyophilized liposome therapeutic composition comprises a liposome that comprises a therapeutic agent, a liposomal bilayer, and a potentiating agent, and a lyoprotectant that comprises a cyclic polysaccharide.

In some embodiments of the present invention, a method comprises providing a liquid formulation that comprises a liposome that comprises a therapeutic agent, a liposomal bilayer, and a potentiating agent, and a lyoprotectant that comprises a cyclic polysaccharide; and lyophilizing the liquid formulation to provide a lyophilized liposome therapeutic composition.

In some embodiments of the present invention, a method of treating cancer comprises providing a lyophilized liposome therapeutic composition that comprises a liposome that comprises a therapeutic agent, a liposomal bilayer, and a potentiating agent, and a lyoprotectant that comprises a cyclic polysaccharide; and administering an effective amount of such composition to a subject in need thereof.

In some embodiments of the present invention, a lyophilized liposome therapeutic composition comprises a liposome that comprises a therapeutic agent, a liposomal bilayer, and a potentiating agent, and a lyoprotectant that comprises a cyclic polysaccharide, and prepared by a rapid cycle lyophilization process.

In some embodiments of the present invention, a lyophilized liposome therapeutic composition comprises a liposome that comprises a therapeutic agent, a liposomal bilayer, and a potentiating agent, and a lyoprotectant that comprises a cyclic polysaccharide, and prepared by a lyophilization process that does not include an annealing step.

In some embodiments of the present invention, a device has disposed therein, a lyophilized liposome therapeutic composition that comprises a liposome that comprises a therapeutic agent, a liposomal bilayer, and a potentiating agent, and a lyoprotectant that comprises a cyclic polysaccharide.

In some embodiments of the present invention, a method of providing a liquid preparation comprises providing a lyophilized liposome therapeutic composition comprising a liposome that comprises a therapeutic agent, a liposomal bilayer, and a potentiating agent, and a lyoprotectant that comprises a polysaccharide, and combining said liquid preparation with a reconstitution reagent, to provide a reconstituted preparation.

In some embodiments of the present invention, a method of treating cancer comprises providing a lyophilized liposome therapeutic composition that comprises: a liposome that comprises a therapeutic agent, a liposomal bilayer, and a potentiating agent, and a lyoprotectant that comprises a cyclic polysaccharide; and administering an effective amount of such composition to a subject.

In some embodiments of the present invention, a method for producing an inclusion body comprises providing a liquid formulation that comprises a lyophilized liposome composition comprising a liposome that comprises a therapeutic agent, a liposomal bilayer, and a potentiating agent, and a lyoprotectant that comprises a cyclic polysaccharide, and lyophilizing the liquid formulation to produce a lyophilized preparation that comprises an inclusion body formed between the cyclic polysaccharide of the lyoprotectant and the liposome.

In some embodiments of the present invention, a method comprises providing a liquid formulation that comprises a lyophilized liposome composition comprising a liposome that comprises a therapeutic agent, a liposomal bilayer, and a potentiating agent, and a lyoprotectant that comprises a cyclic polysaccharide, and lyophilizing the liquid formulation to produce a lyophilized preparation that comprises a hydrogen bond formed between the cyclic polysaccharide of the lyoprotectant and the liposome.

To facilitate a better understanding of the present invention, the following examples of certain aspects of some embodiments are given. In no way should the following examples be read to limit, or define, the scope of Invention.

### EXAMPLES

The following examples are submitted for the purpose of illustrating certain aspects of the nanoparticles, liposomes, and micelles suitable for delivery of a therapeutic agent to a subject, described by the present invention.

### Example 1

5050 PLGA was purified and characterized using the following series of steps. In step 1, a 3-L round-bottom flask equipped with a mechanical stirrer was charged with 5050PLGA (300 g, Mw: 7.8 KDa; Mn: 2.7 KDa) and acetone (900 mL). The mixture was stirred for 1 h at ambient temperature to form a clear yellowish solution. In step 2, a 22-L jacket reactor with a bottom-outlet valve equipped with a mechanical stirrer was charged with MTBE (9.0 L, 30 vol. to the mass of 5050 PLGA). Celite® (795 g) was added to the solution with overhead stirring at ˜200 rpm to produce a suspension. To this suspension was slowly added the solution from Step A over 1 h. The mixture was agitated for an additional one hour after addition of the polymer solution and filtered through a polypropylene filter. The filter cake was washed with MTBE (3×300 mL), conditioned for 0.5 h, air-dried at ambient temperature (typically 12 h) until residual MTBE was ≦5 wt % (as determined by 1H NMR analysis. In step 3, a 12-L jacket reactor with a bottom-outlet valve equipped with a mechanical stirrer was charged with acetone (2.1 L, 7 vol. to the mass of 5050 PLGA). The polymer/Celite® complex from Step B was charged into the reactor with overhead stirring at ˜200 rpm to produce a suspension. The suspension was stirred at ambient temperature for an additional 1 h and filtered through a polypropylene filter. The filter cake was washed with acetone (3×300 mL) and the combined filtrates were clarified through a 0.45 mM in-line filter to produce a clear solution. This solution was concentrated to ˜1000 mL. In the final step, a 22-L jacket reactor with a bottom-outlet valve equipped with a mechanical stirrer was charged with water (9.0 L, 30 vol.) and was cooled down to 0-5° C. using a chiller. The solution from Step C was slowly added over 2 h with overhead stirring at ˜200 rpm. The mixture was stirred for an additional one hour after addition of the solution and filtered through a polypropylene filter. The filter cake was conditioned for 1 h, air-dried for 1 day at ambient temperature, and then vacuum-dried for 3 days to produce the purified 5050 PLGA as a white powder [258 g, 86%]. The ¹H NMR analysis was consistent with that of the desired product and Karl Fisher analysis showed 0.52 wt % of water. The HPLC results indicated that the product purity was >99% (AUC, 230 nm). The GPC results showed that the purity of the polymer was >99% (AUC, 230 nm) and the process produced a more narrow polymer polydispersity, i.e. Mw: 8.8 kDa and Mn: 5.8 kDa.

### Example 2

5050 PLGA lauryl ester was purified and characterized using the following protocol. A 12-L round-bottom flask equipped with a mechanical stirrer was charged with MTBE (4 L) and heptanes (0.8 L), The mixture was agitated at ˜300 rpm, to which a solution of 5050 PLGA lauryl ester (65 g) in acetone (300 mL) was added dropwise. Gummy solids were formed over time and finally clumped up on the bottom of the flask. The supernatant was decanted off and the solid was dried under vacuum at 25° C. for 24 h to afford 40 g of purified 5050 PLGA lauryl ester as a white powder [yield: 61.5%]. ¹H NMR (CDCl₃, 300 MHz): δ 5.25-5.16 (m, 53H), 4.86-4.68 (m, 93H), 4.18 (m, 7H), 1.69-1.50 (m, 179H), 1.26 (bs, 37H), 0.88 (t, J=6.9 Hz, 6H), The ¹H NMR analysis was consistent with that of the desired product. The GPC results showed that the purity of the polymer was >99% (AUC, 230 nm 6.02-9.9 min. t_{R}=7.91 min.

### Example 3

7525 PLGA was purified and characterized using the following protocol. A 22-L round-bottom flask equipped with a mechanical stirrer was charged with 12 L of MTBE, to which a solution of 7525 PLGA (150 g. approximately 6.6 kD) in dichloromethane (DCM, 750 mL) was added dropwise over an hour with an agitation of ˜300 rpm, resulting in a gummy solid. The supernatant was decanted off and the gummy solid was dissolved in DCM (3 L). The solution was transferred to a round-bottom flask and concentrated to a residue, which was dried under vacuum at 25° C. for 40 h to afford 94 g of purified 7525 PLGA as a white foam [yield: 62.7%]. ¹H NMR (CDCl₃, 300 MHz): δ 5.24-5.15 (m, 68H), 4.91-4.68 (m, 56H), 3.22 (s, 2.3H, MTBE), 1.60-1.55 (m, 206H), 1.19 (s, 6.6H, MTBE). The ¹H NMR analysis was consistent with that of the desired product. The GPC results showed that the purity of the polymer was >99% (AUC, 230 nm): 6.02-9.9 min, t_{R}=7.37 min.

### Example 4

O-acetyl-5050-PLGA was synthesized, purified and characterized using the following protocol. A 2000-mL, round-bottom flask equipped with an overhead stirrer was charged with purified 5050 PLGA [220 g, Mn of 5700] and DCM (660 mL). The mixture was stirred for 10 min to form a clear solution. Ac2O (11.0 mL, 116 mmol) and pyridine (9.4 mL, 116 mmol) were added to the solution, resulting in a minor exotherm of 0.5° C. The reaction was stirred at ambient temperature for 3 h and concentrated to ˜600 mL. The solution was added to a suspension of Celite® (660 g) in MTBE (6.6 L, 30 vol.) over 1 h with overhead stirring at ˜200 rpm. The suspension was filtered through a polypropylene filter and the filter cake was air-dried at ambient temperature for 1 day. It was suspended in acetone (1.6 L, ˜8 vol) with overhead stirring for 1 h. The slurry was filtered though a fritted funnel (coarse) and the filter cake was washed with acetone (3×300 mL). The combined filtrates were clarified though a Celite pad to afford a clear solution. It was concentrated to ˜700 mL and added to cold water (7.0 L, 0-5° C.) with overhead stirring at 200 rpm over 2 h. The suspension was filtered though a polypropylene filter. The filter cake was washed with water (3×500 mL), and conditioned for 1 h to afford 543 g of wet cake. It was transferred to two glass trays and air-dried at ambient temperature overnight to afford 338 g of wet product, which was then vacuum-dried at 25° C. for 2 days to constant weight to afford 201 g of product as a white powder [yield: 91%]. The ¹H NMR analysis was consistent with that of the desired product. The HPLC results confirmed that the purity was >99% (AUC, 230 nm). The GPC results showed that the purity of the polymer was >99% (AUC, 230 nm), Mw: 9.0 kDa and Mn: 6.3 kDa.

### Example 5

Doxorubicin 5050-PLGA amide was synthesized, purified and characterized using the following protocol. A 1000-ml round-bottom flask with a magnetic stirrer was charged with purified 5050 PLGA [55.0 g, 10.4 mmol, 1.0 equiv.], doxorubicin.HCl (6.7 g, 11.4 mmol, 1.1 equiv, 2-chloro-N-methylpyridinium iodide (3.45 g, 13.5 mmol, 1.3 equiv, and DMF (250 mL, anhydrous) under N2. The suspension was stirred for 15 min and triethylamine (4.6 mL, 32.2 mmol, 3.15 equiv.) was added dropwise over 10 min. The reaction mixture became a dark red solution after theddition of TEA and an exotherm from 23.2° C. to 26.2° C. was observed. The reaction was complete after 1.5 h as indicated by HPLC analysis. The mixture was filtered through a 0.50 □M PTFE membrane and the filtrate was added dropwise into water (5.50 L) containing 11 mL of AcOH over 20 min via addition funnels. The suspension was stirred for 1 h (pH ˜3-4), filtered over 30 min, and the filter cake was washed with water (3×300 mL). The solid was suspended in water (3.0 L) containing 0.1 vol % of AcOH and 5 vol % of acetone, stirred for 1 h, and filtered (pH ˜4-5) to afford 201.9 g of wet doxorubicin 5050 PLGA amide. The wet doxorubicin 5050 PLGA amide sample was transferred into a glass tray and dried under vacuum with nitrogen bleeding at 25° C. for 16 h to afford 162.9 g of semi-dry solid. The ¹H NMR analysis indicated ˜1.0 wt % of residual DMF. This sample was suspended in H2O (3 L) containing 3 mL of AcOH and 15 mL of acetone and stirred for 6 h, filtered, washed with H2O (0.5 L), and held for 0.5 h to afford 163.3 g of wet doxorubicin 5050 PLGA amide. The wet doxorubicin 5050 PLGA amide (155.8 g) was dried under vacuum with N2 bleeding at 25° C. for 16 h to afford 120.3 g of semi-dry product, which was dried at ambient temperature with N2 purge for 16 h to afford 54.4 g of doxorubicin 5050 PLGA amide [yield: 93%]. ¹H NMR (CDCl₃, 300 MHz): δ 14.00 (s, 1H), 13.27 (s, 1H), 8.05 (d, J=7.8 Hz, 1H), 7.80 (t, J=7.8 Hz, 1H), 7.40 (d, J=8.4 Hz, 1H), 6.44 (bs, 0.8H), 5.51 (bs, 1.2H), 5.22-5.17 (m, 40H), 4.91-4.72 (m, 81H), 4.31-4.08 (m, 7H), 3.64 (bs, 0.9H), 3.30 (d, J=20.4, 1H), 3.04 (d, J=18.9 Hz, 1H), 2.94 (s, 0.1H, DMF), 2.89 (s, 0.1H, DMF), 2.36 (d, J=14.4 Hz, 1H), 2.17 (d, J=14.1 Hz, 1H), 1.84 (bs, 5H), 1.60-1.55 (m, 120H), 1.28 (d, J=6.6 Hz). The 1H NMR analysis was consistent with that of the desired product. The HPLC results confirmed that the purity was >99% (AUC, 480 nm): 13.00-17.80 min, tR 16.8 min. The GPC results showed that the purity of the polymer was >99% (AUC, 480 nm): 5.2-8.6 min. tR 6.51 min. The conjugate could contain free 5050 PLGA and trace amount of drug.

### Example 6

Doxorubicin 7525-PLGA amide was synthesized, purified and characterized using the following protocol. 2-chloro-N-methylpyridinium iodide (1.95 g, 7.63 mmol) and TEA (3.15 mL, 22.6 mmol) were added to a mixture of purified 7525 PLGA [25.0 g, 3.80 mmol] and doxorubicin.HCl (3.08 g, 5.32 mmol) in DMF (125 mL, anhydrous) and stirred at ambient temperature. After 1 h, the reaction was complete by HPLC (0.4% doxorubicin remaining); however, there was 5.2% of an impurity at 12.0 min by HPLC analysis. The mixture was added into 2.50 L of water (25 mL of acetone wash) and 5.0 mL of acetic acid was added (pH=4-5). The resulting slurry was stirred for 30 min and filtered (250 mL water wash). The isolated wet cake was found to have only 1.7% of the 12.0 min impurity by HPLC analysis. The wet cake was slurried in water (1.25 L) and 1.3 mL of acetic acid was added. The mixture was stirred for 45 min, filtered (washed with 250 mL of water), and dried under vacuum for 44 h to afford 25.2 g of doxorubicin 7525 PLGA amide as a red solid [Yield: 93%]. ¹H NMR (CDCl3, 300 MHz): δ 13.99 (s, 1H), 13.26 (s, 1H), 8.04 (d, J=7.8 Hz, 1.2H), 7.79 (t, J=7.8 Hz, 1.1H), 7.40 (d, J=8.4 Hz, 1.1H), 6.44 (bs, 0.8H), 5.50 (bs, 1.3H), 5.22-5.17 (m, 60H), 4.91-4.72 (m, 53H), 4.31-4.08 (m, 8H), 3.64 (bs, 1.1H), 3.30 (d, J=20.4, 1.0H), 3.04 (d, J=18.9 Hz, 1.2H), 2.94 (s, ˜1.0H, DMF), 2.89 (s, 1.1H, DMF), 2.36 (d, J=14.4 Hz, 1.8H), 2.17 (m, 3.4H), 1.84 (bs, 3H), 1.60-1.55 (m, 184H), 1.28 (d, J=4.6 Hz, 6.6H), The 1H NMR analysis was consistent with that of the desired product. The HPLC results confirmed that the purity was 95.6% (AUC, 480 nm): 13.15-18.50 min. tR 17.6 min. The GPC results showed that the purity of the polymer was 95.6% (AUC, 480 nm): 5.2-8.5 min, tR 6.29 min. The conjugate could contain free 7525 PLGA and trace amount of drug.

### Example 7

Paclitaxel-2′-5050-PLGA-O-acetyl was synthesized, purified and characterized using the following protocol. A 250-mL round-bottom flask equipped with an overhead stirrer was charged with 5050 PLGA-O-acetyl [20 g. 2.6 mmol], paclitaxel (1.85 g, 2.1 mmol, 0.8 equiv., N,N′-dicyclohexyl-carbodiimide (DCC, 0.66 g. 3.2 mmol, 1.3 equiv.), 4-dimethylaminopyridine (DMAP, 0.39 g, 3.2 mmol, 1.3 equiv.), and DCM (100 mL. 5 vol). The mixture was agitated at 20° C. for 16 h and filtered to remove the dicyclohexylurea (DCU). The filtrate was concentrated to a residue and the residue was dissolved in acetone (100 mL), resulting in a cloudy suspension. It was filtered to remove residual DCU byproduct. The filtrate was added dropwise to 5:1 MTBE/heptanes (1.2 L) with vigorously stirring. The white precipitates formed a gum shortly after precipitation. The supernatant was decanted off and the gummy solid was isolated. The precipitation was repeated twice and the gummy solid was dried under vacuum at 25° C. for 16 h to afford 15.7 g of paclitaxel-2′-5050 PLGA-O-acetyl [yield: 72%] 1H NMR (CDCl₃, 300 MHz): δ 8.15 (d, J=7.5 Hz, 1H) 7.75 (d, J=6.6 Hz, 1H), 7.54-7.38 (m, 6H), 6.29-6.24 (a singlet overlaps with a triplet, 1H), 6.06 (bs, 0.5H), 5.69 (d, J=6.9 Hz, 0.4H), 5.58 (bs, 0.5H), 5.26-5.17 (m, 40H), 4.93 (d, J=7.8 Hz, 0.5H), 4.90-4.72 (m, 85H), 4.43 (t, J=3.9 Hz, 1H), 4.31 (d, J=8.1 Hz, 0.5H), 4.21 (d, J=8.1 Hz, 0.5H), 3.81 (d, J=6.6 Hz, 0.5H), 2.44 (bs, 2.5H), 2.23 (s, 1.5H), 2.17 (s, 19H, acetone), 1.8-1.7 (bs, 15H), 1.68 (s, 1.5H), 1.60-1.55 (m, 124H), 1.22 (bs, 2.5H), 1.14 (s, 1.5H), The 1H NMR analysis was consistent with that of the desired product. The HPLC results confirmed that the purity was >99% (AUC, 230 nm): 13.00-16.50 min, tR 15.60 min. The GPC results showed that the purity of the polymer was >99% (AUC, 230 nm): 6.0-9.7 min, tR=7.35 min. The conjugate could contain free 5050 PLGA-O-acetyl, some 7 paclitaxel-conjugate and trace amount of drug.

### Example 8

Docetaxel-2′-5050-PLGA-O-acetyl was synthesized, purified and characterized using the following protocol. A 250-mL round-bottom flask equipped with an overhead stirrer was charged with O-acetyl-5050 PLGA (16 g, 2.6 mmol), docetaxel (1.8 g, 2.1 mmol, 0.8 equiv.), DCC (0.66 g, 3.2 mmol, 1.3 equiv.), 4-dimethylaminopyridine (DMAP, 0.35 g, 3.2 mmol. 1.3 equiv.), and EtOAc (80 mL, 5 vol). The mixture was agitated at 20° C. for 2.5 h and an additional 0.5 equivalents of DCC (0.27 g) and DMAP (0.16 g) were added. The reaction was stirred at ambient temperature for 16 h and filtered to remove the dicyclohexylurea (DCU). The filtrate was diluted with EtOAc to 250 mL. It was washed with 1% HCl (2×60 mL) and brine (60 mL). The organic layer was separated, dried over Na2SO4, and filtered. The filtrate was concentrated to a residue and the residue was dissolved in acetone (100 mL), resulting in a cloudy suspension. It was filtered to remove residual DCU byproduct. The filtrate was added dropwise to 5:1 MTBE/heptanes (600 mL) with vigorously stirring. The white precipitates formed a gum shortly after precipitation. The supernatant was decanted off and the gummy solid was isolated. The precipitation was repeated three more times and the gummy solid was dissolved in acetone (300 mL). The solution was concentrated to a residue, which was dried under vacuum at 25° C. for 64 h to afford 14 g of docetaxel-5050 PLGA-O-acetyl [yield: 78%]. 1H NMR (CDCl3, 300 MHz): δ 8.11 (d, J=6.9 Hz, 1H). 7.61 (m, 0.6H), 7.50 (t, J=7.2 Hz, 6H), 7.39 (m, 1.3H), 6.22 (bs, 0.5H), 6.68 (d, J=7.5 Hz, 5.69-5.67 (m, 2.2H), 5.49-5.17 (m, 49H), 4.90-4.72 (m, 102H), 4.43 (m, 1.2H), 3.92 (d, J=5.7 Hz, 0.5H), 2.42 (bs, 2.1H), 2.17 (s, 29.3H, acetone), 1.90 (s, 3H), 1.80 (bs, 3H), 1.72 (s, 2H), 1.64-1.55 (m, 164H), 1.34 (s, 7H), 1.22 (m, 4H), 1.12 (s, 2.4H), The 1H NMR analysis was consistent with that of the desired product. The HPLC results confirmed that the purity was >99% (AUC, 230 nm): 15.50-18.00 min, tR 17.34 min. The GPC results showed that the purity of the polymer was >99% (AUC, 230 nm): 6.0-9.7 min, tR=7.35 min. The conjugate could contain free 5050 PLGA-O-acetyl, some 7 docetaxel-conjugate and trace amount of drug.

### Example 9

Bis(2′-docetaxel)glutamate-5050-PLGA-O-acetyl was synthesized, purified and characterized using the following protocol. A 500-mL, round-bottom flask was charged with 5050 PLGA-O-acetyl [40 g, 5.88 mmol], dibenzyl glutamate (3.74 g, 7.35 mmol), and DMF (120 mL, 3 vol.) and allowed to mix for 10 min to afford a clear solution. CMPI (2.1 g, 8.23 mmol) and TEA (2.52 mL) were added and the solution was stirred at ambient temperature for 3 h. The yellowish solution was added to a suspension of Celite (120 g) in MTBE (2.0 L) over 0.5 h with overhead stirring. The solid was filtered, washed with MTBE (300 mL), and vacuum-dried at 25° C. for 16 h. The solid was then suspended in acetone (400 mL, 10 vol), stirred for 0.5 h, filtered and the filter cake was washed with acetone (3×100 mL). The combined filtrates were concentrated to 150 mL and added to cold water (3.0 L, 0-5° C.) over 0.5 h with overhead stirring. The resulting suspension was stirred for 2 h and filtered through a PP filter. The filter cake was air-dried for 3 h and then vacuum-dried at 28° C. for 16 h to afford the product, dibenzylglutamate 5050 PLGA-O-acetyl [40 g. 95%]. The 1H NMR analysis indicated that the ratio of benzyl aromatic protons to methine protons of lactide was 10:46. HPLC analysis indicated 96% purity (AUC, 227 nm) and GPC analysis showed Mw: 8.9 kDa and Mn: 6.5 kDa.

Dibenzylglutamate 5050 PLGA-O-acetyl (40 g) was dissolved in ethyl acetate (400 mL) to afford a yellowish solution. Charcoal (10 g) was added to the mixture and stirred for 1 h at ambient temperature. The solution was filtered through a pad of Celite (60 mL) to afford a colorless filtrate. The filter cake was washed with ethyl acetate (3×50 mL) and the combined filtrates were concentrated to 400 mL. Palladium on activated carbon (Pd/C, 5 wt %, 4.0 g) was added, the mixture was evacuated for 1 min, filled up with H2 using a balloon and the reaction was stirred at ambient temperature for 3 h. The solution was filtered through a Celite pad (100 mL) and the filter cake was washed with acetone (3×50 mL). The combined filtrates had a grey color and were concentrated to 200 mL. The solution was added to a suspension of Celite (120 g) in MTBE (2.0 L) over 0.5 h with overhead stirring. The suspension was stirred at ambient temperature for 1 h and filtered through a PP filter. The filter cake was dried at ambient temperature for 16 h, suspended in acetone (400 mL), and stirred for 0.5 h. The solution was filtered through a PP filter and the filter cake was washed with acetone (3×50 mL). To remove any residual Pd, macroporous polystyrene-2,4,6-trimercaptotriazine resin (MP-TMT, 2.0 g, Biotage, capacity: 0.68 mmol/g) was added at ambient temperature for 16 h with overhead stirring. The solution was filtered through a Celite pad to afford a light grey solution. The solution was concentrated to 200 mL and added to cold water (3.0 L, 0-5° C.) over 0.5 h with overhead stirring. The resulting suspension was stirred at <5° C. for 1 h and filtered through a PP filter. The filter cake was air-dried for 12 h and vacuum-dried for 2 days to afford a semi-glassy solid [glutamic acid-PLGA5050-O-acetyl, 38 g, yield: 95%]. HPLC analysis showed 99.6% purity (AUC, 227 nm) and GPC analysis indicated Mw: 8.8 kDa and Mn: 6.6 kDa.

To remove any residual water, the glutamic acid-PLGA5050-O-acetyl [38 g] was dissolved in acetonitrile (150 mL) and concentrated to dryness. The residue was vacuum-dried at ambient temperature for 16 h to afford the desired product as a light grey powder [36 g]. A 1000-mL, round-bottom flask equipped with a magnetic stirrer was charged with glutamic acid-PLGA5050-O-acetyl [30 g, 4.5 mmol, Mn: 6.6 kDa], docetaxel (4.3 g, 2.9 mmol, 1.2 equiv), DMF (60 mL), and DCM (60 mL). The mixture was stirred for 10 min to afford a light brown solution. The first portion of EDC.HCl (1.6 g. 8.3 mmol) and DMAP (1.0 g, 8.3 mmol) was added and stirred at ambient temperature to yield a dark brown solution. After 2 h, a second portion of EDC.HCl (0.8 g, 4.2 mmol) and DMAP (0.50 g, 4.2 mmol) was added and stirred for an additional 2 to produce a darker solution. A third portion of EDC.HCl (0.3 g, 1.6 mmol) and DMAP (0.2 g, 1.6 mmol) was added. An additional portion of EDC.HCl (0.3 g, 1.6 mmol) and DMAP (0.2 g, 1.6 mmol) was added and stirred at ambient temperature for 2 h. The reaction mixture was added to a suspension of Celite (100 g) in MTBE (3.0 L) over 0.5 h with overhead stirring. The suspension was filtered through a PP filter and the filter cake was dried under vacuum at 25° C. for 12 h. The solid was suspended in acetone (250 mL) for 0.5 h with overhead stirring. The suspension was filtered and the filter cake was washed with acetone (3×60 mL). The combined filtrates were concentrated to 200 mL and added to cold water (3 L, 0° C.) over 0.5 h with overhead stirring. The suspension was filtered through a PP filter; the filter cake was washed with water (3×100 mL) and the solid was dried under vacuum at 25° C. for 16 h to afford a crude product [33 g]. To reduce any possible residual docetaxel, a second MTBE purification was conducted. The crude product was dissolved in acetone (150 mL) and added to a suspension of Celite (100 g) in MTBE (3 L). The suspension was filtered; the solid was vacuum-dried for 3 h, and suspended in acetone (500 mL) with overhead stirring. The suspension was filtered and the filter cake was washed with acetone (3×100 mL). The combined filtrates were concentrated to 200 mL and co-evaporated with acetonitrile (100 mL) to dryness. The residue was dissolved in acetone (200 mL) and the solution was precipitated into a suspension of Celite® (100 g)/MTBE (3 L) a third time. The mixture was stirred at ambient temperature for 1 h and filtered. The filter cake was washed with MTBE (2×200 mL) and vacuum-dried at ambient temperature overnight. The bis(2′-docetaxel) glutamate-5050 PLGA-O-acetyl/Celite complex was suspended in acetone (300 mL) with overhead stirring. The suspension was filtered and added to cold water (3 L) over 0.5 h with overhead stirring. The suspension was stirred at <5° C. for 1 hand filtered through a PP filter. The filter cake was washed with water (3×200 mL); the filter cake was conditioned for 0.5 h and vacuum-dried for 2 days to afford the desired product as an off-white powder [30 g, yield: 88%;]. This product was purified by another MTBE precipitation without using Celite. The product was dissolved in acetone to afford a solution (200 mL) and added to cold MTBE (2 L, 0° C.) over 1 h with overhead stirring. The resulting suspension was filtered and the filter cake was vacuum-dried at 25° C. for 16 h to afford a product with a tan color [34 g]. This sample was further dried for another 24 h and the residual MTBE was not reduced. To remove the residual MTBE, the product was precipitated into water. The isolated solid was vacuum-dried for 2 days to constant weight to afford the desired product as an off-white powder [bis(2′-docetaxel) glutamate-5050 PLGA-O-acetyl, 28.5 g. yield: 84%]. The 1H NMR analysis indicated that the docetaxel loading was 10% and HPLC analysis showed >99.5% purity (AUC, 227 nm). GPC analysis indicated Mw: 9.9 kDa and Mn: 6.1 kDa. The conjugate could contain free 5050 PLGA-O-acetyl, a mixture of 2′ and 7 docetaxel products and trace amount of drug.

### Example 10

A 250-mL, round-bottom flask equipped with a magnetic stirrer was charged with N-(tert-butoxycarbonyl)-L-glutamic acid (20 g, 40 mmol). (S)-dibenzyl 2-aminopentanedioate (4.85 g, 19.5 mmol), and DMF (100 mL). The mixture was stirred for 5 min to afford a clear solution. EDC.HCl (8.5 g, 44.3 mmol) and DMAP (9.8 g, 80 mmol) were added. The reaction was stirred at ambient temperature for 3 h, at which time HPLC analysis indicated completion of the reaction. The reaction was concentrated to a syrup (˜75 g) and EtOAc (250 mL) was added with overhead stirring. The resulting suspension was filtered to remove the N,N-dimethyl pyridinium p-toluenesulfonate. The filtrate was concentrated to a yellowish oil and water (200 mL) was added with vigorous stirring. White solid was gradually formed and the suspension was filtered. The solid was washed with water (2×50 mL) and dried under vacuum for 24 h to afford the N-Boc-tetrabenzyl-triglutamate product as a white powder [16.5 g, yield: 95%]. The 1H NMR analysis showed the desired product and HPLC analysis indicated a 92% purity (AUC, 254 nm). This crude product was further purified by recrystallization as follows. N-Boc-tetrabenzyl-triglutamate (15 g) was dissolved in hot IPAc (15 mL, 1 vol) and the solution was allowed to cool down to ambient temperature. A hydrogel like solid was formed and it was slurried in MTBE (200 mL) for 1 h, filtered. The filtration was slow owing to the hydrogel-like particles. The hydrogel solid was vacuum-dried at ambient temperature to afford product as a white powder [12.5 g, recovery yield: 83%]. The 1H NMR analysis showed the desired product and HPLC analysis indicated ˜100% purity (AUC, 254 nm).

A 250-mL, round bottom flask was charged with N-tert-butyloxycarbonyl-tetrabenzyl-triglutamate [N-t-BOC-tetrabenzyl-triglutamate, 11 g, 12.7 mmol] and DCM (25 mL) to afford a clear solution. Trifluoroacetic acid (TFA, 25 mL) was added to the solution and the reaction was stirred at ambient temperature. The solution was concentrated to a residue, dissolved in DCM (200 mL) and washed with saturated sodium bicarbonate (NaHCO3, 2×25 mL) and brine (30 mL). The organic layer was separated and dried over sodium sulfate (Na2SO4, 15 g). The solution was filtered and the filtrate was concentrated to a residue and vacuum-dried at ambient temperature for 16 h to afford the desired product (NH2-tetrabenzyl-triglutamate) as a wax-like semi-solid product [9.3 g. yield: 96%]. HPLC analysis indicated a 97% purity (AUC. 254 nm).

A 1000-mL, round-bottom flask equipped with a magnetic stirrer was charged with NH2-tetrabenzyl-triglutamate [4.0 g, 5.3 mmol], o-acetyl PLGA 5050 [30 g, 4.4 mmol, Mn: 6.8 kDa,], and DMF (100 mL). The mixture was stirred for a few minutes to afford a clear solution. 1-chloro-4-methylpyridinium iodide (CMPI, 1.7 g, 6.6 mmol) and trifluoroacetic acid (TEA, 1.3 mL, 8.8 mmol) were added and the reaction was stirred at ambient temperature for 3 h. The reaction mixture was added into cold water (2 L) over 1 h with overhead stirring. The generated suspension was filtered through a PP filter. The filter cake was washed with water (3×300 mL) and air-dried at ambient temperature for 16 h to afford a crude product. It was dissolved in acetonitrile (200 mL) and the solution concentrated to dryness. The residue was dissolved in acetone (100 mL) and the solution was added to cold MTBE (0° C., 2 L) over 0.5 h with overhead stirring to afford a suspension. It was filtered through a PP filter and the filter cake was vacuum-dried for 16 h to afford the product (tetrabenzyl-triglutamate-PLGA 5050-O-acetyl [30 g, yield: 88%]. The 1H NMR analysis indicated the ratio of benzyl aromatic protons over methine protons of lactide was 20:45. HPLC analysis showed>95% purity (AUC, 227 nm) and GPC analysis indicated a Mw: 8.9 kDa and a Mn: 6.7 kDa.

The tetrabenzyl-triglutamate-PLGA 5050-O-acetyl [30 g, 1.5 mmol] was dissolved in ethyl acetate (300 mL) to afford a pale yellowish solution. Charcoal (10 g) was added and the mixture was stirred at ambient temperature for 1 h and filtered through a Celite pad (100 mL). The filtrate became colorless and was transferred to a 1000-mL, round bottom flask equipped with a magnetic stirrer. Palladium on activated carbon (Pd/C, 5 wt. %, 4.0 g) was added, the mixture was evacuated for 1 min, filled up with H2 using a balloon and stirred at ambient temperature for 3 h. It was filtered through a Celite pad (100 mL) and the filter cake was washed with acetone (3×50 mL). The combined filtrates had a grey color and were filtered through multiple 0.45 μM polytetrafluoroethylene (PTFE) filters. The filtrate was concentrated to 150 mL and added to cold water (1.5 L, 0-5° C.) over 0.5 h with overhead stirring. The suspension was filtered and the filter cake was washed with water (3×100 mL), conditioned for 0.5 h, and vacuum-dried for 24 h to afford a white powder [tetra(2′-docetaxel)glutamate-PLGA5050-O-acetyl, 21 g, yield: 72%]. HPLC analysis indicated a 100% purity (AUC, 227 nm) and. GPC analysis showed a Mw: 9.2 kDa and Mn: 6.9 kDa.

A 1000-mL, round-bottom flask equipped with a magnetic stirrer was charged with tetra(2′-docetaxel)glutamate-PLGA5050-O-acetyl [20 g, 2.9 mmol, Mn 6.9 kDa,], docetaxel (5.7 g, 7.0 mmol. 2.4 equiv.), and DMF (75 mL). The mixture was stirred for 5 min to afford a clear solution. EDC.HCl (1.08 g, 5.6 mmol) and DMAP (0.72 g, 5.6 mmol) were added and the reaction was stirred at ambient temperature for 3 h. A second portion EDC.HCl (0.54 g, 2.8 mmol), and DMAP (0.54 g, 2.8 mmol) was added and the reaction was stirred for an additional 3 h. A third portion of EDC.HCl (0.36 g, 1.9 mmol) and DMAP (0.24 g, 1.9 mmol) was added and the reaction was stirred for 14 h. An additional portion of EDC.HCl (0.36 g, 1.9 mmol) and DMAP (0.24 g, 1.9 mmol) was added and the reaction was stirred for another 4 h. The reaction mixture was added to a suspension of Celite (60 g) in MTBE (2.0 L) over 0.5 h with overhead stirring. The suspension was filtered through a PP filter and the crude product/Celite complex was dried under vacuum at 25° C. for 12 h. The product/complex was suspended in acetone (200 mL) for 0.5 h with overhead stirring and filtered. The filter cake was washed with acetone (3×60 mL). The combined filtrates were concentrated to 100 mL. A second Celite/MTBE precipitation was conducted; the filtrate from the acetone extraction was concentrated to 100 mL, added to cold water (1.0 L, 0-5° C.) with overhead stirring and filtered. The solid was vacuum-dried for 2 days to afford crude product as a white powder [24 g]. The crude product was dissolved in acetone (120 mL) and added to a suspension of Celite (70 g, Aldrich, standard supercell, acid washed) in MTBE (2.0 L) at ambient temperature with overhead stirring. The suspension was stirred for 2 h and filtered through a fritted funnel. The filter cake was washed with MTBE (2×200 mL) and vacuum-dried at ambient temperature overnight. The solid was suspended in acetone (200 mL) with overhead stirring for 1 h. The suspension was filtered through a fritted funnel and the filter cake was rinsed with acetone (3×100 mL). The combined filtrates were concentrated to ˜150 mL and precipitated into Celite/MTBE a fourth time. To facilitate the purification, the filtrate was concentrated to −120 mL and added to MTBE (2.0 L) at ambient temperature with vigorous stirring. The suspension was filtered through a fritted funnel and the filter cake was vacuum-dried for 16 h to afford a crude product as a white powder containing ˜30 wt % of residual MTBE [30 g.>100% yield]. The crude product was dissolved in acetone (120 mL) and the solution was precipitated into MTBE (2.0 L). The resultant suspension was stirred at ambient temperature for 3 h and filtered through a fritted funnel. The filter cake was vacuum-dried for 12 h to afford a white solid [30 g]. At this point, a third water precipitation was conducted to isolate the product and reduce the residual MTBE. The above crude product was dissolved in acetone (100 mL) and the solution was added to cold water (1.5 L, 0-5° C.) over 0.5 h with overhead stirring. The suspension was filtered through a fritted funnel. The filter cake was washed with water (3×200 mL), conditioned for 2 h, and vacuum-dried for 2 days to afford the desired product (tetra-(2′-docetaxel) glutamate-5050 PLGA-O-acetyl) as a white powder [20 g, yield: 78%;]. HPLC analysis showed a 99.5% purity along with 0.5% of residual docetaxel. GPC analysis indicated a Mw: 10.8 kDa and Mn: 6.6 kDa.

The major product was tetra(2′-docetaxel) triglutamate-5050 PLGA-O-acetyl (wherein each docetaxel was attached to the triglutamate linker via the 2′ hydroxyl group); the product also included free 5050 PLGA-O-acetyl, monofunctionalized polymers (e.g., mono(2′-docetaxel) triglutamate-5050 PLGA-O-acetyl or monosubstituted products attached via the 7, 10 or 1 hydroxyl groups), difunctionalized polymers (e.g., bis(2′-docetaxel)triglutamate-5050 PLGA-O-acetyl, or disubstituted products with docetaxel molecules attached via other hydroxyl groups or mixtures thereof), trifunctionalized polymers (e.g., tris(2′-docetaxel)triglutamate-5050 PLGA-O-acetyl, or trisubstituted products with docetaxel molecules attached via other hydroxyl groups or mixtures thereof), and/or a trace amount of docetaxel.

### Example 11

Folate-PEG-PLGA-lauryl ester was synthesized, purified and characterized using the following protocol. The synthesis of folate-PEG-PLGA-lauryl ester involves the direct coupling of folic acid to PEG bisamine (Sigma-Aldrich, n=75, MW 3350 Da). PEG bisamine was purified due to the possibility that small molecular weight amines were present in the product. 4.9 g of PEG bisamine was dissolved in DCM (25 mL, 5 vol) and then transferred into MTBE (250 mL, 50 vol) with vigorous agitation. The polymer precipitated as white powder. The mixture was then filtered and the solid was dried under vacuum to afford 4.5 g of the product [92%]. The 1H NMR analysis of the solid gave a clean spectrum; however, not all alcohol groups were converted to amines based on Integration of α-methylene to the amine group (63% bisamine, 37% monoamine).

Folate-(γ)CO—NH-PEG-NH2 was synthesized using the purified PEG bisamine. Folic acid (100 mg, 1.0 equiv.) was dissolved in hot DMSO (4.5 mL, 3 vol to PEG bisamine). The solution was cooled to ambient temperature and (2-(7-Aza-1H-benzotriazole-1-yl)-1,1,3,3-tetramethyluronium hexafluorophosphate) (HATU, 104 mg, 1.2 equiv.) and N,N-Diisopropylethylamine (DIEA, 80 μL, 2.0 equiv.) were added. The resulting yellow solution was stirred for 30 minutes and PEG bisamine (1.5 g, 2 equiv.) in DMSO (3 mL, 2 vol) was added. Excess PEG bisamine was used to avoid the possible formation of di-adduct of PEG bisamine and to improve the conversion of folic acid. The reaction was stirred at 20° C. for 16 h and directly purified by CombiFlash using a C18 column (RediSep, 43 g, C18). The fractions containing the product were combined and the CH3CN was removed under vacuum. The remaining water solution (˜200 mL) was extracted with chloroform (200 mL×2). The combined chloroform phases were concentrated to approximately 10 mL and transferred into MTBE to precipitate the product as a yellow powder. In order to completely remove any unreacted PEG bisamine in the material, the yellow powder was washed with acetone (200 mL) three times. The remaining solid was dried under vacuum to afford a yellow semi-solid product (120 mg). HPLC analysis indicated a purity of 97% and the 1H NMR analysis showed that the product was clean.

Folate-(γ)CO—NH-PEG-NH₂ was reacted with p-nitrophenyl-COO-PLGA-CO₂-lauryl to provide folic acid-PEG-PLGA-lauryl ester. To prepare p-nitrophenyl-COO-PLGA-CO₂-lauryl, PLGA 5050 (lauryl ester) [10.0 g, 1.0 equiv.] and p-nitrophenyl chloroformate (0.79 g, 2.0 equiv.) were dissolved in DCM. To the dissolved polymer solution, one portion of TEA (3.0 equiv.) was added. The resulting solution was stirred at 20° C. for 2 h and the 1H NMR analysis indicated complete conversion. The reaction solution was then transferred into a solvent mixture of 4:1 MTBE/heptanes (50 vol). The product precipitated and gummed up. The supernatant was decanted off and the solid was dissolved in acetone (20 vol). The resulting acetone suspension was filtered and the filtrate was concentrated to dryness to produce the product as a white foam [7.75 g, 78%, Mn=4648 based on GPC]. The 1H NMR analysis indicated a clean product with no detectable p-nitrophenol.

Folate-(γ)CO—NH-PEG-NH₂ (120 mg, 1.0 equiv.) was dissolved in DMSO (5 mL) and TEA (3.0 equiv.) was added. The pH of the reaction mixture was 8-9. p-nitrophenyl-COO-PLGA-CO₂-lauryl (158 mg, 1.0 equiv.) in DMSO (1 mL) was added and the reaction was monitored by HPLC. A new peak at 16.1 min (˜40%, AUC, 280 nm) was observed from the HPLC chromatogram in 1 h. A small sample of the reaction mixture was treated with excess 1,8-diazabicyclo[5.4.0]undec-7-ene (DBU) and the color instantly changed to dark yellow. HPLC analysis of this sample indicated complete disappearance of p-nitrophenyl-COO-PLGA-CO₂-lauryl and the 16.1 min peak. Instead, a peak on the right side of folate-(γ)CO—NH-PEG-NH₂ appeared. It can be concluded that the p-nitrophenyl-COO-PLGA-CO₂-lauryl and the possible product were not stable under strong basic conditions. In order to identify the new peak at 16.1 min, ˜⅓ of the reaction mixture was purified by CombiFlash. The material was finally eluted with a solvent mixture of 1:4 DMSO/CH₃CN. It was observed that this material was yellow which could have indicated folate content. Due to the large amount of DMSO present, this material was not isolated from the solution. The fractions containing unreacted folate-(γ)CO—NH-PEG-NH₂ was combined and concentrated to a residue. A ninhydrin test of this residue gave a negative result, which may imply the lack of amine group at the end of the PEG. This observation can also explain Incomplete conversion of the reaction.

The rest of reaction solution was purified by CombiFlash. Similarly to the previous purification, the suspected yellow product was retained by the column. MeOH containing 0.5% TFA was used to elute the material. The fractions containing the possible product were combined and concentrated to dryness. The 1H NMR analysis of this sample indicated the existence of folate, PEG and lauryl-PLGA and Integration of these segments was close to the desired value of 1:1:1 ratio of all three components. High purities were observed from both HPLC and GPC analyses. The Mn based on GPC was 8.7 kDa. The sample in DMSO was recovered by precipitation into MTBE.

### Example 12

Docetaxel-2′-hexanoate-5050-PLGA-O-acetyl was synthesized, purified and characterized using the following protocol. A 500-mL round-bottom flask equipped with a magnetic stirrer was charged with 6-(carbobenzyloxyamino) caproic acid (4.13 g, 15.5 mmol), docetaxel (12.0 g, 14.8 mmol), and dichloromethane (240 mL). The mixture was stirred for 5 min to afford a clear solution, to which 1-ethyl-3-(3-dimethyllaminopropyl)carbodiimide hydrochloride (EDC.HCl) (3.40 g, 17.6 mmol) and 4 dimethylaminopyridine (DMAP) (2.15 g, 17.6 mmol) were added. The mixture was stirred at ambient temperature for 3 h at which time. IPC analysis showed a 57% conversion along with 34% residual docetaxel. An additional 0.2 equivalents of EDC.HCl and DMAP were added and the reaction was stirred for 3 h, at which time IPC analysis showed 63% conversion. An additional 0.1 equivalents of 6-(carbobenzyloxyamino)caproic acid along with 0.2 equivalents of EDC.HCl and DMAP were added. The reaction was stirred for 12 h and IPC analysis indicated 74% conversion and 12% residual docetaxel. To further increase the conversion, an additional 0.1 equivalents of 6-(carbobenzyloxyamino)caproic acid and 0.2 equivalents of EDC. HCl and DMAP were added. The reaction was continued for another 3 h at which time, IPC analysis revealed 82% conversion and the residual docetaxel dropped to 3%. The reaction was diluted with DCM (200 mL) and washed with 0.01% HCl (2×150 mL) and brine (150 mL). The organic layer was separated, dried over sodium sulfate, and filtered. The filtrate was concentrated to a residue and dissolved in ethyl acetate (25 mL). The solution was divided into two portions, each of which was passed through a 120-g silica column (Biotage F40). The flow rate was adjusted to 20 mL/min and 2000 mL of 55:45 ethyl acetate/heptanes was consumed for each of the column purifications. The fractions containing minor impurities were combined, concentrated, and passed through a column a third time. The fractions containing product (shown as a single spot by TLC analysis) from all three column purifications were combined, concentrated to a residue, vacuum-dried at ambient temperature for 16 h to afford the product, H₂N—(CH₂)₅CO—O-2′-docetaxel as a white powder [10 g, yield: 64%]. The 1H NMR analysis was consistent with thessigned structure of the desired product; however, HPLC analysis (AUC, 227 nm) indicated only a 97% purity along with 3% of bis-adducts. To purify the H₂N—(CH₂)₅CO—O-2′-docetaxel product, ethyl acetate (20 mL) was added to dissolve the batch to produce a clear solution. The solution was divided into two portions, each of which was passed through a 120-g silica column. The fractions containing product were combined, concentrated to a residue, vacuum-dried at ambient temperature for 16 h to afford the desired product (CBZ—NH—(CH₂)₅CO—O-2′-docetaxel) as a white powder [8.6 g, recovery yield: 86%]. HPLC analysis (AUC, 227 nm) indicated>99% purity.

A 1000-mL round-bottom flask equipped with a magnetic stirrer was charged with CBZ—NH—(CH₂)₅CO—O-2′-docetaxel product [5.3 g, 5.02 mmol] and THF (250 mL). To the resultant clear solution, MeOH (2.5 mL) and 5% Pd/C (1.8 g, 10 mol % of Pd) were added. The mixture was cooled to 0° C. and methanesulfonic acid (316 μL, 4.79 mmol) was added. The flask was evacuated for 10 seconds and filled with hydrogen using a balloon. After 3 h, IPC analysis indicated 62% conversion. The ice-bath was removed and the reaction was allowed to warm up to ambient temperature. After an additional 3 h, IPC analysis indicated that the reaction was complete. The solution was filtered through a Celite® pad and the filtrate was black in appearance. To remove the possible residual Pd, charcoal (5 g, Aldrich. Darco®) was added and the mixture was placed in a fridge overnight and filtered through a Celite® pad to produce a clear colorless solution. This was concentrated at <20° C. under reduced pressure to a volume of ˜100 mL, to which methyl tert-butyl ether (MTBE) (100 mL) was added. The resultant solution was added to a solution of cold MTBE (1500 mL) with vigorous stirring over 0.5 h. The suspension was left at ambient temperature for 16 h, the upper clear supernatant was decanted off and the bottom layer was filtered through a 0.45 μm filter membrane. The filter cake was vacuum-dried at ambient temperature for 16 h to afford the desired product (H₂N—(CH₂)₅CO—O-2′-docetaxel) as a white solid [4.2 g, yield: 82%]. HPLC analysis indicated>99% purity and the 1H NMR analysis indicated the desired product.

A 100-mL round-bottom flask equipped with a magnetic stirrer was charged with 5050 PLGA-O-acetyl (5.0 g, 0.7 mmol), H₂N—(CH₂)₅CO—O-2′-docetaxel [0.85 g, 0.84 mmol, GAO-G-28(3)], DCM (5 mL), and DMF (20 mL). The mixture was stirred for 5 min to produce a clear solution. EDC.HCl (0.2 g, 1.05 mmol) and DMAP (0.21 g, 1.75 mmol) were added and the reaction was stirred for 3 h, at which time IPC analysis indicated 79% conversion along with 18% of H₂N—(CH₂)₅CO—O-2′-docetaxel. Two small impurities were observed at 11.6 min and 11.7 min (2.8%, AUC, 227 nm). An additional portion of EDC.HCl (0.1 g, 0.5 mmol) and DMAP (0.15 g, 1.2 mmol) was added and the reaction was stirred overnight. IPC analysis showed 92% conversion along with 6% of H₂N—(CH₂)₅CO—O-2′-docetaxel; the level of the two impurities did not change. To increase the conversion, an additional amount of 5050 PLGA-β-acetyl (0.5 g) along with EDC.HCl (0.1 g) and DMAP (0.15 g) was added and the reaction was stirred at ambient temperature for 3 h. IPC analysis showed a 95.6% conversion along with 3.0% of H₂N—(CH₂)₅CO—O-2′-docetaxel; the two impurities were about 1.3%. The reaction was combined with a previously prepared product and added to a suspension of Celite® (20 g) in MTBE (600 mL) with mechanical stirring over 30 min. The suspension was stirred at ambient temperature for 0.5 h and filtered. The filter cake was air-dried for 30 min and then vacuum-dried such that the residual MTBE contained no more than 5 wt %. The polymer/Celite® complex was then suspended in acetone (50 mL) and the slurry was stirred for 30 min, filtered through a Celite pad. The filter cake was washed with acetone (3×30 mL). The combined filtrates were concentrated to ˜25 mL and this solution was analyzed by HPLC showing that the level of H₂N—(CH₂)₅CO—O-2′-docetaxel or the impurities was identical to these prior to MTBE precipitation. The solution was added to cold water (500 mL) containing 0.05% acetic acid over 30 min. The suspension was stirred at 0° C. for 1 h and filtered through a PP filter. The filter cake was washed with water (3×50 mL), conditioned for 30 min, vacuum-dried at ambient temperature for 48 h to produce docetaxel-2″-hexanoate-5050 PLGA-O-acetyl as a white powder [6.3 g, 85%]. The 1H NMR analysis indicated 10.5 wt % of loading. No DMAP or DMF was observed. GPC analysis indicated a Mw of 8.2 kDa and a Mn of 5.7 kDa. HPLC analysis indicated a purity of 98.6% (AUC, 230 nm) and a 0.75% of H₂N—(CH₂)₅CO—O-2′-docetaxel. The two impurities totaled 0.5% (AUC, 230 nm).

### Example 13

O-acetyl-5050-PLGA-(2′-β-alanine glycolate)-docetaxel was synthesized, purified and characterized using the following protocol. A 1000 mL round-bottom flask equipped with a magnetic stirrer was charged with carbobenzyloxy-β-alanine (Cbz-β-alanine, 15.0 g, 67.3 mmol), tert-butyl bromoacetate (13.1 g, 67.3 mmol), acetone (300 mL), and potassium carbonate (14 g, 100 mmol). The mixture was heated to reflux at 60° C. for 16 h, cooled to ambient temperature and then the solid was removed by filtration. The filtrate was concentrated to a residue, dissolved in ethyl acetate (EtOAc, 300 mL), and washed with 100 mL of water (three times) and 100 mL of brine. The organic layer was separated, dried over sodium sulfate and filtered. The filtrate was concentrated to clear oil [22.2 g, yield: 99%]. HPLC analysis showed 97.4% purity (AUC, 227 nm) and 1H NMR analysis confirmed the desired intermediate product, t-butyl (carbobenzyloxy-β-alanine) glycolate.

Intermediate product, carbobenzyloxy-β-alanine glycolic acid (Cbz-β-alanine glycolic acid), was prepared using a 100 mL round-bottom flask equipped with a magnetic stirrer that was charged with t-butyl (Cbz-β-alanine) glycolate [7.5 g, 22.2 mmol] and formic acid (15 mL, 2 vol). The mixture was stirred at ambient temperature for 3 h to give a red-wine color and HPLC analysis showed 63% conversion. The reaction was continued stirring for an additional 2 h, at which point HPLC analysis indicated 80% conversion. An additional portion of formic acid (20 mL, 5 vol in total) was added and the reaction was stirred overnight, at which time HPLC analysis showed that the reaction was complete. The reaction was concentrated under vacuum to a residue and redissolved in ethyl acetate (7.5 mL, 1 vol.). The solution was added to the solvent heptanes (150 mL, 20 vol.) and this resulted in the slow formation of the product in the form of a white suspension. The mixture was filtered and the filter cake was vacuum-dried at ambient temperature for 24 h to afford the desired product. Cbz-β-alanine glycolic acid as a white powder [5.0 g, yield: 80%]. HPLC analysis showed 98% purity. The 1H NMR analysis in DMSO-d6 was consistent with the assigned structure of Cbz-β-alanine glycolic acid [δ 10.16 (s, 1H), 7.32 (bs, 5H), 5.57 (bs, 1H), 5.14 (s, 2H), 4.65 (s, 2H), 3.45 (m, 2H), 2.64 (m, 2H)].

Intermediate, docetaxel-2′-carbobenzyloxy-β-alanine glycolate (docetaxel-2′-Cbz-β-alanine glycolate), was prepared using a 250-mL round-bottom flask equipped with a magnetic stirrer that was charged with docetaxel (5.03 g, 6.25 mmol). Cbz-β-alanine glycolic acid [1.35 g, 4.80 mmol] and dichloromethane (DCM, 100 mL). The mixture was stirred for 5 min to produce a clear solution, to which N-(3-Dimethylaminopropyl)-N′-ethylcarbodiimide hydrochloride (EDC.HCl, 1.00 g, 5.23 mmol) and 4-(dimethylamino)pyridine (DMAP, 0.63 g, 5.23 mmol) were added. The mixture was stirred at ambient temperature for 3 h, at which point HPLC analysis showed 48% conversion along with 46% of residual docetaxel. A second portion of Cbz-β-alanine glycolic acid (0.68 g, 2.39 mmol), EDC.HCl (0.50 g, 1.04 mmol) and DMAP (0.13 g, 1.06 mmol) were added and the reaction was allowed to stirred overnight. At this point, HPLC analysis showed 69% conversion along with 12% of residual docetaxel. The solution was diluted to 200 mL with DCM and then washed with 80 mL of water (twice) and 80 mL of brine. The organic layer was separated, dried over sodium sulfate, and then filtered. The filtrate was concentrated to a residue, re-dissolved in 10 mL of chloroform, and purified using a silica gel column. The fractions containing product (shown as a single spot by TLC analysis) were combined, concentrated to a residue, vacuum-dried at ambient temperature for 16 h to produce docetaxel-2′-Cbz-β-alanine glycolate as a white powder [3.5 g, yield: 52%]. HPLC analysis (AUC, 227 nm) indicated>99.5% purity. The 1H NMR analysis confirmed the corresponding peaks.

Intermediate, docetaxel-2′β-alanine glycolate, was prepared using a 250 mL round-bottom flask equipped with a magnetic stirrer that was charged with docetaxel-2′-Cbz-β-alanine glycolate [3.1 g, 2.9 mmol] and tetrahydrofuran (THF, 100 mL). To the clear solution, methanol (MeOH, 4 mL), methanesulfonic acid (172 μL, 2.6 mmol), and 5% palladium on activated carbon (Pd/C, 1.06 g, 10 mol % of Pd) were added. The mixture was evacuated for 15 seconds and filled with hydrogen using a balloon. After 3 h, HPLC analysis indicated that the reaction was complete. Charcoal (3 g, Aldrich, Darco®#175) was then added and the mixture was stirred for 15 min and filtered through a Celite® pad to produce a clear colorless solution. It was concentrated under reduced pressure at <20° C. to ˜5 mL, to which 100 mL of heptanes was added slowly resulting in the formation of a white gummy solid. The supernatant was decanted and the gummy solid was vacuum-dried for 0.5 h to produce a white solid. A volume of 100 mL of heptanes were added and the mixture was triturated for 10 min and filtered. The filter cake was vacuum-dried at ambient temperature for 16 h to produce docetaxel-2′-β-alanine glycolate as a white powder [2.5 g, yield: 83%]. The HPLC analysis indicated>99% purity (AUC, 230 nm). MS analysis revealed the correct molecular mass (m/z: 936.5).

A 100 mL round bottom equipped with a magnetic stirrer was charged with O-acetyl-5050-PLEA [5.0 g, 0.7 mmol], docetaxel-2′-β-alanine glycolate [0.80 g, 0.78 mmol], dichloromethane (DCM, 5 mL) and dimethylformamide (DMF, 20 mL). The mixture was stirred for 5 min to produce a clear solution. EDC.HCl (0.22 g, 1.15 mmol) and DMAP (0.22 g, 1.80 mmol) were added to the mixture and the reaction was stirred for 3 hours, at which time HPLC analysis indicated completion of the reaction. The reaction was concentrated under vacuum to remove DCM and then DCM was twice exchanged with 10 mL of acetone. The residue was diluted with acetone to 30 mL and precipitated in cold water containing 600 mL of 0.1% acetic acid. The resulting suspension was filtered and the filter cake was vacuum-dried for 24 h to afford a crude product as a white powder [yield=5.0 g]. The 1H NMR analysis indicated the presence of trace amounts of DMF and DMAP. The docetaxel loading was estimated to be approximately 10 wt % and HPLC analysis indicated>99% purity (AUC, 230 nm). To purify the crude product, it was dissolved in 20 mL of acetone and precipitated in 500 mL of cold water. The suspension was filtered through a polypropylene (PP) filter and the filter cake was vacuum-dried for 48 h to produce O-acetyl-5050-PLGA-(2′-β-alanine glycolate)-docetaxel as a white powder [4.8 g, yield: 84%]. GPC analysis showed that Mw=7.4 kDa, Mn=5.0 kDa and PDI=1.48. ¹H NMR analysis indicated a docetaxel loading of 10.7 wt % and HPLC analysis showed>99% purity (AUC, 230 nm).

### Synthetic scheme of O-acetyl-5050-PLGA-(2′-β-alanine glycolate)-docetaxel

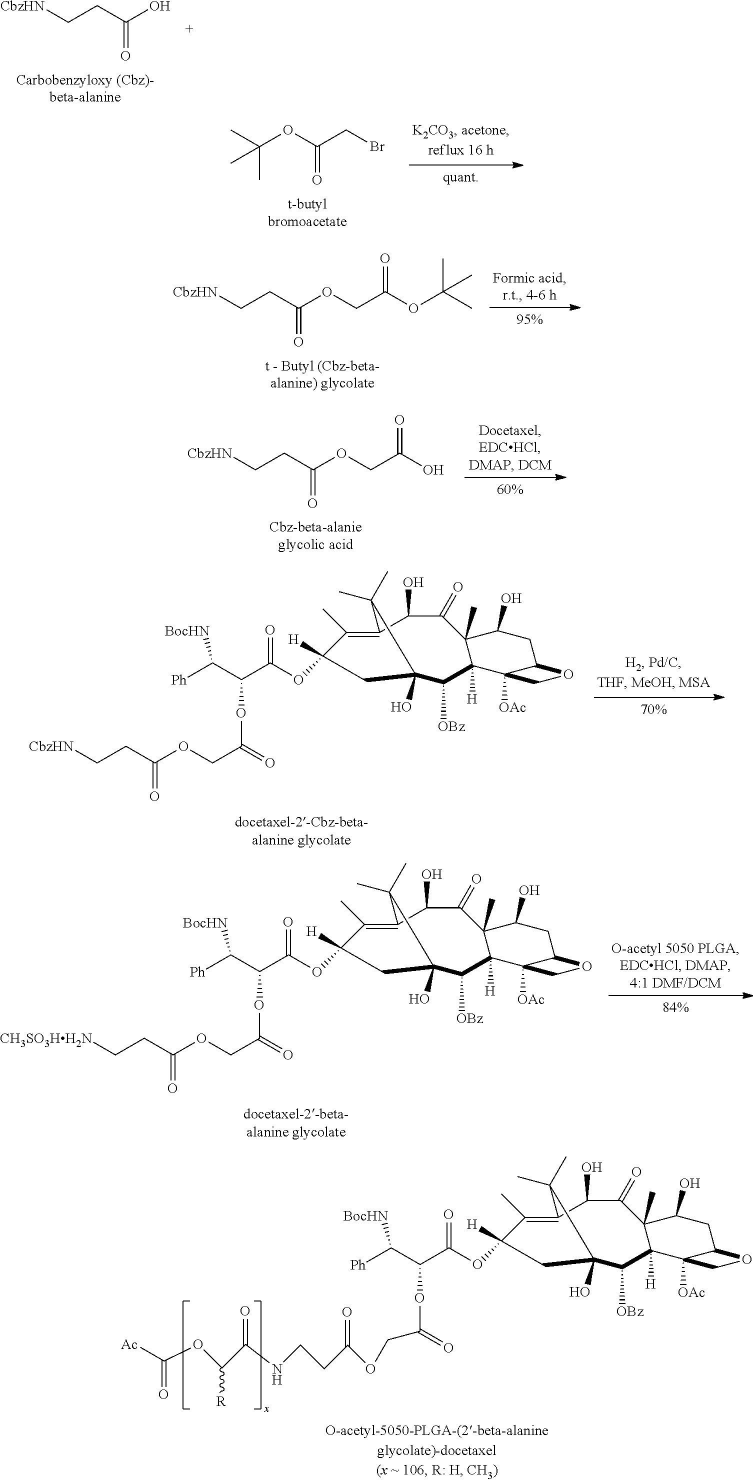

### Example 14

To synthesize lauryl-PLA-O—CO—O-docetaxel, PLA-lauryl ester (inherent viscosity: 1-2 dL/g) was first purified. A mass of 25 g of PLA lauryl ester was dissolved in a 1:1 MTBE/heptanes mixture (100 vol.) with mechanical stirring at ambient temperature. The entire solution was concentrated to dryness and further dried under vacuum at ambient temperature to afford a white powder (18 g). The ¹H NMR analysis indicated 1.44 equivalents of lauryl segment. GPC analysis indicated a Mn and Mw of 8.5 kDa and 10.7 kDa respectively.

A 250-mL round-bottom flask was charged with purified PLA-lauryl ester (10.0 g, 1.18 mmol] and anhydrous DCM (50 mL) under nitrogen. The mixture was stirred for 10 min to afford a clear solution. p-Nitrophenyl chloroformate (0.5 g, 2.4 mmol) was added to the solution and the mixture was stirred for an additional 10 min. A solution of TEA (0.5 mL) was then added dropwise and the reaction was stirred at ambient temperature for 6 h. An additional one equivalent of p-nitrophenyl chloroformate (0.25 g, 1.2 mmol) and TEA (0.25 mL) were added and the reaction was stirred for 12 h. IPC analysis (¹H NMR) indicated completion of the reaction. The solution was concentrated to a residue and dissolved in acetone (20 mL), resulting in a cloudy mixture. This mixture was filtered to remove TEA.HCl and the filtrate was precipitated into a solution of 2:1 MTBE/heptanes (1000 mL). The resulting gummy solid was dissolved in acetone (20 mL) and concentrated to a residue, which was dried under vacuum at ambient temperature for 24 h to afford 5.6 g of p-NO₂-phenyl-COO-PLA-CO₂-lauryl [yield: ˜50%]. The ¹H NMR analysis confirmed the desired product and GPC analysis showed a Mn and Mw of 9.3 and 11.1 kDa respectively.

A 100-mL round-bottom flask was charged with p-NO₂-phenyl-COO-PLA-007-lauryl [2.5 g, 0.28 mmol], docetaxel (0.20 g, 0.25 mmol) and 1:1 DCM/EtOAc (15 mL). The entire mixture was stirred for 10 min. A catalyst, dialkylaminopyridine (DMAP, 61 mg, 0.5 mmol) was added to the mixture and allowed to stir at ambient temperature under N₂ for 6 h. The reaction was stirred for another 10 h to reach completion as confirmed by IPC analysis (¹H NMR). The reaction was then filtered through a 0.45 μM PTFE membrane and the filtrate was added dropwise into 2:1 MTBE/heptanes (600 mL) with vigorous agitation, resulting in a suspension. The milky supernatant was decanted off and the gummy solid was dissolved in acetone (15 mL). The solution was then added dropwise into an ice-cold solution of 0.1% sodium bicarbonate (300 mL) with agitation. The resulting suspension was filtered and the solid was dried under vacuum at ambient temperature for 24 h to afford 1.34 g of lauryl-PLA-O—CO—O-docetaxel [yield: 51%]. The ¹H NMR analysis indicated 9.3 wt % of docetaxel loading. GPC analysis showed a Mn and Mw of 12.4 and 14.3 kDa respectively.

### Example 15

The triblock copolymer PLGA-PEG-PLGA will be synthesized using the protocol reported by Zentner et al. Journal of Controlled Release, 72, 2001, 203-215. The molecular weight of PGLA obtained using this method would be about 3 kDa. A similar method reported by Chen et al., International Journal of Pharmaceutics, 288, 2005, 207-218 will also be used to synthesize PLGA molecular weights ranging from about 1 to about 7 kDa. The LA/GA ratio typically will be 1:1. The minimum PEG molecular weight will be about 2 kDa with an upper limit of about 30 kDa. The preferred range of PEG will be about 3 to about 12 kDa. The PLGA molecular weight would be a minimum value of about 4 kDa and a maximum value of about 30 kDa. The preferred range of PLGA would be about 7 to about 20 kDa. Drug (e.g. docetaxel, paclitaxel, doxorubicin, etc.) could be conjugated to the PLGA through an appropriate linker (i.e. as listed in the previous examples) to form a polymer-drug conjugate. In addition, the same drug or a different drug could be attached to the other PLGA to form a dual drug polymer conjugate with two same drugs or two different drugs. Nanoparticles could be formed from either the PLGA-PEG-PLGA alone or from a single drug or dual polymer conjugate composed of this triblock copolymer.

### Example 16

The triblock polycaprolactone-poly(ethylene glycol)-polycarpolactone (PCL-PEG-PCL) will be synthesized using a ring open polymerization method in the presence of a catalyst (i.e. stannous octoate) as reported in Hu et al., Journal of Controlled Release, 118, 2007, 7-17. The molecular weights of PCL obtained from this synthesis range from about 2 to about 22 kDa. A non-catalyst method shown in the article by Ge et al. Journal of Pharmaceutical Sciences, 91, 2002, 1463-1473 will also be used to synthesize PCL-PEG-PCL. The molecular weights of PCL obtained from this particular synthesis range from about 9 to about 48 kDa. Similarly, another catalyst free method developed by Cerrai et al., Polymer, 30, 1989, 338-343 will be used to synthesize the triblock copolymer with molecular weights of PCL ranging from about 1 to about 9 kDa. The minimum PEG molecular weight would be about 2 kDa with an upper limit of about 30 kDa. The preferred range of PEG would be about 3 to about 12 kDa. The PCL molecular weight would be a minimum value of about 4 kDa and a maximum value of about 30 kDa. The preferred range of PCL would be about 7 to about 20 kDa. Drug (e.g. docetaxel, paclitaxel, doxorubicin, etc.) could be conjugated to the PCL through an appropriate linker (i.e. as listed in the previous examples) to form a polymer-drug conjugate. In addition, the same drug or a different drug could be attached to the other PCL to form a dual drug polymer conjugate with two same drugs or two different drugs. Nanoparticles could be formed from either the PCL-PEG-PCL alone or from a single drug or dual polymer conjugate composed of this triblock copolymer.

### Example 17

The triblock polylactide-poly(ethylene col)-polylactide (PLA-PEG-PLA) copolymer will be synthesized using a ring opening polymerization using a catalyst (i.e. stannous octaote) reported in Chen et al., Polymers for Advanced Technologies, 14, 2003, 245-253. The molecular weights of PLA that can be formed ranged from about 6 to about 46 kDa. A lower molecular weight range of about 1 to about 8 kDa could be achieved by using the method shown by Zhu et al., Journal of Applied Polymer Science, 39, 1990, 1-9. The minimum PEG molecular weight would be about 2 kDa with an upper limit of about 30 kDa. The preferred range of PEG would be about 3 to about 12 kDa. The PCL molecular weight would be a minimum value of about 4 kDa and a maximum of about 30 kDa. The preferred range of PCL would be about 7 to about 20 kDa. Drug (e.g. docetaxel, paclitaxel, doxorubicin, etc.) could be conjugated to the PLA through an appropriate linker (i.e. as listed in the previous examples) to form a polymer-drug conjugate. In addition, the same drug or a different drug could be attached to the other PLA to form a dual drug polymer conjugate with two same drugs or two different drugs. Nanoparticles could be formed from either the PLA-PEG-PLA alone or from a single drug or dual polymer conjugate composed of this triblock copolymer.

### Example 18

The triblock p-dioxanone-co-lactide-poly(ethylene glycol)-p-dioxanone-co-lactide (PDO-PEG-PDO) will be synthesized in the presence of a catalyst (stannous 2-ethylhexanoate) using a method developed by Bhattari et al., Polymer International, 52, 2003, 6-14. The molecular weight of PDO obtained from this method could range from about 2 to about 19 kDa. The minimum PEG molecular weight would be about 2 kDa with an upper limit of about 30 kDa. The preferred range of PEG would be about 3 to about 12 kDa. The PDO molecular weight could be a minimum value of about 4 kDa and a maximum of about 30 kDa. The preferred range of PDO would be about 7 to about 20 kDa. Drug (e.g. docetaxel, paclitaxel, doxorubicin, etc.) could be conjugated to the PDO through an appropriate linker (i.e. as listed in the previous examples) to form a polymer-drug conjugate. In addition, the same drug or a different drug could be attached to the other PDO to form a dual drug polymer conjugate with two same drugs or two different drugs. Nanoparticles could be formed from either the PDO-PEG-PDO alone or from a single drug or dual polymer conjugate composed of this triblock copolymer.

### Example 19

Synthesis of docetaxel-PLGA nanoparticles using PVA as a surfactant via nanoprecipitation was completed using the following protocol. Docetaxel-2′-5050 PLGA-O-acetyl (700 mg, 70 wt % or 600 mg, 60 wt %,) and mPEG-PLGA (300 mg, 30 wt % or 400 mg, 40 wt %. Mw 12.9 kDa) were dissolved to form a total concentration of 1.0% polymer in acetone. In a separate solution, 0.5% w/v PVA (80% hydrolyzed, Mw 9-10 kDa) in water was prepared. The polymer acetone solution was added using a syringe pump at a rate of 1 mL/min to the aqueous solution (v/v ratio of organic to aqueous phase=1:10), with stirring at 500 rpm. Acetone was removed by stirring the solution for 2-3 hours. The nanoparticles were then washed with 10 volumes of water and concentrated using a tangential flow filtration system (300 kDa MW cutoff, membrane area=50 cm²). The solution was then passed through a 0.22 μm filter, and adjusted to a final concentration of 10% sucrose. The nanoparticles were further lyophilized into powder form. The nanoparticles contained about half the amount of PEG and 15-30% PVA.

The nanoparticle properties were evaluated by using a plurality of nanoparticles made in the method above: (prior to passing through 0.22 μm filter). The results are shown in Table 1 below.

TABLE 1
Properties of Docetaxel-PLGA Nanoparticles

| | Docetaxel-2′-5050 | Docetaxel-2′-5050 |
|---|---|---|
| | PLGA-O-acetyl/ | PLGA-O-acetyl/ |
| | PLGA-mPEG | PLGA-mPEG |
| | Starting amt: | Starting amt: |
| | (70/30 wt %) | (60/40 wt %) |
| Z-average (nm) | 93 | 84 |
| Particle PDI | 0.09 | 0.06 |
| Dv₅₀ (nm) | 76 | 71 |
| Dv₉₀ (nm) | 124 | 109 |

### Example 20

The synthesis of PEGylated docetaxel-2′-5050 PLGA-O-acetyl nanoparticles using Tween 80 as the surfactant via nanopercipitation was completed using the following protocol. Docetaxel-2′-5050 PLGA-O-acetyl (672 mg, 84 wt %) and mPEG-PLGA (128 mg, 16 wt %, Mw 12.9 kDa,) were dissolved to form a total concentration of 2.0% polymer in acetone. In a separate solution, 0.5% w/v Tween in water was prepared. The polymer acetone solution was added using a syringe pump at a rate of 1 mL/min to the aqueous solution (v/v ratio of organic to aqueous phase=1:10), with stirring at 500 rpm. Acetone was removed by stirring the solution for 2-3 hours. The nanoparticles were then washed with 10 volumes of 0.5% w/v Tween 80 and concentrated using a tangential flow filtration system (300 kDa MW cutoff, membrane area=50 cm²). The solution was then passed through a 0.22 μm Nylon filter, and adjusted to a final concentration of 10% sucrose. The nanoparticles could be lyophilized into powder form. The nanoparticles contain about half the amount of PEG and 5-15% surfactant. The nanoparticle properties were evaluated by using a plurality of the nanoparticles made by the method described above and the results are listed in Table 2.

TABLE 2
Properties of PEGylated docetaxel-2′-5050 PLGA-O-acetyl Nanoparticles

| | |
|---|---|
| Z-average (nm) | 107 |
| Particle PDI | 0.112 |
| Dv₅₀ (nm) | 89 |
| Dv₉₀ (nm) | 150 |

### Example 21

Synthesis of PEGylated Docetaxel-2′-5050 PLGA-O-acetyl nanoparticles using Solutol as the surfactant via nanoprecipitation was completed using the following protocol. Docetaxel-2′-5050 PLGA-O-acetyl (672 mg, 84 wt %) and mPEG-PLGA (128 mg, 16 wt %, Mw 12.9 kDa,) were dissolved to form a total concentration of 2.0% polymer in acetone. In a separate solution, 0.5% w/v Solutol HS 15 in water was prepared. The polymer acetone solution was added using a syringe pump at a rate of 1 mL/min to the aqueous solution (v/v ratio of organic to aqueous phase=1:10), with stirring at 500 rpm. Acetone was removed by stirring the solution for 2-3 hours. The nanoparticles were then washed with 10 volumes of 0.5% w/v Solutol HS 15 and concentrated using a tangential flow filtration system (300 kDa MW cutoff, membrane area=50 cm²). The solution was then passed through a 0.22 μm Nylon filter, and adjusted to a final concentration of 10% sucrose. The nanoparticles were further lyophilized into powder form. The nanoparticles contained about half the amount of PEG and 5-15% surfactant. The nanoparticle properties were evaluated by using a plurality of nanoparticles made by the method above. The results are shown in Table 3 below.

TABLE 3
Properties of PEGylated docetaxel-2′-5050 PLGA-O-acetyl Nanoparticles

| | |
|---|---|
| Z-average (nm) | 106 |
| Particle PDI | 0.093 |
| Dv₅₀ (nm) | 91 |
| Dv₉₀ (nm) | 147 |

### Example 22

Synthesis of PEGylated Docetaxel-2′-5050 PLGA-O-acetyl/Doxorubicin 5050 PLGA amide nanoparticles using PVA as the surfactant via Nanoprecipitation was completed. Docetaxel-2′-5050 PLGA-O-acetyl (400 mg, 59 wt %), doxorubicin 5050 PLGA amide (200 mg, 8.9 wt %) and mPEG-PLGA (40 mg, 6.25 wt %, Mwt. 8232 Da) were dissolved to form a total concentration of 1.0% polymer in acetone. In a separate solution, 0.5% w/v PVA (viscosity 2.5-3.5 cp) in water was prepared. The polymer acetone solution was added using a syringe pump at a rate of 1 mL/min to the aqueous solution (v/v ratio of organic to aqueous phase=1:10), with stirring at 500 rpm. Acetone was removed by stirring the solution for 2-3 hours. The nanoparticles were then washed with 10 volumes of water and concentrated using a tangential flow filtration system (300 kDa MW cutoff, membrane area=50 cm²). The nanoparticle solution was adjusted to a final concentration of 10% sucrose. The nanoparticles could be lyophilized into powder form. The nanoparticles contained about half the amount of PEG and 15-30% PVA. The nanoparticle properties were further evaluated by using a plurality of nanoparticles made by the method described above. The results are shown in Table 4 below.

TABLE 4
Properties of PEGylated Docetaxel-2′-5050 PLGA-O-acetyl/ Doxorubicin
5050 PLGA amide Nanoparticles

| | |
|---|---|
| Z-average (nm) | 146.6 |
| Particle PDI | 0.146 |
| Dv₅₀ (nm) | 137 |
| Dv₉₀ (nm) | 211 |

### Example 23

Synthesis of Rhodamine labeled PEGylated Docetaxel-2′-5050 PLGA-O-acetyl via nanoprecipitation using PVA as the surfactant was completed using the following protocol. Para-nitrophenyl protected PEG-PLGA 5050-lauryl ester (150 mg, 1.36×10⁻⁵ moles) was added to rhodamine B ethylene diamine (8 mg, 1.36×10⁻⁵ moles) in N,N dimethylformamide (DMF) in the presence of triethylamine (4 uL, 2.72×10⁻⁵ moles). The reaction mixture was stirred at room temperature overnight. DMF was removed from the reaction mixture under vacuum. Purification of the product was obtained through 3 times precipitation of the crude product dissolved in dichloromethane in methyl tert-butyl ether. The product (shown by Scheme 1 below) was then dried under vacuum overnight.

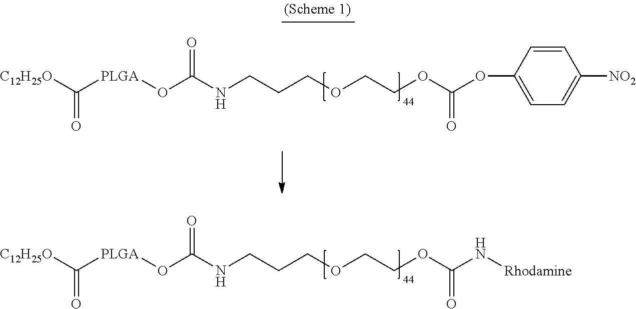

Docetaxel-2′-5050 PLGA-O-acetyl (120 mg, 59 wt %), PEG-PLGA (18 mg, 8.9 wt %, Mw 12.9 kDa), Rhodamine B-labeled-PEG-PLGA-lauryl ester (4 mg, 1.9 wt %) and purified PLGA (60 mg, 30 wt %) were dissolved to form a total concentration of 1.0% polymer in acetone. In a separate solution, 0.5% w/v PVA (viscosity 2.5-3.5 cp) in water was prepared. The polymer acetone solution was added using a syringe pump at a rate of 1 mL/min to the aqueous solution (v/v ratio of organic to aqueous phase=1:10), with stirring at 500 rpm. Acetone was removed by stirring the solution for 2-3 hours. The nanoparticles were then washed with 10 volumes of water and concentrated using a tangential flow filtration system (300 kDa MW cutoff, membrane area=50 cm²). The nanoparticle solution was adjusted to a final concentration of 10% sucrose. The nanoparticles were further lyophilized into powder form. The nanoparticles contained half the amount of PEG and 15-30% PVA.

### Example 24

Synthesis of Docetaxel-2′-5050 PLGA-O-acetyl nanoparticles via Micro-Mixer using PVA as the surfactant was completed using the following protocol. 5050 purified PLGA (211 mg, 32 μmol), docetaxel-2′-5050 PLGA-O-acetyl (633 mg, 71 μmol) and mPEG-PLGA (Mw 8.3 kDa, 5 wt % total polymer) were combined at a total concentration of 1.0% polymer in acetone. A separate solution of 0.5% polyvinylalcohol (80% hydrolyzed, Mw 9-10 kDa) in water was prepared. The organic and aqueous solutions were then blended using a Caterpillar MicroMixer, using flow rates of 5 mL/min and 15 mL/min respectively. The acetone was removed from the resulting nanoparticle dispersion by rotary evaporation. The aqueous nanoparticle dispersion was washed with 10 volumes of water using a tangential flow filtration system (300 kDa MW cutoff, membrane area=50 cm²). The dispersion was then concentrated using a tangential flow filtration system (300 kDa MW cutoff, membrane area=50 cm²). The solution was then passed through a 0.22 μm filter, and adjusted to a final concentration of 10% sucrose. The solution was then lyophilized to provide the particles. The nanoparticles contained half the amount of PEG and 15-30% PVA. The nanoparticle properties were evaluated and the results shown in Table 5 below.

TABLE 5
Properties of Docetaxel-2′-5050 PLGA-O-acetyl Nanoparticles

| | |
|---|---|
| Z-average (nm) | 133.9 |
| Particle PDI | 0.199 |
| Dv₅₀ (nm) | 110 |
| Dv₉₀ (nm) | 237 |

### Example 25

Synthesis of Doxorubicin 5050 PLGA amide nanoparticles via emulsion using PVA as the surfactant was completed using the following protocol. Doxorubicin 5050 PLGA amide (100 mg, 100 wt %) was dissolved to form a total concentration of 1.0% polymer in dichloromethane. In a separate solution, 0.5% w/v PVA (viscosity 2.5-3.5 cp) in water was prepared. The dissolved polymer solution in dichloromethane was mixed with the aqueous PVA solution and emulsified through a microfluidizer processor for three cycles at a pressure of 8500 psi. Dichloromethane was removed by stirring the solution for 12 hours. The nanoparticles were then washed with 10 volumes of water and concentrated using a tangential flow filtration system (300 kDa MW cutoff, membrane area=50 cm²). The nanoparticle solution was adjusted to a final concentration of 10% sucrose. The nanoparticles could be lyophilized into powder form and were prepared for purposes of comparison. The nanoparticles contained half the amount of PEG and 15-30% PVA. The nanoparticle properties were evaluated and the results shown in Table 6 below.

TABLE 6
Properties of Doxorubicin-5050 PLGA amide Nanoparticles

| | |
|---|---|
| Z-average (nm) | 91.19 |
| Particle PDI | 0.135 |
| Dv₅₀ (nm) | 70.5 |
| Dv₉₀ (nm) | 120 |

### Example 26

Synthesis of embedded Docetaxel/Paclitaxel in Docetaxel-2′-5050 PLGA-β-acetyl nanoparticles via emulsion using PVA as the surfactant was completed using the following protocol. Docetaxel-2′-5050 PLGA-O-acetyl (90 wt %), mPEG-PLGA (10 wt %) and either docetaxel or paclitaxel (30 mg) were dissolved in dichloromethane (DCM, 14 mL). A separate solution of 0.5% polyvinylalcohol (PVA, 80% hydrolyzed, Mw 9-10 kDa) in water was prepared. The dissolved polymer-drug solution was transferred with a syringe into a beaker containing the 0.5% PVA (96 mL, v/v ratio of organic to aqueous phase=˜1:7) and sonicated using a micro-tip horn (tip diameter=½ inch) for 5 minutes to form an emulsion. The emulsion is then transferred to a microfluidizer processor and passed through seven times with processing pressures ranging from 13,000-16,100 psi. The DCM was removed from the resulting nanoparticle dispersion by rotary evaporation. The aqueous nanoparticle dispersion was washed with 10-20 times volumes of water and concentrated using a tangential flow filtration system (300 kDa MW cutoff, membrane area=50 cm²). The solution was passed through a 0.22 μm filter, and for lyoprotection, 10% sucrose was added. The nanoparticles were lyophilized to form a white powder. The nanoparticles contained half the amount of PEG and 15-30% PVA. The particle properties were evaluated and shown in Table 7 below.

TABLE 7
Properties of Embedded Docetaxel/Paclitaxel in
Docetaxel-2′-5050 PLGA-O-acetyl Nanoparticles

| | Docetaxel | Paclitaxel |
|---|---|---|
| Zavg (nm) | 94 | 102 |
| Particle PDI | 0.107 | 0.103 |
| Dv₅₀ (nm) | 75 | 82 |
| Dv₉₀ (nm) | 128 | 142 |
| Embedded drug (% w/w) | 1.9 | 4.5 |
| Conjugate docetaxel (% w/w) | 4.0 | 4.1 |

### Example 27

The formulation of PEGylated O-acetyl-5050-PLGA-(2′β-alanine glycolate)-docetaxel nanoparticles was carried out according to the following protocol. O-acetyl-5050-PLGA-(2′-β-alanine glycolate)-docetaxel (600 mg, 60 wt %) and mPEG-PLGA (400 mg, 40 wt %) were dissolved to form a total concentration of 1.0% polymer in acetone. In a separate solution, 0.5% w/v PVA (viscosity 2.5-3.5 cp) in water was prepared. The organic and aqueous solutions were then mixed together using a nanoprecipitation method at an organic to aqueous ratio of 1:10. Acetone was removed from the resulting nanoparticle dispersion by passive evaporation. The nanoparticles were then washed with 12 volumes of water and concentrated using a tangential flow filtration system (300 kDa MW cutoff, membrane area=50 cm²). The nanoparticle solution was adjusted to a final concentration of 10% sucrose. The nanoparticles could be lyophilized into powder form. The nanoparticles contain half Initial amount of mPEG-PLGA, and 15-30% PVA.

TABLE 8
Properties of PEGylated O-acetyl-5050-PLGA-(2′-β-alanine glycolate)-
Docetaxel Nanoparticles

| | |
|---|---|
| Z-average (nm) | 74.3 |
| Particle PDI | 0.097 |
| Dv₅₀ (nm) | 57.5 |
| Dv₉₀ (nm) | 94.4 |

### Example 28

The formulation of PEGylated bis(docetaxel) glutamate-5050 PLGA-O-acetyl nanoparticles was carried out according to the following protocol. Bis(docetaxel) glutamate-5050 PLGA-O-acetyl (600 mg, 60 wt %) and mPEG-PLGA (400 mg, 40 wt %) were dissolved to form a total concentration of 1.0% polymer in acetone. In a separate solution, 0.5% w/v PVA (viscosity 2.5-3.5 cp) in water was prepared. The organic and aqueous solutions were then mixed together using a nanoprecipitation method at an organic to aqueous ratio of 1:10. Acetone was removed from the resulting nanoparticle dispersion by passive evaporation. The nanoparticles were then washed with 12 volumes of water and concentrated using a tangential flow filtration system (300 kDa MW cutoff, membrane area=50 cm²). The nanoparticle solution was adjusted to a final concentration of 10% sucrose. The nanoparticles could be lyophilized into powder form. The nanoparticles contain half Initial amount of mPEG-PLGA, and 15-30% PVA.

TABLE 9
Properties of PEGylated bis(docetaxel) glutamate-5050
PLGA-O-acetyl Nanoparticles

| | |
|---|---|
| Z-average (nm) | 68.6 |
| Particle PDI | 0.082 |
| Dv₅₀ (nm) | 55.9 |
| Dv₉₀ (nm) | 87.2 |

### Example 29

The formulation of PEGylated O-acetyl-5050-PLGA-(2′-β-alanine glycolate)-docetaxel/docetaxel-2′5050 PLGA-o-acetyl nanoparticles could be carried out according to the following protocol. O-acetyl-5050-PLGA-(2′-β-alanine glycolate)-docetaxel, docetaxel-5050 PLGA-o-acetyl and mPEG-PLGA could be combined at a weight ratio of 84-60/16-40 wt % (polymer drug conjugates/mPEG-PLGA) with a total concentration of 1% polymer in acetone. In a separate solution, 0.5% w/v PVA (viscosity 2.5-3.5 cp) in water could be prepared. The polymer drug conjugates could vary from a ratio of 10:1 to 1:10. The organic and aqueous solutions could then be mixed together using a nanoprecipitation method at an organic to aqueous ratio of 1:10. The acetone could be removed from the resulting nanoparticle dispersion by passive evaporation. The nanoparticles could be washed with 15 volumes of water and concentrated using a tangential flow filtration system (300 kDa MW cutoff, membrane area=50 cm²). The nanoparticle solution could be adjusted to a final concentration of 10% sucrose. The nanoparticles could be lyophilized into powder form. This particular nanoparticle configuration could allow for different release rates of docetaxel.

### Example 30

Synthesis of docetaxel-2′-hexanoate-5050 PLGA-O-acetyl nanoparticles could be completed by the following protocol. Docetaxel-2′-hexanoate-5050 PLGA-O-acetyl and mPEG-PLGA could be combined at a weight ratio ranging from 84-60/16-40 wt % with a total concentration of 1% polymer in acetone. In a separate solution, 0.5% w/v PVA (viscosity 2.5-3.5 cp) in water could be prepared. The polymer acetone solution could be added using a syringe pump at a rate of 1 mL/min to the aqueous solution (v/v ratio of organic to aqueous phase=1:10), with stirring at 500 rpm. Acetone could be removed by stirring the solution for 2-3 hours. The nanoparticles could then be washed with 10 volumes of water and concentrated using a tangential flow filtration system (300 kDa MW cutoff, membrane area=50 cm²). For lyoprotection, standard cryoprotectants could be used (e.g. sucrose) and the nanoparticles could be further lyophilized into powder form. The nanoparticles could contain half the amount of PEG and 15-30% PVA.

### Example 31

Docetaxel-PLGA Nanoparticles samples were prepared for Imaging using Cryo Scanning Electron Microscopy (Cryo-SEM) by the following protocol. Lyophilized samples of docetaxel-PLGA nanoparticles containing PVA were reconstituted and fixed in 0.5% osmium tetroxide (OsO₄) in water for ca. 15 min prior to centrifugation and washing with water. Sample droplets were placed into a rivet holder, which was fast frozen in liquid nitrogen slush (ca, −210° C.) A vacuum was pulled and the sample was transferred to a Gatan Alto 2500-pre chamber (cooled to ca. −160° C.). The sample was fractured, sublimated at −90° C. for 7-10 minutes and coated with platinum using a sputter coating for 120 sec. Finally the samples were transferred to the microscope cryostage which is maintained at −130° C. The samples were imaged with an FEI NOVA nanoSEM field emission scanning electron microscope operating at an accelerating velocity of 5 kV. The cryo-SEM images showed that the docetaxel-PLGA nanoparticles containing PVA were spherical with no apparent surface structure was evident. The particle sizes ranged from 50-75 nm.

### Example 32

Docetaxel-PLGA Nanoparticles samples were prepared for Imaging using Transmission Electron Microscopy (TEM) by the following protocol. Carbon coated formvar grids (400 mesh) were glow-discharged prior to use. A droplet sample of docetaxel-PLGA nanoparticles containing PVA was added to the carbon grids and allowed to sit for ca. 2 min. The grids were then quickly touched to droplets for 2% uranyl acetate. The excess stain was removed with filter paper and allowed to dry. The samples were imaged with a Phillips CM-100 transmission electron microscope operating at an accelerating velocity of 80 kV.

The TEM images showed that the docetaxel-PLGA nanoparticles containing PVA were spherical and relatively uniform in size. The particle size from the TEM micrograph were typically less than 150 nm.

### Example 33

Doxorubicin Tosylate was synthesized, purified and characterized using the following protocol. In a 250-mL round-bottom flask equipped with a magnetic bar and a thermocouple, doxorubicin.HCl (NetQem, 1.43 g, 2.46 mmol) was suspended in anhydrous THF (143 mL, 100 vol). The mixture was evacuated for 15 seconds while being stirred and filled up with nitrogen (1 atm). 1 M potassium tert-butoxide (KOtBu)/THF solution (2.7 mL, 2.70 mmol) was added dropwise with stirring within 10 min. The solution turned a purple color and a slight exotherm was observed. The reaction temperature rose from 19° C. to 21.7° C. within 15 min and then slightly climbed up to a maximum of 22.4° C. in half hour. The mixture was stirred for another hour at 22.4° C. and then p-Toluenesulfonic acid (p-TSA, 0.70 g, 3.96 mmol) was added in one portion. The solution immediately turned a red color along with the precipitation of fine particles. The mixture was stirred for an additional half hour at ambient temperature and then cooled to 5° C. and stirred for 1 h. The resulting red suspension was filtered under nitrogen. The filter cake was washed with THF (3-10 mL) and dried under vacuum at 25° C. for 16 h to produce doxorubicin tosylate [1.73 g, 97% yield)]. HPLC analysis indicated a 97% purity (AUC, 480 nm).

To remove the excess p-TSA, the product was slurried in 5:1 MTBE/MeOH (60 mL) at ambient temperature for 3 h. The filtered solid was dried under vacuum at 25° C. for 16 h to afford 1.32 g of product. HPLC analysis indicated 99% purity (AUC, 480 nm); however, the ¹H NMR analysis showed that the equivalents of p-TSA were still ˜1.2. DSC analysis of doxorubicin tosylate showed a sharp peak with a melting range of 188.5-196.5° C.

### Example 34

Doxorubicin Octanesulfonate was synthesized and characterized using the following protocol. In a 250 mL round-bottom flask equipped with a magnetic stirrer, 1-octanesulfonic acid sodium salt monohydrate (0.44 g, 1.86 mmol) was dissolved in water (50 mL). The mixture was stirred for 10 min to afford a clear solution, to which doxorubicin.HCl (1.08 g, 1.86 mmol) was added in one portion. The solution became a dark red color after being stirred for a few minutes. After about 30 min, an orange powder formed. The mixture was stirred at ambient temperature for 2 h. The suspension was stored in fridge for 16 h and filtered through a Sharkskin® filter paper. The filtrate had a slightly red color and contained trace amounts of doxorubicin as evidenced by HPLC analysis. The presence of chloride in the filtrate was confirmed by the silver nitrate test. The filter cake was dried under vacuum at 28° C. for 16 h to afford doxorubicin octanesulfonate [1.16 g, yield: 85%] as an orange powder. The ¹H NMR analysis indicated the desired product and HPLC analysis indicated>99.5% purity. DSC analysis of doxorubicin octanesulfonate showed a sharp peak with a melting range of 198.7-202.0° C.

### Example 35

Doxorubicin Naphthalene-2-Sulfonate was synthesized, purified and characterized using the following protocol. A 250-mL round-bottom flask equipped with a magnetic bar and a thermocouple was charged with doxorubicin.HCl (NetQem, 1.47 g, 2.53 mmol) and anhydrous THF (150 mL, 100 vol). The suspension was evacuated for 15 seconds with stirring and tilled up with nitrogen (1 atm). 1 M (KOtBu)/THF solution (2.7 mL, 2.70 mmol) was added dropwise with stirring over 10 min. The mixture turned a purple color and a slight exotherm was observed, causing the reaction temperature to rise from 20.2° C. to 21.4° C. within 15 min. The solution was stirred at 21.1° C. for one hour and 2-naphthalenesulfonic acid (0.63 g, 3.04 mmol) was added in one portion. The mixture immediately turned to a red color and the precipitation of fine particles was observed. The solution was stirred for an hour at ambient temperature and then filtered under nitrogen. The filtration was slow and took about 1 h. The filter cake was washed with THF (3×10 mL) and dried under vacuum at 25° C. for 16 h to afford 2.1 g of doxorubicin naphthalene-2-sulfonate as a dark red solid [yield: >100%]. HPLC analysis indicated a 98% purity (AUC, 480 nm). The ¹H NMR analysis showed that the ratio of 2-naphthalenesulfonic acid to doxorubicin was ˜1.08.

To remove residual 2-naphthalenesulfonic acid, the doxorubicin naphthalene-2-sulfonate was slurried in 5:1 MTBE/MeOH (60 mL) for 3 h. The suspension was filtered and the filter cake was dried under vacuum at 25° C. for 24 h to afford 1.90 g of the product as a fine red powder [yield: 100%]. The ¹H NMR analysis indicated a clean product with a 1:1 ratio of doxorubicin to 2-naphthalenesulfonic acid. HPLC analysis showed>98% purity (AUC, 480 nm). The physical appearance of the product was similar to doxorubicin.HCl. DSC analysis of doxorubicin naphthalene-2-sulfonate showed a sharp peak with a melting range of 203.1-207.4° C.

### Example 36

The Cytotoxicity of nanoparticles formed from polymer drug conjugates was evaluated using the following protocol. To measure the cytotoxic effect of nanoparticles formed from doxorubicin 5050 PLGA amide, paclitaxel-2′-5050 PLGA-O-acetyl, docetaxel-2′-5050 PLGA-O-acetyl or bis(2′-docetaxel) glutamate-5050 PLGA-O-acetyl, the CellTiter-Glo Luminescent Cell Viability Assay (CTG) was used. Briefly, ATP and oxygen in viable cells reduce luciferin to oxyluciferin in the presence of luciferase to produce energy in the form of light. B16.F10 cells, grown to 85-90% confluency in 150 cm² flasks (passage<30), were resuspended in media (MEM-alpha, 10% HI-FBS, 1× antibiotic-antimycotic) and added to 96-well opaque-clear bottom plates at a concentration of 1500 cells/well in 200 μL/well. The cells were incubated at 37° C. with 5% CO₂ for 24 hours. The following day, serial dilutions of 2×concentrated particles and 2× concentrated free drug were made in 12-well reservoirs with media to specified concentrations. The media in the plates was replaced with 100 μL of fresh media and 100 μL of the corresponding serially diluted drug. Three sets of plates were prepared with duplicate treatments. Following 24, 48 and 72 hours of incubation at 37° C. with 5% CO₂, the media in the plates was replaced with 100 μL of fresh media and 100 μA of CTG solution, and then incubated for 5 minutes on a plate shaker at room temperature set to 450 rpm and allowed to rest for 15 minutes. Viable cells were measured by luminescence using a microtiter plate reader. The data was plotted as % viability vs. concentration and standardized to untreated cells. The doxorubicin 5050 PLGA amide, paclitaxel-2′-5050 PLGA-O-acetyl, docetaxel-2′-5050 PLGA-O-acetyl and bis(2′-docetaxel) glutamate-5050 PLGA-O-acetyl polymer drug conjugates inhibited the growth of B16.F10 cells in a dose and time dependent manner. Also, in comparison to the corresponding free drug, the polymer drug conjugates exhibited a slower release profile as shown by Table 10.

TABLE 10
IC₅₀ values for Nanoparticles comprising polymer-drug conjugates

| Group | IC₅₀ (μM) |
|---|---|
| Free doxorubicin | 14 |
| Doxorubicin 5050 PLGA amide nanoparticles | 2.9 |
| Free paclitaxel | 7 |
| Paclitaxel-2′-5050 PLGA-O-acetyl nanoparticles | 480 |
| Free docetaxel | 0.13 |
| Docetaxel-2′-5050 PLGA-O-acetyl nanoparticles | 20 |
| bis(2′-docetaxel) glutamate-5050 PLGA-O-acetyl | 25 |
| nanoparticles | |

### Example 37

Bioburden tests for contamination of nanoparticles formed from polymer drug conjugate were performed. To measure the formulation sterility for PEGylated docetaxel-2′-5050 PLGA-O-acetyl nanoparticles, the spot colony forming units per gram (CFU) assay, a modified plate count method, was used. A positive control was prepared by inoculating 10 mL of trypticase soy broth (TSB) with an isolated colony from an in house bacterial stock and grown at 37° C. in a shaking incubator at 350 rpm for 24 hours. A subculture (1:100) was then prepared and grown at 37° C. in a shaking incubator (350 rpm for 3 hours). The bacteria were then pelleted, washed with PBS and resuspended with fresh TSB. A 0.5 McFarland standard bacterial solution (equal to 1.5×10⁶ CFU/mL based on turbidity measurement) was then prepared. An aliquot of 100 μL was sampled from each of the following solutions: a cu. 1.5 mg/ml nanoparticle solution (4-5 mL batch size), a positive control and TSB, as well as a negative control. These were each mixed with 400 μLof TSB in a 1.5 mL microcentrifuge tube and cultured in a shaking incubator at 37° C. (450 rpm for 3 days). On days 0 and 3, 50 μL of each sample were removed from the sample mix and serially diluted at a ratio of 1:10 with TSB in a 96-well plate. The diluted samples (6 μL) were spotted onto pre-dried trypticase soy agar (TSA) plates using a multichannel pipet. The spots were allowed to dry and the plates were incubated at 37° C. for 24 hours. After 24 hours, the isolated colonies were counted and the CFU/mL calculated and the results are shown in Table 11 below. To detect very low concentrations of contaminants, 200 of each sample mix were spread onto agar plates on day 3 and incubated at 37° C. for 24 hours. The tests were carried out over an open flame.

TABLE 11
Bioburden measurement by colony forming units per gram

| | T₀ Spot | T₇₂ Spot | T₇₂ Plate |
|---|---|---|---|
| | CFU | CFU | CFU |
| Description | CFU/mL | CFU/mL | CFU/mL |
| PEGylated docetaxel-2′-5050 | 0 | 0 | 0 |
| PLGA-O-acetyl nanoparticles, | | | |
| Filtered with 0.22 μm Steriflip | | | |
| PEGylated docetaxel-2′-5050 | 0 | 0 | 0 |
| PLGA-O-acetyl nanoparticles, | | | |
| Filtered with 0.45 μm Steriflip | | | |
| Positive control, 1.5 × 10⁶ | 6.67 × 10⁵ | 3.80 × 10¹¹ | Lawn |
| CFU/mL standardized stock | | | |
| solution in TSB | | | |
| Negative control, TSB | 0 | 0 | 0 |

### Example 38

In vivo efficacy of PEGylated Doxorubicin 5050 PLGA amide nanoparticles in a B16.F10 mouse model of melanoma was measured using the following protocol. B16.F10 cells were grown in culture to 85-90% confluency in MEM-α medium supplemented with 10% FBS and 1% penicillin/streptomycin (passage=4) and then resuspended in PBS. B16.F10 cells (density=5×10⁶ cells/mL) were implanted subcutaneously (SC) into the right flank of male C57BL/6 mice (20-22 g) on day 1.

The five treatment groups that were administered to the mice were: 1) 0.9% NaCl solution; 2) Doxil (liposomal formulation of doxorubicin HCl containing 2 mg/mL doxorubicin HCl, Ortho Biotech) at 1 mg/kg dose; 3) three PEGylated doxorubicin 5050 PLGA amide nanoparticles with 1, 2 and 3 mg/kg doxorubicin equivalent doses.

The treatments were administered IV into the tail vein of the mouse at a dose volume of 6 mL/kg, beginning on day 5 post-implantation, when the mean tumor volume was 50 mm³. The treatments were administered on days 5, 8, and 12 (biweekly×3 injections) post tumor implantation. Health status of the animals was monitored and the tumor was measured three times a week. On day-17 post-tumor implantation, mice were euthanized by CO₂ inhalation according to the IUCAC procedure guideline. Tumor from each animal was dissected and tumor volume as well as tumor growth inhibition (TGI) was measured. Tumor volume was calculated using the formula: (width×width×length)/2 mm³. TGI represented as % was calculated using the formula: (1−(treated tumor volume/control tumor volume))×100.

The treatment groups of Doxil and all the PEGylated doxorubicin 5050 PLGA amide nanoparticles showed inhibition of tumor growth on day-17 and the results are shown in Table 12. A dose-dependent tumor growth inhibition was seen with PEGylated doxorubicin 5050 PLGA amide nanoparticles; 37% TGI at 1 mg/kg, 48% TGI at 2 mg/kg and 57% TGI at 3 mg/kg. Doxil at only 1 mg/kg exhibited 60% TGI on day 17.

TABLE 12
Tumor Growth Inhibition Using the PEGylated Doxorubicin
5050 PLGA amide Nanoparticles

| | Dose | Day-17 |
|---|---|---|
| Group | mg/kg | TGI, % |
| 0.9% NaCl control | — | — |
| Doxil | 1 | 60% |
| PEGylated doxorubicin 5050 PLGA amide | 1 | 37% |
| nanoparticles | | |
| PEGylated doxorubicin 5050 PLGA amide | 2 | 48% |
| nanoparticles | | |
| PEGylated doxorubicin 5050 PLGA amide | 3 | 58% |
| nanoparticles | | |

### Example 39

In vivo efficacy of PEGylated paclitaxel-2′-5050 PLGA-O-acetyl nanoparticles in a B16.F10 mouse model of melanoma was measured by the following protocol. B16.F10 cells were grown in culture to 85-90% confluency in MEM-α medium supplemented with 10% FBS and 1% penicillin/streptomycin (passage=4) and then resuspended in PBS. B16.F10 cells (density=5×10⁶ cells/mL) were implanted subcutaneously (SC) into the right flank of male C57BL/6 mice (20-22 g on day 1. The four treatment groups that were administered to the mice were: 1) 0.9% NaCl solution; 2) Abraxane® (Abraxis) at 1.5, 6 and 15 mg/kg dose; 3) free paclitaxel at doses of 1.5, 6 and 15 mg/kg and 4) PEGylated paclitaxel-2′-5050 PLGA-O-acetyl nanoparticles at doses of 1.5, 3, 6, 9, and 15 mg/kg paclitaxel equivalent.

The treatments were administered IV into the tail vein at a dose volume of 6 mL/kg, beginning on day-5 post-implantation, when the mean tumor volume was 55 mm³. The treatments were administered on days 5, 8, and 12 (biweekly×3 injections) post tumor implantation. Health status of the animals was monitored and tumor size was measured three times a week. On day 17, post-tumor implantation, mice were euthanized by CO, inhalation according to the IUCAC procedure guideline. Tumors from each animal were dissected and tumor size was measured. Tumor volume was calculated using the formula: (width×width×length)/2 mm³. TGI represented as % was calculated using the formula: (1−(treated tumor volume/control tumor volume))×100. Abraxane®, free paclitaxel and all PEGylated paclitaxel-2′-5050 PLGA-O-acetyl nanoparticles groups showed inhibition of tumor growth on day 17. A dose-dependent TGI was seen with the free paclitaxel treated groups; 37% TGI at 1.5 mg/kg, 57% % TGI at 6 mg/kg and 83% TGI at 15 mg/kg doses. However, Abraxane® did not show a dose dependent TGI, i.e. 36% TGI at 1.5 mg/kg, 13% % TGI at 6 mg/kg and 70% TGI at 15 mg/kg doses. At the lowest dose of 1.5 mg/kg, PEGylated paclitaxel-2′-5050 PLGA-O-acetyl nanoparticles exhibited a 42% TGI, which is similar to free paclitaxel and Abraxane® treated groups at the same dose. However, PEGylated paclitaxel-2′-5050 PLGA-O-acetyl nanoparticles did not show dose-dependent inhibition of tumor growth, i.e. 42% TGI at 1.5 mg/kg, 40% TGI at 3 mg/kg, 46% TGI at 6 mg/kg, 61% TGI at 9 mg/kg and 58% TGI at 15 mg/kg doses. The results are shown in Table 13 below.

TABLE 13
Tumor Growth Inhibition Using Paclitaxel-2′-5050
PLGA-O-acetyl Nanoparticles

| | Dose | Day- |
|---|---|---|
| Group | mg/kg | 17 TGI, % |
| 0.9% NaCl control | — | — |
| Abraxane ® | 1.5 | 36% |
| Abraxane ® | 6 | 13% |
| Abraxane ® | 15 | 70% |
| Free paclitaxel | 1.5 | 37% |
| Free paclitaxel | 6 | 57% |
| Free paclitaxel | 15 | 83% |
| PEGylated paclitaxel-2′-5050 PLGA-O-acetyl | 1.5 | 42% |
| nanoparticles | | |
| PEGylated paclitaxel-2′-5050 PLGA-O-acetyl | 3 | 40% |
| nanoparticles | | |
| PEGylated paclitaxel-2′-5050 PLGA-O-acetyl | 6 | 46% |
| nanoparticles | | |
| PEGylated paclitaxel-2′-5050 PLGA-O-acetyl | 9 | 61% |
| nanoparticles | | |
| PEGylated paclitaxel-2′-5050 PLGA-O-acetyl | 15 | 58% |
| nanoparticles | | |

### Example 40

Tolerability and in vivo efficacy of PEGylated docetaxel-2′-5050 PLGA-O-acetyl nanoparticles in a B16.F10 mouse model of melanoma was measured using the following protocol. B16F10 cells were grown in culture to 85% confluency in MEM-α medium containing 10° A FBS and 1% penicillin/streptomycin (passage=4) and then resuspended in PBS. B1610 cells (density=10×10⁶ cells) were implanted subcutaneously (SC) into the right flank of male C57BL/6 mice on Day 1. On Day 5 following tumor inoculations, animals were assigned to different treatment groups according to the tumor size.

The three treatment groups that were administered to the mice included: 1) a docetaxel vehicle formulation consisting of a 10 mg/mL stock solution (prepared with 20 mg of docetaxel, 0.2 mL ethanol, 0.5 mL Tween 80 and 1.3 mL water, added in that specific order and vortexed to ensure proper mixing). The stock solution was diluted further with PBS to 0.6 and 1.5 mg/mL (for a corresponding dose of 6 and 15 mg/kg) so that all the groups received the same amount of ethanol, Tween 80, water and PBS. 2) PEGylated (10 mol %) docetaxel-2′-5050 PLGA-O-acetyl nanoparticles at doses of 6, 15 and 30 mg/kg). 3) Docetaxel vehicle.

Animals were treated with different concentrations of docetaxel and PEGylated docetaxel-2′-5050 PLGA-O-acetyl nanoparticles as per the schedule (on Days 5, 8 and 12 following inoculation). The schedule consisted of 3 injections biweekly. The animals were monitored three times a week for health status and adverse effects from tumor cell inoculation to the end of the study. The body weight and tumor volume were also measured three times a week to evaluate the effect of the treatment.

On Day 17, the PEGylated (10 mol %) docetaxel-2′-5050 PLGA-O-acetyl nanoparticles showed dose-dependent tumor growth inhibition. At 6, 15 and 30 mg/kg, the TGI was 53%, 88% and 93% after biweekly×3 injections.

### Example 41

The tolerability and maximum tolerated dose of PEGylated bis(2′-docetaxel) glutamate-5050 PLGA-O-acetyl nanoparticles in a B16.F10 mouse model of melanoma was measured. B16F10 cells were grown in culture to confluency in MEM-α medium containing 10% FBS and 1% penicillin/streptomycin (passage=4) and then resuspended in PBS. B1610 cells (density=1×10⁶ cells/mL in a 0.1 mL volume) were subcutaneously (SC) implanted into the right flank of male C57BL/6 mice on Day 1.

The five treatment groups that were administered to the mice included: 1) a docetaxel vehicle formulation consisting of a 10 mg/mL stock solution (prepared with 20 mg of docetaxel, 0.2 mL ethanol, 0.5 mL Tween 80 and 1.3 mL water, added in that specific order and vortexed to ensure proper mixing). The stock solution was diluted further with PBS to 0.6, 1.5, 3, 4.5 and 6 mg/mL (for a corresponding dose of 6, 15, 30, 45 and 60 mg/kg) so that all the groups received the same amount of ethanol, Tween 80, water and PBS. 2) PEGylated bis(2′-docetaxel) glutamate-5050 PLGA-O-acetyl nanoparticles at doses of 6, 15, 30, 45 and 60 mg/kg. 3) Docetaxel vehicle at the highest concentration of 6 mg/mL consisting of 6% ethanol/15% Tween 80/39% water and 40% PBS. 4) sucrose vehicle (100 mg/kg). 5) PEGylated O-acetyl-5050-PLGA nanoparticle vehicle at the highest concentration of 6 mg/mL.

The treatments were administered IV into the tail vein at a dose volume of 10 mL/kg, beginning on post-implantation Day 5, when the mean tumor volume was 55 mm³. The treatments were administered 4 times, on Days 5, 8, 12 and 15 (biweekly×4 injections). On Day 17 post-tumor implantation, mice were euthanized by CO₂ inhalation according to the procedure guideline. Blood was collected by cardiac puncture and put into ethylenediaminetetraacetic acid (EDTA) or serum separation blood collection tubes. Whole blood was analyzed on the day of collection for CBC analyses. After the blood clotted and was centrifuged, serum was frozen immediately on dry ice for serum chemistry analyses. The tumors were removed by dissection, frozen immediately on dry ice and stored at −80° C., in which they were later analyzed for bis(2′-docetaxel) glutamate-5050 PLGA-O-acetyl and free docetaxel levels.

Tolerability was determined by changes in body weight, expressed as a percent of Initial body weight on post-implantation Day 5. The criterion at which a group was removed from the study was a mean of 20% body weight loss. Health monitoring was conducted daily, but no mice warranted removal due to indications of lethargy, tremors, hypothermia, etc. The maximum tolerated dose (MTD) was determined as the highest dose that did not cause a 20% body weight loss. Other indices of toxicity, complete blood count (CBC) and serum chemistry were determined from blood collected from animals that were euthanized on Day 17 by CO₂ inhalation, according to the procedure guideline.

The groups administered 6, 15, 30 and 45 mg/kg of PEGylated bis(2′-docetaxel) glutamate-5050 PLGA-O-acetyl nanoparticles all gained weight on Day 17, a mean of 111%, 112%, 106% and 106°/0, 112% of Initial body weight was observed respectively. For the 60 mg/kg, at Day 17, a mean of 91% of Initial body weight was observed. In comparison, the three vehicle-treated groups all gained weight similarly, i.e. the docetaxel vehicle treatment gained 14.8%, the sucrose vehicle gained 13.8% and the PEGylated O-acetyl-5050-PLGA vehicle gained 16.2%. In contrast, there was a dose-related decline in body weights of mice administered docetaxel, i.e. the higher doses (e.g. 45 and 60 mg/kg) caused a mean 20% of body weight loss earlier (Day 15) compared to the lower doses (e.g. 30 mg'kg occurred at Day 17). The 6 and 15 mg/kg of docetaxel groups caused a mean of 4 and 8% body weight respectively by Day 17.

On Day 17, all PEGylated bis(2′-docetaxel) glutamate-5050 PLGA-O-acetyl nanoparticles groups showed inhibition of tumor growth. The lower 2 doses, 6 and 15 mg/kg caused similar inhibition of tumor growth, 49% and 48% tumor growth inhibition, respectively. For 15 mg/kg, there was a dose-related inhibition of tumor growth. For 30, 45 and 60 mg/kg, a 73%, 83% and 93% tumor growth inhibition was shown. The tumor growth inhibition was directly related to the tumor docetaxel content, r>0.9. In comparison, for the docetaxel control, at 6 and 15 mg/kg, a 78% and 94% TGI, respectively was observed. In contrast, there was no effect by any vehicle on tumor growth, compared to the other vehicle-treated groups.

PEGylated bis(2′-docetaxel) glutamate-5050 PLGA-O-acetyl nanoparticles showed a trend for a decline in the white blood cell (WBC) number, lymphocyte number and neutrophil number. However, there was no significant effect on either the WBC number (ranged from 10.8-6.2×1000 cells/μL for 6-60 mg/kg doses), lymphocyte number (ranged from 6221-4317 cells/μL for 6-60 mg/kg doses) or neutrophil number (ranged from 4404-1889 cells/μL for 6-60 mg/kg doses). In addition, other CBC parameters were not affected by PEGylated bis(2′-docetaxel) glutamate-5050 PLGA-O-acetyl nanoparticles at doses up to 60 mg/kg. In comparison, for the 3 vehicle treated groups (sucrose, docetaxel, O-acetyl-5050-PLGA PEGylated nanoparticle), the WBC (ranged from 11.4-14.1×1000 cells/μL), lymphocyte number (7592-10222 cells/μL) and neutrophil number (3524-4557 cells/μL) all were within the normal range for mice.

The PEGylated bis(2′-docetaxel) glutamate-5050 PLGA-O-acetyl nanoparticles did not affect any serum chemistry parameter at doses up to 15 mg/kg and 60 mg/kg respectively. In comparison, docetaxel did not affect any serum chemistry parameter at doses up to 30 mg/kg. The vehicle formulations did not affect any serum chemistry parameter.

The maximum tolerated dose (MTD) of PEGylated bis(2′-docetaxel) glutamate-5050 PLGA-O-acetyl nanoparticles was 60 mg/kg at the 4-dose treatment schedule administered, 4-fold greater than free docetaxel (MTD=15 mg/kg when administered IV biweekly for weeks). The results are shown in Table 14 below.

TABLE 14
Tumor growth inhibition of B16F10 tumor-bearing
mice administered treatments.

| | Dose | Day 17 |
|---|---|---|
| Group | mg/kg | Tumor Growth Inhibition, % |
| Sucrose Vehicle control | 0 | — |
| PNP Vehicle | 0 | 107% |
| Free docetaxel | 6 | 78% |
| Free docetaxel | 15 | 96% |
| Free docetaxel | 30 | 95% |
| bis(2′-docetaxel) glutamate-5050 | 6 | 49% |
| PLGA-O-acetyl nanoparticles | | |
| bis(2′-docetaxel) glutamate-5050 | 15 | 48% |
| PLGA-O-acetyl nanoparticles | | |
| bis(2′-docetaxel) glutamate-5050 | 30 | 73% |
| PLGA-O-acetyl nanoparticles | | |
| bis(2′-docetaxel) glutamate-5050 | 45 | 83% |
| PLGA-O-acetyl nanoparticles | | |
| bis(2′-docetaxel) glutamate-5050 | 60 | 93% |
| PLGA-O-acetyl nanoparticles | | |

### Example 42

In vivo efficacy of PEGylated docetaxel-2′-5050 PLGA-O-acetyl nanoparticles in a A2780 ovarian human xenograft model was measured. A2780 cells were grown in culture in RPMI-1640 containing 10% FBS and 1% penicillin/streptomycin (passage=2). When confluent, the cells were removed using 0.05% trypsin and suspended in 1:1 mixture of RPMI-1640/Matrigel at a density of 50×10⁶ cells/mL. The tumors were implanted SC by injecting 5×10⁶ A2780 cells in a 0.1 mL volume into the mammary fat pad of female CD-1 nude mice that were 6-8 weeks old.

The three treatment groups that were administered to the mice consisted of: 1) a docetaxel vehicle formulation consisting of a 10 mg/mL stock solution (prepared with 20 mg of docetaxel, 0.2 mL ethanol, 0.5 mL Tween 80 and 1.3 mL water, added in that specific order and vortexed to ensure proper mixing). The stock solution was diluted further with PBS to 1.5 mg/mL (for a dose of 15 mg/kg at 10 mL/kg and 30 mg/kg at 20 mL/kg). This formulation was made within 30 minutes of administration to mice. 2) Filtered PEGylated O-acetyl-5050-PLGA nanoparticles at a dose of 30 mg/kg, 3) docetaxel vehicle at the highest concentration of 1.5 mg/mL consisting of 1.5% ethanol, 3.8% Tween 80, 9.8% water and 85% PBS.

The treatments were administered IV into the tail vein at a dose volume of 10 mL/kg for the 15 mg/kg group and 20 mL/kg for the other groups, beginning on post-implantation Day 8, when the mean tumor volume was 128 mm³. The treatments were administered 2 times, on Day 8 and Day 15 (weekly×2 injections) for n=8 mice per group. The study endpoint for the vehicle-treated and the docetaxel 15 mg/kg groups was a group mean tumor size of 1000 mm³. The study endpoint for the docetaxel 30 mg/kg and the nanoparticles groups was an individual mouse tumor size of 1000 mm³. On Day 50, the study was ended for all remaining mice. When removed from the study, mice were euthanized by CO2 inhalation.

On Day 8, the PEGylated O-acetyl-5050-PLGA nanoparticles (dose=30 mg/kg) treatment group had a mean body weight of 27.6±1.0 g. On Day 29, this group had a mean body weight of 26.1±1.1 g, representing a maximum body weight loss of 5±3%. On the last day in the study (i.e. Day 50), the mean body weight was 27.2±1.7 g. The mice were regaining weight, to 97±3% of this group's initial body weight. The formulation administered as a treatment to the mice was shown to be sterile using a bioburden assay. Initial mean body weight of the docetaxel vehicle treated group was 26.3±1.9 g on Day 8. When this group was removed from the study on Day 25, the mean body weight was 27.8±2.3 g. This represented a 106±2% of Initial mean body weight. In comparison for the mice administered with docetaxel, on Day 8, the mean body weight of the docetaxel administered 15 mg/kg group was 27.3±2.3 g. On Day 22, this group decreased in body weight to 25.3±1.7 g, representing a maximum of 7% body weight loss. On Day 36, when the docetaxel administered 15 mg/kg group was removed from the study, the mean body weight was 30.7±2.5 g, representing a 113±11% of Initial body weight. Similarly, on Day 8, the mean body weight of the docetaxel administered 30 mg/kg group was 26.3±1.3 g. On Day 22, the mean body weight decreased to 23.7±1.9 g, representing a maximum of 10% body weight loss. On Day 36, this group weighed 30.7±2.5 g, representing a 105±9% of Initial body weight. Overall, there was a dose-related decline in body weights of mice administered with docetaxel.

For the PEGylated O-acetyl-5050-PLGA nanoparticles administered at a dose of 30 mg/kg, on Day 25, the tumor volume was 110±135 mm3 (range 30-408 mm3), with a TGI of 91%. The group mean tumor volume did not reach the endpoint during the duration of the study. One individual mouse reached 1000 mm3 on Day 29, however 6 mice remained in the study on Day 50. The TGD could not be calculated, but is estimated to be greater than 25 days.

For the docetaxel treatment group, on Day 25, the tumor volume of the 15 mg/kg group was 349±470 mm3 (range 68-1481 mm3), with a TGI of 71%. This group surpassed the endpoint on Day 32 with a tumor volume of 1477±1730 mm3 (range 165-5692 mm3). No difference in the slope of the growth curve was apparent. The TGD was determined to be 5 days for the docetaxel treatment group (15 mg/kg) by extrapolating to when the tumor growth curve crossed 1000 mm3. On Day 25, the tumor volume of the 30 mg/kg group was 63±68 mm3 (range 7-218 mm3), with a TGI of 95%. This group reached the endpoint on Day 39 with a tumor volume of 950±1239 (0-3803 mm3). Individual mice reached 1000 mm3 on Day 32 (1 mouse), Day 39 (1 mouse), Day 42 (3 mice) and Day 46 (1 mouse). On Day 50, 2 mice still remained in the study. No difference in the slope of the growth curve was apparent. The TGD was calculated to be 14 days. There was a dose-related inhibition of tumor growth of mice administered with the docetaxel treatment groups.

In contrast, on Day 25, the mean tumor volume was 1000 mm3 for the docetaxel vehicle treatment group and the tumor doubling time was 4 days. There was no effect by the docetaxel vehicle on tumor growth, compared to the other treatment groups. The PEGylated O-acetyl-5050-PLGA nanoparticles administered at a dose of 30 mg/kg showed improved efficacy and a greater TGD, compared to docetaxel, at the same dose and schedule. The results are shown in Table 15 below.

TABLE 15
Tumor growth inhibition and tumor growth delay of A2780
tumor-bearing mice administered treatments.

| | | Day 25 | Tumor |
|---|---|---|---|
| | | Tumor Growth | Growth |
| | Dose | Inhibition | Delay |
| Group | (mg/kg) | (%) | (day) |
| Docetaxel Vehicle control | 0 | — | |
| Free docetaxel | 15 | 71 | 5 |
| Free docetaxel | 30 | 95 | 14 |
| PEGylated O-acetyl-5050-PLGA | 30 | 91 | >25 |
| nanoparticles | | | |

### Example 43

Synthesis of Polyfunctionalized PLGA/PLA based polymers could be completed by the following protocol. One could synthesize a PLGA/PLA related polymer with functional groups that are dispersed throughout the polymer chain that is readily biodegradable and whose components are all bioacceptable components (i.e. known to be safe in humans). Specifically, PLA/PLA related polymers derived from 3-S-[benxyloxycarbonyl)methyl]-1,4-dioxane-2,5-dione (BMD) could be synthesized (see Structures 1-3 below).

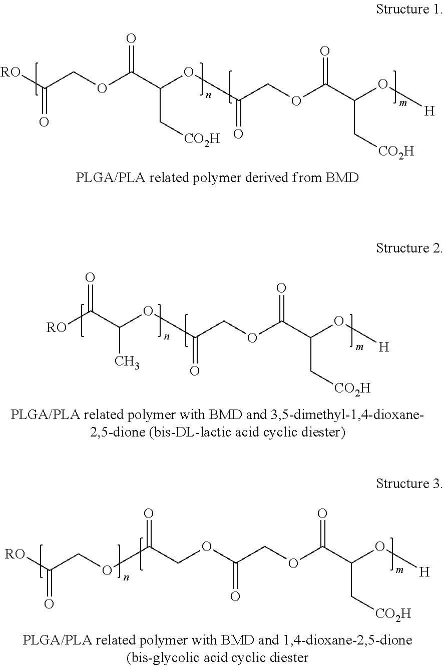

In a preferred embodiment, PLGA/PLA polymers derived from BMD and bis-DL-lactic acid cyclic diester will be prepared with a number of different pendent functional groups by varying the ratio of BMD and lactide. For reference, if it is assumed that each polymer has a number average molecular weight (Mn) of 8 kDa, then a polymer that is 100 wt % derived from BMD has approximately 46 pendant carboxylic acid groups (1 acid group per 0.174 kDa). Similarly, a polymer that is 25 wt % derived from BMD and 75 w t % derived from 3,5-dimethyl-1,4-dioxane-2,5-dione (bis-DL-lactic acid cyclic diester) has approximately 11 pendant carboxylic acid groups (1 acid group per 0.35 kDa). This compares to just 1 acid group for an 8 kDa PLGA polymer that is not functionalized and 1 acid group/2 kDa if there are 4 sites added during functionalization of the terminal groups of a linear PLGA/PLA polymer or 1 acid group/1 kDa if a 4 kDa molecule has four functional groups attached.

Specifically, the PLGA/PLA related polymers derived from BMD will be developed using a method by Kimura et al., Macromolecules, 21, 1988, 3338-3340. This polymer would have repeating units of glycolic and malic acid with a pendant carboxylic acid group on each unit [RO(COCH₂OCOCHR¹O)ₙH where R is H, or alkyl or PEG unit etc. and R¹ is CO₂H]. There is one pendant carboxylic acid group for each 174 mass units. The molecular weight of the polymer and the polymer polydispersity can vary with different reaction conditions (i.e. type of initiator, temperature, processing condition). The Mn could range from 2 to 21 kDa. Also, there will be a pendant carboxylic acid group for every two monomer components in the polymer. Based on the reference previously sited, NMR analysis showed no detectable amount of the β-malate polymer was produced by ester exchange or other mechanisms.

Another type of PLGA/PLA related polymer derived from BMD and 3,5-dimethyl-1,4-dioxane-2,5-dione (bis-DL-lactic acid cyclic diester) will be synthesized using a method developed by Kimura et al., Polymer, 1993, 34, 1741-1748. They showed that the highest BMD ratio utilized was 15 mol % and this translated into a polymer containing 14 mol % (16.7 wt %) of BMD-derived units. This level of BMD incorporation represents approximately 8 carboxylic acid residues per 8 kDa polymer (1 carboxylic acid residue/kDa of polymer). Similarly to the use of BMD alone, no β-malate derived polymer was detected. Also, Kimura et al. reported that the glass transition temperatures (Tg) were in the low 20° C.'s despite the use of high polymer molecular weights (36-67 kDa). The Tg's were in the 20-23° C. for these polymers whether the carboxylic acid was free or still a benzyl group. Inclusion of more rigidifying elements (i.e. carboxylic acids which can form strong hydrogen bonds) should increase the Tg. Possible prevention of aggregation of any nanoparticles formed from a polymer drug conjugate derived from this specific polymer will have to be evaluated due to possible lower Tg values.

Another method for synthesizing a PLA-PEG polymer that contains varying amounts of glycolic acid malic acid benzyl ester involves the polymerization of BMD in the presence of 3,5-dimethyl-1,4-dioxane-2,5-dione (bis-DL-lactic acid cyclic diester), reported by Lee et al. Journal of Controlled Release, 94, 2004, 323-335. They reported that the synthesized polymers contained 1.3-3.7 carboxylic acid units in a PLA chain of approximately 5-8 kDa (total polymer weight was approximately 11-13 kDa with PEG being 5 kDa) depending on the quantity of BMD used in the polymerization. In one polymer there were 3.7 carboxylic acid units/hydrophobic block in which the BMD represents approximately 19 wt % of the weight of the hydrophobic block. The ratio of BMD to lactide was similar to that observed by Kimura et al., Polymer, 1993, 34, 1741-1748 and thecid residues were similar in the resulting polymers (approximately 1 acid unit/kDa of hydrophobic polymer).

Polymers functionalized with BMD that are more readily hydrolysable will be prepared using the method developed by Kimura et al., International Journal of Biological Macromolecules, 25, 1999, 265-271. They reported that the rate of hydrolysis was related to the number of free acid groups present (with polymers with more acid groups hydrolyzing faster). The polymers had approximately 5 or 10 mol % BMD content. Also, in the reference by Lee et al. Journal of Controlled Release, 94, 2004, 323-335, the rate of hydrolysis of the polymer was fastest with the highest concentration of pendent acid groups (6 days for polymer containing 19.5 wt % of BMD and 20 days for polymer containing 0 wt % of BMD. Drug (e.g. docetaxel, paclitaxel, doxorubicin, etc.) was conjugated to a PLGA/PLA related polymer with BMD (refer to previous examples above). Similarly, nanoparticles were prepared from such a polymer drug conjugate.

### Example 44

Synthesis of polymers prepared using β-lactone of malic acid benzyl esters could be completed by the following protocol. MePEGOH could be polymerized with RS-β-benzyl malolactonate (a β-lactone) with DL-lactide (cyclic diester of lactic acid) to afford a polymer containing MePEG (lactic acid) (malic acid) Me(OCH₂CH₂O)[OCCCH(CH₃)O]ₘ[COCH₂CH(CO₂H)0], as developed by Wang et al., Colloid Polymer Sci., 2006, 285, 273-281. These polymers potentially degrade faster because they contain higher levels of acidic groups. It should be noted that the use of β-lactones could generate a different polymer from that obtained using 3-[benzyloxycarbonyl)methyl]-1,4-dioxane-2,5-dione. In these polymers, the carboylic acid group could be directly attached to the polymer chain without a methylene spacer.

Another polymer that could be prepared directly from a β-lactone was reported by Ouhib et al. Ch. Des. Monoeres. Polym, 2005, 1, 25. The resulting polymer (i.e. poly-3,3-dimethylmalic acid) is water soluble as the free acid, has pendant carboxylic acid groups on each unit of the polymer chain. It has been noted that 3,3-dimethylmalic acid is a nontoxic molecule.

One could polymerize 4-benzyloxycarbonyl-,3,3-dimethyl-2-oxetanone in the presence of 3,5-dimethyl-1,4-dioxane-2,5-dione (DDD) and β-butyrolactone to generate a block copolymer with pendant carboxlylic acid groups as shown by Coulembier et al., Macromolecules, 2006, 39, 4001-4008. This polymerization reaction could be carried out with a carbene catalyst in the presence of ethylene glycol. The catalyst used could be a triazole carbene catalyst which leads to polymers with narrow polydispersities.

### Example 45

Nanoparticles and liposomes comprising therapeutic agents were lyophilized using three different techniques. The first technique was a simple freeze drying technique where the liquid formulations were frozen with liquid nitrogen followed by drying under vacuum overnight at room temperature. During this simple lyophilization technique a Labconco® freeze dryer (available from Labconco Corp. of Kansas City, Mo.) was used. The second technique involved a rapid cycle lyophilization program that is shown below in Table 16. Instead of conventional multi-step ramping and holding, one step slow ramping was used in this approach. As a result, the length of lyophilization cycle was shorten to ⅓ of the conventional one. The particle size was well maintained for PEGylated nanoparticles comprising the following components: mPEG2K-PLGA (40 wt. %); docetaxel conjugated to 5050 PLGA, wherein the hydroxyl end of polymer was modified with an acetyl group and the polymer has a molecular weight of 7-11 kDa (see Example 9)) (60 wt. %); and PVA (9-10 kDa, 80% hydrolyzed, viscosity 2.5-3.5 cps, used as a 0.5% w/v solution) (Referred to herein as “PEGylated nanoparticles A”), at the same weight ratio of HP-β-CD/nanoparticle as shown below in Table 17.

TABLE 16
Rapid Cycle Lyophilization Control System Conditions
Thermal Treatment

| | Temp | Time | R/H |
|---|---|---|---|
| Step 1 | 5 | 120 | H |
| Step 2 | −45 | 60 | R |
| Step 3 | −45 | 180 | H |
| Step 4 | 0 | 0 | H |
| Step 5 | 0 | 0 | R |
| Step 6 | 0 | 0 | |
| Step 7 | 0 | 0 | |
| Step 8 | 0 | 0 | |
| Step 9 | 0 | 0 | |
| Step 10 | 0 | 0 | |
| Step 11 | 0 | 0 | |
| Step 12 | 0 | 0 | |

Primary Drying

| | Temp | Time | Vacuum | R/H |
|---|---|---|---|---|
| Step 1 | −45 | 120 | 100 | |
| Step 2 | −20 | 720 | 100 | R |
| Step 3 | 0 | 0 | 0 | H |
| Step 4 | 0 | 0 | 0 | R |
| Step 5 | 0 | 0 | 0 | H |
| Step 6 | 0 | 0 | 0 | R |
| Step 7 | 0 | 0 | 0 | H |
| Step 8 | 0 | 0 | 0 | R |
| Step 9 | 0 | 0 | 0 | H |
| Step 10 | 0 | 0 | 0 | R |
| Step 11 | 0 | 0 | 0 | R |
| Step 12 | 0 | 0 | 0 | |
| Step 13 | 0 | 0 | 0 | |
| Step 14 | 0 | 0 | 0 | |
| Step 15 | 0 | 0 | 0 | |
| Step 16 | 0 | 0 | 0 | |

TABLE 17
Rapid Cycle Lyophilization Data Summary

| | [HP-β- | [Polymer] | HP-β-CD: | | | | Filtration |
|---|---|---|---|---|---|---|---|
| | CD] | (Doce.) | Polymer | Zave | Dv₉₀ | | Potency |
| Sample | mg/mL | mg/mL | (w/w) | (nm) | (nm) | PDI | Loss (%) |
| Prior to | | | | 89.57 | 119 | 0.091 | |
| Lyophilization | | | | | | | |
| Post | 40 | 31.25 (1.5) | 1.28:1 | 89.70 | 119 | 0.096 | 5 |
| Lyophilization | | | | | | | |

The third technique used to lyophilize the liquid formulations was a conventional cycle lyophilization program that lasted 72 hours and is shown in Table 18 below. The particle size is well maintained for PEGylated nanoparticles A, at the same weight ratio of HP-β-CD/nanoparticle (see Table 19). Both the rapid cycle and conventional cycle lyophilization reactions were performed using a VirTis advantage freeze dryer.

TABLE 18
Conventional Cycle Lyophilization Control System Conditions
Thermal Treatment

| | Temp | Time | R/H |
|---|---|---|---|
| Step 1 | 5 | 120 | H |
| Step 2 | −45 | 120 | R |
| Step 3 | −45 | 180 | H |
| Step 4 | 0 | 0 | H |
| Step 5 | 0 | 0 | R |
| Step 6 | 0 | 0 | |
| Step 7 | 0 | 0 | |
| Step 8 | 0 | 0 | |
| Step 9 | 0 | 0 | |
| Step 10 | 0 | 0 | |
| Step 11 | 0 | 0 | |
| Step 12 | 0 | 0 | |

Primary Drying

| | Temp | Time | Vacuum | R/H |
|---|---|---|---|---|
| Step 1 | −45 | 120 | 100 | |
| Step 2 | −20 | 120 | 100 | R |
| Step 3 | −20 | 1200 | 100 | H |
| Step 4 | −10 | 120 | 100 | R |
| Step 5 | −10 | 720 | 100 | H |
| Step 6 | 0 | 120 | 100 | R |
| Step 7 | 0 | 540 | 100 | H |
| Step 8 | 10 | 120 | 100 | R |
| Step 9 | 10 | 480 | 100 | H |
| Step10 | 20 | 120 | 100 | R |
| Step11 | 0 | 0 | 0 | H |
| Step12 | 0 | 0 | 0 | |
| Step13 | 0 | 0 | 0 | |
| Step14 | 0 | 0 | 0 | |
| Step15 | 0 | 0 | 0 | |
| Step16 | 0 | 0 | 0 | |

TABLE 19
Conventional Cycle Lyophilization Data Summary

| | [HP-β- | [Polymer] | HP-β-CD: | | | | Filtration |
|---|---|---|---|---|---|---|---|
| | CD] | (Doce.) | Polymer | Zave | Dv₉₀ | | Potency |
| Sample | mg/mL | mg/mL | (w/w) | (nm) | (nm) | PDI | Loss (%) |
| Prior to | | | | 89.57 | 119 | 0.091 | |
| Lyophilization | | | | | | | |
| Post | 40 | 31.25 (1.5) | 1.28:1 | 90.93 | 121 | 0.095 | 7 |
| Lyophilization | | | | | | | |

### Example 46

A cryoprotectant screen was performed as follows. The critical point for design of a lyophilization cycle was to keep the temperature below the glass transition temperature (Tg′) of the cryoprotectant during the primary drying stage. Table 20 summarizes the Tg's for the above sugars chosen for screen.

TABLE 20
Glass Transition Temperatures

| | Glass Transition |
|---|---|
| Lyoprotectant | or Eutectic T (° C.) |
| Trehalose | −29.5 |
| Sucrose | −37 |
| Lactose | −32 |
| Mannitol | −1.0 |

The Tg's for trehalose and lactose and the eutectic temperature of mannitol are equal to or higher than sucrose's Tg′ and therefore the lyophlization cycle control system conditions developed for sucrose applied to all the above sugars selected. These conditions are shown below in Table 21.

TABLE 21
Sucrose Cycle Lyophilization Control System Conditions
Thermal Treatment

| | Temp | Time | R/H |
|---|---|---|---|
| Step 1 | 5 | 120 | H |
| Step 2 | −45 | 120 | R |
| Step 3 | −45 | 450 | H |
| Step 4 | 0 | 0 | H |
| Step 5 | 0 | 0 | R |
| Step 6 | 0 | 0 | |
| Step 7 | 0 | 0 | |
| Step 8 | 0 | 0 | |
| Step 9 | 0 | 0 | |
| Step10 | 0 | 0 | |
| Step11 | 0 | 0 | |
| Step12 | 0 | 0 | |

Primary Drying

| | Temp | Time | Vacuum | R/H |
|---|---|---|---|---|
| Step 1 | −45 | 120 | 100 | |
| Step 2 | −35 | 120 | 100 | R |
| Step 3 | −35 | 1200 | 100 | H |
| Step 4 | −30 | 120 | 100 | R |
| Step 5 | −30 | 720 | 100 | H |
| Step 6 | −20 | 120 | 100 | R |
| Step 7 | −20 | 540 | 100 | H |
| Step 8 | 0 | 120 | 100 | R |
| Step 9 | 0 | 480 | 100 | H |
| Step 10 | 25 | 120 | 100 | R |
| Step 11 | 25 | 480 | 100 | H |
| Step 12 | 0 | 0 | 0 | |
| Step 13 | 0 | 0 | 0 | |
| Step 14 | 0 | 0 | 0 | |
| Step 15 | 0 | 0 | 0 | |
| Step 16 | 0 | 0 | 0 | |

The liquid formulation used for screen contained PEGylated nanoparticles A. The data as summarized in Tables 22 and 23 shown below gave rise to the following conclusions. Particle size significantly increased in the absence of cryoprotectant. Amorphous sugars (sucrose, treholose and lactose) provided better lyoprotection than crystalline sugar (mannitol). Trehalose did not give sufficient lyoprotection even at weight ratio of 9.6:1 sugar/nanoparticle. Sucrose was the most effective cryoprotectant.

TABLE 22
Cyoprotectant Screen Lyophilization Data Summary

| | [Cryo- | [Polymer] | Cryoprotectant/ | Lyophilized | Recon. | | | |
|---|---|---|---|---|---|---|---|---|
| Cryo- | protectant] | (Doce.) | Polymer | preparation | Solution | Zave | Dv₉₀ | |
| protectant | mg/mL | mg/mL | (w/w) | Appearance | Appearance | (nm) | (nm) | PDI |
| Prior to | | | | | | 90.31 | 118 | 0.059 |
| Lyophilization | | | | | | | | |
| None | 0 | 31.25 (1.5) | 0 | good | precipitation | 11.94 | 741 | 0.885 |
| Sucrose | 100 | 31.25 (1.5) | 3.2:1 | good | slight | 94.72 | 124 | 0.125 |
| | | | | | precipitation | | | |
| Lactose | 100 | 31.25 (1.5) | 3.2:1 | some | cloudy | 183.2 | 178 | 0.352 |
| | | | | foams | | | | |
| Mannitol | 30 | 31.25 (1.5) | 0.96:1 | good | precipitation | 499.2 | 340 | 0.638 |
| | 100 | 31.25 (1.5) | 3.2:1 | some | cloudy | 472.7 | 2540 | 0.544 |
| | | | | foams | | | | |
| Trehalose | 20 | 31.25 (1.5) | 0.64:1 | good | precipitation | 236.1 | 188 | 0.381 |
| | 60 | 31.25 (1.5) | 1.92:1 | good | cloudy | 276.9 | 169 | 0.464 |
| | 100 | 31.25 (1.5) | 3.2:1 | good | cloudy | 294.2 | 286 | 0.417 |
| | 200 | 31.25 (1.5) | 6.4:1 | good | slight | 192.2 | 186 | 0.348 |
| | | | | | precipitation. | | | |
| | 300 | 31.25 (1.5) | 9.6:1 | good | slight | 154.8 | 205 | 0.325 |
| | | | | | precipitation. | | | |

TABLE 23
Cyoprotectant Screen Weight Ratio Data Summary

| | | | | | | | | | Filtration |
|---|---|---|---|---|---|---|---|---|---|
| | Cryo- | [Polymer] | Cryprotectant/ | Lyophilized | Recon. | | | | Loss (0.2 |
| Cryo- | protectant | (Doce.) | Polymer | preparation | Solution | Zave | Dv₉₀ | | μm PES |
| protectant | (mg/mL) | (mg/mL) | (w/w) | Appearance | Appearance | (nm) | (nm) | PDI | Filter) |
| Prior to | | | | | | 90.31 | 118 | 0.059 | |
| Lyophilization | | | | | | | | | |
| Sucrose | 100 | 31.25 | 3.2:1 | good | slight | 94.72 | 124 | 0.125 | 15% |
| | | (1.50) | | | precipitation. | | | | |
| | 100 | 26.05 | 3.8:1 | good | good | 92.79 | 124 | 0.110 | 10% |
| | | (1.25) | | | | | | | |
| | 100 | 20.83 | 4.8:1 | good | good | 91.37 | 120 | 0.081 | 2% |
| | | (1.00) | | | | | | | |
| | 100 | 10.42 | 9.6:1 | good | good | 90.62 | 120 | 0.081 | 2% |
| | | (0.50) | | | | | | | |
| | 100 | 31.25 | 3.2:1 | good | cloudy | 294.2 | 286 | 0.417 | |
| | | (1.50) | | | | | | | |
| Trehalose | 100 | 26.05 | 3.8:1 | good | cloudy | 259.1 | 379 | 0.372 | |
| | | (1.25) | | | | | | | |
| | 100 | 20.83 | 4.8:1 | good | cloudy | 606.5 | 189 | 0.725 | |
| | | (1.00) | | | | | | | |
| | 100 | 10.42 | 9.6:1 | good | slight | 108.1 | 160 | 0.0166 | |
| | | (0.50) | | | precipitation. | | | | |

### Example 47

Crystallization of PEG is likely the reason for particle size increase during lyophilization. In this example, a new strategy of using cyclodextrins and their derivatives as a cryoprotectant was tested. Initially, HP-β-CD was evaluated using simple process of freezing with liquid nitrogen followed by lyophilization under vacuum at room temperature. For instance, each intravenous dose of 200 mg itraconazole injection (Sporamox®) contains 8 g of HP-β-CD. The data is shown in Table 24 lead to the following conclusions. A cryoprotectant is needed to lyophilize liquid formulation that contain PEGylated nanoparticles A (Entries #1 and #2). HP-β-CD was effective at weight ratio as low as 1.28:1 (Entries #1, #3, #5, #6 and #7 as a cryoprotectant. HP-β-CD give excellent reproducibility (Entries #4 and #5). Sucrose and trehalose were less effective cryoprotectants than HP-β-CD (Entries #9, #10 and #5). Other cyclodextrins were likely to also be effective as cryoprotectants (Entries #8 and #3).

TABLE 24
Data Summary for Lyophilization Using HP-β-CD

| | | [Cryo- | [Polymer] | Cryoprotectant/ | Reconstituted | | | | Filtration |
|---|---|---|---|---|---|---|---|---|---|
| Entry | | protectant] | (Doce) | Polymer | Solution | Zave | Dv₉₀ | | Loss |
| # | Cyoprotectant | mg/mL | mg/mL | (w/w) | appearance | (nm) | (nm) | PDI | (%) |
| 1. | Prior to | | | | | 90.31 | 118 | 0.059 | N/A |
| | Lyophilization | | | | | | | | |
| 2. | None | 0 | 31.25 | 0 | precipitation | 202.6 | 853 | 0.426 | N/A |
| | | | (1.5) | | | | | | |
| 3. | HP-beta-CD | 20 | 31.25 | 0.64:1 | some | 90.93 | 121 | 0.095 | 4 |
| | | | (1.5) | | precipitation | | | | |
| 4. | HP-beta-CD | 40 | 31.25 | 1.28:1 | good | 89.43 | 118 | 0.077 | 6 |
| | | | (1.5) | | dispersion | | | | |
| 5. | HP-beta-CD | 40 | 31.25 | 1.28:1 | good | 90.66 | 119 | 0.075 | 1 |
| | | | (1.5) | | dispersion | | | | |
| 6. | HP-beta-CD | 60 | 31.25 | 1.92:1 | good | 89.84 | 119 | 0.089 | 2 |
| | | | (1.5) | | dispersion | | | | |
| 7. | HP-beta-CD | 80 | 31.25 | 2.56:1 | good | 90.60 | 119 | 0.095 | 3 |
| | | | (1.5) | | dispersion | | | | |
| 8. | Alfa-CD | 15 | 31.25 | 0.48:1 | good | 92.05 | 122 | 0.088 | 8 |
| | | | (1.5) | | dispersion | | | | |
| 9. | Sucrose | 40 | 31.25 | 1.28:1 | precipitation | 197.2 | 155 | 0.207 | N/A |
| | | | (1.5) | | | | | | |
| 10. | Trehalose | 40 | 31.25 | 1.28:1 | precipitation | 114.1 | 130 | 0.260 | N/A |
| | | | (1.5) | | | | | | |

### Example 48

Other CDs were also evaluated at the similar weight ratio of cryoprotectant/nanoparticle. As shown in Tables 25 and 26, α-CD, γ-CD and SB-β-CD were as effective as HP-β-CD as a cryoprotectant for PEGylated nanoparticles A.

TABLE 25
Data Summary for Lyophilization Using Other Cyclodextrins

| | | | [Polymer] | | | | |
|---|---|---|---|---|---|---|---|
| | Lyopro- | [CD] | (Doce.) | HP-β-CD: | Zave | Dv₉₀ | |
| Sample | tactant | mg/mL | mg/mL | Polymer (w/w) | (nm) | (nm) | PDI |
| 42-150 | | | | | 89.57 | 119 | 0.091 |
| Prior to | | | | | | | |
| Lyophilization | | | | | | | |
| 42-189 #3 | α-CD | 40 | 31.25 | 1.28:1 | 92.06 | 121 | 0.070 |
| Post | | | 91.5) | | | | |
| Lyophilization | | | | | | | |
| 42-189 #1 | β-CD | 40 | 31.25 | 1.28:1 | Beta-CD is not soluble at this | | |
| Post | | | 91.5) | | concentration | | |
| Lyophilization | | | | | | | |
| 42-170 #3 | HP-β- | 40 | 31.25 | 1.28:1 | 90.66 | 119 | 0.075 |
| Post | CD | | 91.5) | | | | |
| Lyophilization | | | | | | | |
| 42-189 #2 | y-CD | 40 | 31.25 | 1.28:1 | 91.06 | 121 | 0.097 |
| Post | | | 91.5) | | | | |
| Lyophilization | | | | | | | |

TABLE 26
Data Summary for Lyophilization Using Other Cyclodextrins

| | [SB- | | | | | | Fil- |
|---|---|---|---|---|---|---|---|
| | β- | [Poly- | | | | | tration |
| | CD] | mer] | SB-β- | | | | Potency |
| | mg/ | (Doce.) | CD:Polymer | Zave | Dv₉₀ | | Loss |
| Sample | mL | mg/mL | (w/w) | (nm) | (nm) | PDI | (%) |
| 42-150 | | | | 105.5 | 139 | | |
| Prior to | | | | | | | |
| Lyophili- | | | | | | | |
| zation | | | | | | | |
| 42- | 40 | 28.87 | 1.39:1 | 106.9 | 149 | | 2 |
| 189 #3 | | (1.94) | | | | | |
| Post | | | | | | | |
| Lyophili- | | | | | | | |
| zation | | | | | | | |
| 42- | 60 | 28.87 | 2.08:1 | 108.5 | 151 | | 8 |
| 189 #1 | | (1.94) | | | | | |
| Post | | | | | | | |
| Lyophili- | | | | | | | |
| zation | | | | | | | |

### Example 49

The conventional cycle also worked for liquid formulations containing PEGylated nanoparticles comprising the following components: mPEG2K-PLGA (16 wt. %); docetaxel conjugated to 5050 PLGA, wherein the hydroxyl end of polymer was modified with an acetyl group and the polymer has a molecular weight of 7-11 kDa (see Example 9)) (84 wt. %); and PVA (9-10 kDa, 80% hydrolyzed, viscosity 2.5-3.5 cps, used as a 0.5% w/v solution) (Referred to herein as “PEGylated nanoparticles B”) at the same weight ratio of cryoprotectant/nanoparticle (See Table 27 below). Overall, the cycle worked for all nanoparticle formulations containing PEG from 16% to 40% (w/w).

TABLE 27
Data Summary for Lyophilization Using Other Nanoparticles

| | [HP- | | | | | | Fil- |
|---|---|---|---|---|---|---|---|
| | β- | [Poly- | | | | | tration |
| | CD] | mer] | HP-β- | | | | Potency |
| | mg/ | (Doce.) | CD:Polymer | Zave | Dv₉₀ | | Loss |
| Sample | mL | mg/mL | (w/w) | (nm) | (nm) | PDI | (%) |
| Prior to | | | | 105.5 | 139 | 0.130 | |
| Lyophili- | | | | | | | |
| zation | | | | | | | |
| Post | 36.95 | 28.87 | 1.28:1 | 105.5 | 143 | 0.116 | 3 |
| Lyophili- | | (1.94) | | | | | |
| zation | | | | | | | |
| | 28.57 | 22.32 | 1.28:1 | 105.8 | 146 | 0.077 | 8 |
| | | (1.50) | | | | | |

A concentrated concentration of the liquid formulation was also tested. The conventional cycle also worked for concentrated formulation at the same weight ratio of cryoprotectant/nanoparticle as shown in Table 27 above. Alternatively, the concentrated formulation (>3.5 mg/mL docetaxel Equivalent) was also prepared by reconstitution of the lyophilized 1.5 mg/mL docetaxel equivalent formulation with less amount of water (40% of fill volume as shown in Table 28 below.

TABLE 28
Data Summary for Lyophilization of a Concentrated Liquid Formulation

| | | | | | | | Post- | |
|---|---|---|---|---|---|---|---|---|
| | | | [HP-β- | | | | Reconstitution | |
| | [HP-β- | | CD] | | | | BF-AF | |
| | CD] | [Polymer] | Polymer | Zave | Dv₉₀ | | Filtration | Filtration |
| Sample | mg/mL | mg/mL | (w/w) | (nm) | (nm) | PDI | (mg/mL) | Loss (%) |
| #1 | | 26.79 | | 90.66 | 120 | 0.88 | | |
| (30% PEG2K) | | | | | | | | |
| Prior to | | | | | | | | |
| Lyophilization | | | | | | | | |
| #1 | 34.29 | 26.79 | 1.28:1 | 90.67 | 120 | 0.090 | 3.82/3.72 | 3 |
| (30% PEG2K) | | | | | | | | |
| Post | | | | | | | | |
| Lyophilization. | | | | | | | | |
| #2 | | 26.79 | | 87.26 | 115 | 0.101 | | |
| (40% PEG2K) | | | | | | | | |
| Prior to | | | | | | | | |
| Lyophilization | | | | | | | | |
| #2 | 34.29 | 26.79 | 1.28:1 | 87.55 | 115 | 0.108 | 3.59/3.61 | 0 |
| (40% PEG2K) | | | | | | | | |
| Post | | | | | | | | |
| Lyophilization. | | | | | | | | |

Table 29 below shows additional data for example 45 with a wide range of reconstitution volumes.

TABLE 29
Data Summary for Lyophilization of a Concentrated Liquid Formulation

| | [HP- | [Polymer] | [HP-B- | Reconstiution | | | |
|---|---|---|---|---|---|---|---|
| | B-CD] | (Doce.) | CD]:Polymer | Concentration | Zave | Dv₉₀ | |
| Sample | mg/ml | mg/ml | (w/w) | (mg/ml) | (nm) | (nm) | PDI |
| Prior to | | | | | 80.26 | 104 | 0.083 |
| lyophilization | | | | | | | |
| #1 Post | 40.53 | 19 | 1.28:1 | 1.4 | 87.49 | 116 | 0.121 |
| lyophilization | | (1.52) | | | | | |
| #2 Post | 40.53 | 19 | 1.28:1 | 2 | 88.26 | 115 | 0.136 |
| lyophilization | | (1.52) | | | | | |
| #3 Post | 40.53 | 19 | 1.28:1 | 2.7 | 86.01 | 112 | 0.157 |
| lyophilization | | (1.52) | | | | | |
| #4 Post | 40.53 | 19 | 1.28:1 | 4 | 86.01 | 112 | 0.148 |
| lyophilization | | (1.52) | | | | | |
| #5 Post | 40.53 | 19 | 1.28:1 | 4.9 | 84.42 | 110 | 0.123 |
| lyophilization | | (1.52) | | | | | |

### Example 50

Lyophilization of 5K-PEG liquid formulations were performed to test the effects of lengthening PEG. It was previously reported in literature that more cryoprotectant was needed when the length of PEG increased. However, we have discovered that HP-β-CD was effective at the same weight ratio under conventional lyophilization cycle regardless of the length of PEG as shown in Table 30 below.

TABLE 30
Data Summary for Lyophilization of PEGylated Nanoparticles with Long PEG chains

| | [HP-β- | | [HP-β- | | | | | |
|---|---|---|---|---|---|---|---|---|
| | CD] | [Polymer] | CD]:Polymer | Zave | Dv₉₀ | | Post-Recon | Filtration |
| Sample | mg/mL | mg/mL | (w/w) | (nm) | (nm) | PDI | (mg/mL) | Loss (%) |
| #1 | | | | 97.92 | 133 | 0.076 | | |
| (30% PEG5K) | | | | | | | | |
| Prior to | | | | | | | | |
| Lyophilization | | | | | | | | |
| #1 | 28.56 | 22.32 | 1.28:1 | 99.11 | 133 | 0.059 | 1.41/1.24 | 12 |
| (30% PEG5K) | | | | | | | | |
| Post | | | | | | | | |
| Lyophilization. | | | | | | | | |
| #2 | | | | 95.19 | 129 | 0.093 | | |
| (40% PEG5K) | | | | | | | | |
| Prior to | | | | | | | | |
| Lyophilization | | | | | | | | |
| #2 | 31.25 | 40 | 1.28:1 | 95.48 | 128 | 0.074 | 1.50/1.37 | 9 |
| (40% PEG5K) | | | | | | | | |
| Post | | | | | | | | |
| Lyophilization. | | | | | | | | |
| #3 | | | | 106.1 | 150 | 0.092 | | |
| (40% PEG5K) | | | | | | | | |
| Post | | | | | | | | |
| Lyophilization. | | | | | | | | |
| #3 | 26.79 | 34.29 | 1.28:1 | 106.7 | 151 | 0.094 | 1.53/1.50 | 2 |
| (40% PEG5K) | | | | | | | | |
| Post | | | | | | | | |
| Lyophilization. | | | | | | | | |

### Example 51

The lyophilized preparations were used for an in vivo study. PEGylated nanoparticles comprising the following components: mPEG2K-PLGA (30 wt. %); docetaxel conjugated to 5050 PLGA, wherein the hydroxyl end of polymer was modified with an acetyl group and the polymer has a molecular weight of 7-11 kDa (see Example 9)) (60 wt. %); and PVA (9-10 kDa, 70% hydrolyzed, viscosity 2.5-3.5 cps, used as a 0.5% w/v solution) (Referred to herein as “PEGylated nanoparticles C”) were lyophilized in the presence of 1.28 fold of HP-β-CD. The particle size was well maintained under conventional lyophilization cycle (see Table 31).

TABLE 31
Data Summary for Lyophilization of PEGylated nanoparticles C

| | [HP-β- | [Polymer] | [HP-β- | | | |
|---|---|---|---|---|---|---|
| | CD] | (Doce.) | CD]:Polymer | Zave | Dv₉₀ | |
| Sample | mg/mL | mg/mL | (w/w) | (nm) | (nm) | PDI |
| Prior to | | | | 87.84 | 115 | 0.055 |
| Lyophilization | | | | | | |
| Post | 45.71 | 35.71 | 1.28:1 | 90.56 | 120 | 0.091 |
| Lyophilization. | | (2.0) | | | | |

### Example 52

PLGA7K-PVA-PEG2K-30 and PLGA7K-PVA-PEG5K-30 PEGylated nanoparticle formulations were also examined by the simple lyophilization process of freezing with liquid nitrogen followed by drying under vacuum overnight at room temperature. As shown in Table 32, particle size was well maintained for both 2K-PEG and 5 K-PEG based formulations at HP-β-CD/nanoparticle weight ratio as low as 1:1. Table 33 below shows that α-CD and γ-CD but not SB-β-CD also worked at the same weight ratio. None of mannitol, sucrose and trehalose worked at the same ratio. The results are similar to that obtained for PEGylated nanoparticles A except for SB-β-CD. The result from SB-β-CD supported the H-bonding mechanism for cryoprotection of PEGylated PLGA nanoparticles since SB-β-CD has less hydroxyl groups than α-CD, γ-CD and HP-β-CD (about ⅓ of —OH groups of β-CD are substituted by sulfobutyl groups).

TABLE 32
Data Summary for Lyophilization of PLGA PEGylated Nanoparticles

| | [HP-β- | | HP- | | | |
|---|---|---|---|---|---|---|
| | CD] | [Nanoparticle] | CD/NP | Zave | Dv₉₀ | |
| Sample | mg/mL | mg/mL | (w/w) | (nm) | (nm) | PDI |
| Prior to | | | | 99.51 | 139 | 0.115 |
| Lyophili- | | | | | | |
| zation | | | | | | |
| 1 | 0 | 20 | 0 | 160.8 | 340 | 0.210 |
| 2 | 10 | 20 | 0.5 | 106.5 | 155 | 0.115 |
| 3 | 20 | 20 | 1 | 101.5 | 140 | 0.091 |
| 4 | 30 | 20 | 1.5 | 101.0 | 140 | 0.095 |
| 5 | 40 | 20 | 2 | 99.43 | 137 | 0.097 |

TABLE 33
Data Summary for Lyophilization of PLGA PEGylated Nanoparticles

| | MW | | | | Lyop. | | | |
|---|---|---|---|---|---|---|---|---|
| | of | | [Cryoprotectant] | [Nanoparticle] | NP | Zave | Dv₉₀ | |
| Sample | PEG | Cryoprotectant | mg/mL | mg/mL | (w/w) | (nm) | (nm) | PDI |
| 2K BF | | | | | | 99.51 | 139 | 0.115 |
| Lyo | | | | | | | | |
| 1 | 2K | Mannitol | 20 | 20 | 1 | Precipitated | | |
| 2 | 2K | Sucrose | 20 | 20 | 1 | Precipitated | | |
| 3 | 2K | Trehalose | 20 | 20 | 1 | Precipitated | | |
| 4 | 2K | α-CD | 20 | 20 | 1 | 101.0 | 139 | 0.087 |
| 5 | 2K | γ-CD | 20 | 20 | 1 | 101.8 | 139 | 0.080 |
| 6 | 2K | HP-β-CD | 20 | 20 | 1 | 102.0 | 140 | 0.084 |
| 7 | 2K | SB-β-CD | 20 | 20 | 1 | Precipitated | | |
| 5K BF | 5K | | | | | 84.26 | 110 | 0.114 |
| Lyo | | | | | | | | |
| 8 | 5K | HP-β-CD | 20 | 20 | 1 | 85.92 | 113 | 0.127 |

### Example 53

Lyophilization of Stealth Liposome was also performed. PEGylated liposomes-Doxil® were lyophilized using HP-β-CD under conventional cycle conditions shown in Table 16 above. Particle size was well maintained at HP-β-CD/lipids ratio as low as 2.5:1 (see Table 34 below). In contrast, sucrose can not maintain particle size even at sucrose/lipids ratio as high as 18.5:1 under a comparable lyophilization cycle designed for sucrose and the conditions shown in Table 21 above.

TABLE 34
Data Summary for Lyophilization of PEGylated Liposomes

| | | [Cryoprotectant] | [Lipids] | Cryoprotectant/Lipid | Zave | Dv₉₀ | | Lyophilization |
|---|---|---|---|---|---|---|---|---|
| Sample | Cryoprotectant | mg/mL | mg/mL | (w/w) | (nm) | (nm) | PDI | Cycle |
| Doxil ® | | | 16 | | 88.35 | 117 | 0.084 | N/A |
| Prior to | | | | | | | | |
| Lyophilization | | | | | | | | |
| 42-183 #4 | | | 16 | | 227.6 | 1990 | 0.543 | HP-β-CD |
| Post | | | | | | | | |
| Lyophilization | | | | | | | | |
| 42-183 #5 | HP-β-CD | 40 | 16 | 2.5:1 | 90.32 | 119 | 0.071 | HP-β-CD |
| Post | | | | | | | | |
| Lyophilization | | | | | | | | |
| 18-202 #2 | Sucrose | 100 | 16 | 6.25:1 | 142.4 | 58.5 | 0.390 | Sucrose |
| Post | | | | | | | | |
| Lyophilization | | | | | | | | |
| 18-202 #3 | Sucrose | 150 | 16 | 12.5:1 | 219.3 | 533 | 0.484 | Sucrose |
| Post | | | | | | | | |
| Lyophilization | | | | | | | | |
| 18-202 #4 | Sucrose | 200 | 16 | 18.5:1 | 200.2 | 1640 | 0.506 | Sucrose |
| Post | | | | | | | | |
| Lyophilization | | | | | | | | |

### Examples 54-58

In examples 54 through 58 when reference is made to “mPEG(Xk)-PLGA Y wt %”, Xk indicates the weight average molecular weight of the mPEG portion of the mPEG-PLGA polymer (e.g., mPEG(2 k) indicates that 2 kDa mPEG is conjugated to PLGA), and Y indicates the weight percentage of mPEG-PLGA as compared to the PLGA-drug conjugate in the initial mixture used to make the nanoparticles. For example, 16 wt % indicates that an 84:16 weight ratio of PLGA-drug conjugate to mPEG-PLGA was prepared and added to surfactant in order to prepare the nanoparticles. Typically, approximately half of the mPEG-PLGA used in the reaction is incorporated in to the product nanoparticles. Thus the approximate components of the nanoparticles in the following examples are as follows:

mPEG(2 k)-PLGA 16%=In the particle: mPEG(2 k)-PLGA ˜8 wt %, PVA ˜23 wt %, Docetaxel-5050 PLGA-O-acetyl ˜69 wt %

mPEG(2 k)-PLGA 30 with %=In the particle: mPEG(2 k)-PLGA ˜17 wt %, PVA ˜23 wt %, Docetaxel-5050 PLGA-O-acetyl ˜60 wt %

mPEG(2 k)-PLGA 40 wt %=In the particle: mPEG(2 k)-PLGA ˜23 wt %, PVA ˜26 wt %, Docetaxel-5050 PLGA-O-acetyl ˜51 wt %

mPEG(5 k)-PLGA 16 wt %=In the particle: mPEG(5 k)-PLGA ˜8 wt %, PVA ˜22%, Docetaxel-5050 PLGA-O-acetyl ˜70%

mPEG(5 k)-PLGA 30 wt %=In the particle: mPEG(5 k)-PLGA ˜16, PVA ˜24%, Docetaxel-5050 PLGA-O-acetyl ˜60%

mPEG(5 k)-PLGA 40 wt %=In the particle: mPEG(5 k)-PLGA ˜18 wt %, PVA ˜24%, Docetaxel-5050 PLGA-O-acetyl ˜58%

### Example 54

The efficacy and tolerability of PEGylated docetaxel-5050 PLGA-O-acetyl nanoparticles were examined in a B16.F10 murine melanoma model. B16.F10 cells were grown in culture to confluency in MEM-α medium supplemented with 10% fetal bovine serum (FBS, passage 4) and 1% penicillin/streptomycin and then resuspended in PBS. A volume of 0.1 mL containing 1×106 cells was subcutaneously implanted into the right flank of male C57BL/6 mice on day—1.

The seven treatment groups that were administered to the mice included: 1) A docetaxel formulation prepared at 10 mg/mL stock solution (with 20 mg of docetaxel, 0.2 mL ethanol, 0.5 mL polysorbate 80 and 1.3 mL water, added in that specific order and vortexed to ensure proper mixing) diluted further with PBS to 1.5 and 3 mg/mL for a corresponding dose of 15 and 30 mg/kg. For a 60 mg/kg dose, a 20 mL/kg injection volume of a concentration of 3 mg/mL docetaxel formulation was administered. 2) PEGylated docetaxel-5050 PLGA-O-acetyl nanoparticles (mPEG(2 k)-PLGA at 16 wt %) administered at doses of 15 and 30 mg/kg. PEGylated docetaxel-5050 PLGA-O-acetyl nanoparticles (mPEG(2 k)-PLGA at 30 wt %) administered at doses of 15, 30 and 60 mg/kg. 4) PEGylated docetaxel-5050 PLGA-O-acetyl nanoparticles (mPEG(2 k)-PLGA at 40 wt %)) administered at doses of 15 and 30 mg/kg. PEGylated docetaxel-5050 PLGA-O-acetyl nanoparticles (mPEG(5 k)-PLGA at 16 wt %) administered at a dose of 15 mg/kg. 6) PEGylated docetaxel-5050 PLGA-O-acetyl nanoparticles (mPEG(5 k)-PLGA at 30 wt %) administered at doses of 15 and 30 mg/kg, 7) PEGylated docetaxel-5050 PLGA-O-acetyl nanoparticles (mPEG(5 k)-PLGA at 40 wt %) administered at a dose of 15 mg/kg. Refer to table for detailed description of formulations.

The treatments were administered IV into the tail vein at a dose volume of 10 or 20 mL/kg depending on the treatment group, beginning on post-implantation day 5, when the mean tumor volume was approximately 55 mm3. Animals were monitored for any morbidity and adverse effect three times a week. Body weight and tumor volume were also measured three times a week.

Tumor volume was calculated with the following equation: (width×width×length)/2 mm3. Efficacy was determined by tumor growth inhibition (TGI), tumor growth delay (TGD) and survival. TGI was represented as % and calculated as follows: (1—(treated tumor volume/control tumor volume))×100 when the control group mean tumor volume reached≧3000 mm3. Tolerability was determined by changes in body weight, expressed as a percent of Initial body weight on post-implantation day-5. Health monitoring was conducted three times a week to evaluate lethargy, tremors, hypothermia, ataxia, hind limb paralysis etc. The criteria at which a mouse was removed from the study were >20% body weight loss or severe morbidity or hind limb paralysis.

The PEGylated nanoparticles (mPEG(2 k)-PLGA at 16 wt %) were administered using a q3dq4d schedule. The docetaxel control group and the PEGylated nanoparticles were administered three times over a two week schedule at a dose of 15 mg/kg and 30 mg/kg respectively. The docetaxel group showed a TGI of 90% in comparison to the PEGylated nanoparticles, which had a TGI of 84%. The docetaxel group exhibited a similar TGD of 12 days compared to 13 days for the PEGylated nanoparticles. The PEGylated nanoparticles did not cause any body weight loss and was better tolerated than the docetaxel group which caused a 12% maximum body weight loss.

The PEGylated nanoparticles (mPEG(2 k)-PLGA at 30 wt %) were administered using a q3dq4d schedule. The docetaxel control group and the PEGylated nanoparticles were administered three times over a two week schedule at a dose of 15 mg/kg. Both the PEGylated nanoparticles and the docetaxel groups were equally efficacious. The TGI of the docetaxel and PEGylated groups were 90% and 86% respectively. Similarly both groups exhibited the same TGD of 11 days. The PEGylated nanoparticles did not show any body weight loss and was better tolerated than docetaxel, which caused a 11% maximum body weight loss.

The PEGylated nanoparticles (mPEG(2 k)-PLGA at 30 wt %) were administered using a q7d schedule. Both the docetaxel control group and the PEGylated nanoparticles were administered three times, once every week at a dose of 30 mg/kg. The TGI for the docetaxel and PEGylated nanoparticles group was 90% and 96% respectively. The PEGylated nanoparticles showed a greater TGD (25 days) and survival compared to the docetaxel group (17 days). In addition, the PEGylated nanoparticles were better tolerated and caused no body weight loss, whereas the docetaxel group had a maximum body weight loss of 11%

The PEGylated nanoparticles (mPEG(2 k)-PLGA at 30 wt %) were administered using a q14d schedule. Both the docetaxel control group and the PEGylated nanoparticles were administered two times, once every two weeks at a dose of 60 mg/kg. The TGI for the PEGylated nanoparticles group was greater (i.e. 97%) than that of the docetaxel group (i.e. 71%). The PNP also exhibited an increased TGD and survival compared to docetaxel. The docetaxel group reached the tumor volume end point on day 29 and showed a TGD of 11 days. In the case of the PEGylated nanoparticles group, the average tumor volume was 118 mm3 on day 42. A TGD for the PEGylated nanoparticles could not be determined because at the time of measurement, the group still had not reached the tumor volume end point (i.e. on day 56, the average tumor volume was 840 mm3). In addition, the PEGylated nanoparticles were well tolerated and caused only 8% maximum body weight loss. The control group docetaxel did not show any body weight loss.

The PEGylated nanoparticles (mPEG(2 k)-PLGA at 40 wt %) were administered using a q7d schedule. Both the docetaxel control group and the PEGylated nanoparticles were administered three times, once every week at a dose of 15 mg/kg. The TGI of the docetaxel group and the PEGylated nanoparticles was shown to be similar (approximately 90%). The TGD of the free docetaxel and the PEGylated nanoparticles was 11 and 13 days respectively. There was no body weight loss associated with the PEGylated nanoparticles; in contrast, the docetaxel group showed a maximum body weight loss of 11%.

The PEGylated nanoparticles (mPEG(5 k)-PLGA at 16 wt %) were administered using a q3dq4d schedule. The docetaxel and the PEGylated nanoparticles groups were administered three times over a two week schedule at a dose of 15 mg/kg. The docetaxel group had a TGI of 90% compared to the PEGylated nanoparticles group which had a TGI of 71%. The TGD of the docetaxel and PEGylated nanoparticles groups were 11 and 7 days respectively. The PEGylated nanoparticles were better tolerated and showed no body weight loss compared to the docetaxel group, which exhibited an 11% maximum body weight loss.

The PEGylated nanoparticles (mPEG(5 k)-PLGA at 30 wt %) were administered using a q3dq4d schedule. The docetaxel and the PEGylated nanoparticles groups were administered three times over a two week schedule at a dose of 15 mg/kg. The docetaxel and PEGylated nanoparticles groups showed a similar TGI (i.e. 90%). In terms of the TGD, the docetaxel group showed 11 days compared to the PEGylated nanoparticles (i.e. 13 days). The PEGylated nanoparticles were better tolerated than the docetaxel control group. Also, the docetaxel group exhibited a maximum body weight loss of 11% compared to no body weight loss shown by the PEGylated nanoparticles group.

The PEGylated nanoparticles (mPEG(5 k)-PLGA at 30 wt %) were administered using a q7d schedule. Both the docetaxel and PEGylated nanoparticles groups were administered three times, once a week at a dose of 30 mg/kg. The TGI of the docetaxel and PEGylated nanoparticles groups were 90% and 97% respectively. The TGD of the docetaxel group was determined to be 17 days as the average tumor volume reached the end point of 3000 mm3 at day 37. A TGD for the PEGylated nanoparticles could not be determined because at the time of measurement, the group still had not reached the tumor volume end point (i.e. on day 47, the average tumor volume was 2100 mm3). The PEGylated nanoparticles did not cause any body weight loss and was better tolerated than free docetaxel which caused a 11% body weight loss.

The PEGylated nanoparticles (mPEG(5 k)-PLGA at 40 wt %) were administered using a q4dq3d schedule. The docetaxel and PEGylated nanoparticles groups were administered three times over a two week schedule at a dose of 15 mg/kg. The TGI for both groups was similar (approximately 90-92%). The TGD for the PEGylated nanoparticles (i.e. 15 days) was greater than that for the docetaxel group (i.e. 11 days). The PEGylated nanoparticles did not cause any body weight loss to the mice and were better tolerated compared to the docetaxel group which resulted in a 11% maximum body weight loss. Table 35 shows a comparison of efficacy and tolerability of different PEGylated nanoparticles (2 k) formulation and the control docetaxel treatment group.

TABLE 35
Comparison of efficacy and tolerability of different PEGylated
nanoparticles (2k) formulation and the control docetaxel
treatment group

| | | | Tumor | Tumor | Maximum |
|---|---|---|---|---|---|
| | | | growth | growth | body |
| | | | inhibition | delay | weight |
| | | Dose | (TGI) | (TGD) | loss |
| Formulation | Schedule | (mg/kg) | (%) | (days) | (%) |
| Docetaxel | q3dq4dx3 | 15 | 90 | 12 | 12 |
| PEGylated nps | q3dq4dx3 | 30 | 84 | 13 | 0 |
| (mPEG(2k)- | | | | | |
| PLGA | | | | | |
| 16 wt %) | | | | | |
| Docetaxel | q3dq4dx3 | 15 | 90 | 11 | 11 |
| PEGylated nps | q3dq4dx3 | 15 | 86 | 11 | 0 |
| (mPEG(2k)- | | | | | |
| PLGA | | | | | |
| 30 wt %) | | | | | |
| Docetaxel | q7dx3 | 30 | 90 | 17 | 11 |
| PEGylated nps | q7dx3 | 30 | 96 | 25 | 0 |
| (mPEG(2k)- | | | | | |
| PLGA | | | | | |
| 30 wt %) | | | | | |
| Docetaxel | q14dx2 | 60 | 71 | 11 | 0 |
| PEGylated nps | q14dx2 | 60 | 97 | >38 | 8 |
| (mPEG(2k)- | | | | | |
| PLGA | | | | | |
| 30 wt %) | | | | | |
| Docetaxel | q3dq4dx3 | 15 | 90 | 11 | 11 |
| PEGylated nps | q3dq4dx3 | 15 | 89 | 13 | 0 |
| (mPEG(2k)- | | | | | |
| PLGA | | | | | |
| 40 wt %) | | | | | |

q3dq4dx3 - three injections administered over 2 weeks (3 days in between 1^{st} and 2^{nd} injection, 4 days in between 2^{nd} and 3^{rd} injection).
q7dx3 - three injections seven days apart.
q14dx2 - two injections 14 days apart.

TABLE 36
Comparison of efficacy and tolerability of different PEGylated
nanoparticles (5k) formulation and the control docetaxel
treatment group

| | | | Tumor | Tumor | Maximum |
|---|---|---|---|---|---|
| | | | growth | growth | body |
| | | | inhibition | delay | weight |
| | | Dose | (TGI) | (TGD) | loss |
| Formulation | Schedule | (mg/kg) | (%) | (days) | (%) |
| Docetaxel | q3dq4dx3 | 15 | 90 | 11 | 11 |
| PEGylated nps | q3dq4dx3 | 15 | 71 | 7 | 0 |
| (PEG(5k)- | | | | | |
| PLGA | | | | | |
| 16 wt %) | | | | | |
| Docetaxel | q3dq4dx3 | 15 | 90 | 11 | 11 |
| PEGylated nps | q3dq4dx3 | 15 | 90 | 13 | 0 |
| (PEG(5k)- | | | | | |
| PLGA | | | | | |
| 30 wt %) | | | | | |
| Docetaxel | q7dx3 | 30 | 90 | 17 | 11 |
| PEGylated nps | q7dx3 | 30 | 97 | >38 | 0 |
| (PEG(5k)- | | | | | |
| PLGA | | | | | |
| 30 wt %) | | | | | |
| Docetaxel | q4dq3dx3 | 15 | 90 | 11 | 11 |
| PEGylated nps | q4dq3dx3 | 15 | 92 | 15 | 0 |
| (PEG(5k)- | | | | | |
| PLGA | | | | | |
| 40 wt %) | | | | | |

q3dq4dx3 - three injections administered over 2 weeks (3 days in between 1^{st} and 2^{nd} injection, 4 days in between 2^{nd} and 3^{rd} injection).
q4dq3dx3 - three injections administered over 2 weeks (4 days in between 1^{st} and 2^{nd} injection, 3 days in between 2^{nd} and 3^{rd} injection).
q7dx3 - three injections seven days apart.

### Example 55

In vivo efficacy of PEGylated docetaxel-5050 PLGA-O-acetyl nanoparticles were examined in a HCT-116 colon xenograft model. HCT-116 cells were grown in culture to confluency in McCoy's 5a medium containing 10% FBS and 1% penicillin/streptomycin and then resuspended in McCoy's 5a (passage 4). This suspension of HCT-116 cells (density=3.7×106 cells/mL) was implanted subcutaneously above the right hind leg of male CD-1 nude mice on day 1.

The three treatment groups that were administered to HCT-116 tumor bearing mice (n=6-7 per group) included: 1) a docetaxel vehicle formulation consisting of 1.5% ethanol/3.75% polysorbate 80/9.75% water/85% PBS at 20 mL/kg; 2) 10 mg/mL docetaxel stock solution (prepared with 20 mg of docetaxel, 0.2 mL ethanol, 0.5 mL polysorbate 80 and 1.3 mL water, added in that specific order and vortexed to ensure proper mixing) diluted in PBS to 1.5 mg/mL for a corresponding dose of 30 mg/kg at an injection volume of 20 mL/kg respectively; 3) PEGylated docetaxel-5050 PLGA-O-acetyl nanoparticle formulation (mPEG(2 k)-PLGA with initial amount of 16 wt %) at a docetaxel equivalent concentration of 1.5 mg/mL for a corresponding dose of 30 mg/kg at an injection volume of 20 mL/kg

The treatments were administered 1V into the tail vein at the respective dose volumes (refer to previous paragraph), beginning on post-implantation Day 13, when the mean tumor volume was 131 mm3. The vehicle and docetaxel treatments were administered two times, on Days 13 and 20 (weekly×two injections).

The mice that were administered docetaxel at a dose of 30 mg/kg lost a maximum body weight of 14%. In comparison, the PEGylated formulation administered at a dose of 30 mg/kg, did not lose any weight during the study.

The tumor growth inhibition (TGI) of the mice treated with docetaxel at a dose of 30 mg/kg was 88%. Extrapolating to where the tumor growth curve reached the end point at a tumor volume of 1000 mm3, the TGD was calculated to be 22 days. For the PEGylated nanoparticles at a dose of 30 mg/kg, the TGI was 77%. The TGD was determined to be 21 days.

### Example 56

In vivo efficacy of PEGylated docetaxel-5050 PLGA-O-acetyl nanoparticles were examined in a SK-OV-3 ovarian human xenograft model. SK-OV-3 cells were grown in culture to confluency in RPMI medium containing 10% FBS and 1% penicillin/streptomycin and then resuspended in RPMI (passage 4) for implantation into mice. This suspension of SK-OV-3 cells (density=30×106 cells/mL) was implanted into the mammary gland of female CD-1 nude mice on Day 1.

Treatment groups that were administered to SK-OV-3 tumor-bearing mice (n=4-5 per group) included: 1) a docetaxel vehicle formulation consisting of 1.5% ethanol/3.75% polysorbate 80/9.75% water/85% PBS at 20 mL/kg; 2) 10 mg/mL docetaxel stock solution (prepared with 20 mg of docetaxel, 0.2 mL ethanol, 0.5 mL polysorbate 80 and 1.3 mL water, added in that specific order and vortexed to ensure proper mixing) diluted in PBS to A) 1.5 mg/mL for a corresponding dose of 15 mg/kg and 30 mg/kg at an injection volume of 10 mL/kg and 20 mL/kg respectively, and B) 3 mg/mL for a dose of 60 mg/kg at an injection volume of 20 mL kg; 3) PEGylated docetaxel-5050 PLGA-O-acetyl nanoparticle formulation (mPEG(2 k)-PLGA with initial amount of 16 wt %) at a docetaxel equivalent concentration of 2.9 mg/mL for a corresponding dose of 60 mg/kg at an injection volume of 21 mL/kg.

The treatments were administered IV into the tail vein at the dose volumes stated above, beginning on post-implantation Day 51, when the mean tumor volume was 232 mm3. The vehicle and docetaxel treatments were administered two times, on Days 51 and 58 (weekly×two injections). The PEGylated nanoparticles treatment was administered once, on Day 51.

The high dose of docetaxel, 60 mg/kg, caused greater than 20% body weight loss. Ataxia, which is defined as Inability to coordinate voluntary muscular movements that is symptomatic of some CNS disorders and injuries and not due to muscle weakness, was observed in all the mice four days after the second treatment of docetaxel. This group was removed 18 days after the second treatment, despite supportive measures (fluid replacement, easier access to food), due to the ataxia becoming more severe and affecting the forelimbs. The lower dose of docetaxel, 30 mg/kg, did not cause ataxia. Maximum body weight loss in the group administered docetaxel 30 mg/kg was 13%. The group administered the PEGylated nanoparticles at a dose of 60 mg/kg was only administered that treatment one time. No ataxia developed in this group, but this could not be compared to the high dose of docetaxel because of the different numbers of treatments. Maximum body weight loss in the group administered the PEGylated nanoparticles at 60 mg/kg was 11%, equivalent to the free drug (i.e. docetaxel) at 30 mg/kg.

All treatments inhibited tumor growth. The tumor growth delay (TGD) for docetaxel at a dose of 15 mg/kg was 18 days. The TGD for docetaxel at a dose of 30 mg/kg was 42 days. At this time, this group had a large variation, with two mice>1000 mm3 and three mice

<50 mm3. The TGD for PEGylated nanoparticles at 60 mg/kg was 94 days, with a large intragroup variation with two mice>1000 mm3 and three mice<325 mm3, a similar pattern to free drug at a dose of 30 mg/kg, but delayed approximately 54 days relative to free drug.

### Example 57

In vivo efficacy of PEGylated docetaxel-5050 PLGA-O-acetyl nanoparticles were examined in a MDA-MB-435 melanoma human xenograft model. MDA-MB-435 cells were grown in culture to confluency in RPMI medium containing 10% FBS and 1% penicillin/streptomycin and then resuspended in RPMI (passage 4) for implantation into mice. A volume of 0.1 mL containing 4.0×106 cells MDA-MB-435 cells were implanted into the mammary gland of female CD-1 nude mice on Day 1.

Treatments that were administered to the mice (n=6-7/group) included: 1) a docetaxel vehicle formulation consisting of 1.5% ethanol/3.75% polysorbate 80/9.75% water/85% PBS at 20 mL/kg; 2) 10 mg/mL docetaxel stock solution (prepared with 20 mg of docetaxel, 0.2 mL ethanol, 0.5 mL polysorbate 80 and 1.3 mL water, added in that specific order and vortexed to ensure proper mixing) diluted in PBS to A) 1.5 mg/mL for a corresponding dose of 15 and 30 mg/kg at an injection volume of 10 mL/kg and 20 mL/kg, respectively, B) 3.0 mg/mL for a dose of 60 mg/kg at an injection volume of 20 mL/kg; 3) PEGylated docetaxel-5050 PLGA-O-acetyl nanoparticle formulation (mPEG(2 k)-PLGA with initial amount of 16 wt %) made at a docetaxel equivalent concentration of 1.1 mg/mL for a corresponding dose of 30 mg/kg at an injection volume of 26 mL/kg; 4) PEGylated docetaxel-5050 PLGA-O-acetyl nanoparticle formulation (mPEG(2 k)-PLGA with initial amount of 30 wt %) made at a docetaxel equivalent of 1.5 and 2.85 mg/mL for corresponding doses of A) 15 mg/kg at an injection volume of 10 mL/kg, B) 30 and 60 mg/kg at an injection volume of 11 mL/kg and 21 mL/kg, respectively.

The treatments were administered IV into the tail vein at the dose volumes stated above, beginning on post-implantation Day 21, when the mean tumor volume was 150 mm3 or, for one group, on Day 37, when the mean tumor volume for that group was 433 mm3. The treatments were administered two times, on Days 21 and 28 (weekly×two injections) for the vehicle, docetaxel and PEGylated nanoparticles groups and on Days 37 and 44 for a group that was administered PEGylated nanoparticles when the tumors were at a larger tumor volume (i.e. 433 mm3).

For groups administered the free docetaxel, the high dose. 60 mg/kg, caused greater than 20% body weight loss. Ataxia was observed four days after the second treatment. This group was removed nine days after the second treatment, despite supportive measures (fluid replacement, easier access to food), due to severe ataxia. The docetaxel group administered at a dose of 30 mg/kg did not cause ataxia. Maximum body weight loss in the docetaxel dosed at 30 mg/kg group was 14% and in the case of the 15 mg/kg group, it was 10% of initial body weight.

Groups administered PEGylated nanoparticles had different responses depending on the wt % and dose. The PEGylated nanoparticles (PEG at initial amount of 16 wt %) administered at a dose of 30 mg/kg did not show any weight loss. The PEGylated nanoparticles (PEG at initial amount of 30 wt %) administered at a dose of 15 mg/kg also did not show any weight loss. At a higher dose (30 mg/kg), the PEGylated nanoparticles treatment group lost 6% of its initial body weight. At an even higher dosage (60 mg/kg), the treatment group receiving PEGylated nanoparticles administered starting on Day 21 (i.e. when the mean tumor size was 150 mm3) lost 11% body weight, which was equivalent to the free drug at a dose of 30 mg/kg. The treatment group receiving same PEGylated nanoparticles at a dose of 60 mg/kg were also administered on Day 37 (i.e. when the mean tumor size was 433 mm3) lost 19% body weight. This exaggerated weight loss was likely due to undetermined necrotic factors released from a relatively large amount of dead tumor tissue. One mouse in this latter group was found dead on Day 64 despite supportive measures (fluid replacement, easier access to food). The other mice in that group almost fully recovered their lost body weight and do not appear to be at any health risk at this time (Day 76).

Mice administered docetaxel at a dose of 60 mg/kg developed ataxia. The entire group showed abnormal gait and lack of coordination of the front limbs nine days after the second treatment. No other doses of docetaxel were observed to cause ataxia. In contrast to docetaxel, none of the mice administered PEGylated nanoparticles at any dose developed ataxia.

All treatments groups resulted in tumor growth inhibition. The mean tumor volume of vehicle-treated group reached the endpoint of 1000 mm3 on Day 58 post-tumor implantation. As of Day 76, it appears that the treatment at a dose of 15 mg/kg resulted in the same TGI for free docetaxel and PEGylated nanoparticles. At a dose of 30 mg/kg, the TGI for free docetaxel was greater than that for PEGylated nanoparticles (mPEG-PLGA initial amount of 30wt %>mPEG-PLGA initial amount of 16 wt %). At a dose of 60 mg/kg, free docetaxel was equivalent to PEGylated nanoparticles until the free drug group was removed from the study. As the study continues, docetaxel at a dose of 30 mg/kg is equivalent to PEGylated nanoparticles at a dose of 60 mg/kg.

### Example 58

The tolerability of the free drug docetaxel and PEGylated docetaxel-5050 PLGA-O-acetyl nanoparticles were examined in normal male C57BL/6 non-tumor-bearing mice. Treatments that were administered to the male C57BL/6 mice (n=5/group) included: 1) a docetaxel vehicle formulation consisting of 1.5% ethanol/3.75% polysorbate 80/9.75% water/85% PBS at 20 mL/kg; 2) 10 mg/mL docetaxel stock solution (prepared with 20 mg of docetaxel, 0.2 mL ethanol, 0.5 mL polysorbate 80 and 1.3 mL water, added in that specific order and vortexed to ensure proper mixing) diluted in PBS to 1.5, 2.25 and 3 mg/mL for a corresponding dose of 30, 45 and 60 mg/kg at an injection volume of 20 mL/kg; 3) PEGylated docetaxel-5050 PLGA-O-acetyl nanoparticles formulation (mPEG(2 k)-PLGA initial amount of 30 wt %) at a docetaxel equivalent of 2.85 mg/mL for a dose of 60 mg/kg at an injection volume of 21 mL/kg.

Treatments were administered intravenously on a q7d×2 schedule, i.e., two treatments seven days apart (the first treatment was on Day one). The study ended on Day 14, six days after the 2nd treatment. Blood was collected for complete blood count (CBC) and serum chemistry. Leg muscles were collected so that nerve degeneration could be assessed from the sciatic nerve.

The vehicle-treated group gained 23% of its initial body weight by the end of the study. Docetaxel administered at doses of 30 and 45 mg/kg gained weight, up to 7% at the second treatment, weighing 3% and 2% respectively more than Initial on Day 14. The group administered docetaxel at a dose of 60 mg/kg did not gain weight after the first treatment and lost weight (19%) after the second treatment, by the end of the study. The group administered PEGylated nanoparticles at a dose of 60 mg/kg did not gain weight after the first treatment and lost weight (16%) after the second treatment, by the end of the study.

From Table 37 below, the CBC analyses showed that the white blood cell number, neutrophil number and lymphocyte number were lower in the groups administered docetaxel and PNP at a dose of 60 mg/kg. The white blood cells are expressed in units of ×1000 cells/μL, the neutrophils and lymphocytes are expressed in units of cells/μL.

TABLE 37
CBC Analaysis Results

| | WBC # | | Neutrophil | | Lymphocyte | |
|---|---|---|---|---|---|---|
| Treatment | mean | SD | mean | SD | mean | SD |
| Docetaxel vehicle group | 8.3 | 1.0 | 1474 | 390 | 6563 | 757 |
| Docetaxel, 30 mg/kg | 5.1 | 1.7 | 556 | 254 | 4350 | 1394 |
| Docetaxel, 45 mg/kg | 7.8 | 1.7 | 752 | 266 | 6780 | 1855 |
| Docetaxel, 60 mg/kg | 6.2 | 1.0 | 470 | 159 | 5590 | 938 |
| PEGylated docetaxel-5050 | 4.6 | 0.9 | 488 | 162 | 3958 | 1001 |
| PLGA-O-acetyl | | | | | | |
| nanoparticles (mPEG(2k)- | | | | | | |
| PLGA initial amount of | | | | | | |
| 30 wt %) | | | | | | |

Both the free docetaxel group and the PEGylated docetaxel-5050 PLGA-O-acetyl nanoparticles formulation (mPEG(2 k)-PLGA initial amount of 30 wt %) did not affect any serum chemistry parameter at doses up to 60 mg/kg.

Mice administered the free docetaxel was observed to develop ataxia during the study with a dose-related effect. Specifically, no mice in the 30 mg/kg group were seen to develop ataxia or any overt signs of nerve damage. One mouse in the 45 mg/kg group was observed to develop ataxia on Day 14, while the others in that group had a normal gait. Five out of five mice in the 60 mg/kg group was observed to develop ataxia—one on Day 12, all on Day 14. None of the mice in the group administered PEGylated nanoparticles at a dose of 60 mg/kg was shown to develop ataxia. Table 38 below shows the results.

TABLE 38
Ataxia Results

| | Dose | Ataxia |
|---|---|---|
| Group | (mg/kg) | (%) |
| Docetaxel vehicle control | 0 | — |
| Free docetaxel | 30 | 0 |
| Free docetaxel | 45 | 20 |
| Free docetaxel | 60 | 100 |
| PEGylated docetaxel-5050 PLGA-O-acetyl | 60 | 0 |
| nanoparticles (mPEG(2k)-PLGA initial | | |
| amount of 30 wt %) | | |

These data showed that, contrary to the MDA-MB-435 study described above and historical data, free docetaxel and PEGylated docetaxel-5050 PLGA-O-acetyl nanoparticles (mPEG(2 k)-PLGA initial amount of 30 wt %) at a dose of 60 mg/kg q7d×2 (i.e. two treatments seven days apart) are equivalent regarding body weight loss. Further, and also contrary to historical data, these treatments were similar regarding effects on the CBC.

A pathologist's assessment of the sciatic nerve histology found no treatment effects in any animals. Since ataxia was observed to be severe in the docetaxel group at a dose of 60 mg/kg, and damage by taxanes of the sciatic nerve at the level of the muscle was shown previously in published studies, it was suggested by the pathologist that the section of sciatic nerve that was examined was too far from the spinal chord, and damage did not yet develop in that part of the sciatic nerve at the time of tissue collection.

### Example 59

Docetaxel-2′-5050 PLGA-O-acetyl could be regioselectively prepared as illustrated in the following scheme. The 2′ hydroxyl group of docetaxel is first protected using benzylchloroformate. Following purification of the 2′ Cbz-protected docetaxel, the product may be orthogonally protected on the 7 and 10 hydroxyl groups using a silyl chloride (e.g., tert-butyldimethylsilyl chloride). The Cbz group may then be removed using hydrogenation over Pd/C, followed by coupling of PLGA-O-acetyl using EDC and DMAP. Final deprotection of the silyl protecting groups using TBAF would produce the docetaxel-2′-5050 PLGA-O-acetyl selectively coupled via the 2′ hydroxyl group.

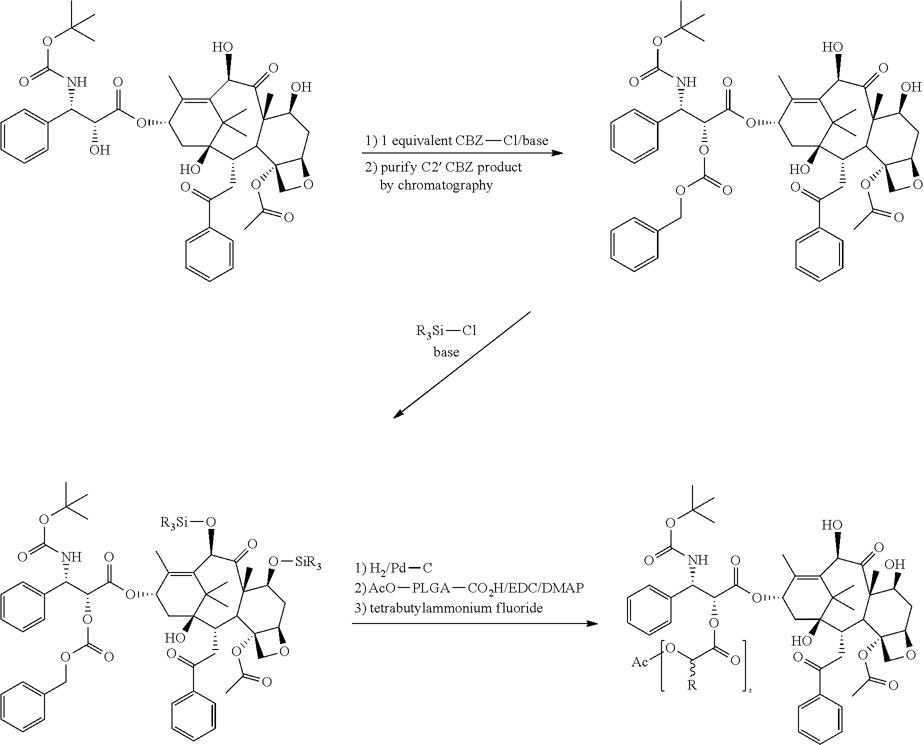

Alternatively, docetaxel-2′-5050 PLGA-O-acetyl could be regioselectively prepared as illustrated in the scheme below. The 2′ hydroxyl group of docetaxel is first protected using tert-butyldimethylsilyl chloride. Following purification of the 2′ TBDMS-protected docetaxel, the product may be orthogonally protected on the 7 and 10 hydroxyl groups using a benzylchloroformate. The TBDMS group may then be removed using TBAF, followed by coupling of PLGA-O-acetyl using EDC and DMAP. Final deprotection of the Cbz protecting groups via hydrogenation over Pd/C would produce the docetaxel-2′-5050 PLGA-O-acetyl selectively coupled via the 2′ hydroxyl group.

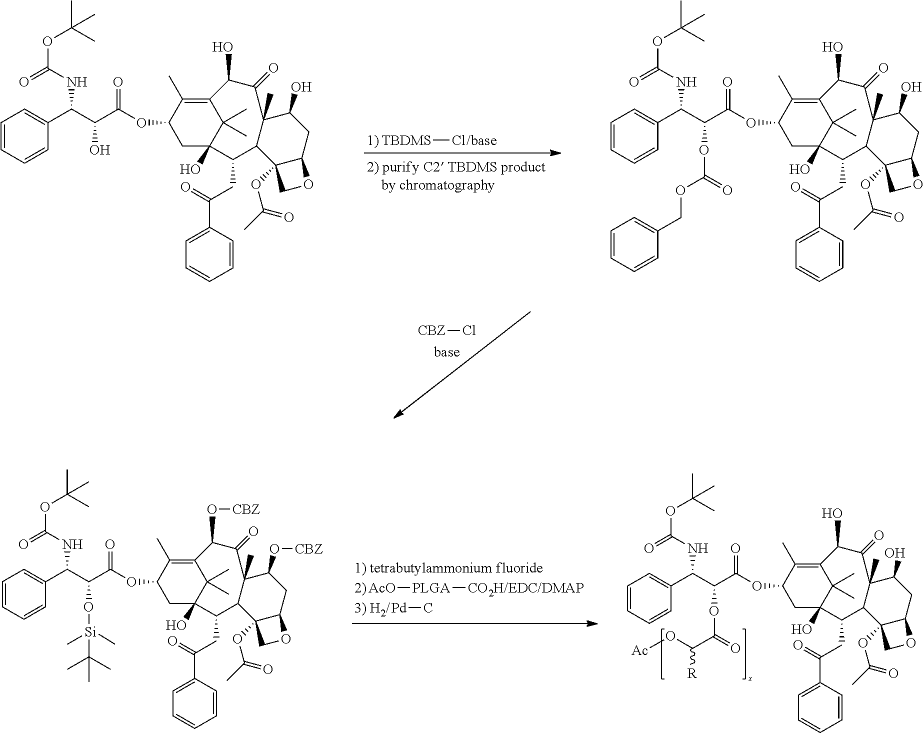

### Example 60

Docetaxel-7-5050 PLGA-O-acetyl and docetaxel-10-5050 PLGA-O-acetyl could be regioselectively prepared as illustrated in the following scheme. Docetaxel is first protected using two equivalents of benzylchloroformate, yielding a mixture of products. Two products, C2′/C7-bis-Cbz-docetaxel, and C2′/C10-bis-Cbz-docetaxel, can each be selectively purified.

C2′/C7-bis-Cbz-docetaxel can then be coupled to PLGA-O-acetyl using EDC and DMAP, which would result in attachment of PLGA-O-acetyl to the hydroxyl group at the 10-position of docetaxel. Deprotection of the Cbz protecting groups via hydrogenation over Pd/C would produce the docetaxel-10-5050 PLGA-O-acetyl selectively coupled via the 10 hydroxyl group.

C2′/C10-bis-Cbz-docetaxel can then be coupled to PLGA-O-acetyl using EDC and DMAP, which would result in attachment of PLGA-O-acetyl to the hydroxyl group at the 7-position of docetaxel. Deprotection of the Cbz protecting groups via hydrogenation over Pd/C would produce the docetaxel-7-5050 PLGA-O-acetyl selectively coupled via the 7 hydroxyl group.

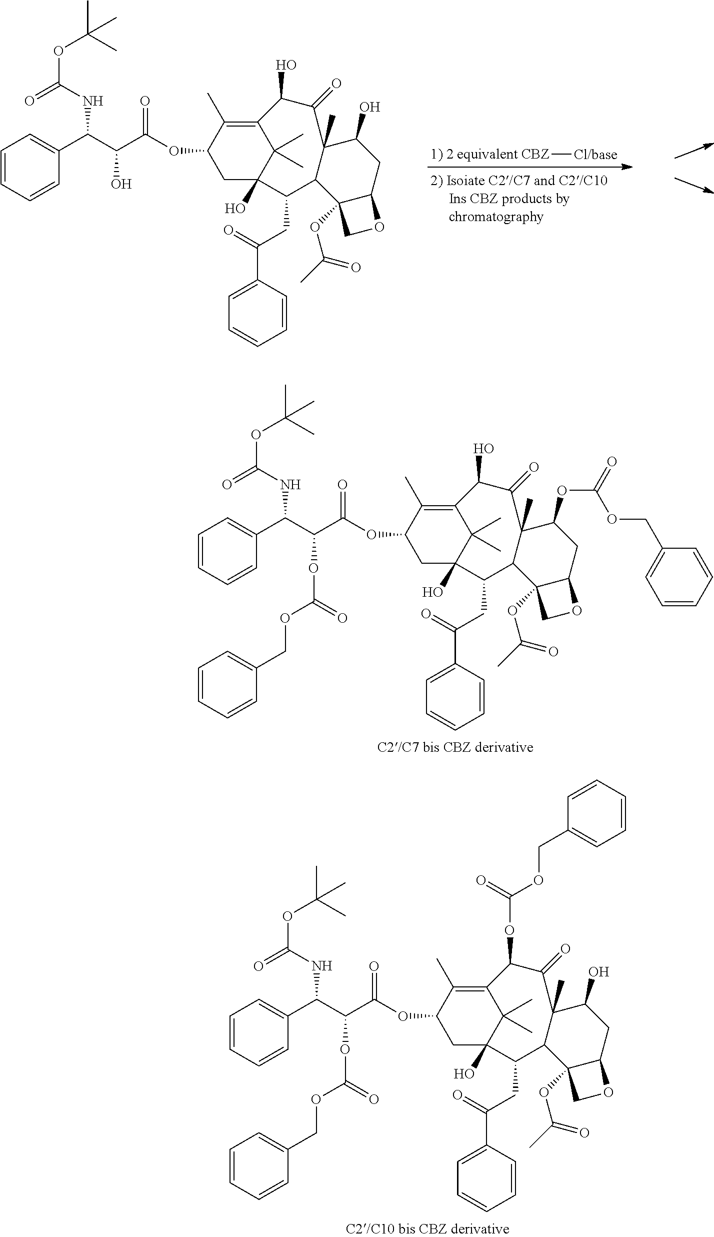

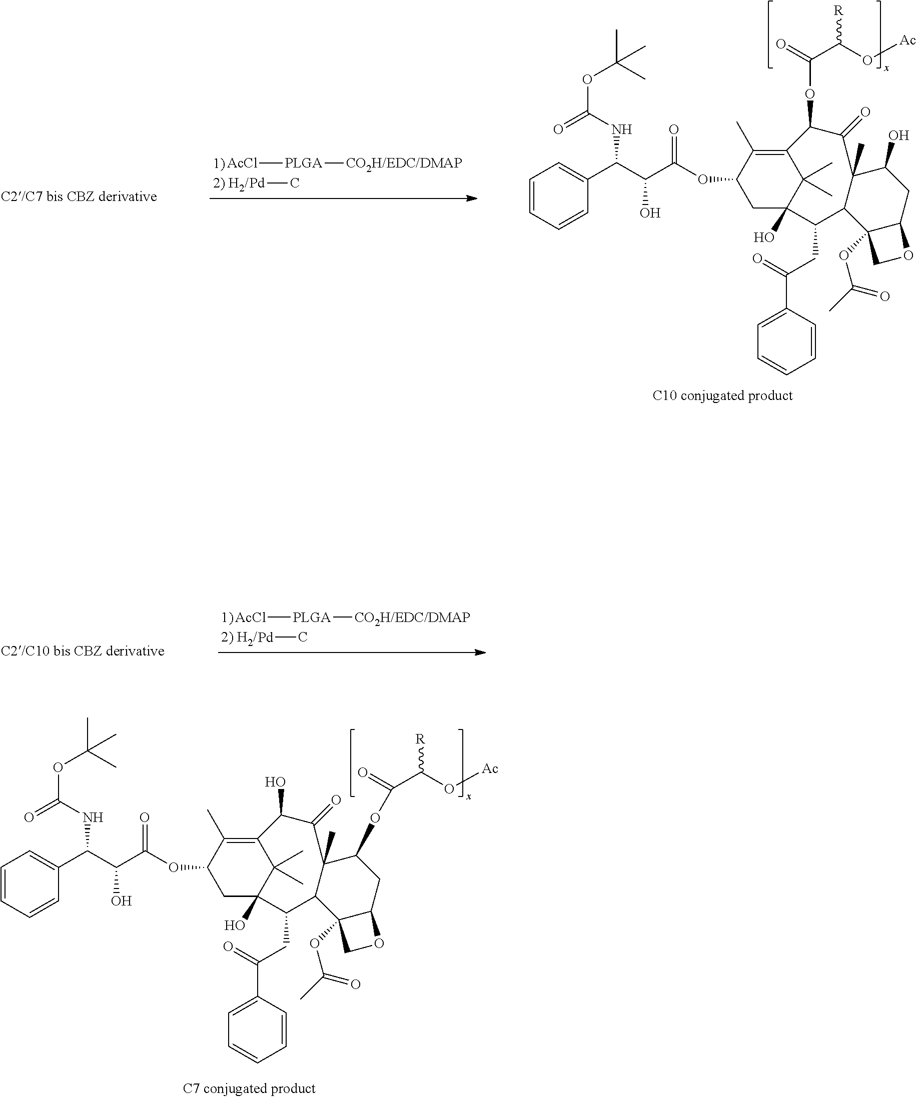

### Example 61

As shown in Examples 47 and 48, cyclodextrins are effective lyoprotectants for PEGylated nanoparticles. However, it is often desirable to lyophilize concentrated formulations or to resuspend a lyophilized preparation to produce a concentrated solution, e.g., by resuspending in a smaller volume than the volume of the liquid formulation that was lyophilized. Further studies using HP-β-CD indicated that good lyophilization was limited to formulations that contained a polymer concentration of less than about 31.25 mg/mL. This example demonstrates that the combination of cyclodextrin lyoprotectants with a wetting agent was effectively used to lyophilize PEGylated nanoparticles at a polymer concentration of up to about 62.5 mg/mL, and the resulting lyophilized preparations could resuspended to create a solution with a polymer concentration of about 83.3 mg/mL. The wetting agents used in this study, sucrose and trehalose, in combination with cyclodextrins effectively produced lyophilized preparations that were resuspended at high polymer concentrations. This was surprising as the polymer concentrations achieved were at least twice as high the polymer concentrations that were achieved using cyclodextrins, sucrose or trehalose alone.

PEGylated nanoparticles A were used in this example. HP-β-CD was prepared as a 60% (w/v) filtered solution. Sucrose and trehalose were added s solids to PEGylated nanoparticle formulations. Lyophilization was performed using a VirTis advantage freeze dryer using a 72-hour lyophilization program. The lyophilization program is shown in Tables 39A-39D.

TABLE 39A
Thermal Treatment

| Step | Temp | Time | Ramp/Hold |
|---|---|---|---|
| 1 | 5 | 120 | H |
| 2 | −45 | 120 | R |
| 3 | −45 | 180 | H |
| 4 | 0 | 0 | H |
| 5 | 0 | 0 | R |

TABLE 39B
Primary Drying

| Step | Temp | Time | Vacuum | Ramp/hold |
|---|---|---|---|---|
| 1 | −45 | 120 | 100 | |
| 2 | −20 | 120 | 100 | R |
| 3 | −20 | 1200 | 100 | H |
| 4 | −10 | 120 | 100 | R |
| 5 | −10 | 720 | 100 | H |
| 6 | 0 | 120 | 100 | R |
| 7 | 0 | 540 | 100 | H |
| 8 | 10 | 120 | 100 | R |
| 9 | 10 | 480 | 100 | H |
| 10 | 20 | 120 | 100 | R |
| 11 | 0 | 0 | 0 | H |

TABLE 39C
Post Ht

| Temp | Time | Vacuum |
|---|---|---|
| 20 | 240 | 100 |

TABLE 39D

| | Temp |
|---|---|
| Freeze | −45 |
| Extra freeze | 0 |
| Condenser | −45 |
| Vacuum | 500 |
| Secondary SP | 65 |

PEGylated nanoparticle formulations were analyzed for nanoparticle size prior to lyophilization, and lyophilized preparations that were completely resuspended by hand shaking were analyzed for nanoparticle size with a Zetasizer particle sizer. PEGylated nanoparticle formulations were also analyzed for active drug content (docetaxel) using C18 reversed phase (Agilent XBD C18 column, 4.6×150 mm, 5 μm) HPLC. Prior to lyophilization, lyoprotectants and wetting agents were added to PEGylated nanoparticle formulations at different weight ratios.

Study A. In this study, combinations of HP-β-CD and sucrose or trehalose, at different weight ratios, were tested for improved lyophilization and reconstitution of the lyophilized preparations in comparison to employing HP-β-CD alone. As shown in Tables 40A and B, 41A and B, 42A and B, and 43A and B, a combination of HP-β-CD and sucrose or trehalose achieved lyophilization at a higher polymer concentration of 83.3 mg/mL (in comparison to 31.25 mg/mL of polymer) than HP-β-CD alone. This result was obtained over a range of HP-β-CD:sucrose or trehalose ratios (w/w) and a range of HP-β-CD plus sucrose or trehalose:polymer ratios (w/w).

TABLE 40A
Pre-lyophilization

| | | | | Conc. |
|---|---|---|---|---|
| | | | | docetaxel |
| | Zave | PDI | Dv₉₀ | mg/mL |
| Pre-lyophilization | 80.13 | 0.075 | 103 | 3.2 |
| Pre-lyophilization | 84.76 | 0.089 | 111 | 4.0 |

TABLE 40B
Post-lyophilization and reconstitution

| | | | | Reconstitution | | | | |
|---|---|---|---|---|---|---|---|---|
| | | | | (assessed 5 | | | | |
| | | | | minutes after | | | | |
| | | | | addition of | | | | Conc. |
| | Lyoprotectant | Polymer | Lyoprotectant/Polymer | reconstitution | | | | docetaxel |
| | (mg/mL) | (mg/mL) | ratio | reagent) | Zave | PDI | Dv₉₀ | mg/mL |
| 1. | 81.25 | 62.5 | 1.3 HP-β-CD:1 | incomplete | | | | |
| | HP-β-CD | | | dissolution. | | | | |
| 2. | 108.3 | 83.3 | 1.3 HP-β-CD | complete | 84.15 | 0.085 | 109 | 4.0 |
| | HP-β-CD | | 0.7 sucrose:1 | dissolution | | | | |
| | 58.28 | | | | | | | |
| | sucrose | | | | | | | |
| 3. | 81.25 | 62.5 | 1.3 HP-β-CD | complete | 79.09 | 0.078 | 102 | 3.2 |
| | HP-β-CD | | 0.7 sucrose:1 | dissolution. | | | | |
| | 43.75 | | | | | | | |
| | sucrose | | | | | | | |
| 4. | 81.25 | 62.5 | 1.3 HP-β-CD | complete | 79.18 | 0.081 | 103 | 3.2 |
| | HP-β-CD | | 0.7 trehalose:1 | dissolution. | | | | |
| | 43.75 | | | | | | | |
| | trehalose | | | | | | | |

TABLE 41A
Pre-lyophilization

| | | | | Conc. |
|---|---|---|---|---|
| | | | | docetaxel |
| | Zave | PDI | Dv₉₀ | mg/mL |
| Pre-lyophilization | 80.13 | 0.075 | 103 | 3.2 |

TABLE 41B
Post-lyophilization and reconstitution

| | | | | Reconstitution | | | | |
|---|---|---|---|---|---|---|---|---|
| | | | | (assessed 5 | | | | |
| | | | | minutes after | | | | |
| | | | | addition or | | | | Conc. |
| | Lyoprotectant | Polymer | Lyoprotectant/polymer | reconstitution | | | | docetaxel |
| | (mg/mL) | (mg/mL) | ratio | reagent) | Zave | PDI | Dv₉₀ | mg/mL |
| 1. | 43.75 | 62.5 | 0.7 HP-β-CD | Complete | 79.4 | 0.076 | 102 | 3.2 |
| | HP-β-CD | | 1.3 sucrose:1 | dissolution | | | | |
| | 81.25 | | | | | | | |
| | sucrose | | | | | | | |

TABLE 42A
Pre-lyophilization

| | | | | Conc. |
|---|---|---|---|---|
| | | | | docetaxel |
| | Zave | PDI | Dv₉₀ | mg/mL |
| Pre-lyophilization | 82.02 | 0.094 | 105 | 3.0 |

TABLE 42B
Post-lyophilization and reconstitution

| | | | | Reconstitution | | | | |
|---|---|---|---|---|---|---|---|---|
| | | | | (assessed 5 | | | | |
| | | | | minutes after | | | | |
| | | | | addition of | | | | Conc. |
| | Lyoprotectant | Polymer | Lyoprotectant/polymer | reconstitution | | | | docetaxel |
| | (mg/mL) | (mg/mL) | ratio | reagent) | Zave | PDI | Dv₉₀ | mg/mL |
| 1. | 62.5 | 62.5 | 1.0 HP-β-CD | Complete | 79.4 | 0.076 | 102 | 3.0 |
| | HP-β-CD | | 0.7 sucrose:1 | dissolution | | | | |
| | 43.75 | | | | | | | |
| | sucrose | | | | | | | |
| 2. | 62.5 | 62.5 | 1.0 HP-β-CD | Complete | 83.92 | 0.081 | 109 | 3.0 |
| | HP-β-CD | | 1.0 sucrose:1 | dissolution | | | | |
| | 62.5 | | | | | | | |
| | sucrose | | | | | | | |

TABLE 43A
Pre-lyophilization

| | | | | Conc. |
|---|---|---|---|---|
| | | | | docetaxel |
| | Zave | PDI | Dv₉₀ | mg/mL |
| Pre-lyophilization | 80.88 | 0.088 | 104 | 3.0 |

TABLE 43B
Post-lyophilization and reconstitution

| | | | | Reconstitution | | | | |
|---|---|---|---|---|---|---|---|---|
| | | | | (assessed 5 | | | | |
| | | | | minutes after | | | | |
| | | | | addition of | | | | Conc. |
| | Lyoprotectant | Polymer | Lyoprotectant/polymer | reconstitution | | | | docetaxel |
| | (mg/mL) | (mg/mL) | ratio | reagent) | Zave | PDI | Dv₉₀ | mg/mL |
| 1. | 93.75 | 62.5 | 1.5 HP-β-CD | Complete | 82.38 | 0.113 | 106 | 3.0 |
| | HP-β-CD | | 0.75 sucrose:1 | dissolution | | | | |
| | 46.88 | | | | | | | |
| | sucrose | | | | | | | |
| 2. | 62.5 | 62.5 | 1.0 HP-β-CD | Complete | 83.65 | 0.110 | 110 | 3.0 |
| | HP-β-CD | | 1.5 sucrose:1 | dissolution | | | | |
| | 93.75 | | | | | | | |
| | sucrose | | | | | | | |

Study B. In this study, PEGylated nanoparticle formulations were lyophilized at 62.5 mg/mL polymer (3 mg docetaxel/mL concentration). The lyophilized preparations were reconstituted in a volume of water (0.75 mL) to achieve a final concentration of 83.3 mg/mL polymer (4 mg docetaxel/mL concentration). The results in Table 44 show that easy and complete reconstitution of lyophilized preparation at 83.3 mg/mL polymer concentration (4 mg docetaxel/mL) was achieved with a combination of HP-β-CD and sucrose in the weight ratio of 1.3:0.7 to 1 total polymer weight.

TABLE 44

| | | | Reconstitution | | | | |
|---|---|---|---|---|---|---|---|
| | | | at 4 mg | | | | |
| | | | (docetaxel)/mL | | | | |
| | | | (assessed 5 | | | | |
| | | | minutes after | Polymer | Zave | | Dv₉₀ |
| | | | addition of | (mg/mL) | (nm) | PDI | (nm) |
| | Lyoprotectant | Lyoprotectant/Polymer | reconstitution | post | post | post | post |
| | (mg/mL) | ratio | reagent) | resuspension | resuspension | resuspension | resuspension |
| 1. | 81.25 | 1.3 HP-β-CD:1 | Incomplete | | | | |
| | HP-β-CD | | dissolution | | | | |
| 2. | 81.25 | 1.3 HP-β-CD | Incomplete | | | | |
| | HP-β-CD | 0.7 trehalose:1 | dissolution | | | | |
| | 43.75 | | | | | | |
| | trehalose | | | | | | |
| 3. | 43.75 | 0.7 HP-β-CD | Incomplete | | | | |
| | HP-β-CD | 1.3 sucrose:1 | dissolution | | | | |
| | 81.25 | | | | | | |
| | sucrose | | | | | | |
| 4. | 81.25 | 1.3 HP-β-CD | Complete | 83.3 | 79.7 | 0.076 | 103 |
| | HP-β-CD | 0.7 sucrose:1 | dissolution | | | | |
| | 43.75 | | | | | | |
| | sucrose | | | | | | |

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover. Indefinite articles “a” or “an”, as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A formulation comprising:
a lyoprotectant comprising a cyclic oligosaccharide; and
a particulate construct, the particulate construct comprising:
a polymer,
a therapeutic agent, and
a potentiating agent, wherein the therapeutic agent and the potentiating agent are associated with the polymer.

2. The formulation of claim 1 wherein, the cyclic oligosaccharide comprises a polysaccharide moiety selected from the group consisting of: an α-cyclodextrin, a β-cyclodextrin, a 2-hydroxypropyl-β-cyclodextrin, a β-cyclodextrin sulfobutylether, any derivative thereof, and any combination thereof.

3. The formulation claim 1, further comprising a wetting agent.

4. The formulation of claim 3, wherein the wetting agent comprises a monosaccharide, a disaccharide, a surfactant, an amino acid, any derivative thereof, and any combination thereof.

5. The formulation of claim 4, wherein the wetting agent is a disaccharide selected from the group consisting of sucrose, trehalose, lactose and combinations thereof.

6. The formulation of any one of claims 3-5, wherein the ratio of cyclic oligosaccharide to the wetting agent (w/w) is about 0.5:1.5 to about 1.5:0.5.

7. The formulation of claim 3, wherein the ratio of cyclic oligosaccharide plus wetting agent to polymer (w/w) is about 1:1 to about 10:1.

8. The formulation of claim 3, wherein the wetting agent is sucrose.

9. The formulation of claim 1, wherein at least one therapeutic agent is covalently bonded to the polymer.

10. The formulation of claim 1, wherein the potentiating agent is covalently bonded to the polymer.

11. The formulation of claim 10, wherein the potentiating agent comprises a hydrophilic polymer.

12. The formulation of claim 11, wherein the hydrophilic polymer comprises a poly(alkylene glycol).

13. The formulation of claim 1, wherein the particulate construct is a nanoparticle.

14. The formulation of claim 1, wherein the particulate construct further comprises a stabilizing polymer, an excipient, a surfactant, a derivative thereof, and any combination thereof.

15. The formulation of claim 14, wherein the particulate construct further comprises a stabilizing polymer selected from the group consisting of: a poly(vinyl alcohol), a poly(vinyl pyrrolidone), a poly(vinyl acetate), a crown ether, any derivative thereof, and any combination thereof.

16. The formulation of claim 1, wherein the particulate construct comprises a polymer selected from the group consisting of: a polyester, a polysaccharide, a polyamide, a polyether, a polycarbonate, a polyacrylate, a polylactic acid, a polyglycolic acid, a polydioxanone, a poly-(lactide-co-glycolide), a polyethylenimine, a copolymer of methoxy-poly(ethylene glycol)-block-poly(lactide-co-glycolide), a chitin, a chitosan, a poly(glycolide), a poly(ε-caprolactone), a poly(hydroxy ester ether), a poly(hydroxybutyrate), a poly(anhydride), a poly(orthoester), a poly(amino acids), a poly(ethylene oxides), a poly(phosphazene), a poly etheresters, a polyester amides, a polyamide, derivatives, copolymers, blends, and other combinations thereof.

17. The formulation of claim 1, wherein the particulate construct comprises a polymer that is a block copolymer.

18. The formulation of claim 17, wherein the block copolymer comprises poly-(lactide-co-glycolide).

19. The formulation of claim 1, wherein
the lyoprotectant comprises a cyclodextrin, any derivative thereof, and any combination thereof:
the potentiating agent comprises a polyalkylene glycol; and
the formulation further comprises a wetting agent.

20. The formulation of claim 19, wherein the wetting agent is a disaccharide selected from the group consisting of sucrose, trehalose, lactose and combinations thereof.

21. The formulation of claim 19, wherein the ratio of lyoprotectant to wetting agent (w/w) is about 0.5:1.5 to about 1.5:0.5.

22. The formulation of claim 19, wherein the ratio of lyoprotectant plus wetting agent to polymer (w/w) is about 1:1 to about 3:1.

23. The formulation of claim 1, wherein the formulation further comprises a physiologically acceptable solvent selected from the group consisting of: an aqueous solvent, an organic solvent, and any combination thereof.

24. The formulation of claim 1, wherein the formulation is a liquid formulation.

25. The formulation of claim 24, wherein the formulation comprises a polymer concentration of at least about 32 mg/mL.

26. The formulation of claim 24, wherein the formulation is a resuspended lyophilized preparation.

27. The formulation of claim 24, wherein the formulation contains a polymer concentration of at least about 70 mg/mL.

28. The formulation of claim 26, wherein the particulate constructs in the resuspended lyophilized preparation have a property selected from the group consisting of Z-average diameter, poly-dispersity index, Dv₉₀ and combinations thereof that differ from the property of the particulate constucts in the formulation that was lyophilized to produce said lyophilized preparation by no more than 20%.

29. The formulation of claim 1, wherein the formulation is a lyophilized preparation.

30. The formulation of claim 29, wherein the lyophilized preparation can be dissolved in water to produce a solution that has a polymer concentration of at least 70 mg/mL.

31. A method for producing a lyophilized preparation comprising, providing a liquid formulation of claim 24 and lyophilizing the liquid formulation to produce a lyophilized preparation.

32. A method for treating cancer comprising, providing a lyophilized preparation of claim 29, and administering an effective amount of the lyophilized preparation to a subject in need thereof.

33. The method of claim 32, wherein the lyophilized preparation is resuspended in a suitable carrier before it is administered to the subject.

34. A method for producing a liquid formulation comprising combining a lyophilized preparation of claim 29 with a reconstitution reagent to produce a liquid formulation.
